SEQUENCE LISTING (1117 sequences)

SEQ ID NO: 1 - DNA, 2876 bp, Betatorquevirus sp.
  source 1..2876: mol_type = genomic DNA
cgggagccgaaggtgagtgcaaccaccgtagtctaggggcaattcgggctagttcagtatggcggaacgggcaagaaacttaaatattattattttacagatgcaaatacaaccacctattagaaccttcaaacaaacaatttcagattggaaaaacttaattgtccacgttcacgacaacatttgcaactgcaataaaccattagaacacactattgatacctgtatcaccaatccagatgaattaagattaaacaaatctactaaacaacaactacaaaaatgccttggtaccccagaagaagatacccaagaagacgttatcgatggcttcgcagatggagagctagacgcccttttcgcccaagatacagaagaagatactgggtaagaaactattctcgaaagagaaaactatttaaaataacaaccaaagaatggcaaccaaaagttataagaaagactcatgtaaagggcacctatcctttgtttctttgtacaaagcacagaattaacaataatatgatacaatatttagactctatagctccagaacactattacggaggaggaggattttcaataatgcaattttccttacaagccttatatgaagaatttataaaagcaaaaaactggtggactaatacaaactgctttttaccacttgtaagatatatgggttgctcattcaaattttataaaactgaattttatgattatattgtactaattgaaagatgttatccacttgcttgtactgatgaaatgtacttatctactcaacctagtattatgatgcttacaagaaaatgtatttttgtaccatgcaaacaaaacagcaaaggtaaaaaaccttacaaaaaagttagagtaagaccaccttcacaaatgactacaggatggcatttctcacaagacttagcaaacatgccacttgtagtactaaaaacttcagtatgcagctttgacagatattacacagacagtacagctaaatcaaccacaataggctttaaaacacttaacacacaaacatttagatatcatgactggcaggaaccacctacaacaggatacaaaccacaaaacctactatggttttatggagcagaaaacggatcaccagtagaccccaacaacacaatagtatcaaacctaatatacttaggaggcacaggaccttatgaaaaaggcacaccaataaaaacaaacataagcaattacttttcagagcctaaactgtggggaaatatatttcacgatgattatacatcaggaacatcacccgtgtttgttacaaacaaatcaccatcagaaattaaaaccgcatggaacactataaaagacttaactgttaaagctagcggtgtatttacattaagaacaattccactatggctaccttgcagatacaacccatttgcagacaaagcaaccaacaacaaaatatggctagtttctatacattcagaccacacagaatggaaaccaatagacaatccattactacaacgaacagaccttcctttatggttacttgtatggggttggcaagattggcagaaaaaaaaccaacaaacttcacaacctgatattaattatttaacagtaatatcttcaccatatatatcatgctacccaaaattagattactatgtgttactagatgaaggattttgggagggtcactcaacatacatagagtcaattacagactcagacaaaaaacactggtaccctaaaaatagatttcaaatagaaacacttaatctaatagctaacacaggtccaggaactgtaaaactaagagaaaaccaagcagcagaaggtcacatggtatatcgctttaattttaagcttggaggatgtcccgcaccgatggaaaaaatatgtgaccctagcaaacaatccaaatatcctattcccaataaccagcaacaaacaacttcgttgcagagtccagaaaacccaattcaaacctatctctacgacttcgacgaaaggaggggcctacttacagaaagagctacaaaaagaatcaaacaagatcacacatctgaaaaaactgttttgccatttacaggagcagcaacagacctccccatactccaaacaacatcacaggaggaaagctcctcggaagaagaagaagagcaacaagcggagaagaaactactccagctccgaagaaagcagcaccgactccgggagcgaatcctccagctattagacatacaaaatacataataaaacaaagtactgtaaaaattgatatgtttggagatactcatgtacctaaccgtagaatgaccccagaagaatttgaacaagaactaattgtcgctggtgtttttcgcagacctccttgttactatataaaagatagacctacttatccttatgtaccaaaacctactgatgaaaaatgtatggtaaactttgacttaaactttccttaataaactacgcctgcaaactttcactctcggtgtccatttatataagataaaacttaaataaacatccaccactctcccaaatacgcaggcgcacaagggggctccgcccccttaaacccccaagggggctccgcccccttaaacccccaagggggctccgcccccttacaccccctaataaatattcaacaggaaaaccacctaattagaattgccgaccacaaaccgtcacttacttctcctttttgcacttacttcctcttttacttattattattcattacattaattaataatcactgtaattccggggaggagctaacaatctatataactaactacacttccgaatggctgagtttatgccgccagacggagacgggatcacttcagtgactccaggctgaacttggg

SEQ ID NO: 2 - protein, 655 aa, Betatorquevirus sp.
MPWYPRRRYPRRRYRWLRRWRARRPFRPRYRRRYWVRNYSRKRKLFKITTKEWQPKVIRKTHVKGTYPLFLCTKHRINNNMIQYLDSIAPEHYYGGGGFSIMQFSLQALYEEFIKAKNWWTNTNCFLPLVRYMGCSFKFYKTEFYDYIVLIERCYPLACTDEMYLSTQPSIMMLTRKCIFVPCKQNSKGKKPYKKVRVRPPSQMTTGWHFSQDLANMPLVVLKTSVCSFDRYYTDSTAKSTTIGFKTLNTQTFRYHDWQEPPTTGYKPQNLLWFYGAENGSPVDPNNTIVSNLIYLGGTGPYEKGTPIKTNISNYFSEPKLWGNIFHDDYTSGTSPVFVTNKSPSEIKTAWNTIKDLTVKASGVFTLRTIPLWLPCRYNPFADKATNNKIWLVSIHSDHTEWKPIDNPLLQRTDLPLWLLVWGWQDWQKKNQQTSQPDINYLTVISSPYISCYPKLDYYVLLDEGFWEGHSTYIESITDSDKKHWYPKNRFQIETLNLIANTGPGTVKLRENQAAEGHMVYRFNFKLGGCPAPMEKICDPSKQSKYPIPNNQQQTTSLQSPENPIQTYLYDFDERRGLLTERATKRIKQDHTSEKTVLPFTGAATDLPILQTTSQEESSSEEEEEQQAEKKLLQLRRKQHRLRERILQLLDIQNT

SEQ ID NO: 3 - skipped

SEQ ID NO: 4 - protein, 61 aa, Betatorquevirus sp.
MFGDTHVPNRRMTPEEFEQELIVAGVFRRPPCYYIKDRPTYPYVPKPTDEKCMVNFDLNFP

SEQ ID NO: 5-53 - skipped (49 entries)

SEQ ID NO: 54 - DNA, 2979 bp, Betatorquevirus sp.
  source 1..2979: mol_type = genomic DNA
taataaatattcaacaggaaaaccacctaatttaaattgccgaccacaaaccgtcacttagttcccctttttgcaacaacttctgcttttttccaactgccggaaaaccacataatttgcatggctaaccacaaactgatatgctaattaacttccacaaaacaacttccccttttaaaaccacacctacaaattaattattaaacacagtcacatcctgggaggtactaccacactataataccaagtgcacttccgaatggctgagtttatgccgctagacggagaacgcatcagttactgactgcggactgaacttgggcgggtgccgaaggtgagtgaaaccaccgaagtcaaggggcaattcgggctagttcagtctagcggaacgggcaagaaacttaaaattattttatttttcagatgagcgactgctttaaaccaacatgctacaacaacaaaacaaagcaaactcactggattaataacctgcatttaacccacgacctgatctgcttctgcccaacaccaactagacacttattactagctttagcagaacaacaagaaacaattgaagtgtctaaacaagaaaaagaaaaaataacaagatgccttattactacagaagaagacggtacaactacagacgtcctagatggtatggacgaggttggattagacgcccttttcgcagaagatttcgaagaaaaagaagggtaagacctacttatactactattcctctaaagcaatggcaaccgccatataaaagaacatgctatataaaaggacaagactgtttaatatactatagcaacttaagactgggaatgaatagtacaatgtatgaaaaaagtattgtacctgtacattggccgggagggggttctttttctgtaagcatgttaactttagatgccttgtatgatatacataaactttgtagaaactggtggacatccacaaaccaagacttaccactagtaagatataaaggatgcaaaataacattttatcaaagcacatttacagactacatagtaagaatacatacagaactaccagctaacagtaacaaactaacatacccaaacacacatccactaatgatgatgatgtctaagtacaaacacattatacctagtagacaaacaagaagaaaaaagaaaccatacacaaaaatatttgtaaaaccacctccgcaatttgaaaacaaatggtactttgctacagacctctacaaaattccattactacaaatacactgcacagcatgcaacttacaaaacccatttgtaaaaccagacaaattatcaaacaatgttacattatggtcactaaacaccataagcatacaaaatagaaacatgtcagtggatcaaggacaatcatggccatttaaaatactaggaacacaaagcttttatttttacttttacaccggagcaaacctaccaggtgacacaacacaaataccagtagcagacctattaccactaacaaacccaagaataaacagaccaggacaatcactaaatgaggcaaaaattacagaccatattactttcacagaatacaaaaacaaatttacaaattattggggtaacccatttaataaacacattcaagaacacctagatatgatactatactcactaaaaagtccagaagcaataaaaaacgaatggacaacagaaaacatgaaatggaaccaattaaacaatgcaggaacaatggcattaacaccatttaacgagccaatattcacacaaatacaatataacccagatagagacacaggagaagacactcaattatacctactctctaacgctacaggaacaggatgggacccaccaggaattccagaattaatactagaaggatttccactatggttaatatattggggatttgcagactttcaaaaaaacctaaaaaaagtaacaaacatagacacaaattacatgttagtagcaaaaacaaaatttacacaaaaacctggcacattctacttagtaatactaaatgacacctttgtagaaggcaatagcccatatgaaaaacaacctttacctgaagacaacattaaatggtacccacaagtacaataccaattagaagcacaaaacaaactactacaaactgggccatttacaccaaacatacaaggacaactatcagacaatatatcaatgttttataaattttactttaaatggggaggaagcccaccaaaagcaattaatgttgaaaatcctgcccaccagattcaatatcccataccccgtaacgagcatgaaacaacttcgttacagagtccaggggaagccccagaatccatcttatactccttcgactatagacacgggaactacacaacaacagctttgtcacgaattagccaagactgggcacttaaagacactgtttctaaaattacagagccagatcgacagcaactgctcaaacaagccctcgaatgcctgcaaatctcggaagaaacgcaggagaaaaaagaaaaagaagtacagcagctcatcagcaacctcagacagcagcagcagctgtacagagagcgaataatatcattattaaaggaccaataacttttaactgtgtaaaaaaggtgaaattgtttgatgataaaccaaaaaaccgtagatttacacctgaggaatttgaaactgagttacaaatagcaaaatggttaaagagacccccaagatcctttgtaaatgatcctcccttttacccatggttaccacctgaacctgttgtaaactttaagcttaattttactgaataaaggccagcattaattcacttaaggagtctgtttatttaagttaaaccttaataaacggtcaccgcctccctaatacgcaggcgcagaaagggggctccgccccctttaacccccagggggctccgccccctgaaacccccaagggggctacgcccccttacaccccc

SEQ ID NO: 55 - protein, 99 aa, Betatorquevirus sp.
MSDCFKPTCYNNKTKQTHWINNLHLTHDLICFCPTPTRHLLLALAEQQETIEVSKQEKEKITRCLITTEEDGTTTDVLDGMDEVGLDALFAEDFEEKEG

SEQ ID NO: 56 - protein, 203 aa, Betatorquevirus sp.
MSDCFKPTCYNNKTKQTHWINNLHLTHDLICFCPTPTRHLLLALAEQQETIEVSKQEKEKITRCLITTEEDGTTTDVLDGMDEVGLDALFAEDFEEKEGFNIPYPVTSMKQLRYRVQGKPQNPSYTPSTIDTGTTQQQLCHELAKTGHLKTLFLKLQSQIDSNCSNKPSNACKSRKKRRRKKKKKYSSSSATSDSSSSCTESE

SEQ ID NO: 57 - protein, 219 aa, Betatorquevirus sp.
MSDCFKPTCYNNKTKQTHWINNLHLTHDLICFCPTPTRHLLLALAEQQETIEVSKQEKEKITRCLITTEEDGTTTDVLDGMDEVGLDALFAEDFEEKEGARSTATAQTSPRMPANLGRNAGEKRKRSTAAHQQPQTAAAAVQRANNIIIKGPITFNCVKKVKLFDDKPKNRRFTPEEFETELQIAKWLKRPPRSFVNDPPFYPWLPPEPVVNFKLNFTE

SEQ ID NO: 58 - protein, 666 aa, Betatorquevirus sp.
MPYYYRRRRYNYRRPRWYGRGWIRRPFRRRFRRKRRVRPTYTTIPLKQWQPPYKRTCYIKGQDCLIYYSNLRLGMNSTMYEKSIVPVHWPGGGSFSVSMLTLDALYDIHKLCRNWWTSTNQDLPLVRYKGCKITFYQSTFTDYIVRIHTELPANSNKLTYPNTHPLMMMMSKYKHIIPSRQTRRKKKPYTKIFVKPPPQFENKWYFATDLYKIPLLQIHCTACNLQNPFVKPDKLSNNVTLWSLNTISIQNRNMSVDQGQSWPFKILGTQSFYFYFYTGANLPGDTTQIPVADLLPLTNPRINRPGQSLNEAKITDHITFTEYKNKFTNYWGNPFNKHIQEHLDMILYSLKSPEAIKNEWTTENMKWNQLNNAGTMALTPFNEPIFTQIQYNPDRDTGEDTQLYLLSNATGTGWDPPGIPELILEGFPLWLIYWGFADFQKNLKKVTNIDTNYMLVAKTKFTQKPGTFYLVILNDTFVEGNSPYEKQPLPEDNIKWYPQVQYQLEAQNKLLQTGPFTPNIQGQLSDNISMFYKFYFKWGGSPPKAINVENPAHQIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 59 - protein, 148 aa, Betatorquevirus sp.
MPYYYRRRRYNYRRPRWYGRGWIRRPFRRRFRRKRRIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 60 - protein, 82 aa, Betatorquevirus sp.
MPYYYRRRRYNYRRPRWYGRGWIRRPFRRRFRRKRRSQIDSNCSNKPSNACKSRKKRRRKKKKKYSSSSATSDSSSSCTESE

SEQ ID NO: 61-104 - skipped (44 entries)

SEQ ID NO: 105 - DNA, 71 bp, synthetic construct
  source 1..71: mol_type = other DNA
cgggtgccgkaggtgagtttacacaccgmagtcaaggggcaattcgggctcrggactggccgggcyhtggg

SEQ ID NO: 106-111 - skipped (6 entries)

SEQ ID NO: 112 - DNA, 71 bp, Alphatorquevirus sp.
  source 1..71: mol_type = genomic DNA
cgggtgccggaggtgagtttacacaccgcagtcaaggggcaattcgggctcgggactggccgggcyhtggg

SEQ ID NO: 113-119 - skipped (7 entries)

SEQ ID NO: 120 - DNA, 117 bp, synthetic construct
  source 1..117: mol_type = other DNA
  misc_difference 10: note = May be absent
  misc_difference 12: note = May be absent
  misc_difference 30: note = May be absent
  misc_difference 31: note = May be absent
  misc_difference 32: note = May be absent
  misc_difference 34: note = May be absent
  misc_difference 43: note = May be absent
  misc_difference 44: note = May be absent
  misc_difference 45: note = May be absent
  misc_difference 46: note = May be absent
  misc_difference 52: note = May be absent
  misc_difference 53: note = May be absent
  misc_difference 54: note = May be absent
  misc_difference 70: note = May be absent
  misc_difference 71: note = May be absent
  misc_difference 89: note = May be absent
  misc_difference 90: note = May be absent
  misc_difference 103: note = May be absent
cggcggsggsgcsscgcgctdcgcgcgcsgcccrsyrggggrdssmmwgcskcscccccccscgcgcatgcgcrcgggkcccccccccyvsggggggctccgcccccccggcccccc

SEQ ID NO: 121-172 - skipped (52 entries)

SEQ ID NO: 173 - protein, 96 aa, Betatorquevirus sp.
MQIQPPIRTFKQTISDWKNLIVHVHDNICNCNKPLEHTIDTCITNPDELRLNKSTKQQLQKCLGTPEEDTQEDVIDGFADGELDALFAQDTEEDTG

SEQ ID NO: 174-212 - skipped (39 entries)

SEQ ID NO: 213 - DNA, 3733 bp, Alphatorquevirus sp.
  source 1..3733: mol_type = genomic DNA
cacgtgactcccgcaggccaaccagagtctacgtcgtgcacttcctgggcatggtctacatcataatataagaaggcgcacttccgaatggctgagttttccacgcccgtccgcagcgagaacgccacggagggagatcctcgcgtcccgagggcgggtgccggaggtgagtttacacaccgcagtcaaggggcaattcgggctcgggactggccgggccccgggcaaggctcttaaaaaatgcgctttcgcagggttgctgagaaaaggaaagtgcttctgcaaactgtgcgagctgcagagaagactaggcggcttctaggtatgtggcagccccccgcgcacaatgtccccggcatcgagagaaactggtacgagagctgttttcgatcccatgctgctgtttgcggctgtggcgactttgttggccatcttagttatctggcaactactctgggtcgtcctccgcgtcctgggcccccaggcggaccccgcacaccgcagataagaaacctgccagcgctcccggcgccccagggcgagcccggtgacagagcgccatggcgtggggcttctggggccgacgccgccggtggagacggtggagaccacggcgcagacggtggagaccccgcagacgtaggagacgacgccctgctcgccgctttcgagctcgtcgaagagtaaggaggcgcggggggcggtggcgcagacgctacagaaaatggcgacggggcagacgcagacgaactcacagaaaaaagatagtcataaaacagtggcaaccaaactttataagacgctgctacatcatagggtacctacctttaatattctgtggcgaaaacacaaccgcccagaactatgccactcactcagacgacatgataagcaaaggaccatacggggggggcatgactaccacaaaatttactctgagaatactgtacgacgagtttaccaggtttatgaacttttggactgttagtaacgaagacctagacctgtgtagatacgtgggctgcaaactcatattttttaaacatcccacagtggactttatagtacagataaacactcagcctcctttcttagacacgcaccttaccgcggccagcatacacccgggcatcatgatgctcagcaagagacgcatactaataccctctctaaaaacccggccaagcagaaaacacagggtggttgttagggtgggcgccccaagactttttcaggacaagtggtacccccagtcagacctgtgtgacacagttctgctttccatatttgcaaccgcctgtgacttgcaatatccgttcggctcaccactaactgacaacccttgcgtcaacttccagattctggggccccagtacaaaaaacaccttagtattagctctactatggatacaactaacaaacagcactatgacagcaatttgtttaaccaaactcagctatacaacacctttcaaactatagctcagcttaaagagacaggacaaactgcaaacatatctcctagttggagtgcagtgcaaaataatatggcccttagtaataccggtgaaaatgcaacccaaagcaaagacacttggtacaaaggaaacacatacaacaaccacattacaacgttagcacaaaaaaccagagaaagatttaaaggtgcaacaaaagcagcactacaaaactaccccaccataatgtccacagacttatatgaataccactcaggcatatactccagcatatttctatcagctggcaggagctactttgaaaccaccggggcctactctgacattatatacaaccctttcacagacaaaggcacaggcaacataatctggatagactacctcacaaaagaagacaccatttttgtaaaaaacaaaagcaaatgcgaaataatggacatgcccctgtgggcggcctgcacaggatatacagagttctgtgcaaagtatacaggagactctgccattatttacaatgccagagtactaataagatgcccgtacactgaacccatgctaatagaccactcagacccgaacaaaggcttcgtaccctactcatttaactttggcaacggaaaaatgcccggaggcagctccaacgtacccataagaatgagagccaaatggtacgtgaacatattccaccaaaaagaggttctagagactatagtacagagcggaccgttcgggtacaagggcgacataaaatcagctgtactggccatgaaatacagatttcactggaaatggggtggaaaccctatatccaaacaggtcgtcaggaatccctgctccaactccagctcctccgcggcccatagaggacctcgcagcgtacaagcagttgacccgaaatacaataccccagaggtcacgtggcactcgtgggacatcagacgaggactctttggcaaagcaggtattaaaagaatgcaacaagaatcagatgctctttacattcctccaggaccattcaagagacctcgcagagacacgaacgcccaagacccagaagagcaaaacgaaagctcaggtttcagagtccagcagcgactcccgtgggtccactccagccaagagacgcaaagctcccaagaagaaacggaggcgcaggggtcggtacaagaccaactactcctccagctccgagagcagcgagtactccgactccagctccagcaactcgcaacccaagtcctcaaagtccaagcagggcacggcatacaccccctattatcttcccaagcgtaaacaaagtctttatgtttgagccccacggtcctaaacccatacagggctacaacgattggctagaggagtacactgcttgtaaattctgggacagacccccaagaaagctacacacagacttacccttttacccctgggcaccaaaaccccaagaccaagtcagggtaagctttaaactcaactttcaataaaaattctaggccgtgggagtttcacttgtcggtgtctgcttcttaaggtcgccaagcactccgagcgccagcgaggagtgcgaccccccctccggtagcaacgccttcggagccgcgcgctacgccttcggctgcgcgcggcacctcagaccccccctccacccgaaacgcttgcgcgtttcggaccttcggcgtcgggggggtcgggagctttattaaacagactccgagttgccattggacactggagctgtgaatcagtaacgaaagtgagtggggccagacttcgccatagggcctttatcttctcgccattggatagtgtccggggtcgccgtaggcttcggcctcgtttttaggccttccggactacaaaaatggcgaacttggtgacgtcacggccgccattttaagtaaggcggaagcagctccactttctcacaaaatggcggcggagcacttccggcttgcccaaaatagcgggcaagctcttccgggtcaaaggtcagcagctacgtcacaagtcacctgactgaggaggagctaaaacccggaagtcctcctcggtcacgtggctagtcacgtgactactacgtcatcggcgccatcttgtgtgacaaaatggcggacaacttccgcttttttgaaaaaaggcgcgaaaaaacggcggcggcggcgcgcgcgctgcgcgcgcgcgccgagggggcgccagcgcccccactgtgcggtcccccgcggggctccggcccccccccgaagtccgtcactaac

SEQ ID NO: 214 - protein, 199 aa, Alphatorquevirus sp.
MAWGFWGRRRRWRRWRSRRRRWRPRRRRRRRPARRFRARRRVVRNPCSNSSSSAAHRGPRSVQAVDPKYNTPEVTWHSWDIRRGLFGKAGIKRMQQESDALYIPPGPFKRPRRDTNAQDPEEQNESSGFRVQQRLPWVHSSQETQSSQEETEAQGSVQDQLLLQLREQRVLRLQLQQLATQVLKVQAGHGIHPLLSSQA

SEQ ID NO: 215 - protein, 141 aa, Alphatorquevirus sp.
MAWGFWGRRRRWRRWRPRRRRWRPRRRRRRRPARRFRARRRDHSRDLAETRTPKTQKSKTKAQVSESSSDSRGSTPAKRRKAPKKKRRRRGRYKTNYSSSSESSEYSDSSSSNSQPKSSKSKQGTAYTPYYLPKRKQSLYV

SEQ ID NO: 216 - protein, 120 aa, Alphatorquevirus sp.
MWQPPAHNVPGIERNWYESCFRSHAAVCGCGDFVGHLSYLATTLGRPPRPGPPGGPRTPQIRNLPALPAPQGEPGDRAPWRGASGADAAGGDGGDHGADGGDPADVGDDALLAAFELVEE

SEQ ID NO: 217 - protein, 284 aa, Alphatorquevirus sp.
MWQPPAHNVPGIERNWYESCFRSHAAVCGCGDFVGHLSYLATTLGRPPRPGPPGGPRTPQIRNLPALPAPQGEPGDRAPWRGASGADAAGGDGGDHGADGGDPADVGDDALLAAFELVEESSGIPAPTPAPPRPIEDLAAYKQLTRNTIPQRSRGTRGTSDEDSLAKQVLKECNKNQMLFTFLQDHSRDLAETRTPKTQKSKTKAQVSESSSDSRGSTPAKRRKAPKKKRRRRGRYKTNYSSSSESSEYSDSSSSNSQPKSSKSKQGTAYTPYYLPKRKQSLYV

SEQ ID NO: 218 - protein, 277 aa, Alphatorquevirus sp.
MWQPPAHNVPGIERNWYESCFRSHAAVCGCGDFVGHLSYLATTLGRPPRPGPPGGPRTPQIRNLPALPAPQGEPGDRAPWRGASGADAAGGDGGDHGADGGDPADVGDDALLAAFELVEETIQETSQRHERPRPRRAKRKLRFQSPAATPVGPLQPRDAKLPRRNGGAGVGTRPTTPPAPRAASTPTPAPATRNPSPQSPSRARHTPPIIFPSVNKVFMFEPHGPKPIQGYNDWLEEYTACKFWDRPPRKLHTDLPFYPWAPKPQDQVRVSFKLNFQ

SEQ ID NO: 219-499 - skipped (281 entries)

SEQ ID NO: 500 - DNA, 9391 bp, synthetic construct
  source 1..9391: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagagaattcgagctcggtacctcgcgaatacatctagatatggttggctccggtgcccgtcagtgggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccaccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtggatgtttatgccgccagacggagacgggatcacttcagtgactccaggctgaacttgggcgggagccgaaggtgagtgcaaccaccgtagtctaggggcaattcgggctagttcagtatggcggaacgggcaagaaacttaaatattattattttacagatgcaaatacaaccacctattagaaccttcaaacaaacaatttcagattggaaaaacttaattgtccacgttcacgacaacatttgcaactgcaataaaccattagaacacactattgatacctgtatcaccaatccagatgaattaagattaaacaaatctactaaacaacaactacaaaaatgccttggtaccccagaagaagatacccaagaagacgttatcgatggcttcgcagatggagagctagacgcccttttcgcccaagatacagaagaagatactgggtaagaaactattctcgaaagagaaaactatttaaaataacaaccaaagaatggcaaccaaaagttataagaaagactcatgtaaagggcacctatcctttgtttctttgtacaaagcacagaattaacaataatatgatacaatatttagactctatagctccagaacactattacggaggaggaggattttcaataatgcaattttccttacaagccttatatgaagaatttataaaagcaaaaaactggtggactaatacaaactgctttttaccacttgtaagatatatgggttgctcattcaaattttataaaactgaattttatgattatattgtactaattgaaagatgttatccacttgcttgtactgatgaaatgtacttatctactcaacctagtattatgatgcttacaagaaaatgtatttttgtaccatgcaaacaaaacagcaaaggtaaaaaaccttacaaaaaagttagagtaagaccaccttcacaaatgactacaggatggcatttctcacaagacttagcaaacatgccacttgtagtactaaaaacttcagtatgcagctttgacagatattacacagacagtacagctaaatcaaccacaataggctttaaaacacttaacacacaaacatttagatatcatgactggcaggaaccacctacaacaggatacaaaccacaaaacctactatggttttatggagcagaaaacggatcaccagtagaccccaacaacacaatagtatcaaacctaatatacttaggaggcacaggaccttatgaaaaaggcacaccaataaaaacaaacataagcaattacttttcagagcctaaactgtggggaaatatatttcacgatgattatacatcaggaacatcacccgtgtttgttacaaacaaatcaccatcagaaattaaaaccgcatggaacactataaaagacttaactgttaaagctagcggtgtatttacattaagaacaattccactatggctaccttgcagatacaacccatttgcagacaaagcaaccaacaacaaaatatggctagtttctatacattcagaccacacagaatggaaaccaatagacaatccattactacaacgaacagaccttcctttatggttacttgtatggggttggcaagattggcagaaaaaaaaccaacaaacttcacaacctgatattaattatttaacagtaatatcttcaccatatatatcatgctacccaaaattagattactatgtgttactagatgaaggattttgggagggtcactcaacatacatagagtcaattacagactcagacaaaaaacactggtaccctaaaaatagatttcaaatagaaacacttaatctaatagctaacacaggtccaggaactgtaaaactaagagaaaaccaagcagcagaaggtcacatggtatatcgctttaattttaagcttggaggatgtcccgcaccgatggaaaaaatatgtgaccctagcaaacaatccaaatatcctattcccaataaccagcaacaaacaacttcgttgcagagtccagaaaacccaattcaaacctatctctacgacttcgacgaaaggaggggcctacttacagaaagagctacaaaaagaatcaaacaagatcacacatctgaaaaaactgttttgccatttacaggagcagcaacagacctccccatactccaaacaacatcacaggaggaaagctcctcggaagaagaagaagagcaacaagcggagaagaaactactccagctccgaagaaagcagcaccgactccgggagcgaatcctccagctattagacatacaaaatacataataaaacaaagtactgtaaaaattgatatgtttggagatactcatgtacctaaccgtagaatgaccccagaagaatttgaacaagaactaattgtcgctggtgtttttcgcagacctccttgttactatataaaagatagacctacttatccttatgtaccaaaacctactgatgaaaaatgtatggtaaactttgacttaaactttccttaataaaatgaatgcaattgttgttgttaacggggatcctcatcgcggccgctacgtaaattccgccccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggataaagttttaaacagagaggaatctttgcagctaatggaccttctaggtcttgaaaggagtgcctgggggaatattcctctgatgagaaaggcatatttaaaaaaatgcaaggagtttcatcctgataaaggaggagatgaagaaaaaatgaagaaaatgaatactctgtacaagaaaatggaagatggagtaaaatatgctcatcaacctgactttggaggcttctgggatgcaactgagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatccattttctgttattgaggaaagtttgccaggtgggttaaaggagcatgattttaatccagaagaagcagaggaaactaaacaagtgtcctggaagcttgtaacagagtatgcaatggaaacaaaatgtgatgatgtgttgttattgcttgggatgtacttggaatttcagtacagttttgaaatgtgtttaaaatgtattaaaaaagaacagcccagccactataagtaccatgaaaagcattatgcaaatgctgctatatttgctgacagcaaaaaccaaaaaaccatatgccaacaggctgttgatactgttttagctaaaaagcgggttgatagcctacaattaactagagaacaaatgttaacaaacagatttaatgatcttttggataggatggatataatgtttggttctacaggctctgctgacatagaagaatggatggctggagttgcttggctacactgtttgttgcccaaaatggattcagtggtgtatgactttttaaaatgcatggtgtacaacattcctaaaaaaagatactggctgtttaaaggaccaattgatagtggtaaaactacattagcagctgctttgcttgaattatgtggggggaaagctttaaatgttaatttgcccttggacaggctgaactttgagctaggagtagctattgaccagtttttagtagtttttgaggatgtaaagggcactggaggggagtccagagatttgccttcaggtcagggaattaataacctggacaatttaagggattatttggatggcagtgttaaggtaaacttagaaaagaaacacctaaataaaagaactcaaatatttccccctggaatagtcaccatgaatgagtacagtgtgcctaaaacactgcaggccagatttgtaaaacaaatagattttaggcccaaagattatttaaagcattgcctggaacgcagtgagtttttgttagaaaagagaataattcaaagtggcattgctttgcttcttatgttaatttggtacagacctgtggctgagtttgctcaaagtattcagagcagaattgtggagtggaaagagagattggacaaagagtttagtttgtcagtgtatcaaaaaatgaagtttaatgtggctatgggaattggagttttagattggctaagaaacagtgatgatgatgatgaagacagccaggaaaatgctgataaaaatgaagatggtggggagaagaacatggaagactcagggcatgaaacaggcattgattcacagtcccaaggctcatttcaggcccctcagtcctcacagtctgttcatgatcataatcagccataccacatttgtagaggttttacttgctttaaaaaacctcccacacctccccctgaacctgaaacataataagcttgcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagtttcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggccatcggatcccgggcccgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 501 - DNA, 5128 bp, synthetic construct
  source 1..5128: mol_type = other DNA
gtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcgaaagatatctaataaatattcaacaggaaaaccacctaatttaaattgccgaccacaaaccgtcacttagttcccctttttgcaacaacttctgcttttttccaactgccggaaaaccacataatttgcatggctaaccacaaactgatatgctaattaacttccacaaaacaacttccccttttaaaaccacacctacaaattaattattaaacacagtcacatcctgggaggtactaccacactataataccaagtgctaatccgaatggctgagtttatgccgctagacggagaacgcatcagttactgactgcggactgaacttgggcgggtgccgaaggtgagtgaaaccaccgaagtcaaggggcaattcgggctagttcagtctagcggaacgggcaagaaacttaaaattattttatttttcagatggatggctgatcgagtgtagccagatctgcgatcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactggatctacaaaaaagcagatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttgcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcatgaatgaataaaggccagcattaattcacttaaggagtctgtttatttaagttaaaccttaataaacggtcaccgcctccctaatacgcaggcgcagaaagggggctccgccccctttaacccccagggggctccgccccctgaaacccccaagggggctacgcccccttacacccccgatatccccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcga

SEQ ID NO: 502 - DNA, 5452 bp, synthetic construct
  source 1..5452: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagagaattcgagctcggtacctcgcgaatacatctagattaccgttcgtatagcatacattatacgaagttattaaactacgcctgcaaactttcactctcggtgtccatttatataagataaaacttaaataaacatccaccactctcccaaatacgcaggcgcacaagggggctccgcccccttaaacccccaagggggctccgcccccttaaacccccaagggggctccgcccccttacaccccctaataaatattcaacaggaaaaccacctaattagaattgccgaccacaaaccgtcacttacttctcctttttgcacttacttcctcttttacttattattattcattacattaattaataatcactgtaattccggggaggagctaacaatctatataactaactacacttccgaatggctgagtttatgccgccagacggagacgggatcacttcagtgactccaggctgaacttgggcgggagccgaaggtgagtgcaaccaccgtagtctaggggcaattcgggctagttcagtatggcggaacgggcaagaaacttaaatattattattttacagatgggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactggatctacaaaaaagcagatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggttccgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggtagacaatagcaggcatgctggggatgcggtgggctctatggtcgagttagtttgctccagtaaagttgtttataataactactaaatccgcatgttacggaatttcttattaatttttttttcgtaaggaacaacggatcttgaaataacttcgtatagcatacattatacgaacggtaatcggatcccgggcccgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 503 - DNA, 6473 bp, synthetic construct
  source 1..6473: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattcgccaccatggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaaaaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgcgccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacatagtgatgaactacatcagaaacctggactctgagactggggccatggtgaggctgctcgaggatggggactgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 504 - protein, 708 aa, Simian virus 40
MDKVLNREESLQLMDLLGLERSAWGNIPLMRKAYLKKCKEFHPDKGGDEEKMKKMNTLYKKMEDGVKYAHQPDFGGFWDATEIPTYGTDEWEQWWNAFNEENLFCSEEMPSSDDEATADSQHSTPPKKKRKVEDPKDFPSELLSFLSHAVFSNRTLACFAIYTTKEKAALLYKKIMEKYSVTFISRHNSYNHNILFFLTPHRHRVSAINNYAQKLCTFSFLICKGVNKEYLMYSALTRDPFSVIEESLPGGLKEHDFNPEEAEETKQVSWKLVTEYAMETKCDDVLLLLGMYLEFQYSFEMCLKCIKKEQPSHYKYHEKHYANAAIFADSKNQKTICQQAVDTVLAKKRVDSLQLTREQMLTNRFNDLLDRMDIMFGSTGSADIEEWMAGVAWLHCLLPKMDSVVYDFLKCMVYNIPKKRYWLFKGPIDSGKTTLAAALLELCGGKALNVNLPLDRLNFELGVAIDQFLVVFEDVKGTGGESRDLPSGQGINNLDNLRDYLDGSVKVNLEKKHLNKRTQIFPPGIVTMNEYSVPKTLQARFVKQIDFRPKDYLKHCLERSEFLLEKRIIQSGIALLLMLIWYRPVAEFAQSIQSRIVEWKERLDKEFSLSVYQKMKFNVAMGIGVLDWLRNSDDDDEDSQENADKNEDGGEKNMEDSGHETGIDSQSQGSFQAPQSSQSVHDHNQPYHICRGFTCFKKPPTPPPEPET

SEQ ID NO: 505-828 - skipped (324 entries)

SEQ ID NO: 829 - protein, 11 aa, unidentified
  VARIANT 4: note = Any amino acid
  VARIANT 5: note = Any amino acid
  VARIANT 7: note = Any amino acid
  VARIANT 9: note = Any amino acid
  VARIANT 10: note = Any amino acid
YNPXXDXGXXN

SEQ ID NO: 830-948 - skipped (119 entries)

SEQ ID NO: 949 - protein, 21 aa, unidentified
  VARIANT 1: note = W or F
  VARIANT 2: note = Any amino acid
  VARIANT 3: note = Any amino acid
  VARIANT 4: note = Any amino acid
  VARIANT 5: note = Any amino acid
  VARIANT 6: note = Any amino acid
  VARIANT 7: note = Any amino acid
  VARIANT 8: note = Any amino acid
  VARIANT 10: note = Any amino acid
  VARIANT 11: note = Any amino acid
  VARIANT 12: note = Any amino acid
  VARIANT 14: note = Any amino acid
  VARIANT 16: note = Any amino acid
  VARIANT 17: note = Any amino acid
  VARIANT 18: note = Any amino acid
  VARIANT 19: note = Any amino acid
  VARIANT 20: note = Any amino acid
XXXXXXXXHXXXCXCXXXXXH

SEQ ID NO: 950-1099 - skipped (150 entries)

SEQ ID NO: 1100 - protein, 736 aa, Alphatorquevirus sp.
MAWGFWGRRRRWRRWRPRRRRWRPRRRRRRRPARRFRARRRVRRRGGRWRRRYRKWRRGRRRRTHRKKIVIKQWQPNFIRRCYIIGYLPLIFCGENTTAQNYATHSDDMISKGPYGGGMTTTKFTLRILYDEFTRFMNFWTVSNEDLDLCRYVGCKLIFFKHPTVDFIVQINTQPPFLDTHLTAASIHPGIMMLSKRRILIPSLKTRPSRKHRVVVRVGAPRLFQDKWYPQSDLCDTVLLSIFATACDLQYPFGSPLTDNPCVNFQILGPQYKKHLSISSTMDTTNKQHYDSNLFNQTQLYNTFQTIAQLKETGQTANISPSWSAVQNNMALSNTGENATQSKDTWYKGNTYNNHITTLAQKTRERFKGATKAALQNYPTIMSTDLYEYHSGIYSSIFLSAGRSYFETTGAYSDIIYNPFTDKGTGNIIWIDYLTKEDTIFVKNKSKCEIMDMPLWAACTGYTEFCAKYTGDSAIIYNARVLIRCPYTEPMLIDHSDPNKGFVPYSFNFGNGKMPGGSSNVPIRMRAKWYVNIFHQKEVLETIVQSGPFGYKGDIKSAVLAMKYRFHWKWGGNPISKQVVRNPCSNSSSSAAHRGPRSVQAVDPKYNTPEVTWHSWDIRRGLFGKAGIKRMQQESDALYIPPGPFKRPRRDTNAQDPEEQNESSGFRVQQRLPWVHSSQETQSSQEETEAQGSVQDQLLLQLREQRVLRLQLQQLATQVLKVQAGHGIHPLLSSQA

SEQ ID NO: 1101 - protein, 351 aa, synthetic construct
MVPKKKRKVSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 1102 - protein, 343 aa, synthetic construct
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 1103 - protein, 518 aa, synthetic construct
MPKKKRKVYPYDVPDYAGSRALVVIRLSRVTDATTSPERQLESCQQLCAQRGWDVVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDGEWRLVPDPVQRERILEVYHRVVDNHEPLHLVAHDLNRRGVLSPKDYFAQLQGREPQGREWSATALKRSMISEAMLGYATLNGKTVRDDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSPQREALDARIAALAARQEELEGLEARPSGWEWRETGQRFGDWWREQDTAAKNTWLRSMNVRLTFDVRGGLTRTIDFGDLQEYEQHLRLGSVVERLHTGMS

SEQ ID NO: 1104 - protein, 500 aa, synthetic construct [sequence omitted]

SEQ ID NO: 1105 - DNA, 34 bp, synthetic construct
  source 1..34: mol_type = other DNA
ataacttcgtatagcatacattatacgaagttat

SEQ ID NO: 1106 - DNA, 34 bp, synthetic construct
  source 1..34: mol_type = other DNA
ataacttcgtatagcatacattatacgaacggta

SEQ ID NO: 1107 - DNA, 34 bp, synthetic construct
  source 1..34: mol_type = other DNA
taccgttcgtatagcatacattatacgaagttat

SEQ ID NO: 1108 - DNA, 34 bp, synthetic construct
  source 1..34: mol_type = other DNA
taccgttcgtatagcatacattatacgaacggta

SEQ ID NO: 1109 - DNA, 50 bp, synthetic construct
  source 1..50: mol_type = other DNA
tcggccggcttgtcgacgacggcggtctccgtcgtcaggatcatccgggc

SEQ ID NO: 1110 - DNA, 58 bp, synthetic construct
  source 1..58: mol_type = other DNA
gtcgtggtttgtctggtcaaccaccgcggtctcagtggtgtacggtacaaaccccgac

SEQ ID NO: 1111 - DNA, 54 bp, synthetic construct
  source 1..54: mol_type = other DNA
tcggccggcttgtcgacgacggcggtctcagtggtgtacggtacaaaccccgac

SEQ ID NO: 1112 - DNA, 54 bp, synthetic construct
  source 1..54: mol_type = other DNA
gtcgtggtttgtctggtcaaccaccgcggtctccgtcgtcaggatcatccgggc

SEQ ID NO: 1113 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 1114 - protein, 286 aa, synthetic construct
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 1115 - DNA, 18 bp, synthetic construct
  source 1..18: mol_type = other DNA
gaaccgcatcgagctgaa

SEQ ID NO: 1116 - DNA, 21 bp, synthetic construct
  source 1..21: mol_type = other DNA
tgcttgtcggccatgatatag

SEQ ID NO: 1117 - DNA, 24 bp, synthetic construct
  source 1..24: mol_type = other DNA
atcgacttcaaggaggacggcaac

(19) United States
(12) Patent Application Publication
(10) Pub. No.: US 2024/0415978 A1
(43) Pub. Date: Dec. 19, 2024

(54) ANELLOVECTORS FOR DELIVERY OF EFFECTORS TO THE CENTRAL NERVOUS SYSTEM

(71) Applicant: FLAGSHIP PIONEERING INNOVATIONS V, INC., Cambridge, MA (US)

(72) Inventors: Roger Joseph Hajjar, Lexington, MA (US); Nathan Lawrence Yozwiak, Newton, MA (US); Simon Delagrave, Sudbury, MA (US); Dhananjay Maniklal Nawandar, Waltham, MA (US); Bryan W. Vought, Framingham, MA (US); Christopher Ian Wright, Cambridge, MA (US)

(21) Appl. No.: 18/742,660

(22) Filed: Jun. 13, 2024

Related U.S. Application Data
(60) Provisional application No. 63/508,149, filed on Jun. 14, 2023.

(51) Int. Cl. A61K 48/00; A61K 9/00; C12N 15/86

(52) U.S. Cl. CPC A61K 48/0041; A61K 9/0085; A61K 48/0075; A61K 48/0083; C12N 15/86; C12N 2750/00043

(57) ABSTRACT

This invention relates generally to Anelloviridae family vectors (e.g., anellovectors) and compositions and uses thereof.

### CROSS REFERENCE TO RELATED APPLICATIONS

This application claims the benefit of U.S. Provisional Application No. 63/508,149, filed Jun. 14, 2023. The contents of the aforementioned application are hereby incorporated by reference in their entirety.

### SEQUENCE LISTING

The instant application contains a Sequence Listing which has been submitted electronically in XML file format and is hereby incorporated by reference in its entirety. Said XML copy, created on May 31, 2024, is named V2057-703110_SL.xml and is 311,526 bytes in size.

### BACKGROUND

There is an ongoing need to develop suitable vectors to deliver therapeutic genetic material to patients.

### SUMMARY

The present disclosure provides an Anelloviridae family vector (e.g., anellovector), e.g., a synthetic Anelloviridae family vector (e.g., anellovector), that can be used as a delivery vehicle, e.g., for delivering genetic material, for delivering an effector, e.g., a payload, or for delivering a therapeutic agent or a therapeutic effector to a eukaryotic cell (e.g., a human cell or a human tissue).

Additional features of any of the aforesaid Anelloviridae family vectors (e.g., anellovectors), compositions or methods include one or more of the following enumerated embodiments.

Those skilled in the art will recognize, or be able to ascertain using no more than routine experimentation, many equivalents to the specific embodiments of the invention described herein. Such equivalents are intended to be encompassed by the following enumerated embodiments.

### ENUMERATED EMBODIMENTS

1. A method of delivering an exogenous effector to the central nervous system (CNS) of a subject, the method comprising administering to the CNS of the subject an Anelloviridae family vector (e.g., an anellovector).

2. A method of delivering a DNA capable of encoding (e.g., encoding) an exogenous effector to the CNS of a subject, the method comprising administering to the CNS of the subject an Anelloviridae family vector (e.g., an anellovector).

3. The method of embodiment 1 or 2, which comprises delivery of the Anelloviridae family vector to the brain of the subject.

4. The method of any of embodiments 1-3, which comprises production of the exogenous effector and/or DNA capable of encoding (e.g., encoding) the exogenous effector in the brain of the subject.

5. A method of modulating a biological function in the CNS of a subject, the method comprising administering to the CNS of the subject an Anelloviridae family vector (e.g., an anellovector).

6. The method of embodiment 5, which comprises delivery of the Anelloviridae family vector to the brain of the subject.

7. The method of embodiment 5 or 6, which comprises modulating a biological function in the brain of the subject.

8. A method of treating a CNS disease or disorder in a subject in need thereof, the method comprising administering to the subject an Anelloviridae family vector (e.g., an anellovector).

9. The method of embodiment 8, wherein the CNS disease or disorder is a brain disease or disorder.

10. The method of embodiment 8 or 9, wherein the CNS disease or disorder is chosen from: ataxia imbalance (e.g., Friedreich's ataxia), spinal cerebellar atrophy, frontotemporal dementia, Alzheimer's Disease, Parkinson's Disease, Lewy body dementia, a genetic seizure disorder, Huntington's disease, a psychiatric disorder (e.g., schizophrenia, bipolar disorder, or depression), spinal muscular atrophy (SMA), multiple sclerosis, traumatic brain injury, a brain tumor, a genetic disease (e.g., a rare genetic disease), Batten disease, Rett syndrome, Duchenne muscular dystrophy, Amyotrophic Lateral Sclerosis (ALS), a neural development disease (e.g., autism, Rett syndrome, or Fragile X syndrome), or a leukodystrophy (e.g., adrenal leukodystrophy or metachromatic leukodystrophy).

11. The method of any of embodiments 8-10, wherein the subject has an impairment of one or more of: memory, behavior, or language.

12. The method of any of embodiments 1-11, which results in delivery of anellovector DNA in one or more cell types selected from: neurons (e.g., GABAergic neurons, glutamatergic neurons, spinal motor neurons), glial cells, microglia, oligodenroglia, and astrocytes.

13. The method of any of embodiments 1-12, which results in expression of the exogenous effector in one or more cell types selected from: neurons (e.g., GABAergic neurons, glutamatergic neurons, spinal motor neurons), glial cells, microglia, oligodenroglia, and astrocytes.

14. The method of any of embodiments 1-13, which comprises contacting (e.g., directly or indirectly) the anellovector to one or more cell types selected from: neurons (e.g. GABAergic neurons, glutamatergic neurons, spinal motor neurons), glial cells, microglia, oligodenroglia, and astrocytes.

15. The method of any of embodiments 1-14, which results in delivery of anellovector DNA in one or more of: cerebellum, frontotemporal lobe (e.g., in the hippocampus), parietal lobe, brain stem, and spinal cord.

16. The method of any of embodiments 1-15, which results in expression of the exogenous effector in one or more of: cerebellum, frontotemporal lobe (e.g., in the hippocampus), parietal lobe, brain stem, and spinal cord.

17. The method of any of embodiments 1-16, which comprises contacting (e.g., directly or indirectly) the anellovector to one or more of: cerebellum, frontotemporal lobe (e.g., in the hippocampus), parietal lobe, brain stem, and spinal cord.

18. The method of any of embodiments 1-17, which results in greater delivery of the exogenous effector and/or the DNA encoding the exogenous effector to the brain than to the spinal cord.

19. The method of any of embodiments 1-18, wherein the Anelloviridae family vector (e.g., an anellovector) is administered according to a route of administration chosen from: intrathecal (IT), intracerebroventricular (ICV), intra cisterna magna (ICM), or intraparenchymal (IPa).

20. The method of embodiment 18, wherein the Anelloviridae family vector (e.g., an anellovector) is administered according to an intracerebroventricular (ICV) route of administration.

21. The method of any of the preceding embodiments, wherein the Anelloviridae family vector is an anellovector comprising a proteinaceous exterior comprising an ORF1 molecule and a genetic element enclosed by the proteinaceous exterior, wherein the genetic element comprises a promoter element operably linked to a nucleic acid sequence (e.g., a DNA sequence) encoding the exogenous effector.

22. The method of any of the preceding embodiments, wherein the Anelloviridae family vector is an Anellovector comprising a proteinaceous exterior comprising an ORF1 molecule having an ORF1 sequence as listed in any of Tables A1-A3, or a polypeptide comprising an amino acid sequence having at least about 70%, 75%, 80%, 85%, 90%, 95%, 96%, 97%, 98%, or 99% sequence identity thereto.

23. The method of any of the preceding embodiments, wherein the ORF1 molecule comprises at least one difference relative to the wild-type ORF1 protein of Table A1-A3.

24. The method any of the preceding embodiments, wherein the difference comprises a mutation (e.g., an insertion, a substitution, or a deletion), a chemical modification, or an enzymatic modification.

25. The method of any of the preceding embodiments, wherein the mutation comprises a deletion, e.g., a deletion of a domain (e.g., one or more of an arginine-rich region, jelly-roll domain, HVR, N22, or CTD, e.g., as described herein).

26. The method of any of the preceding embodiments, wherein the ORF1 molecule comprises an ORF1 domain and an exogenous moiety, e.g., an exogenous surface moiety.

27. The method of any of the preceding embodiments, wherein the Anelloviridae family vector is an Anellovector comprising a proteinaceous exterior comprising a polypeptide encoded by an Anellovirus ORF1 nucleic acid sequence as listed in Table N1-N3, or a polypeptide encoded by a nucleic acid sequence having at least about 70%, 75%, 80%, 85%, 90%, 95%, 96%, 97%, 98%, or 99% sequence identity to the Anellovirus ORF1 nucleic acid sequence.

28. The method of any of the preceding embodiments, wherein the genetic element comprises an Anellovirus 5′ UTR conserved domain having a sequence of the reverse complement of nucleotides 323-393 of SEQ ID NO: 54, or a nucleic acid sequence having at least 70%, 75%, 80%, 85%, 90%, 95%, 96%, 97%, 98%, 99%, or 100% sequence identity thereto, or a functional portion thereof.

29. The method of any of the preceding embodiments, wherein the genetic element comprises an Anellovirus GC-rich region having a sequence of the reverse complement of nucleotides 2868-2929 of SEQ ID NO: 54, or a nucleic acid sequence having at least 70%, 75%, 80%, 85%, 90%, 95%, 96%, 97%, 98%, 99%, or 100% sequence identity thereto, or a functional portion thereof.

30. The method of embodiments 1-27, wherein the genetic element comprises an Anellovirus 5′ UTR conserved domain having a sequence of the reverse complement of nucleotides 1-71 of SEQ ID NO: 1, or a nucleic acid sequence having at least 70%, 75%, 80%, 85%, 90%, 95%, 96%, 97%, 98%, 99%, or 100% sequence identity thereto, or a functional portion thereof.

31. The method of embodiments 1-27 or 30, wherein the genetic element comprises an Anellovirus GC-rich region having a sequence of the reverse complement of nucleotides 2515-2615 of SEQ ID NO: 1, or a nucleic acid sequence having at least 70%, 75%, 80%, 85%, 90%, 95%, 96%, 97%, 98%, 99%, or 100% sequence identity thereto, or a functional portion thereof.

32. The method of any of the preceding embodiments, wherein the portion of the Anellovirus GC-rich region is at least 20, 30, 40, 50, 60, 70, 80, 90, or 100 nucleotides.

33. The method of any of the preceding embodiments, wherein the genetic element is DNA.

34. The method of any of the preceding embodiments, wherein the genetic element is circular, single stranded DNA.

35. The method of any of embodiments 1-32, wherein the genetic element is RNA, e.g., mRNA.

36. The method of any of the preceding embodiments, wherein the exogenous effector comprises: an intracellular peptide or intracellular polypeptide, a secreted polypeptide, or a protein replacement therapeutic.

37. The method of any of the preceding embodiments, wherein the Anelloviridae family vector (e.g., an anellovector) is a Betatorquevirus.

38. The method of any of the preceding embodiments, which further comprises administering an additional dose of an Anelloviridae family vector (e.g., anellovector) to the CNS of the subject.

39. The method of embodiment 38, wherein the Anelloviridae family vector and the Anelloviridae family vector of the additional dose are the same.

40. The method of embodiment 38, wherein the Anelloviridae family vector and the Anelloviridae family vector of the additional dose are different.

41. The method of any of the preceding embodiments, which results in greater delivery of the DNA encoding the exogenous effector to the CNS than to muscle.

42. The method of any of the preceding embodiments, which results in greater delivery of the DNA encoding the exogenous effector to the CNS than to liver.

43. The method of any of the preceding embodiments, which results in greater delivery of the DNA encoding the exogenous effector to the spinal cord than to muscle.

44. The method of any of the preceding embodiments, which results in greater delivery of the DNA encoding the exogenous effector to the spinal cord than to liver.

45. The method of any of embodiments 41-44, wherein the greater delivery is 2 times, 5 times, or 10 times higher.

46. A delivery system suitable for delivery to the CNS, wherein the delivery system comprises an Anelloviridae family vector (e.g., an anellovector).

47. The Anelloviridae family vector (e.g., anellovector) of any of the preceding embodiments, wherein the proteinaceous exterior (e.g., ORF1 molecule of the proteinaceous exterior) comprises the amino acid sequence YNPX²DXGX²N (SEQ ID NO: 829), wherein X″ is a contiguous sequence of any n amino acids.

48. The Anelloviridae family vector (e.g., anellovector) of embodiment 47, wherein the amino acid sequence YNPX²DXGX²N (SEQ ID NO: 829) is comprised in an N22 domain of the ORF1 molecule.

49. The Anelloviridae family vector (e.g., anellovector) of any of the preceding embodiments, wherein the ORF1 molecule comprises one or more (e.g., 1, 2, 3, 4, or all 5) of the following Anellovirus ORF1 subdomains: an arginine-rich region, a jelly-roll region, a hypervariable region, an N22 domain, a C-terminal domain (CTD) (e.g., as described herein), e.g., of an Anellovirus ORF1 protein as listed in Table A1-A3 (or a sequence having at least about 70%, 75%, 80%, 85%, 90%, 95%, 96%, 97%, 98%, 99%, or 100% sequence identity thereto).

50. The Anelloviridae family vector (e.g., anellovector) of any of the preceding embodiments, which comprises at least one difference relative to a wild type Anellovirus genome sequence, wherein the at least one difference comprises a deletion (e.g., lacks one or more of: an Anellovirus 5′ UTR conserved domain, an ORF1 gene, ORF2 gene, an Anellovirus GC-rich region, an ORF3 gene, or a functional fragment thereof).

51. The Anelloviridae family vector (e.g., anellovector) of any of the preceding embodiments, wherein the genetic element further comprises a promoter.

52. The Anelloviridae family vector (e.g., anellovector) of any of the preceding embodiments, wherein the genetic element further comprises a poly A sequence.

53. A pharmaceutical composition comprising an Anelloviridae family vector (e.g., anellovector) of any of the preceding embodiments.

54. The pharmaceutical composition of embodiment 53, wherein the pharmaceutical composition has one or more of the following characteristics:
a) the pharmaceutical composition meets a pharmaceutical or good manufacturing practices (GMP) standard;
b) the pharmaceutical composition was made according to good manufacturing practices (GMP);
c) the pharmaceutical composition has a pathogen level below a predetermined reference value, e.g., is substantially free of pathogens;
d) the pharmaceutical composition has a contaminant level below a predetermined reference value, e.g., is substantially free of contaminants;
e) the pharmaceutical composition has a predetermined level of non-infectious particles or a predetermined ratio of particles:infectious units (e.g., <300:1, <200:1, <100:1, or <50:1), or
f) the pharmaceutical composition has low immunogenicity or is substantially non-immunogenic, e.g., as described herein.

55. The pharmaceutical composition of any one of embodiments 53-54, wherein the pharmaceutical composition has a contaminant level below a predetermined reference value, e.g., is substantially free of contaminants.

56. The pharmaceutical composition of embodiment 55, wherein the contaminant is selected from the group consisting of: mycoplasma, endotoxin, host cell nucleic acids (e.g., host cell DNA and/or host cell RNA), animal-derived process impurities (e.g., serum albumin or trypsin), replication-competent agents (RCA), e.g., replication-competent virus or unwanted Anelloviridae family vector (e.g., anellovector) (e.g., an Anelloviridae family vector other than the desired Anelloviridae family vector, e.g., a synthetic Anelloviridae family vector as described herein), free viral capsid protein, adventitious agents, and aggregates.

57. The pharmaceutical composition of embodiment 55, wherein the contaminant is host cell DNA and the threshold amount is about 10 ng of host cell DNA per dose of the pharmaceutical composition.

58. The pharmaceutical composition of any one of embodiments 53-57, wherein the pharmaceutical composition comprises less than 10% (e.g., less than about 10%, 5%, 4%, 3%, 2%, 1%, 0.5%, or 0.1%) contaminant by weight.

59. The Anelloviridae family vector (e.g., anellovector) or method of any of the preceding embodiments, wherein the genetic element is single-stranded.

60. The Anelloviridae family vector (e.g., anellovector or method of any of the preceding embodiments, wherein the genetic element is circular.

61. The Anelloviridae family vector (e.g., anellovector) or method of any of the preceding embodiments, wherein the genetic element comprises DNA.

62. The Anelloviridae family vector (e.g., anellovector) or method of any of the preceding embodiments, wherein the genetic element is a negative strand DNA.

63. The Anelloviridae family vector (e.g., anellovector) or method of any of the preceding embodiments, wherein the genetic element is double-stranded.

64. The Anelloviridae family vector (e.g., anellovector) or method of any of the preceding embodiments, wherein the genetic element is linear.

65. The Anelloviridae family vector (e.g., anellovector) or method of any of the preceding embodiments, wherein the genetic element comprises RNA.

66. The Anelloviridae family vector (e.g., anellovector) or method of any of the preceding embodiments, wherein the genetic element comprises a nucleic acid sequence encoding an Anelloviridae capsid protein, e.g., an Anellovirus ORF1 molecule (e.g., an ORF1 protein as listed in Table A1-A3 or an amino acid sequence having at least 70%, 75%, 80%, 85%, 90%, 95%, 96%, 97%, 98%, or 99% sequence identity thereto).

67. The Anelloviridae family vector (e.g., anellovector) or method of any of the preceding embodiments, wherein the genetic element does not comprise a nucleic acid sequence encoding an Anelloviridae capsid protein, e.g., an Anellovirus ORF1 molecule (e.g., an ORF1 protein as listed in Table A1-A3 or an amino acid sequence having at least 70%, 75%, 80%, 85%, 90%, 95%, 96%, 97%, 98%, or 99% sequence identity thereto).

68. The Anelloviridae family vector (e.g., anellovector) or method of any of the preceding embodiments, wherein the genetic element comprises a nucleic acid sequence encoding an Anellovirus ORF2 molecule (e.g., an ORF2 protein as listed in Table A1-A3, or an amino acid sequence having at least 70%, 75%, 80%, 85%, 90%, 95%, 96%, 97%, 98%, or 99% sequence identity thereto).

69. The Anelloviridae family vector (e.g., anellovector), nucleic acid molecule, or method of any of the preceding embodiments, wherein the genetic element does not comprise a nucleic acid sequence encoding an Anellovirus ORF2 molecule (e.g., an ORF2 protein as listed in Table A1-A3 or an amino acid sequence having at least 70%, 75%, 80%, 85%, 90%, 95%, 96%, 97%, 98%, or 99% sequence identity thereto).

70. The Anelloviridae family vector (e.g., anellovector) or method of any of the preceding embodiments, wherein the genetic element comprises at least 20, 25, 30, 31, 32, 33, 34, 35, 36, 37, 38, 39, or 40 consecutive nucleotides having a GC content of at least 70%, 75%, 80%, 85%, 90%, 95%, or 99%.

71. The Anelloviridae family vector (e.g., anellovector) or method of any of the preceding embodiments, wherein the proteinaceous exterior comprises the amino acid sequence YNPX²DXGX²N (SEQ ID NO: 829), wherein X″ is a contiguous sequence of any n amino acids.

72. The Anelloviridae family vector (e.g., anellovector), nucleic acid molecule, or method of embodiment 71, wherein the amino acid sequence YNPX²DXGX²N (SEQ ID NO: 829) is comprised in an N22 domain.

73. The Anelloviridae family vector (e.g., anellovector), nucleic acid molecule, or method of any of the preceding embodiments, wherein the ORF1 molecule comprises an arginine-rich region (e.g., having at least 70%, 80%, 85%, 90%, 95%, 96%, 97%, 98%, 99%, or 100% identity to an arginine-rich region sequence of an ORF1 protein listed in Table A1-A3).

74. The Anelloviridae family vector (e.g., anellovector) or method of any of the preceding embodiments, wherein the proteinaceous exterior comprises an amino acid sequence of at least 15, 20, 25, 26, 27, 28, 29, 30, 31, 32, 33, 34, 35, 36, 37, 38, 39, 40, 45, or 50 consecutive nucleotides comprising at least 40% (e.g., at least 40%, 41%, 42%, 43%, 44%, 45%, 46%, 47%, 48%, 49%, 50%, 55%, 60%, 65%, 66%, 67%, 68%, 69%, 70%, 75%, 80%, 85%, 90%, or 95%) arginine residues.

75. The Anelloviridae family vector (e.g., anellovector), nucleic acid molecule, or method of embodiment 73 or 74, wherein the arginine-rich region is located at the N-terminal or C-terminal end of the ORF1 molecule.

76. The Anelloviridae family vector (e.g., anellovector) or method of any of the preceding embodiments, wherein the genetic element comprises one or more of: a TATA box, an initiator element, a cap site, a transcriptional start site, an ORF1/1-encoding sequence, an ORF1/2-encoding sequence, an ORF2/2-encoding sequence, an ORF2/3-encoding sequence, an ORF2/3t-encoding sequence, a three open-reading frame region, a poly(A) signal, and/or a GC-rich region from an Anellovirus described herein, or a sequence having at least 70%, 75%, 80%, 85%, 90%, 95%, 96%, 97%, 98%, 99%, or 100% sequence identity thereto.

77. The Anelloviridae family vector (e.g., anellovector) or method of any of the preceding embodiments, wherein the genetic element comprises at least 75% (e.g., at least 75, 76, 77, 78, 79, 80, 85, 90, 91, 92, 93, 94, 95, 96, 97, 98, 99, or 100%) sequence identity to an Anellovirus 5′ UTR conserved domain sequence as described herein.

78. The Anelloviridae family vector (e.g., anellovector), nucleic acid molecule, or method of any of the preceding embodiments, wherein the genetic element comprises at least 75% (e.g., at least 75, 76, 77, 78, 79, 80, 85, 90, 91, 92, 93, 94, 95, 96, 97, 98, 99, or 100%) sequence identity to an Anellovirus GC-rich region sequence as described herein.

79. The Anelloviridae family vector (e.g., anellovector) or method of any of the preceding embodiments, wherein the effector encodes a therapeutic agent, e.g., a therapeutic peptide or polypeptide or a therapeutic nucleic acid.

80. The Anelloviridae family vector (e.g., anellovector), nucleic acid molecule, or method of any of the preceding embodiments, wherein the effector is an exogenous effector.

81. The Anelloviridae family vector (e.g., anellovector), nucleic acid molecule, or method of any of the preceding embodiments, wherein the effector is an endogenous effector (e.g., wherein the anellovector overexpresses the endogenous effector in a target cell).

82. The Anelloviridae family vector (e.g., anellovector) or method of any of the preceding embodiments, wherein the effector modulates expression or activity of a gene or protein, e.g., increases or decreases expression or activity of the gene or protein.

83. The Anelloviridae family vector (e.g., anellovector), nucleic acid molecule, or method of any of the preceding embodiments, wherein the Anelloviridae family vector is capable of replicating autonomously.

84. The Anelloviridae family vector (e.g., anellovector), nucleic acid molecule, or method of any of the preceding embodiments, wherein the Anelloviridae family vector is replication-deficient (e.g., incapable of replicating autonomously).

85. The Anelloviridae family vector (e.g., anellovector), nucleic acid molecule, or method of any of the preceding embodiments, wherein the genetic element integrates into the genome of a eukaryotic cell at a frequency of less than about 0.001%, 0.005%, 0.01%, 0.05%, 0.1%, 0.5%, 1%, 1.5%, or 2% of the genetic element that enters the cell.

86. The Anelloviridae family vector (e.g., anellovector) or method of any of the preceding embodiments, wherein the Anelloviridae family vector is substantially non-pathogenic, e.g., does not induce a detectable deleterious symptom in a subject (e.g., elevated cell death or toxicity, e.g., relative to a subject not exposed to the anellovector).

87. The Anelloviridae family vector (e.g., anellovector), nucleic acid molecule, or method of any of the preceding embodiments, wherein the Anelloviridae family vector is substantially non-immunogenic, e.g., does not induce a detectable and/or unwanted immune response.

88. The Anelloviridae family vector (e.g., anellovector) or method of any of the preceding embodiments, wherein a population of at least 1000 of the Anelloviridae family vectors is capable of delivering at least about 100 copies (e.g., at least 1, 2, 3, 4, 5, 10, 20, 30, 40, 50, 100, 200, 300, 400, 500, 600, 700, 800, 900, or 1000 copies) of the genetic element into one or more eukaryotic cells (e.g., mammalian cells, e.g., human cells).

89. The Anelloviridae family vector (e.g., anellovector), nucleic acid molecule, or method of any of the preceding embodiments, wherein a population of the Anelloviridae family vectors (e.g., at least 1, 2, 3, 4, 5, 10, 20, 30, 40, 50, 100, 200, 300, 400, 500, 600, 700, 800, 900, or 1000 genome equivalents of the genetic element per cell) is capable of delivering the genetic element into at least 10%, 20%, 30%, 40%, 50%, 60%, 70%, 80%, 90%, 95%, 99%, or more of a population of eukaryotic cells (e.g., mammalian cells, e.g., human cells).

90. The Anelloviridae family vector (e.g., anellovector) or method of any of the preceding embodiments, wherein a population of the Anelloviridae family vectors (e.g., at least 1, 2, 3, 4, 5, 10, 20, 30, 40, 50, 100, 200, 300, 400, 500, 600, 700, 800, 900, or 1000 genome equivalents of the genetic element per cell) is capable of delivering at least 1, 2, 3, 4, 5, 6, 7, 8, 9, 10, 20, 50, 100, 200, 500, 1000, 2000, 5000, 8,000, 1×10⁴, 1×10⁵, 1×10⁶, 1×10⁷ or greater copies of the genetic element per cell to a population of eukaryotic cells (e.g., mammalian cells, e.g., human cells).

91. The Anelloviridae family vector (e.g., anellovector) or method of any of the preceding embodiments, wherein a population of the Anelloviridae family vectors (e.g., at least 1, 2, 3, 4, 5, 10, 20, 30, 40, 50, 100, 200, 300, 400, 500, 600, 700, 800, 900, or 1000 genome equivalents of the genetic element per cell) is capable of delivering 1-3, 1-4, 1-5, 1-6, 1-7, 1-8, 1-9, 1-10, 5-10, 10-20, 20-50, 50-100, 100-1000, 1000-10⁴, 1×10⁴-1×10⁵, 1×10⁴-1×10⁶, 1×10⁴-1×10⁷, 1×10⁵-1×10⁶, 1×10⁵-1×10⁷, or 1×10⁶-1×10⁷ copies of the genetic element per cell to a population of eukaryotic cells (e.g., mammalian cells, e.g., human cells).

92. The Anelloviridae family vector (e.g., anellovector) or method of any of the preceding embodiments, wherein the target cells into which the genetic element is delivered each receive at least 10, 50, 100, 500, 1000, 10,000, 50,000, 100,000, or more copies of the genetic element.

93. The Anelloviridae family vector (e.g., anellovector) or method of any of the preceding embodiments, wherein the Anelloviridae family vector is resistant to degradation by a detergent (e.g., a mild detergent, e.g., a biliary salt, e.g., sodium deoxycholate) relative to a viral particle comprising an external lipid bilayer, e.g., a retrovirus.

94. The Anelloviridae family vector (e.g., anellovector) or method of any of the preceding embodiments, wherein the genetic element enclosed by the proteinaceous exterior is resistant to degradation by a nuclease enzyme (e.g., a DNase).

95. The Anelloviridae family vector (e.g., anellovector) or method of any of the preceding embodiments, wherein the Anelloviridae family vector is capable of infecting mammalian cells, e.g., human cells, e.g., in vitro, in vivo, or ex vivo.

96. The Anelloviridae family vector (e.g., anellovector) or method of any of the preceding embodiments, wherein the Anelloviridae family vector selectively delivers the effector to, or is present at higher levels in (e.g., preferentially accumulates in), a desired cell type, tissue, or organ

97. The Anelloviridae family vector (e.g., anellovector) or method of any of the preceding embodiments, wherein the proteinaceous exterior is provided in cis relative to the genetic element.

98. The Anelloviridae family vector (e.g., anellovector) or method of any of the preceding embodiments, wherein the proteinaceous exterior is provided in trans relative to the genetic element.

99. The Anelloviridae family vector of any of the previous embodiments, which is substantially free of wild-type Anellovirus genomes.

Other features, objects, and advantages of the invention will be apparent from the description and drawings, and from the claims.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In addition, the materials, methods, and examples are illustrative only and not intended to be limiting.

### BRIEF DESCRIPTION OF THE DRAWINGS

The following detailed description of the embodiments of the invention will be better understood when read in conjunction with the appended drawings. For the purpose of illustrating the invention, there are shown in the drawings embodiments that are presently exemplified. It should be understood, however, that the invention is not limited to the precise arrangement and instrumentalities of the embodiments shown in the drawings.

FIG. 1A and FIG. 1B are representative merged (GFP and brightfield) images of MOLT4 cells transfected with the pRTx-2847 (R19-eGFP vector, SEQ ID NO: 502), pRTx-2848 (iCre plasmid, SEQ ID NO: 503), and pRTx-3525 (R19 SRR, SEQ ID NO: 500) 72 hours post-electroporation. Images were taken at 10× for Study #1 producer cells (FIG. 1A) and Study #2 producer cells (FIG. 1B). White cells exhibit GFP expression.

FIG. 2A and FIG. 2B are plots depicting number of DNase-protected eGFP copies per fraction of cell lysate on post-iodixanol fractions for Study #1 Ring19-eGFP Anellovector material (FIG. 2A) and Study #2 Ring19-eGFP Anellovector material (FIG. 2B).

FIG. 3A and FIG. 3B are plots depicting the number of DNase-protected Ring19-eGFP copies pre- and post-concentration of viral material for Study #1 Ring19-eGFP Anellovector material (FIG. 3A) and Study #2 Ring19-eGFP Anellovector material (FIG. 3B).

FIG. 4A and FIG. 4B depict gels showing a Coomassie Stain on pre- and post-concentration viral material for Study #1 Ring19-eGFP Anellovector material (FIG. 4A) and Study #2 Ring19-eGFP Anellovector material (FIG. 4B).

FIG. 5A and FIG. 5B depict plots showing the quantitation of eGFP gDNA in mouse brain (FIG. 5A) and spinal cord (FIG. 5B) by qPCR. Each dot represents one brain hemisphere or spinal cord. n=5 per group.

FIG. 6 depicts a plot showing the quantitation of eGFP mRNA in the mouse brain by RT-ddPCR. Each dot represents one brain hemisphere. n=5 brain hemispheres per group.

FIG. 7A-7F depict fluorescence microscopy images of representative brain sections of ICV-injected mice stained for eGFP protein from the PBS control group (FIG. 7A, FIG. 7D), the R19-eGFP group (FIG. 7B, FIG. 7E), and the AAV9-eGFP group (FIG. 7C, FIG. 7F). Images were taken at 2× (FIG. 7A-C) or 20× (FIG. 7D-F) magnification. The white square in FIGS. 7A-C indicates the location of the 20× magnification in the respective 2× image. eGFP protein expression is detected in the R19-eGFP and AAV9-eGFP groups, while no staining is detected in the PBS control group. n=2 brains per group.

FIG. 8A and FIG. 8B depict plots showing the quantitation of eGFP gDNA by qPCR in the spinal cord (FIG. 8A) and brain (FIG. 8B) in mice administered with PBS, R19-fCMV-eGFP, or AAV9-fCMV-eGFP by intrathecal (IT) injection. n=5 per group.

FIGS. 9A-9D depict plots showing the quantitation of eGFP gDNA by qPCR in the spinal cord (FIG. 9A), brain (FIG. 9B), liver (FIG. 9C), and muscle (FIG. 9D) in mice from Group 1 (“PBS-D21”), Group 2 (“PBS×1 D42”), Group 3 (“PBS×2 D42”), Group 4 (“R19-eGFP—D21”), Group 5 (“R19-eGFP×1 D42”), Group 6 (“R19-eGFP×2 D42”), Group 7 (“AAV9-eGFP-D21”), Group 8 (“AAV-eGFP×1 D42”), and Group 9 (“AAV-eGFP×2 D42”).

FIGS. 10A-10D depict plots showing the quantitation of eGFP mRNA by RT-ddPCR in the spinal cord (FIG. 10A), liver (FIG. 10B), brain (FIG. 10C), and muscle (FIG. 10D) in mice from Group 1 (“PBS-D21”), Group 2 (“PBS×1 D42”), Group 3 (“PBS×2 D42”), Group 4 (“R19-eGFP—D21”), Group 5 (“R19-eGFP×1 D42”), Group 6 (“R19-eGFP×2 D42”), Group 7 (“AAV9-eGFP-D21”), Group 8 (“AAV-eGFP×1 D42”), or Group 9 (“AAV-eGFP×2 D42”). In FIGS. 10C and 10D, “PBS” corresponds to Group 1, “Ring19-eGFP” corresponds to Group 4, and “AAV9-eGFP” corresponds to Group 7.

FIGS. 11A-11J depict fluorescence microscopy images of representative spinal cord sections of IT-injected mice stained for eGFP protein from Group 1 (“PBS 21 day) (FIG. 11A), Group 2 (“PBS 42 day”) (FIG. 11B), Group 3 (“PBS redose”) (FIG. 11C), Group 4 (“R19 21 day”) (FIG. 11D), Group 5 (“R19 42 day”) (FIG. 11E), Group 6 (“R19 redose”) (FIG. 11F), Group 7 (“AAV 21 day”) (FIG. 11G), Group 8 (“AAV 42 day”) (FIG. 11H), and Group 9 (“AAV redose”) (FIGS. 11I and 11J).

FIGS. 11A-11I are shown at 20× magnification. The square in FIG. 11J (2× magnification) indicates the location of the tissue magnified to 20× in FIG. 11I.

### DETAILED DESCRIPTION OF CERTAIN EMBODIMENTS

### Definitions

The present invention will be described with respect to particular embodiments and with reference to certain figures, but the invention is not limited thereto but only by the claims. Terms as set forth hereinafter are generally to be understood in their common sense unless indicated otherwise.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is to be understood to preferably also disclose a group which consists only of these embodiments.

Where an indefinite or definite article is used when referring to a singular noun, e.g. “a”, “an” or “the”, this includes a plural of that noun unless something else is specifically stated.

The wording “compound, composition, product, etc. for treating, modulating, etc.” is to be understood to refer a compound, composition, product, etc. per se which is suitable for the indicated purposes of treating, modulating, etc. The wording “compound, composition, product, etc. for treating, modulating, etc.” additionally discloses that, as an embodiment, such compound, composition, product, etc. is for use in treating, modulating, etc.

The wording “compound, composition, product, etc. for use in . . . ”, “use of a compound, composition, product, etc. in the manufacture of a medicament, pharmaceutical composition, veterinary composition, diagnostic composition, etc. for . . . ”, or “compound, composition, product, etc. for use as a medicament . . . ” indicates that such compounds, compositions, products, etc. are to be used in therapeutic methods which may be practiced on the human or animal body. They are considered as an equivalent disclosure of embodiments and claims pertaining to methods of treatment, etc. If an embodiment or a claim thus refers to “a compound for use in treating a human or animal being suspected to suffer from a disease”, this is considered to be also a disclosure of a “use of a compound in the manufacture of a medicament for treating a human or animal being suspected to suffer from a disease” or a “method of treatment by administering a compound to a human or animal being suspected to suffer from a disease”. The wording “compound, composition, product, etc. for treating, modulating, etc.” is to be understood to refer a compound, composition, product, etc. per se which is suitable for the indicated purposes of treating, modulating, etc.

If hereinafter examples of a term, value, number, etc. are provided in parentheses, this is to be understood as an indication that the examples mentioned in the parentheses can constitute an embodiment. For example, if it is stated that “in embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1-encoding nucleotide sequence of Table 1 (e.g., nucleotides 571-2613 of the nucleic acid sequence of Table 1)”, then some embodiments relate to nucleic acid molecules comprising a nucleic acid sequence having at least about 70%, 75%, 80%, 85%, 90%, 95%, 96%, 97%, 98%, 99%, or 100% sequence identity to nucleotides 571-2613 of the nucleic acid sequence of Table 1.

As used herein, the term “Anelloviridae family vector” refers to a vehicle derived from or similar to a virus of the Anelloviridae family (e.g., an Alphatorquevirus, Betatorquevirus, Gammatorquevirus, or chicken anemia virus), wherein the vehicle comprises a genetic element enclosed in a proteinaceous exterior (e.g., the genetic element is substantially protected from digestion with DNAse I by a proteinaceous exterior). In some embodiments, an Anelloviridae family vector comprises a genetic element derived from or highly similar to (e.g., at least 85%, 90%, 95%, 96%, 97%, 98%, 99%, or 100% identical to) that of an Alphatorquevirus, Betatorquevirus, or Gammatorquevirus. In some embodiments, an Anelloviridae family vector comprises a proteinaceous exterior comprising a protein derived from or similar to (e.g., at least 50%, 60%, 70%, 75%, 80%, 85%, 90%, 95%, 96%, 97%, 98%, 99%, or 100% identical to) a capsid protein of an Alphatorquevirus, Betatorquevirus, or Gammatorquevirus (e.g., an Alphatorquevirus ORF1, Betatorquevirus ORF1, or Gammatorquevirus ORF1). In some embodiments, enclosed within a proteinaceous exterior encompasses 100% coverage by a proteinaceous exterior, as well as less than 100% coverage, e.g., 95%, 90%, 85%, 80%, 70%, 60%, 50% or less. For example, gaps or discontinuities (e.g., that render the proteinaceous exterior permeable to water, ions, peptides, or small molecules) may be present in the proteinaceous exterior, so long as the genetic element is retained in the proteinaceous exterior or protected from digestion with DNAse I, e.g., prior to entry into a host cell. In some embodiments, the Anelloviridae family vector is purified, e.g., it is separated from its original source and/or substantially free (>50%, >60%, >70%, >80%, >90%) of other components. In some embodiments, the Anelloviridae family vector is capable of introducing the genetic element into a target cell (e.g., via infection). In some embodiments, the Anelloviridae family vector is an infective synthetic viral particle.

As used herein, the term “anellovector” refers to a vehicle comprising a genetic element, e.g., e.g., circular DNA, enclosed in a proteinaceous exterior. A “synthetic anellovector,” as used herein, generally refers to an anellovector that is not naturally occurring, e.g., has a sequence that is different relative to a wild-type virus (e.g., a wild-type Anellovirus as described herein). In some embodiments, the synthetic anellovector is engineered or recombinant, e.g., comprises a genetic element that comprises a difference or modification relative to a wild-type viral genome (e.g., a wild-type Anellovirus genome as described herein). In some embodiments, enclosed within a proteinaceous exterior encompasses 100% coverage by a proteinaceous exterior, as well as less than 100% coverage, e.g., 95%, 90%, 85%, 80%, 70%, 60%, 50% or less. For example, gaps or discontinuities (e.g., that render the proteinaceous exterior permeable to water, ions, peptides, or small molecules) may be present in the proteinaceous exterior, so long as the genetic element is retained in the proteinaceous exterior, e.g., prior to entry into a host cell. In some embodiments, the anellovector is purified, e.g., it is separated from its original source and/or substantially free (>50%, >60%, >70%, >80%, >90%) of other components.

An anellovector may, in some embodiments, comprise a nucleic acid vector that comprises sufficient nucleic acid sequence derived from or highly similar to (e.g., at least 85%, 90%, 95%, 96%, 97%, 98%, 99%, or 100% identical to) an Anellovirus genome sequence or a contiguous portion thereof to allow packaging into a proteinaceous exterior (e.g., a capsid), and further comprises a heterologous sequence. In some embodiments, the nucleic acid vector is a viral vector or a naked nucleic acid. In some embodiments, the nucleic acid vector comprises at least about 50, 60, 70, 71, 72, 73, 74, 75, 80, 90, 100, 150, 200, 300, 400, 500, 600, 700, 800, 900, 1000, 1100, 1200, 1300, 1400, 1500, 1600, 1700, 1800, 1900, 2000, 2500, 3000, or 3500 consecutive nucleotides of a native Anellovirus sequence or a sequence highly similar (e.g., at least 85%, 90%, 95%, 96%, 97%, 98%, 99%, or 100% identical) thereto. In some embodiments, the anellovector further comprises one or more of an Anellovirus ORF1, ORF2, or ORF3. In some embodiments, the heterologous sequence comprises a multiple cloning site, comprises a heterologous promoter, comprises a coding region for a therapeutic protein, or encodes a therapeutic nucleic acid. In some embodiments, the capsid is a wild-type Anellovirus capsid. In embodiments, an anellovector comprises a genetic element described herein, e.g., comprises a genetic element comprising a promoter, a sequence encoding a therapeutic effector, and a capsid binding sequence.

As used herein, the term “Anellovirus non-coding region (NCR)” refers to a sequence of an untranslated region of an Anellovirus genome sequence that extends from just upstream of the ORF2 start codon to the untranslated region of the Anellovirus genome sequence just downstream of the ORF3 stop codon in a circular genome. The Anellovirus NCR may comprise the “Anellovirus 5′ NCR sequence” and “Anellovirus 3′ NCR sequence.” In some embodiments, the Anellovirus NCR sequence is contiguous. In some embodiments, the Anellovirus NCR sequence is non-contiguous. In some embodiments, the portions of a non-contiguous Anellovirus NCR sequence are separated by a heterologous insertion (e.g., comprising a recombinase hybrid site, e.g., as described herein). In some embodiments, the Anellovirus NCR sequence comprises a sequence found in a wild-type Anellovirus, and in other embodiments, the Anellovirus NCR comprises one or more mutations relative to the closest Anellovirus sequence. In some embodiments, the Anellovirus NCR is comprised by a nucleic acid molecule that does not comprise Anellovirus ORF2 or ORF3 coding sequences. In some instances, the Anellovirus NCR refers to the sense strand, the antisense strand, or a double-stranded DNA comprising both the sense strand and antisense strands. In some instances, a listing of an Anellovirus NCR sequence herein can refer to the sequence listed and/or its reverse complement.

As used herein, the term “Anellovirus 5′ NCR” refers to a sequence of an untranslated region of an Anellovirus genome sequence that extends from just upstream of the ORF2 start codon through the 5′ UTR conserved domain to the Anellovirus 3′ NCR sequence, and sequences with homology thereto. In some embodiments, an Anellovirus 5′ NCR sequence comprises origin of replication activity. In some embodiments, the Anellovirus 5′ NCR sequence is contiguous. In some embodiments, the Anellovirus 5′ NCR sequence is non-contiguous. In some embodiments, the portions of a non-contiguous Anellovirus 5′ NCR sequence are separated by a heterologous insertion (e.g., comprising a recombinase hybrid site, e.g., as described herein). In some embodiments, the Anellovirus 5′ NCR sequence comprises a sequence found in a wild-type Anellovirus, and in other embodiments, the Anellovirus 5′ NCR comprises one or more mutations relative to the closest Anellovirus sequence. In some instances, the Anellovirus 5′ NCR refers to the sense strand, the antisense strand, or a double-stranded DNA comprising both the sense strand and antisense strands. In some instances, a listing of an Anellovirus 5′ NCR sequence herein can refer to the sequence listed and/or its reverse complement. In a circular genetic element, the Anellovirus 5′ NCR and Anellovirus 3′ NCR may be directly adjacent to each other (e.g., to form an Anellovirus NCR). Exemplary dividing points between the Anellovirus 5′ NCR and the Anellovirus 3′ NCR are shown, e.g., as described herein.

As used herein, the term “Anellovirus 3′ NCR” refer to a sequence of an untranslated region of an Anellovirus genome sequence that extends from just downstream of the ORF3 stop codon through the GC-rich region to the Anellovirus 5′ NCR sequence, and sequences with homology thereto. In some embodiments, the Anellovirus 3′ NCR sequence is contiguous. In some embodiments, the Anellovirus 3′ NCR sequence is non-contiguous. In some embodiments, the portions of a non-contiguous Anellovirus 3′ NCR sequence are separated by a heterologous insertion (e.g., comprising a recombinase hybrid site, e.g., as described herein). In some embodiments, the Anellovirus 3′ NCR sequence comprises a sequence found in a wild-type Anellovirus, and in other embodiments, the Anellovirus 3′ NCR comprises one or more mutations relative to the closest Anellovirus sequence. In some instances, the Anellovirus 3′ NCR refers to the sense strand, the antisense strand, or a double-stranded DNA comprising both the sense strand and antisense strands. In some instances, a listing of an Anellovirus 3′ NCR sequence herein can refer to the sequence listed and/or its reverse complement.

As used herein, the term “Anellovirus GC-rich region” refers to a wild-type or engineered sequence that has an activity and a structural feature of a GC-rich region of a wild-type Anellovirus, or a functional fragment thereof. In some embodiments, the functional fragment has a length of at least 20, 30, 40, 50, 60, 70, 80, 90, or 100 nucleotides. Typically, the negative strand comprising the Anellovirus GC-rich region is packaged into a particle (e.g., an Anelloviridae family vector) as described herein. In some embodiments, the Anellovirus GC-rich region is a wild-type Anellovirus GC-rich region. In some embodiments, the Anellovirus GC-rich region is an engineered Anellovirus GC-rich region having a nucleic acid sequence with at least one difference relative to the closest wild-type Anellovirus GC-rich region sequence.

As used herein, the term “Anellovirus 5′ UTR conserved domain” refers to a wild-type or engineered sequence that has an activity and a structural feature of an Anellovirus 5′ UTR conserved domain of a wild-type Anellovirus, or a functional fragment thereof. In some embodiments, the functional fragment has a length of at least 15, 20, 30, 40, 50, 60, or 70 nucleotides. Typically, the negative strand comprising the Anellovirus 5′ UTR conserved domain is packaged into a particle (e.g., an Anelloviridae family vector) as described herein. In some embodiments, the Anellovirus 5′ UTR conserved domain is a wild-type Anellovirus 5′ UTR conserved domain. In some embodiments, the Anellovirus 5′ UTR conserved domain is an engineered Anellovirus 5′ UTR conserved domain having a nucleic acid sequence with at least one difference relative to the closest wild-type Anellovirus 5′ UTR conserved domain sequence.

As used herein, the term “antibody molecule” refers to a protein, e.g., an immunoglobulin chain or fragment thereof, comprising at least one immunoglobulin variable domain sequence. The term “antibody molecule” encompasses full-length antibodies and antibody fragments (e.g., scFvs). In some embodiments, an antibody molecule is a multispecific antibody molecule, e.g., the antibody molecule comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In embodiments, the multispecific antibody molecule is a bispecific antibody molecule. A bispecific antibody molecule is generally characterized by a first immunoglobulin variable domain sequence which has binding specificity for a first epitope and a second immunoglobulin variable domain sequence that has binding specificity for a second epitope.

The term “in vitro assembly,” as used herein with respect to an Anelloviridae family vector or an anelloVLP, refers to the formation of a proteinaceous exterior comprising an ORF1 molecule, wherein the formation does not take place inside of a cell (e.g., takes place in a cell-free system such as a cell-free suspension, a lysate, or a supernatant). In some instances, in vitro assembly of an Anelloviridae family vector comprises enclosure, outside of a cell, of a genetic element (e.g., as described herein) within the proteinaceous exterior. In some instances, in vitro assembly of an anelloVLP comprises association, outside of a cell, of an effector (e.g., an exogenous effector, e.g., as described herein) with the proteinaceous exterior (e.g., enclosed within the proteinaceous exterior). In vitro assembly of a proteinaceous exterior may occur, in some instances, under conditions suitable for multimerization of a plurality of ORF1 molecules (e.g., nondenaturing conditions), e.g., to form a multimer of more than 10, 11, 12, 13, 14, 15, 16, 17, 18, 19, or 20 ORF1 molecules. In some instances, in vitro assembly results in the formation of a proteinaceous exterior comprising at least about 20, 30, 40, 50, or 60 ORF1 molecules, or about 20-30, 30-40, 40-50, 50-60, or 60-70 ORF1 molecules). In some instances, the proteinaceous exterior is formed from ORF1 molecules that were produced in a cell and then purified therefrom. In some instances, the in vitro assembly takes place in a solution free of cells or constituents thereof. In other instances, the in vitro assembly takes place in a solution comprising cell debris (e.g., from lysed cells). In some instances, the in vitro assembly takes place in a solution substantially free of cellular nucleic acid molecules (e.g., genomic DNA, mitochondrial DNA, mRNA, and/or noncoding RNA from a cell).

As used herein, a nucleic acid “encoding” refers to a nucleic acid sequence encoding an amino acid sequence or a functional polynucleotide (e.g., a non-coding RNA, e.g., an siRNA or miRNA).

An “exogenous” agent (e.g., an effector, a nucleic acid (e.g., RNA), a gene, payload, protein) as used herein refers to an agent that is either not comprised by, or not encoded by, a corresponding wild-type virus, e.g., an Anellovirus as described herein. In some embodiments, the exogenous agent does not naturally exist, such as a protein or nucleic acid that has a sequence that is altered (e.g., by insertion, deletion, or substitution) relative to a naturally occurring protein or nucleic acid. In some embodiments, the exogenous agent does not naturally exist in the host cell. In some embodiments, the exogenous agent exists naturally in the host cell but is exogenous to the virus. In some embodiments, the exogenous agent exists naturally in the host cell, but is not present at a desired level or at a desired time.

A “heterologous” agent or element (e.g., an effector, a nucleic acid sequence, an amino acid sequence), as used herein with respect to another agent or element (e.g., an effector, a nucleic acid sequence, an amino acid sequence), refers to agents or elements that are not naturally found together, e.g., in a wild-type virus, e.g., an Anellovirus. In some embodiments, a heterologous nucleic acid sequence may be present in the same nucleic acid as a naturally occurring nucleic acid sequence (e.g., a sequence that is naturally occurring in the Anellovirus). In some embodiments, a heterologous agent or element is exogenous relative to an Anellovirus from which other (e.g., the remainder of) elements of the anellovector are based.

As used herein, the term “genetic element” refers to a nucleic acid sequence, generally in an anellovector. It is understood that the genetic element can be produced as naked DNA and optionally further assembled into a proteinaceous exterior. It is also understood that an anellovector can insert its genetic element into a cell, resulting in the genetic element being present in the cell and the proteinaceous exterior not necessarily entering the cell.

As used herein, the term “ORF1 molecule” refers to a polypeptide having an activity and/or a structural feature of an Anellovirus ORF1 protein (e.g., an Anellovirus ORF1 protein as described herein, e.g., as listed in Table A1-A3), or a functional fragment thereof. An ORF1 molecule may, in some instances, comprise one or more of (e.g., 1, 2, 3 or 4 of): a first region comprising at least 60% basic residues (e.g., at least 60% arginine residues), a second region comprising at least about six beta strands (e.g., at least 4, 5, 6, 7, 8, 9, 10, 11, or 12 beta strands), a third region comprising a structure or an activity of an Anellovirus N22 domain (e.g., as described herein, e.g., an N22 domain from an Anellovirus ORF1 protein as described herein), and/or a fourth region comprising a structure or an activity of an Anellovirus C-terminal domain (CTD) (e.g., as described herein, e.g., a CTD from an Anellovirus ORF1 protein as described herein). In some instances, the ORF1 molecule comprises, in N-terminal to C-terminal order, the first, second, third, and fourth regions. In some instances, an anellovector comprises an ORF1 molecule comprising, in N-terminal to C-terminal order, the first, second, third, and fourth regions. An ORF1 molecule may, in some instances, comprise a polypeptide encoded by an Anellovirus ORF1 nucleic acid (e.g., as listed in any of Tables N1-N3). An ORF1 molecule may, in some instances, further comprise a heterologous sequence, e.g., a hypervariable region (HVR), e.g., an HVR from an Anellovirus ORF1 protein, e.g., as described herein. An “Anellovirus ORF1 protein,” as used herein, refers to an ORF1 protein encoded by an Anellovirus genome (e.g., a wild-type Anellovirus genome, e.g., as described herein), e.g., an ORF1 protein having the amino acid sequence as listed in Table A1-A3, or as encoded by the ORF1 gene as listed in any of Tables N1-N3.

The term “ORF1 domain,” as used herein with respect to an ORF1 molecule, refers to the portion of the ORF1 molecule having the structure or function of an Anellovirus ORF1 protein. The ORF1 domain is generally capable of forming a multimer with other copies of the ORF1 domain (e.g., in other ORF1 molecules), or with other ORF1 molecules, e.g., to form a proteinaceous exterior (e.g., of an anellovector or anelloVLP as described herein). In some instances, the ORF1 molecule may comprise one or more additional domains other than the ORF1 domain (for example, a domain comprising or attached to a surface effector). In some instances, the amino acid sequence of an ORF1 domain comprises an insertion (e.g., an insertion encoding a surface moiety or a domain capable of binding to a surface moiety), e.g., between the N-terminal end and C-terminal end of the ORF1 domain. In certain instances, the insertion does not substantially disrupt the structure and/or function of the ORF1 domain, e.g., such that the ORF1 domain remains capable of forming a multimer with other ORF1 domains or ORF1 molecules. The position within the ORF1 domain sequence into which the insertion is made is referred to herein as the “insertion point.” An insertion can be made into an ORF1 domain by any genetic or polypeptide engineering method known in the art. In some embodiments, an ORF1 molecule consists of an ORF1 domain. In other embodiments, an ORF1 molecule comprises an ORF1 domain and a heterologous domain (e.g., a surface moiety as described herein). In some embodiments, an ORF1 domain is connected to a surface moiety by a polypeptide linker region.

As used herein, the term “ORF2 molecule” refers to a polypeptide having an activity and/or a structural feature of an Anellovirus ORF2 protein (e.g., an Anellovirus ORF2 protein as described herein, e.g., as listed in Table A1-A3), or a functional fragment thereof. An “Anellovirus ORF2 protein,” as used herein, refers to an ORF2 protein encoded by an Anellovirus genome (e.g., a wild-type Anellovirus genome, e.g., as described herein), e.g., an ORF2 protein having the amino acid sequence as listed in Table A1-A3, or as encoded by the ORF2 gene as listed in any of Tables N1-N3.

As used herein, the term “particle” refers to a vehicle having a diameter of less than 100 nm (e.g., about 20-25, 25-30, 30-35, or 35-40 nm) comprising a proteinaceous exterior. In some instances, the particle comprises a plurality of ORF1 molecules. The proteinaceous exterior of the particle generally forms an enclosure capable of limiting or preventing movement of certain molecules between the inside and outside of the proteinaceous exterior. In some embodiments, gaps or discontinuities (e.g., that render the proteinaceous exterior permeable to water, ions, peptides, or small molecules) may be present in the proteinaceous exterior. In certain embodiments, the gaps or discontinuities are of a sufficiently small size (e.g., diameter) that the proteinaceous exterior limits or prevents one or more large macromolecules (e.g., peptides, polypeptides, polynucleotides, lipids, or polysaccharides) from passing through the proteinaceous exterior.

As used herein, the term “proteinaceous exterior” refers to an exterior component that is predominantly (e.g., >50%, >60%, >70%, >80%, >90%) protein.

As used herein, the term “regulatory nucleic acid” refers to a nucleic acid sequence that modifies expression, e.g., transcription and/or translation, of a DNA sequence that encodes an expression product. In embodiments, the expression product comprises RNA or protein.

As used herein, the term “regulatory sequence” refers to a nucleic acid sequence that modifies transcription of a target gene product. In some embodiments, the regulatory sequence is a promoter or an enhancer.

As used herein, the term “replication protein” refers to a protein, e.g., a viral protein, that is utilized during infection, viral genome replication/expression, viral protein synthesis, and/or assembly of the viral components.

As used herein, a “substantially non-pathogenic” organism, particle, or component, refers to an organism, particle (e.g., a virus or an anellovector, e.g., as described herein), or component thereof that does not cause or induce a detectable disease or pathogenic condition, e.g., in a host organism, e.g., a mammal, e.g., a human. In some embodiments, administration of an anellovector to a subject can result in minor reactions or side effects that are acceptable as part of standard of care.

As used herein, the term “non-pathogenic” refers to an organism or component thereof that does not cause or induce a detectable disease or pathogenic condition, e.g., in a host organism, e.g., a mammal, e.g., a human.

As used herein, a “substantially non-integrating” genetic element refers to a genetic element, e.g., a genetic element in a virus or anellovector, e.g., as described herein, wherein less than about 0.01%, 0.05%, 0.1%, 0.5%, or 1% of the genetic element that enter into a host cell (e.g., a eukaryotic cell) or organism (e.g., a mammal, e.g., a human) integrate into the genome. In some embodiments the genetic element does not detectably integrate into the genome of, e.g., a host cell. In some embodiments, integration of the genetic element into the genome can be detected using techniques as described herein, e.g., nucleic acid sequencing, PCR detection and/or nucleic acid hybridization.

As used herein, a “substantially non-immunogenic” organism, particle, or component, refers to an organism, particle (e.g., a virus or anellovector, e.g., as described herein), or component thereof, that does not cause or induce an undesired or untargeted immune response, e.g., in a host tissue or organism (e.g., a mammal, e.g., a human). In some embodiments, the substantially non-immunogenic organism, particle, or component does not produce a detectable immune response. In some embodiments, the substantially non-immunogenic anellovector does not produce a detectable immune response against a protein comprising an amino acid sequence or encoded by a nucleic acid sequence shown in any of Tables N1-N3. In some embodiments, an immune response (e.g., an undesired or untargeted immune response) is detected by assaying antibody presence or level (e.g., presence or level of an anti-anellovector antibody, e.g., presence or level of an antibody against an anellovector as described herein) in a subject, e.g., according to the anti-TTV antibody detection method described in Tsuda et al. (1999; J. Virol. Methods 77: 199-206; incorporated herein by reference) and/or the method for determining anti-TTV IgG levels described in Kakkola et al. (2008; Virology 382: 182-189; incorporated herein by reference). Antibodies against an Anellovirus or an anellovector based thereon can also be detected by methods in the art for detecting anti-viral antibodies, e.g., methods of detecting anti-AAV antibodies, e.g., as described in Calcedo et al. (2013; Front. Immunol. 4(341): 1-7; incorporated herein by reference).

A “subsequence” as used herein refers to a nucleic acid sequence or an amino acid sequence that is comprised in a larger nucleic acid sequence or amino acid sequence, respectively. In some instances, a subsequence may comprise a domain or functional fragment of the larger sequence. In some instances, the subsequence may comprise a fragment of the larger sequence capable of forming secondary and/or tertiary structures when isolated from the larger sequence similar to the secondary and/or tertiary structures formed by the subsequence when present with the remainder of the larger sequence. In some instances, a subsequence can be replaced by another sequence (e.g., a subsequence comprising an exogenous sequence or a sequence heterologous to the remainder of the larger sequence, e.g., a corresponding subsequence from a different Anellovirus).

As used herein, the term “surface moiety” refers to a moiety for which at least a portion is exposed on the exterior surface of a particle (e.g., exposed to the solution surrounding the particle). The surface moiety is generally attached, directly or indirectly, to a component of the proteinaceous exterior of the particle (e.g., an ORF1 molecule). In some instances, the surface moiety is covalently attached to the component of the proteinaceous exterior of the particle (e.g., the ORF1 molecule). In some instances, the surface moiety is noncovalently attached to the component of the proteinaceous exterior of the particle (e.g., the ORF1 molecule). In some instances, the surface moiety is bound to a binding moiety that is in turn attached (e.g., covalently or noncovalently) to the component of the proteinaceous exterior of the particle (e.g., the ORF1 molecule). In some instances, the surface moiety is comprised in an ORF1 molecule (e.g., is a heterologous domain of an ORF1 molecule). In some instances, a surface moiety is exogenous relative to an Anellovirus (e.g., the Anellovirus from which the ORF1 molecule was derived and/or an Anellovirus for which the ORF1 protein has at least 50%, 60%, 70%, 75%, 80%, 85%, 90%, 95%, 96%, 97%, 98%, 99%, or 100% sequence identity to the ORF1 molecule). In some instances, a surface moiety is exogenous relative a target cell (e.g., a mammalian cell, e.g., a human cell) to be infected by the particle.

As used herein, “treatment”, “treating” and cognates thereof refer to the medical management of a subject with the intent to improve, ameliorate, stabilize, prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to preventing, minimizing or partially or completely inhibiting the development of the associated disease, pathological condition, or disorder); and supportive treatment (treatment employed to supplement another therapy).

This invention relates generally to Anelloviridae family vectors (e.g., anellovectors), e.g., synthetic Anelloviridae family vectors (e.g., anellovectors), and uses thereof. The present disclosure provides Anelloviridae family vectors (e.g., anellovectors), compositions comprising Anelloviridae family vectors (e.g., anellovectors), and methods of making or using Anelloviridae family vectors (e.g., anellovectors). Anelloviridae family vectors (e.g., anellovectors) are generally useful as delivery vehicles, e.g., for delivering a therapeutic agent to a eukaryotic cell. Generally, an Anelloviridae family vector (e.g., anellovector) will include a genetic element comprising a nucleic acid sequence (e.g., encoding an effector, e.g., an exogenous effector or an endogenous effector) enclosed within a proteinaceous exterior. An Anelloviridae family vector (e.g., anellovector) may include one or more deletions of sequences (e.g., regions or domains as described herein) relative to an Anellovirus sequence (e.g., as described herein). Anelloviridae family vectors (e.g., anellovectors) can be used as a substantially non-immunogenic vehicle for delivering the genetic element, or an effector encoded therein (e.g., a polypeptide or nucleic acid effector, e.g., as described herein), into eukaryotic cells, e.g., to treat a disease or disorder in a subject comprising the cells.

### TABLE OF CONTENTS

I. Anelloviridae Family Vectors (e.g., Anellovectors)
   A. Anelloviridae Family Viruses (e.g., Anelloviruses)
      i. Nucleic acid sequences
      ii. Amino acid sequences encoded by nucleic acid sequences
      iii. Proteins comprising amino acid sequences
      iv. Polypeptides comprising amino acid sequences
   B. Capsid Proteins (e.g., ORF1 molecules)
      i. Conserved ORF1 motif in N22 domain
      ii. Exemplary ORF1 sequences
      iii. Identification of ORF1 protein sequences
   C. ORF2 molecules
      i. Conserved ORF2 motif
   D. Genetic elements
   E. Protein binding sequence
   F. 5′ UTR Conserved Domains
   G. GC-rich regions
   H. Effectors
   I. Surface Moieties
II. Compositions and Methods for Making Anelloviridae Family Vectors
   A. Genetic Element Constructs
      i. Tandem constructs
      ii. Cis/trans constructs
   B. Recombinase-based production of genetic elements and Anellovectors
      i. Self-replicating rescue (SRR) constructs (e.g., SRR plasmids)
      ii. Exemplary site-specific recombinases and recombinase recognition sites
   C. Host Cells and methods of using host cells for producing an Anellovector
      i. Introduction of genetic elements into host cells
      ii. Exemplary cell types
   D. Culture Conditions
   E. Harvest
   F. In vitro assembly methods
   G. Enrichment and Purification
III. Pharmaceutical Compositions
IV. Methods of use
V. Redosing

### I. Anelloviridae Family Vectors (e.g., Anellovectors)

In some aspects, the invention described herein comprises compositions and methods of using and making an Anelloviridae family vector (e.g., anellovector), Anelloviridae family vector (e.g., anellovector) preparations, and therapeutic compositions. In some embodiments, the anellovector has a sequence, structure, and/or function that is based on an Anelloviridae virus (e.g., an Anellovirus as described herein). It is understood that applicable embodiments described herein with respect to anellovectors may also be applied to Anelloviridae family vectors (e.g., a vector based on or derived from a chicken anemia virus (CAV), e.g., as described herein). In some embodiments, the Anelloviridae family vector (e.g., anellovector) comprises a nucleic acid or polypeptide comprising a sequence as shown in Table A1-A3 (e.g., Table A1, A2, or A3); or Table N1-N3 (e.g., Table N1, N2, or N3), or fragments or portions thereof, or other substantially non-pathogenic virus, e.g., a symbiotic virus, commensal virus, native virus. In some embodiments, an Anelloviridae family virus-based vector comprises at least one element exogenous to that Anelloviridae family virus, e.g., an exogenous effector or a nucleic acid sequence encoding an exogenous effector disposed within a genetic element of the vector. In some embodiments, an Anelloviridae family virus-based vector comprises at least one element heterologous to another element from that Anelloviridae family virus, e.g., an effector-encoding nucleic acid sequence that is heterologous to another linked nucleic acid sequence, such as a promoter element. In some embodiments, an Anelloviridae family vector comprises a genetic element (e.g., circular DNA, e.g., single stranded DNA), which comprise at least one element that is heterologous relative to the remainder of the genetic element and/or the proteinaceous exterior (e.g., an exogenous element encoding an effector, e.g., as described herein). An Anelloviridae family vector may be a delivery vehicle (e.g., a substantially non-pathogenic delivery vehicle) for a payload into a host, e.g., a human. In some embodiments, the Anelloviridae family vector is capable of replicating in a eukaryotic cell, e.g., a mammalian cell, e.g., a human cell. In some embodiments, the Anelloviridae family vector is substantially non-pathogenic and/or substantially non-integrating in the mammalian (e.g., human) cell. In some embodiments, the Anelloviridae family vector is substantially non-immunogenic in a mammal, e.g., a human. In some embodiments, the Anelloviridae family vector is replication-deficient. In some embodiments, the Anelloviridae family vector is replication-competent.

In one aspect, the invention includes an Anelloviridae family vector comprising:
a) a genetic element comprising (i) a sequence encoding an exterior protein (e.g., a non-pathogenic exterior protein), (ii) an exterior protein binding sequence that binds the genetic element to the non-pathogenic exterior protein, and (iii) a sequence encoding an effector (e.g., an endogenous or exogenous effector); and
b) a proteinaceous exterior that is associated with, e.g., envelops or encloses, the genetic element.

In some embodiments, the Anelloviridae family vector (e.g. anellovector) includes sequences or expression products from (or having >70%, 75%, 80%, 85%, 90%, 95%, 97%, 98%, 99%, 100% homology to) a non-enveloped, circular, single-stranded DNA virus. Animal circular single-stranded DNA viruses generally refer to a subgroup of single strand DNA (ssDNA) viruses, which infect eukaryotic non-plant hosts, and have a circular genome. Thus, animal circular ssDNA viruses are distinguishable from ssDNA viruses that infect prokaryotes (i.e. Microviridae and Inoviridae) and from ssDNA viruses that infect plants (i.e. Geminiviridae and Nanoviridae). They are also distinguishable from linear ssDNA viruses that infect non-plant eukaryotes (i.e. Parvoviridiae).

In some embodiments, the genetic element comprises a promoter element. In some embodiments, the promoter element is selected from an RNA polymerase II-dependent promoter, an RNA polymerase III-dependent promoter, a PGK promoter, a CMV promoter, an EF-1α promoter, an SV40 promoter, a CAGG promoter, or a UBC promoter, TTV viral promoters, Tissue specific, U6 (pollIII), minimal CMV promoter with upstream DNA binding sites for activator proteins (TetR-VP16, Gal4-VP16, dCas9-VP16, etc.). In some embodiments, the promoter element comprises a TATA box. In some embodiments, the promoter element is endogenous to a wild-type Anelloviridae family virus (e.g., Anellovirus), e.g., as described herein.

In some embodiments, the genetic element comprises one or more of the following characteristics: single-stranded, circular, negative strand, and/or DNA. In some embodiments, the portions of the genetic element excluding the effector have a combined size of about 2.5-5 kb (e.g., about 2.8-4 kb, about 2.8-3.2 kb, about 3.6-3.9 kb, or about 2.8-2.9 kb), less than about 5 kb (e.g., less than about 2.9 kb, 3.2 kb, 3.6 kb, 3.9 kb, or 4 kb), or at least 100 nucleotides (e.g., at least 1 kb).

In some embodiments, a replication deficient, replication defective, or replication incompetent genetic element does not encode all of the necessary machinery or components required for replication of the genetic element. In some embodiments, a replication defective genetic element does not encode a replication factor. In some embodiments, a replication defective genetic element does not encode one or more ORFs (e.g., ORF1, ORF1/1, ORF1/2, ORF2, ORF2/2, ORF2/3, and/or ORF2t/3 e.g., as described herein). In some embodiments, the machinery or components not encoded by the genetic element may be provided in trans (e.g., using a helper, e.g., a helper virus or helper plasmid, or encoded in a nucleic acid comprised by the host cell, e.g., integrated into the genome of the host cell), e.g., such that the genetic element can undergo replication in the presence of the machinery or components provided in trans.

In some embodiments, a packaging deficient, packaging defective, or packaging incompetent genetic element cannot be packaged into a proteinaceous exterior (e.g., wherein the proteinaceous exterior comprises a capsid or a portion thereof, e.g., comprising a polypeptide encoded by an ORF1nucleic acid, e.g., as described herein). In some embodiments, a packaging deficient genetic element is packaged into a proteinaceous exterior at an efficiency less than 10% (e.g., less than 10%, 9%, 8%, 7%, 6%, 5%, 4%, 3%, 2%, 1%, 0.5%, 0.1%, 0.01%, or 0.001%) compared to a wild-type Anelloviridae family virus (e.g., Anellovirus) (e.g., as described herein). In some embodiments, the packaging defective genetic element cannot be packaged into a proteinaceous exterior even in the presence of factors (e.g., ORF1, ORF1/1, ORF1/2, ORF2, ORF2/2, ORF2/3, or ORF2t/3) that would permit packaging of the genetic element of a wild-type Anelloviridae family virus (e.g., Anellovirus) (e.g., as described herein). In some embodiments, a packaging deficient genetic element is packaged into a proteinaceous exterior at an efficiency less than 10% (e.g., less than 10%, 9%, 8%, 7%, 6%, 5%, 4%, 3%, 2%, 1%, 0.5%, 0.1%, 0.01%, or 0.001%) compared to a wild-type Anelloviridae family virus (e.g., Anellovirus) (e.g., as described herein), even in the presence of factors (e.g., ORF1, ORF1/1, ORF1/2, ORF2, ORF2/2, ORF2/3, or ORF2t/3) that would permit packaging of the genetic element of a wild-type Anelloviridae family virus (e.g., Anellovirus) (e.g., as described herein).

In some embodiments, a packaging competent genetic element can be packaged into a proteinaceous exterior (e.g., wherein the proteinaceous exterior comprises a capsid or a portion thereof, e.g., comprising a polypeptide encoded by an ORF1nucleic acid, e.g., as described herein). In some embodiments, a packaging competent genetic element is packaged into a proteinaceous exterior at an efficiency of at least 20% (e.g., at least 20%, 30%, 40%, 50%, 60%, 70%, 80%, 85%, 90%, 95%, 96%, 97%, 98%, 99%, 100%, or higher) compared to a wild-type Anelloviridae family virus (e.g., Anellovirus) (e.g., as described herein). In some embodiments, the packaging competent genetic element can be packaged into a proteinaceous exterior in the presence of factors (e.g., ORF1, ORF1/1, ORF1/2, ORF2, ORF2/2, ORF2/3, or ORF2t/3) that would permit packaging of the genetic element of a wild-type Anelloviridae family virus (e.g., Anellovirus) (e.g., as described herein). In some embodiments, a packaging competent genetic element is packaged into a proteinaceous exterior at an efficiency of at least 20% (e.g., at least 20%, 30%, 40%, 50%, 60%, 70%, 80%, 85%, 90%, 95%, 96%, 97%, 98%, 99%, 100%, or higher) compared to a wild-type Anelloviridae family virus (e.g., Anellovirus) (e.g., as described herein) in the presence of factors (e.g., ORF1, ORF1/1, ORF1/2, ORF2, ORF2/2, ORF2/3, or ORF2t/3) that would permit packaging of the genetic element of a wild-type Anelloviridae family virus (e.g., Anellovirus) (e.g., as described herein).

### Anelloviridae Family Viruses (e.g., Anelloviruses)

In some embodiments, an Anelloviridae family vector, e.g., as described herein, comprises sequences or expression products derived from an Anellovirus. In some embodiments, an Anelloviridae family vector includes one or more sequences or expression products that are exogenous relative to the Anellovirus. In some embodiments, an Anelloviridae family vector includes one or more sequences or expression products that are endogenous relative to the Anellovirus. In some embodiments, an Anelloviridae family vector includes one or more sequences or expression products that are heterologous relative to one or more other sequences or expression products in the Anelloviridae family vector. Anelloviridae family viruses (e.g., Anellovirus) generally have single-stranded circular DNA genomes with negative polarity.

It is understood that applicable embodiments described herein with respect to anellovectors may also be applied to Anelloviridae family vectors (e.g., a vector based on or derived from a chicken anemia virus (CAV), e.g., as described herein). Examples of chicken anemia viruses, and compositions and uses thereof, are described in PCT Publication No. WO/2022/094238, incorporated herein by reference in its entirety, including the sequences of Tables 1A and 1B therein.

In some embodiments, the genetic element comprises a nucleotide sequence encoding an amino acid sequence or a functional fragment thereof or a sequence having at least about 60%, 70% 80%, 85%, 90% 95%, 96%, 97%, 98%, 99%, or 100% sequence identity to any one of the amino acid sequences described herein, e.g., an Anellovirus amino acid sequence.

In some embodiments, an Anelloviridae family vector as described herein comprises one or more nucleic acid molecules (e.g., a genetic element as described herein) comprising a sequence having at least about 70%, 75%, 80%, 85%, 90%, 95%, 96%, 97%, 98%, 99%, or 100% sequence identity to an Anellovirus sequence, e.g., as described herein, or a fragment thereof. In embodiments, the Anelloviridae family vector comprises a nucleic acid sequence selected from a sequence as shown in any of Tables N1-N3, or a sequence having at least 70%, 75%, 80%, 85%, 90%, 95%, 96%, 97%, 98%, 99%, or 100% sequence identity thereto. In embodiments, the Anelloviridae family vector comprises a polypeptide comprising a sequence as shown in Table A1-A3, or a sequence having at least 70%, 75%, 80%, 85%, 90%, 95%, 96%, 97%, 98%, 99%, or 100% sequence identity thereto.

In some embodiments, an Anelloviridae family vector as described herein comprises one or more nucleic acid molecules (e.g., a genetic element as described herein) comprising a sequence having at least about 70%, 75%, 80%, 85%, 90%, 95%, 96%, 97%, 98%, 99%, or 100% sequence identity to one or more of a TATA box, cap site, initiator element, transcriptional start site, 5′ UTR conserved domain, ORF1, ORF1/1, ORF1/2, ORF2, ORF2/2, ORF2/3, ORF2t/3, three open-reading frame region, poly(A) signal, GC-rich region, or any combination thereof, of any of the Anelloviridae family viruses (e.g., Anellovirus) described herein (e.g., an Anelloviridae family virus (e.g., Anellovirus) sequence as annotated, or as encoded by a sequence listed, in any of Tables N1-N3. In some embodiments, the nucleic acid molecule comprises a sequence encoding a capsid protein, e.g., an ORF1, ORF1/1, ORF1/2, ORF2, ORF2/2, ORF2/3, or ORF2t/3sequence of any of the Anelloviruses described herein (e.g., an Anelloviridae family virus (e.g., Anellovirus) sequence as annotated, or as encoded by a sequence listed, in any of Tables N1-N3). In some embodiments, the nucleic acid molecule comprises a sequence encoding a capsid protein comprising an amino acid sequence having at least about 70%, 75%, 80%, 85%, 90%, 95%, 96%, 97%, 98%, 99%, or 100% sequence identity to an Anelloviridae family virus (e.g., Anellovirus) ORF1 or ORF2 protein (e.g., an ORF1 or ORF2 amino acid sequence as shown in Table A1-A3, or an ORF1 or ORF2 amino acid sequence encoded by a nucleic acid sequence as shown in any of Tables N1-N3). In embodiments, the nucleic acid molecule comprises a sequence encoding a capsid protein comprising an amino acid sequence having at least about 70%, 75%, 80%, 85%, 90%, 95%, 96%, 97%, 98%, 99%, or 100% sequence identity to an Anelloviridae family virus (e.g., Anellovirus) ORF1 protein (e.g., an ORF1 amino acid sequence as shown in Table A1-A3, or an ORF1 amino acid sequence encoded by a nucleic acid sequence as shown in any of Tables N1-N3).

### Nucleic Acid Sequences

In some embodiments, the nucleic acid molecule comprises a nucleic acid sequence having at least about 70%, 75%, 80%, 85%, 90%, 95%, 96%, 97%, 98%, 99%, or 100% sequence identity to the Anelloviridae family virus (e.g., Anellovirus) ORF1 nucleotide sequence of any of Tables N1-N3. In some embodiments, the nucleic acid molecule comprises a nucleic acid sequence having at least about 70%, 75%, 80%, 85%, 90%, 95%, 96%, 97%, 98%, 99%, or 100% sequence identity to the Anelloviridae family virus (e.g., Anellovirus) ORF2 nucleotide sequence of any of Tables N1-N3. In some embodiments, the nucleic acid molecule comprises a nucleic acid sequence having at least about 70%, 75%, 80%, 85%, 90%, 95%, 96%, 97%, 98%, 99%, or 100% sequence identity to the Anelloviridae family virus (e.g., Anellovirus) ORF3 nucleotide sequence of any of Tables N1-N3. In some embodiments, the nucleic acid molecule comprises a nucleic acid sequence having at least about 70%, 75%, 80%, 85%, 90%, 95%, 96%, 97%, 98%, 99%, or 100% sequence identity to the Anelloviridae family virus (e.g., Anellovirus) GC-rich region nucleotide sequence of any of Tables N1-N3. In some embodiments, the nucleic acid molecule comprises a nucleic acid sequence having at least about 70%, 75%, 80%, 85%, 90%, 95%, 96%, 97%, 98%, 99%, or 100% sequence identity to the Anelloviridae family virus (e.g., Anellovirus) 5′ UTR conserved domain nucleotide sequence of any of Tables N1-N3.

It is understood that Tables N1-N3 herein provide the positive strand sequence corresponding to a particular Anellovirus. However, as described herein, a genetic element is typically a negative strand (e.g., comprising the reverse complement of a nucleic acid sequence as listed in any of Tables N1-N3, or a portion thereof). Consequently, a 5′ UTR conserved domain of a genetic element as described herein may comprise the reverse complement of a sequence annotated as a 5′ UTR conserved domain (e.g., in any of Tables N1-N3). Consequently, a GC-rich region of a genetic element as described herein may comprise the reverse complement of a sequence annotated as a GC-rich region (e.g., in any of Tables N1-N3).

In some embodiments, the Anellovirus is an Alphatorquevirus. In some embodiments, the Anellovirus is a Betatorquevirus. In some embodiments, the Anellovirus is a Gammatorquevirus.

In some embodiments, the Anellovirus is a Ring1, Ring3.1, Ring4, Ring5.2, Ring6.0, Ring7, Ring9, Ring10, or Ring20 Anellovirus.

The Ring1 Anellovirus genomic sequence is disclosed as SEQ ID NO: 16 of International Application PCT/US2021/037076, and the corresponding amino acid sequences are disclosed in Table A2 of the same International Application, and the sequences are herein incorporated by reference in their entireties. In some embodiments, the genetic element comprises a nucleic acid sequence having at least about 70%, 75%, 80%, 85%, 90%, 95%, 96%, 97%, 98%, 99%, or 100% sequence identity to the reverse complement of the sequence annotated as the GC-rich region of SEQ ID NO: 16 of International Application PCT/US2021/037076. In some embodiments, the genetic element comprises a nucleic acid sequence having at least about 70%, 75%, 80%, 85%, 90%, 95%, 96%, 97%, 98%, 99%, or 100% sequence identity to the reverse complement of the sequence annotated as the 5′ UTR conserved domain nucleotide sequence of SEQ ID NO: 16 of International Application PCT/US2021/037076. The Ring3.1 Anellovirus genomic sequence is disclosed as SEQ ID NO: 878 of International Application PCT/US2022/015499, and the corresponding amino acid sequences are disclosed in Table B4 of the same International Application, and the sequences are herein incorporated by reference in their entireties. In some embodiments, the genetic element comprises a nucleic acid sequence having at least about 70%, 75%, 80%, 85%, 90%, 95%, 96%, 97%, 98%, 99%, or 100% sequence identity to the reverse complement of the sequence annotated as the GC-rich region nucleotide sequence of SEQ ID NO: 878 of International Application PCT/US2022/015499. In some embodiments, the genetic element comprises a nucleic acid sequence having at least about 70%, 75%, 80%, 85%, 90%, 95%, 96%, 97%, 98%, 99%, or 100% sequence identity to the reverse complement of the sequence annotated as the 5′ UTR conserved domain nucleotide sequence of SEQ ID NO: 878 of International Application PCT/US2022/015499. The Ring4 Anellovirus genomic sequence is disclosed as SEQ ID NO: 886 of International Application PCT/US2021/037076, and the corresponding amino acid sequences are disclosed in Table C2 of the same International Application, and the sequences are herein incorporated by reference in their entireties. In some embodiments, the genetic element comprises a nucleic acid sequence having at least about 70%, 75%, 80%, 85%, 90%, 95%, 96%, 97%, 98%, 99%, or 100% sequence identity to the reverse complement of the sequence annotated as the GC-rich region nucleotide sequence of SEQ ID NO: 886 of International Application PCT/US2021/037076. In some embodiments, the genetic element comprises a nucleic acid sequence having at least about 70%, 75%, 80%, 85%, 90%, 95%, 96%, 97%, 98%, 99%, or 100% sequence identity to the reverse complement of the sequence annotated as the 5′ UTR conserved domain nucleotide sequence of SEQ ID NO: 886 of International Application PCT/US2021/037076. The Ring5.2 Anellovirus genomic sequence is disclosed as SEQ ID NO: 894 of International Application PCT/US2022/015499, and the corresponding amino acid sequences are disclosed in Table D2 of the same International Application, and the sequences are herein incorporated by reference in their entireties. In some embodiments, the genetic element comprises a nucleic acid sequence having at least about 70%, 75%, 80%, 85%, 90%, 95%, 96%, 97%, 98%, 99%, or 100% sequence identity to the reverse complement of the sequence annotated as the GC-rich region nucleotide sequence of SEQ ID NO: 894 of International Application PCT/US2022/015499. In some embodiments, the genetic element comprises a nucleic acid sequence having at least about 70%, 75%, 80%, 85%, 90%, 95%, 96%, 97%, 98%, 99%, or 100% sequence identity to the reverse complement of the sequence annotated as the 5′ UTR conserved domain nucleotide sequence of SEQ ID NO: 894 of International Application PCT/US2022/015499. The Ring6.0 Anellovirus genomic sequence is disclosed as SEQ ID NO: 903 of International Application PCT/US2019/065995, and the corresponding amino acid sequences are disclosed in Table C4 of the same International Application, and the sequences are herein incorporated by reference in their entireties. In some embodiments, the genetic element comprises a nucleic acid sequence having at least about 70%, 75%, 80%, 85%, 90%, 95%, 96%, 97%, 98%, 99%, or 100% sequence identity to the reverse complement of the sequence annotated as the GC-rich region nucleotide sequence of SEQ ID NO: 903 of International Application PCT/US2019/065995. In some embodiments, the genetic element comprises a nucleic acid sequence having at least about 70%, 75%, 80%, 85%, 90%, 95%, 96%, 97%, 98%, 99%, or 100% sequence identity to the reverse complement of the sequence annotated as the 5′ UTR conserved domain nucleotide sequence of SEQ ID NO: 903 of International Application PCT/US2019/065995. The Ring7 Anellovirus genomic sequence is disclosed as SEQ ID NO: 911 of International Application PCT/US2019/065995, and the corresponding amino acid sequences are disclosed in Table C5 of the same International Application, and the sequences are herein incorporated by reference in their entireties. In some embodiments, the genetic element comprises a nucleic acid sequence having at least about 70%, 75%, 80%, 85%, 90%, 95%, 96%, 97%, 98%, 99%, or 100% sequence identity to the reverse complement of the sequence annotated as the GC-rich region nucleotide sequence of SEQ ID NO: 911 of International Application PCT/US2019/065995. In some embodiments, the genetic element comprises a nucleic acid sequence having at least about 70%, 75%, 80%, 85%, 90%, 95%, 96%, 97%, 98%, 99%, or 100% sequence identity to the reverse complement of the sequence annotated as the 5′ UTR conserved domain nucleotide sequence of SEQ ID NO: 911 of International Application PCT/US2019/065995. The Ring9 Anellovirus genomic sequence is disclosed as SEQ ID NO: 1001 of International Application PCT/US2022/015499, and the corresponding amino acid sequences are disclosed in Table F2 of the same International Application, and the sequences are herein incorporated by reference in their entireties. In some embodiments, the genetic element comprises a nucleic acid sequence having at least about 70%, 75%, 80%, 85%, 90%, 95%, 96%, 97%, 98%, 99%, or 100% sequence identity to the reverse complement of the sequence annotated as the GC-rich region nucleotide sequence of SEQ ID NO: 1001 of International Application PCT/US2022/015499. In some embodiments, the genetic element comprises a nucleic acid sequence having at least about 70%, 75%, 80%, 85%, 90%, 95%, 96%, 97%, 98%, 99%, or 100% sequence identity to the reverse complement of the sequence annotated as the 5′ UTR conserved domain nucleotide sequence of SEQ ID NO: 1001 of International Application PCT/US2022/015499. The Ring10 Anellovirus genomic sequence is disclosed as SEQ ID NO: 1008 of International Application PCT/US2022/015499, and the corresponding amino acid sequences are disclosed in Table F4 of the same International Application, and the sequences are herein incorporated by reference in their entireties. In some embodiments, the genetic element comprises a nucleic acid sequence having at least about 70%, 75%, 80%, 85%, 90%, 95%, 96%, 97%, 98%, 99%, or 100% sequence identity to the reverse complement of the sequence annotated as the GC-rich region nucleotide sequence of SEQ ID NO: 1008 of International Application PCT/US2022/015499. In some embodiments, the genetic element comprises a nucleic acid sequence having at least about 70%, 75%, 80%, 85%, 90%, 95%, 96%, 97%, 98%, 99%, or 100% sequence identity to the reverse complement of the sequence annotated as the 5′ UTR conserved domain nucleotide sequence of SEQ ID NO: 1008 of International Application PCT/US2022/015499. The Ring20 Anellovirus genomic sequence is disclosed as SEQ ID NO: 1014 of International Application PCT/US2022/015499, and the corresponding amino acid sequences are disclosed in Table F6 of the same International Application, and the sequences are herein incorporated by reference in their entireties. In some embodiments, the genetic element comprises a nucleic acid sequence having at least about 70%, 75%, 80%, 85%, 90%, 95%, 96%, 97%, 98%, 99%, or 100% sequence identity to the reverse complement of the sequence annotated as the GC-rich region nucleotide sequence of SEQ ID NO: 1014 of International Application PCT/US2022/015499. In some embodiments, the genetic element comprises a nucleic acid sequence having at least about 70%, 75%, 80%, 85%, 90%, 95%, 96%, 97%, 98%, 99%, or 100% sequence identity to the reverse complement of the sequence annotated as the 5′ UTR conserved domain nucleotide sequence of SEQ ID NO: 1014 of International Application PCT/US2022/015499.

### Amino Acid Sequences Encoded by Nucleic Acid Sequences

In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anelloviridae family virus (e.g., Anellovirus) ORF1 amino acid sequence of Table A1-A3. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anelloviridae family virus (e.g., Anellovirus) ORF2 amino acid sequence of Table A1-A3. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anelloviridae family virus (e.g., Anellovirus) ORF3 amino acid sequence of Table A1-A3. In some embodiments, the nucleic acid is a genetic element construct or a construct for providing the polypeptide (e.g., an ORF1 molecule and/or an ORF2 molecule) in trans.

### Proteins Comprising Amino Acid Sequences

In some embodiments, the Anelloviridae family vector described herein comprises an Anellovirus ORF or ORF molecule (e.g., an Anellovirus ORF1, ORF2, ORF2/2, ORF2/3, ORF1/1, or ORF1/2) includes a polypeptide comprising an amino acid sequence having at least 70%, 75%, 80%, 85%, 90%, 95%, 96%, 97%, 98%, 99%, or 100% sequence identity to a corresponding Anellovirus ORF sequence, e.g., as described herein). In embodiments, the Anelloviridae family vector described herein comprises a protein having an amino acid sequence having at least about 70%, 75%, 80%, 85%, 90%, 95%, 96%, 97%, 98%, 99%, or 100% sequence identity to the Anelloviridae family virus (e.g., Anellovirus) ORF1 amino acid sequence of Table A1-A3. In embodiments, the Anelloviridae family vector described herein comprises a protein having an amino acid sequence having at least about 70%, 75%, 80%, 85%, 90%, 95%, 96%, 97%, 98%, 99%, or 100% sequence identity to the Anelloviridae family virus (e.g., Anellovirus) ORF2 amino acid sequence of Table A1-A3. In embodiments, the Anelloviridae family vector described herein comprises a protein having an amino acid sequence having at least about 70%, 75%, 80%, 85%, 90%, 95%, 96%, 97%, 98%, 99%, or 100% sequence identity to the Anelloviridae family virus (e.g., Anellovirus) ORF3 amino acid sequence of Table A1-A3.

In some embodiments, an ORF1 molecule has an amino acid sequence having at least about 70%, 75%, 80%, 85%, 90%, 95%, 96%, 97%, 98%, 99%, or 100% sequence identity to the ORF1 molecule of Table A2 of International Application PCT/US2021/037076. In some embodiments, an ORF1 molecule has an amino acid sequence having at least about 70%, 75%, 80%, 85%, 90%, 95%, 96%, 97%, 98%, 99%, or 100% sequence identity to the ORF1 molecule of Table B4 of International Application PCT/US2022/015499. In some embodiments, an ORF1 molecule has an amino acid sequence having at least about 70%, 75%, 80%, 85%, 90%, 95%, 96%, 97%, 98%, 99%, or 100% sequence identity to the ORF1 molecule of Table C2 of International Application PCT/US2021/037076. In some embodiments, an ORF1 molecule has an amino acid sequence having at least about 70%, 75%, 80%, 85%, 90%, 95%, 96%, 97%, 98%, 99%, or 100% sequence identity to the ORF1 molecule of Table D2 of International Application PCT/US2022/015499. In some embodiments, an ORF1 molecule has an amino acid sequence having at least about 70%, 75%, 80%, 85%, 90%, 95%, 96%, 97%, 98%, 99%, or 100% sequence identity to the ORF1 molecule of Table C4 of International Application PCT/US2019/065995. In some embodiments, an ORF1 molecule has an amino acid sequence having at least about 70%, 75%, 80%, 85%, 90%, 95%, 96%, 97%, 98%, 99%, or 100% sequence identity to the ORF1 molecule of Table C5 of International Application PCT/US2019/065995. In some embodiments, an ORF1 molecule has an amino acid sequence having at least about 70%, 75%, 80%, 85%, 90%, 95%, 96%, 97%, 98%, 99%, or 100% sequence identity to the ORF1 molecule of Table F2 of International Application PCT/US2022/015499. In some embodiments, an ORF1 molecule has an amino acid sequence having at least about 70%, 75%, 80%, 85%, 90%, 95%, 96%, 97%, 98%, 99%, or 100% sequence identity to the ORF1 molecule of Table F4 of International Application PCT/US2022/015499. In some embodiments, an ORF1 molecule has an amino acid sequence having at least about 70%, 75%, 80%, 85%, 90%, 95%, 96%, 97%, 98%, 99%, or 100% sequence identity to the ORF1 molecule of Table F6 of International Application PCT/US2022/015499.

In some embodiments, an ORF1 molecule (e.g., comprised in the Anelloviridae family vector) comprises a polypeptide encoded by the Anelloviridae family virus (e.g., Anellovirus) ORF1 nucleic acid sequence of any of Tables N1-N3. In some embodiments, the ORF1 molecule (e.g., comprised in the Anelloviridae family vector) comprises an Anelloviridae family virus (e.g., Anellovirus) ORF1 protein of Table A1-A3 or a splice variant or post-translationally processed (e.g., proteolytically processed) variant thereof. In some embodiments, an ORF2 molecule (e.g., comprised in the Anelloviridae family vector) comprises a polypeptide encoded by the Anelloviridae family virus (e.g., Anellovirus) ORF2 nucleic acid sequence of any of Tables N1-N3. In some embodiments, the ORF2 molecule (e.g., comprised in the Anelloviridae family vector) comprises an Anelloviridae family virus (e.g., Anellovirus) ORF2 protein of Table A1-A3 or a splice variant or post-translationally processed (e.g., proteolytically processed) variant thereof. In some embodiments, an ORF3 molecule (e.g., comprised in the Anelloviridae family vector) comprises a polypeptide encoded by the Anelloviridae family virus (e.g., Anellovirus) ORF3 nucleic acid sequence of any of Tables N1-N3. In some embodiments, the ORF3 molecule (e.g., comprised in the Anelloviridae family vector) comprises an Anelloviridae family virus (e.g., Anellovirus) ORF3 protein of Table A1-A3 or a splice variant or post-translationally processed (e.g., proteolytically processed) variant thereof.

### Polypeptides Comprising Amino Acid Sequences

In some embodiments, the polypeptide described herein comprises an amino acid sequence having at least about 70%, 75%, 80%, 85%, 90%, 95%, 96%, 97%, 98%, 99%, or 100% sequence identity to an Anelloviridae family virus (e.g., Anellovirus) ORF1 amino acid sequence described herein. In embodiments, the polypeptide described herein comprises an amino acid sequence having at least about 70%, 75%, 80%, 85%, 90%, 95%, 96%, 97%, 98%, 99%, or 100% sequence identity to the Anelloviridae family virus (e.g., Anellovirus) ORF1 amino acid sequence of Table A1-A3.

In some embodiments, the polypeptide described herein comprises an amino acid sequence having at least about 70%, 75%, 80%, 85%, 90%, 95%, 96%, 97%, 98%, 99%, or 100% sequence identity to an ORF1 molecule encoded by an Anelloviridae family virus (e.g., Anellovirus) ORF1 nucleic acid described herein. In some embodiments, the polypeptide described herein comprises an amino acid sequence having at least about 70%, 75%, 80%, 85%, 90%, 95%, 96%, 97%, 98%, 99%, or 100% sequence identity to an ORF1 molecule encoded by an Anelloviridae family virus (e.g., Anellovirus) ORF1 nucleic acid as listed in Table N1-N3.

In some embodiments, the polypeptide described herein comprises an amino acid sequence having at least about 70%, 75%, 80%, 85%, 90%, 95%, 96%, 97%, 98%, 99%, or 100% sequence identity to an Anelloviridae family virus (e.g., Anellovirus) ORF2 amino acid sequence described herein. In embodiments, the polypeptide described herein comprises an amino acid sequence having at least about 70%, 75%, 80%, 85%, 90%, 95%, 96%, 97%, 98%, 99%, or 100% sequence identity to the Anelloviridae family virus (e.g., Anellovirus) ORF2 amino acid sequence of Table A1-A3.

In some embodiments, the polypeptide described herein comprises an amino acid sequence having at least about 70%, 75%, 80%, 85%, 90%, 95%, 96%, 97%, 98%, 99%, or 100% sequence identity to an ORF2 molecule encoded by an Anelloviridae family virus (e.g., Anellovirus) ORF2 nucleic acid described herein. In some embodiments, the polypeptide described herein comprises an amino acid sequence having at least about 70%, 75%, 80%, 85%, 90%, 95%, 96%, 97%, 98%, 99%, or 100% sequence identity to an ORF2 molecule encoded by an Anelloviridae family virus (e.g., Anellovirus) ORF2 nucleic acid as listed in Table N1-N3.

In some embodiments, the polypeptide described herein comprises an amino acid sequence having at least about 70%, 75%, 80%, 85%, 90%, 95%, 96%, 97%, 98%, 99%, or 100% sequence identity to an Anelloviridae family virus (e.g., Anellovirus) ORF3 amino acid sequence described herein. In embodiments, the polypeptide described herein comprises an amino acid sequence having at least about 70%, 75%, 80%, 85%, 90%, 95%, 96%, 97%, 98%, 99%, or 100% sequence identity to the Anelloviridae family virus (e.g., Anellovirus) ORF3 amino acid sequence of Table A1-A3.

In some embodiments, the polypeptide described herein comprises an amino acid sequence having at least about 70%, 75%, 80%, 85%, 90%, 95%, 96%, 97%, 98%, 99%, or 100% sequence identity to an ORF3 molecule encoded by an Anelloviridae family virus (e.g., Anellovirus) ORF3 nucleic acid described herein. In some embodiments, the polypeptide described herein comprises an amino acid sequence having at least about 70%, 75%, 80%, 85%, 90%, 95%, 96%, 97%, 98%, 99%, or 100% sequence identity to an ORF3 molecule encoded by an Anelloviridae family virus (e.g., Anellovirus) ORF3 nucleic acid as listed in Table N1-N3.

In some embodiments, the polypeptide comprises an amino acid sequence (e.g., an ORF1, ORF1/1, ORF1/2, ORF2, ORF2/2, ORF2/3, or ORF2t/3 sequence) as shown in Table A1-A3, or a sequence having at least 70%, 75%, 80%, 85%, 90%, 95%, 96%, 97%, 98%, 99%, or 100% sequence identity thereto.

Ring19 is an Anellovirus that was isolated from RPE cells. In some embodiments, a method described herein comprises delivering an Anellovector (e.g., an Anellovector having sequence similarity to Ring19) to eye tissue (e.g., to the eye of a subject), for example, to retinal tissue and/or RPE cells.

TABLE N1
Exemplary Anellovirus nucleic acid sequence (Betatorquevirus).

| | |
|---|---|
| Name | RING 19 |
| Genus/Clade | Betatorquevirus |
| Source tissue | Retinal pigment epithelium |
| Accession | N/A |

Full Sequence: 2876 bp
1 10 20 30 40 50
| | | | | |
CGGGAGCCGAAGGTGAGTGCAACCACCGTAGTCTAGGGGCAATTCGGGCT
AGTTCAGTATGGCGGAACGGGCAAGAAACTTAAATATTATTATTTTACAG
ATGCAAATACAACCACCTATTAGAACCTTCAAACAAACAATTTCAGATTG
GAAAAACTTAATTGTCCACGTTCACGACAACATTTGCAACTGCAATAAAC
CATTAGAACACACTATTGATACCTGTATCACCAATCCAGATGAATTAAGA
TTAAACAAATCTACTAAACAACAACTACAAAAATGCCTTGGTACCCCAGA
AGAAGATACCCAAGAAGACGTTATCGATGGCTTCGCAGATGGAGAGCTAG
ACGCCCTTTTCGCCCAAGATACAGAAGAAGATACTGGGTAAGAAACTATT
CTCGAAAGAGAAAACTATTTAAAATAACAACCAAAGAATGGCAACCAAAA
GTTATAAGAAAGACTCATGTAAAGGGCACCTATCCTTTGTTTCTTTGTAC
AAAGCACAGAATTAACAATAATATGATACAATATTTAGACTCTATAGCTC
CAGAACACTATTACGGAGGAGGAGGATTTTCAATAATGCAATTTTCCTTA
CAAGCCTTATATGAAGAATTTATAAAAGCAAAAAACTGGTGGACTAATAC
AAACTGCTTTTTACCACTTGTAAGATATATGGGTTGCTCATTCAAATTTT
ATAAAACTGAATTTTATGATTATATTGTACTAATTGAAAGATGTTATCCA
CTTGCTTGTACTGATGAAATGTACTTATCTACTCAACCTAGTATTATGAT
GCTTACAAGAAAATGTATTTTTGTACCATGCAAACAAAACAGCAAAGGTA
AAAAACCTTACAAAAAAGTTAGAGTAAGACCACCTTCACAAATGACTACA
GGATGGCATTTCTCACAAGACTTAGCAAACATGCCACTTGTAGTACTAAA
AACTTCAGTATGCAGCTTTGACAGATATTACACAGACAGTACAGCTAAAT
CAACCACAATAGGCTTTAAAACACTTAACACACAAACATTTAGATATCAT
GACTGGCAGGAACCACCTACAACAGGATACAAACCACAAAACCTACTATG
GTTTTATGGAGCAGAAAACGGATCACCAGTAGACCCCAACAACACAATAG
TATCAAACCTAATATACTTAGGAGGCACAGGACCTTATGAAAAAGGCACA
CCAATAAAAACAAACATAAGCAATTACTTTTCAGAGCCTAAACTGTGGGG
AAATATATTTCACGATGATTATACATCAGGAACATCACCCGTGTTTGTTA
CAAACAAATCACCATCAGAAATTAAAACCGCATGGAACACTATAAAAGAC
TTAACTGTTAAAGCTAGCGGTGTATTTACATTAAGAACAATTCCACTATG
GCTACCTTGCAGATACAACCCATTTGCAGACAAAGCAACCAACAACAAAA
TATGGCTAGTTTCTATACATTCAGACCACACAGAATGGAAACCAATAGAC
AATCCATTACTACAACGAACAGACCTTCCTTTATGGTTACTTGTATGGGG
TTGGCAAGATTGGCAGAAAAAAAACCAACAAACTTCACAACCTGATATTA
ATTATTTAACAGTAATATCTTCACCATATATATCATGCTACCCAAAATTA
GATTACTATGTGTTACTAGATGAAGGATTTTGGGAGGGTCACTCAACATA
CATAGAGTCAATTACAGACTCAGACAAAAAACACTGGTACCCTAAAAATA
GATTTCAAATAGAAACACTTAATCTAATAGCTAACACAGGTCCAGGAACT
GTAAAACTAAGAGAAAACCAAGCAGCAGAAGGTCACATGGTATATCGCTT
TAATTTTAAGCTTGGAGGATGTCCCGCACCGATGGAAAAAATATGTGACC
CTAGCAAACAATCCAAATATCCTATTCCCAATAACCAGCAACAAACAACT
TCGTTGCAGAGTCCAGAAAACCCAATTCAAACCTATCTCTACGACTTCGA
CGAAAGGAGGGGCCTACTTACAGAAAGAGCTACAAAAAGAATCAAACAAG
ATCACACATCTGAAAAAACTGTTTTGCCATTTACAGGAGCAGCAACAGAC
CTCCCCATACTCCAAACAACATCACAGGAGGAAAGCTCCTCGGAAGAAGA
AGAAGAGCAACAAGCGGAGAAGAAACTACTCCAGCTCCGAAGAAAGCAGC
ACCGACTCCGGGAGCGAATCCTCCAGCTATTAGACATACAAAATACATAA
TAAAACAAAGTACTGTAAAAATTGATATGTTTGGAGATACTCATGTACCT
AACCGTAGAATGACCCCAGAAGAATTTGAACAAGAACTAATTGTCGCTGG
TGTTTTTCGCAGACCTCCTTGTTACTATATAAAAGATAGACCTACTTATC
CTTATGTACCAAAACCTACTGATGAAAAATGTATGGTAAACTTTGACTTA
AACTTTCCTTAATAAACTACGCCTGCAAACTTTCACTCTCGGTGTCCATT
TATATAAGATAAAACTTAAATAAACATCCACCACTCTCCCAAATACGCAG
GCGCACAAGGGGGCTCCGCCCCCTTAAACCCCCAAGGGGGCTCCGCCCCC
TTAAACCCCCAAGGGGGCTCCGCCCCCTTACACCCCCTAATAAATATTCA
ACAGGAAAACCACCTAATTAGAATTGCCGACCACAAACCGTCACTTACTT
CTCCTTTTTGCACTTACTTCCTCTTTTACTTATTATTATTCATTACATTA
ATTAATAATCACTGTAATTCCGGGGAGGAGCTAACAATCTATATAACTAA
CTACACTTCCGAATGGCTGAGTTTATGCCGCCAGACGGAGACGGGATCAC
TTCAGTGACTCCAGGCTGAACTTGGG (SEQ ID NO: 1)
Annotations:

| Putative Domain | Base range |
|---|---|
| ORF1 | 283-2250 |
| ORF2 | 101-391 |
| ORF3 | 2277-2462 |
| GC-rich region | 2515-2615 |
| 5′ UTR Conserved Domain, or a portion | 1-71 |
| thereof | |

TABLE A1
Exemplary Anellovirus amino acid sequence (Betatorquevirus)
RING 19 (Betatorquevirus)

| | |
|---|---|
| ORF1 | MPWYPRRRYPRRRYRWLRRWRARRPFRPRYRRRYWVRNYSRKRKLFKITT |
| | KEWQPKVIRKTHVKGTYPLFLCTKHRINNNMIQYLDSIAPEHYYGGGGFS |
| | IMQFSLQALYEEFIKAKNWWTNTNCFLPLVRYMGCSFKFYKTEFYDYIVL |
| | IERCYPLACTDEMYLSTQPSIMMLTRKCIFVPCKQNSKGKKPYKKVRVRP |
| | PSQMTTGWHFSQDLANMPLVVLKTSVCSFDRYYTDSTAKSTTIGFKTLNT |
| | QTFRYHDWQEPPTTGYKPQNLLWFYGAENGSPVDPNNTIVSNLIYLGGTG |
| | PYEKGTPIKTNISNYFSEPKLWGNIFHDDYTSGTSPVFVINKSPSEIKTA |
| | WNTIKDLTVKASGVFTLRTIPLWLPCRYNPFADKATNNKIWLVSIHSDHT |
| | EWKPIDNPLLQRTDLPLWLLVWGWQDWQKKNQQTSQPDINYLTVISSPYI |
| | SCYPKLDYYVLLDEGFWEGHSTYIESITDSDKKHWYPKNRFQIETLNLIA |
| | NTGPGTVKLRENQAAEGHMVYRFNFKLGGCPAPMEKICDPSKQSKYPIPN |
| | NQQQTTSLQSPENPIQTYLYDFDERRGLLTERATKRIKQDHTSEKTVLPF |
| | TGAATDLPILQTTSQEESSSEEEEEQQAEKKLLQLRRKQHRLRERILQLL |
| | DIQNT (SEQ ID NO: 2) |
| ORF2 | MQIQPPIRTFKQTISDWKNLIVHVHDNICNCNKPLEHTIDTCITNPDELR |
| | LNKSTKQQLQKCLGTPEEDTQEDVIDGFADGELDALFAQDTEEDTG |
| | (SEQ ID NO: 173) |
| ORF3 | MFGDTHVPNRRMTPEEFEQELIVAGVFRRPPCYYIKDRPTYPYVPKPTDE |
| | KCMVNFDLNFP (SEQ ID NO: 4) |

TABLE N2
Exemplary Anellovirus nucleic acid sequence (Betatorquevirus).

| | |
|---|---|
| Name | Ring2 |
| Genus/Clade | Betatorquevirus |
| Accession Number | JX134045.1 |

Full Sequence: 2797 bp
1 10 20 30 40 50
| | | | | |
TAATAAATATTCAACAGGAAAACCACCTAATTTAAATTGCCGACCACAAA
CCGTCACTTAGTTCCCCTTTTTGCAACAACTTCTGCTTTTTTCCAACTGC
CGGAAAACCACATAATTTGCATGGCTAACCACAAACTGATATGCTAATTA
ACTTCCACAAAACAACTTCCCCTTTTAAAACCACACCTACAAATTAATTA
TTAAACACAGTCACATCCTGGGAGGTACTACCACACTATAATACCAAGTG
CACTTCCGAATGGCTGAGTTTATGCCGCTAGACGGAGAACGCATCAGTTA
CTGACTGCGGACTGAACTTGGGCGGGTGCCGAAGGTGAGTGAAACCACCG
AAGTCAAGGGGCAATTCGGGCTAGTTCAGTCTAGCGGAACGGGCAAGAAA
CTTAAAATTATTTTATTTTTCAGATGAGCGACTGCTTTAAACCAACATGC
TACAACAACAAAACAAAGCAAACTCACTGGATTAATAACCTGCATTTAAC
CCACGACCTGATCTGCTTCTGCCCAACACCAACTAGACACTTATTACTAG
CTTTAGCAGAACAACAAGAAACAATTGAAGTGTCTAAACAAGAAAAAGAA
AAAATAACAAGATGCCTTATTACTACAGAAGAAGACGGTACAACTACAGA
CGTCCTAGATGGTATGGACGAGGTTGGATTAGACGCCCTTTTCGCAGAAG
ATTTCGAAGAAAAAGAAGGGTAAGACCTACTTATACTACTATTCCTCTAA
AGCAATGGCAACCGCCATATAAAAGAACATGCTATATAAAAGGACAAGAC
TGTTTAATATACTATAGCAACTTAAGACTGGGAATGAATAGTACAATGTA
TGAAAAAAGTATTGTACCTGTACATTGGCCGGGAGGGGGTTCTTTTTCTG
TAAGCATGTTAACTTTAGATGCCTTGTATGATATACATAAACTTTGTAGA
AACTGGTGGACATCCACAAACCAAGACTTACCACTAGTAAGATATAAAGG
ATGCAAAATAACATTTTATCAAAGCACATTTACAGACTACATAGTAAGAA
TACATACAGAACTACCAGCTAACAGTAACAAACTAACATACCCAAACACA
CATCCACTAATGATGATGATGTCTAAGTACAAACACATTATACCTAGTAG
ACAAACAAGAAGAAAAAAGAAACCATACACAAAAATATTTGTAAAACCAC
CTCCGCAATTTGAAAACAAATGGTACTTTGCTACAGACCTCTACAAAATT
CCATTACTACAAATACACTGCACAGCATGCAACTTACAAAACCCATTTGT
AAAACCAGACAAATTATCAAACAATGTTACATTATGGTCACTAAACACCA
TAAGCATACAAAATAGAAACATGTCAGTGGATCAAGGACAATCATGGCCA
TTTAAAATACTAGGAACACAAAGCTTTTATTTTTACTTTTACACCGGAGC
AAACCTACCAGGTGACACAACACAAATACCAGTAGCAGACCTATTACCAC
TAACAAACCCAAGAATAAACAGACCAGGACAATCACTAAATGAGGCAAAA
ATTACAGACCATATTACTTTCACAGAATACAAAAACAAATTTACAAATTA
TTGGGGTAACCCATTTAATAAACACATTCAAGAACACCTAGATATGATAC
TATACTCACTAAAAAGTCCAGAAGCAATAAAAAACGAATGGACAACAGAA
AACATGAAATGGAACCAATTAAACAATGCAGGAACAATGGCATTAACACC
ATTTAACGAGCCAATATTCACACAAATACAATATAACCCAGATAGAGACA
CAGGAGAAGACACTCAATTATACCTACTCTCTAACGCTACAGGAACAGGA
TGGGACCCACCAGGAATTCCAGAATTAATACTAGAAGGATTTCCACTATG
GTTAATATATTGGGGATTTGCAGACTTTCAAAAAAACCTAAAAAAAGTAA
CAAACATAGACACAAATTACATGTTAGTAGCAAAAACAAAATTTACACAA
AAACCTGGCACATTCTACTTAGTAATACTAAATGACACCTTTGTAGAAGG
CAATAGCCCATATGAAAAACAACCTTTACCTGAAGACAACATTAAATGGT
ACCCACAAGTACAATACCAATTAGAAGCACAAAACAAACTACTACAAACT
GGGCCATTTACACCAAACATACAAGGACAACTATCAGACAATATATCAAT
GTTTTATAAATTTTACTTTAAATGGGGAGGAAGCCCACCAAAAGCAATTA
ATGTTGAAAATCCTGCCCACCAGATTCAATATCCCATACCCCGTAACGAG
CATGAAACAACTTCGTTACAGAGTCCAGGGGAAGCCCCAGAATCCATCTT
ATACTCCTTCGACTATAGACACGGGAACTACACAACAACAGCTTTGTCAC
GAATTAGCCAAGACTGGGCACTTAAAGACACTGTTTCTAAAATTACAGAG
CCAGATCGACAGCAACTGCTCAAACAAGCCCTCGAATGCCTGCAAATCTC
GGAAGAAACGCAGGAGAAAAAAGAAAAAGAAGTACAGCAGCTCATCAGCA
ACCTCAGACAGCAGCAGCAGCTGTACAGAGAGCGAATAATATCATTATTA
AAGGACCAATAACTTTTAACTGTGTAAAAAAGGTGAAATTGTTTGATGAT
AAACCAAAAAACCGTAGATTTACACCTGAGGAATTTGAAACTGAGTTACA
AATAGCAAAATGGTTAAAGAGACCCCCAAGATCCTTTGTAAATGATCCTC
CCTTTTACCCATGGTTACCACCTGAACCTGTTGTAAACTTTAAGCTTAAT
TTTACTGAATAAAGGCCAGCATTAATTCACTTAAGGAGTCTGTTTATTTA
AGTTAAACCTTAATAAACGGTCACCGCCTCCCTAATACGCAGGCGCAGAA
AGGGGGCTCCGCCCCCTTTAACCCCCAGGGGGCTCCGCCCCCTGAAACCC
CCAAGGGGGCTACGCCCCCTTACACCCCC (SEQ ID NO: 54)
Annotations:

| Putative Domain | Base range |
|---|---|
| TATA Box | 237-243 |
| Cap Site | 260-267 |
| Transcriptional Start Site | 267 |
| 5′ UTR Conserved Domain | 323-393 |
| ORF2 | 424-723 |
| ORF2/2 | 424-719; 2274-2589 |
| ORF2/3 | 424-719; 2449-2812 |
| ORF1 | 612-2612 |
| ORF1/1 | 612-719; 2274-2612 |
| ORF1/2 | 612-719; 2449-2589 |
| Three open-reading frame region | 2441-2586 |
| Poly(A) Signal | 2808-2813 |
| GC-rich region | 2868-2929 |

TABLE A2
Exemplary Anellovirus amino acid sequences (Betatorquevirus)
Ring2 (Betatorquevirus)

| | |
|---|---|
| ORF2 | MSDCFKPTCYNNKTKQTHWINNLHLTHDLICFCPTPTRHLLLALAEQQETIEVSKQE |
| | KEKITRCLITTEEDGTTTDVLDGMDEVGLDALFAEDFEEKEG (SEQ ID NO: 55) |
| ORF2/2 | MSDCFKPTCYNNKTKQTHWINNLHLTHDLICFCPTPTRHLLLALAEQQETIEVSKQE |
| | KEKITRCLITTEEDGTTTDVLDGMDEVGLDALFAEDFEEKEGFNIPYPVTSMKQLRY |
| | RVQGKPQNPSYTPSTIDTGTTQQQLCHELAKTGHLKTLFLKLQSQIDSNCSNKPSNA |
| | CKSRKKRRRKKKKKYSSSSATSDSSSSCTESE (SEQ ID NO: 56) |
| ORF2/3 | MSDCFKPTCYNNKTKQTHWINNLHLTHDLICFCPTPTRHLLLALAEQQETIEVSKQE |
| | KEKITRCLITTEEDGTTTDVLDGMDEVGLDALFAEDFEEKEGARSTATAQTSPRMP |
| | ANLGRNAGEKRKRSTAAHQQPQTAAAAVQRANNIIIKGPITFNCVKKVKLFDDKPK |
| | NRRFTPEEFETELQIAKWLKRPPRSFVNDPPFYPWLPPEPVVNFKLNFTE (SEQ ID |
| | NO: 57) |
| ORF1 | MPYYYRRRRYNYRRPRWYGRGWIRRPFRRRFRRKRRVRPTYTTIPLKQWQPPYKR |
| | TCYIKGQDCLIYYSNLRLGMNSTMYEKSIVPVHWPGGGSFSVSMLTLDALYDIHKL |
| | CRNWWTSTNQDLPLVRYKGCKITFYQSTFTDYIVRIHTELPANSNKLTYPNTHPLM |
| | MMMSKYKHIIPSRQTRRKKKPYTKIFVKPPPQFENKWYFATDLYKIPLLQIHCTACN |
| | LQNPFVKPDKLSNNVTLWSLNTISIQNRNMSVDQGQSWPFKILGTQSFYFYFYTGA |
| | NLPGDTTQIPVADLLPLTNPRINRPGQSLNEAKITDHITFTEYKNKFTNYWGNPENK |
| | HIQEHLDMILYSLKSPEAIKNEWTTENMKWNQLNNAGTMALTPFNEPIFTQIQYNP |
| | DRDTGEDTQLYLLSNATGTGWDPPGIPELILEGFPLWLIYWGFADFQKNLKKVTNID |
| | TNYMLVAKTKFTQKPGTFYLVILNDTFVEGNSPYEKQPLPEDNIKWYPQVQYQLEA |
| | QNKLLQTGPFTPNIQGQLSDNISMFYKFYFKWGGSPPKAINVENPAHQIQYPIPRNE |
| | HETTSLQSPGEAPESILYSFDYRHGNYTTTALSRISQDWALKDTVSKITEPDRQQLLK |
| | QALECLQISEETQEKKEKEVQQLISNLRQQQQLYRERIISLLKDQ (SEQ ID NO: 58) |
| ORF1/1 | MPYYYRRRRYNYRRPRWYGRGWIRRPFRRRFRRKRRIQYPIPRNEHETTSLQSPGE |
| | APESILYSFDYRHGNYTTTALSRISQDWALKDTVSKITEPDRQQLLKQALECLQISEE |
| | TQEKKEKEVQQLISNLRQQQQLYRERIISLLKDQ (SEQ ID NO: 59) |
| ORF1/2 | MPYYYRRRRYNYRRPRWYGRGWIRRPFRRRFRRKRRSQIDSNCSNKPSNACKSRK |
| | KRRRKKKKKYSSSSATSDSSSSCTESE (SEQ ID NO: 60) |

TABLE N3
Exemplary Anellovirus nucleic acid sequence (Alphatorquevirus).

| | |
|---|---|
| Name | Ring18 |
| Genus/Clade | Alphatorquevirus |
| Source tissue | |
| Accession Number | N/A |

Full Sequence: 3733 bp

| | |
|---|---|
| 1 | CACGTGACTC CCGCAGGCCA ACCAGAGTCT ACGTCGTGCA CTTCCTGGGC ATGGTCTACA |
| 61 | TCATAATATA AGAAGGCGCA CTTCCGAATG GCTGAGTTTT CCACGCCCGT CCGCAGCGAG |
| 121 | AACGCCACGG AGGGAGATCC TCGCGTCCCG AGGGCGGGTG CCGGAGGTGA GTTTACACAC |
| 181 | CGCAGTCAAG GGGCAATTCG GGCTCGGGAC TGGCCGGGCC CCGGGCAAGG CTCTTAAAAA |
| 241 | ATGCGCTTTC GCAGGGTTGC TGAGAAAAGG AAAGTGCTTC TGCAAACTGT GCGAGCTGCA |
| 301 | GAGAAGACTA GGCGGCTTCT AGGTATGTGG CAGCCCCCCG CGCACAATGT CCCCGGCATC |
| 361 | GAGAGAAACT GGTACGAGAG CTGTTTTCGA TCCCATGCTG CTGTTTGCGG CTGTGGCGAC |
| 421 | TTTGTTGGCC ATCTTAGTTA TCTGGCAACT ACTCTGGGTC GTCCTCCGCG TCCTGGGCCC |
| 481 | CCAGGCGGAC CCCGCACACC GCAGATAAGA AACCTGCCAG CGCTCCCGGC GCCCCAGGGC |
| 541 | GAGCCCGGTG ACAGAGCGCC ATGGCGTGGG GCTTCTGGGG CCGACGCCGC CGGTGGAGAC |
| 601 | GGTGGAGACC ACGGCGCAGA CGGTGGAGAC CCCGCAGACG TAGGAGACGA CGCCCTGCTC |
| 661 | GCCGCTTTCG AGCTCGTCGA AGAGTAAGGA GGCGCGGGGG GCGGTGGCGC AGACGCTACA |
| 721 | GAAAATGGCG ACGGGGCAGA CGCAGACGAA CTCACAGAAA AAAGATAGTC ATAAAACAGT |
| 781 | GGCAACCAAA CTTTATAAGA CGCTGCTACA TCATAGGGTA CCTACCTTTA ATATTCTGTG |
| 841 | GCGAAAACAC AACCGCCCAG AACTATGCCA CTCACTCAGA CGACATGATA AGCAAAGGAC |
| 901 | CATACGGGGG GGGCATGACT ACCACAAAAT TTACTCTGAG AATACTGTAC GACGAGTTTA |
| 961 | CCAGGTTTAT GAACTTTTGG ACTGTTAGTA ACGAAGACCT AGACCTGTGT AGATACGTGG |
| 1021 | GCTGCAAACT CATATTTTTT AAACATCCCA CAGTGGACTT TATAGTACAG ATAAACACTC |
| 1081 | AGCCTCCTTT CTTAGACACG CACCTTACCG CGGCCAGCAT ACACCCGGGC ATCATGATGC |
| 1141 | TCAGCAAGAG ACGCATACTA ATACCCTCTC TAAAAACCCG GCCAAGCAGA AAACACAGGG |
| 1201 | TGGTTGTTAG GGTGGGCGCC CCAAGACTTT TTCAGGACAA GTGGTACCCC CAGTCAGACC |
| 1261 | TGTGTGACAC AGTTCTGCTT TCCATATTTG CAACCGCCTG TGACTTGCAA TATCCGTTCG |
| 1321 | GCTCACCACT AACTGACAAC CCTTGCGTCA ACTTCCAGAT TCTGGGGCCC CAGTACAAAA |
| 1381 | AACACCTTAG TATTAGCTCT ACTATGGATA CAACTAACAA ACAGCACTAT GACAGCAATT |
| 1441 | TGTTTAACCA AACTCAGCTA TACAACACCT TTCAAACTAT AGCTCAGCTT AAAGAGACAG |
| 1501 | GACAAACTGC AAACATATCT CCTAGTTGGA GTGCAGTGCA AAATAATATG GCCCTTAGTA |
| 1561 | ATACCGGTGA AAATGCAACC CAAAGCAAAG ACACTTGGTA CAAAGGAAAC ACATACAACA |
| 1621 | ACCACATTAC AACGTTAGCA CAAAAAACCA GAGAAAGATT TAAAGGTGCA ACAAAAGCAG |
| 1681 | CACTACAAAA CTACCCCACC ATAATGTCCA CAGACTTATA TGAATACCAC TCAGGCATAT |
| 1741 | ACTCCAGCAT ATTTCTATCA GCTGGCAGGA GCTACTTTGA AACCACCGGG GCCTACTCTG |
| 1801 | ACATTATATA CAACCCTTTC ACAGACAAAG GCACAGGCAA CATAATCTGG ATAGACTACC |
| 1861 | TCACAAAAGA AGACACCATT TTTGTAAAAA ACAAAAGCAA ATGCGAAATA ATGGACATGC |
| 1921 | CCCTGTGGGC GGCCTGCACA GGATATACAG AGTTCTGTGC AAAGTATACA GGAGACTCTG |
| 1981 | CCATTATTTA CAATGCCAGA GTACTAATAA GATGCCCGTA CACTGAACCC ATGCTAATAG |
| 2041 | ACCACTCAGA CCCGAACAAA GGCTTCGTAC CCTACTCATT TAACTTTGGC AACGGAAAAA |
| 2101 | TGCCCGGAGG CAGCTCCAAC GTACCCATAA GAATGAGAGC CAAATGGTAC GTGAACATAT |
| 2161 | TCCACCAAAA AGAGGTTCTA GAGACTATAG TACAGAGCGG ACCGTTCGGG TACAAGGGCG |
| 2221 | ACATAAAATC AGCTGTACTG GCCATGAAAT ACAGATTTCA CTGGAAATGG GGTGGAAACC |
| 2281 | CTATATCCAA ACAGGTCGTC AGGAATCCCT GCTCCAACTC CAGCTCCTCC GCGGCCCATA |
| 2341 | GAGGACCTCG CAGCGTACAA GCAGTTGACC CGAAATACAA TACCCCAGAG GTCACGTGGC |
| 2401 | ACTCGTGGGA CATCAGACGA GGACTCTTTG GCAAAGCAGG TATTAAAAGA ATGCAACAAG |
| 2461 | AATCAGATGC TCTTTACATT CCTCCAGGAC CATTCAAGAG ACCTCGCAGA GACACGAACG |
| 2521 | CCCAAGACCC AGAAGAGCAA AACGAAAGCT CAGGTTTCAG AGTCCAGCAG CGACTCCCGT |
| 2581 | GGGTCCACTC CAGCCAAGAG ACGCAAAGCT CCCAAGAAGA AACGGAGGCG CAGGGGTCGG |
| 2641 | TACAAGACCA ACTACTCCTC CAGCTCCGAG AGCAGCGAGT ACTCCGACTC CAGCTCCAGC |
| 2701 | AACTCGCAAC CCAAGTCCTC AAAGTCCAAG CAGGGCACGG CATACACCCC CTATTATCTT |
| 2761 | CCCAAGCGTA AACAAAGTCT TTATGTTTGA GCCCCACGGT CCTAAACCCA TACAGGGCTA |
| 2821 | CAACGATTGG CTAGAGGAGT ACACTGCTTG TAAATTCTGG GACAGACCCC CAAGAAAGCT |
| 2881 | ACACACAGAC TTACCCTTTT ACCCCTGGGC ACCAAAACCC CAAGACCAAG TCAGGGTAAG |
| 2941 | CTTTAAACTC AACTTTCAAT AAAAATTCTA GGCCGTGGGA GTTTCACTTG TCGGTGTCTG |
| 3001 | CTTCTTAAGG TCGCCAAGCA CTCCGAGCGC CAGCGAGGAG TGCGACCCCC CCTCCGGTAG |
| 3061 | CAACGCCTTC GGAGCCGCGC GCTACGCCTT CGGCTGCGCG CGGCACCTCA GACCCCCCCT |
| 3121 | CCACCCGAAA CGCTTGCGCG TTTCGGACCT TCGGCGTCGG GGGGGTCGGG AGCTTTATTA |
| 3181 | AACAGACTCC GAGTTGCCAT TGGACACTGG AGCTGTGAAT CAGTAACGAA AGTGAGTGGG |
| 3241 | GCCAGACTTC GCCATAGGGC CTTTATCTTC TCGCCATTGG ATAGTGTCCG GGGTCGCCGT |
| 3301 | AGGCTTCGGC CTCGTTTTTA GGCCTTCCGG ACTACAAAAA TGGCGAACTT GGTGACGTCA |
| 3361 | CGGCCGCCAT TTTAAGTAAG GCGGAAGCAG CTCCACTTTC TCACAAAATG GCGGCGGAGC |
| 3421 | ACTTCCGGCT TGCCCAAAAT AGCGGGCAAG CTCTTCCGGG TCAAAGGTCA GCAGCTACGT |
| 3481 | CACAAGTCAC CTGACTGAGG AGGAGCTAAA ACCCGGAAGT CCTCCTCGGT CACGTGGCTA |
| 3541 | GTCACGTGAC TACTACGTCA TCGGCGCCAT CTTGTGTGAC AAAATGGCGG ACAACTTCCG |
| 3601 | CTTTTTTGAA AAAAGGCGCG AAAAAACGGC GGCGGCGGCG CGCGCGCTGC GCGCGCGCGC |
| 3661 | CGAGGGGGCG CCAGCGCCCC CACTGTGCGG TCCCCCGCGG GGCTCCGGCC CCCCCCCGAA |
| 3721 | GTCCGTCACT AAC (SEQ ID NO: 213) |

Annotations

| Putative Domain | Base range |
|---|---|
| TATA Box | 67-71 |
| Cap Site | 88-95 |
| Transcriptional Start Site | 95 |
| 5′ UTR Conserved Domain | 155-225 |
| ORF2 | 325-687 |
| TAIP | 347-508 |
| ORF2/2 | 325-683, 2295-2790 |
| ORF2/3 | 325-683, 2488-2962 |
| ORF1 | 561-2771 |
| ORF1/1 | 561-683, 2295-2771 |
| ORF1/2 | 561-683, 2488-2790 |
| Three open-reading frame region | 2447-2771 |
| Poly(A) Signal | 2958-2963 |
| GC-rich region | 3627-3718 |

TABLE A3
Exemplary Anellovirus amino acid sequences for Ring 18 (Alphatorquevirus)

| | |
|---|---|
| ORF1 | MAWGFWGRRRRWRRWRPRRRRWRPRRRRRRRPARRFRARRRVRRRGGR |
| | WRRRYRKWRRGRRRRTHRKKIVIKQWQPNFIRRCYIIGYLPLIFCGENTTA |
| | QNYATHSDDMISKGPYGGGMTTTKFTLRILYDEFTRFMNFWTVSNEDLDL |
| | CRYVGCKLIFFKHPTVDFIVQINTQPPFLDTHLTAASIHPGIMMLSKRRILIPS |
| | LKTRPSRKHRVVVRVGAPRLFQDKWYPQSDLCDTVLLSIFATACDLQYPF |
| | GSPLTDNPCVNFQILGPQYKKHLSISSTMDTTNKQHYDSNLFNQTQLYNTF |
| | QTIAQLKETGQTANISPSWSAVQNNMALSNTGENATQSKDTWYKGNTYN |
| | NHITTLAQKTRERFKGATKAALQNYPTIMSTDLYEYHSGIYSSIFLSAGRSY |
| | FETTGAYSDIIYNPFTDKGTGNIIWIDYLTKEDTIFVKNKSKCEIMDMPLWA |
| | ACTGYTEFCAKYTGDSAIIYNARVLIRCPYTEPMLIDHSDPNKGFVPYSFNF |
| | GNGKMPGGSSNVPIRMRAKWYVNIFHQKEVLETIVQSGPFGYKGDIKSAV |
| | LAMKYRFHWKWGGNPISKQVVRNPCSNSSSSAAHRGPRSVQAVDPKYNT |
| | PEVTWHSWDIRRGLFGKAGIKRMQQESDALYIPPGPFKRPRRDTNAQDPEE |
| | QNESSGFRVQQRLPWVHSSQETQSSQEETEAQGSVQDQLLLQLREQRVLR |
| | LQLQQLATQVLKVQAGHGIHPLLSSQA (SEQ ID NO: 1100) |
| ORF1/1 | MAWGFWGRRRRWRRWRSRRRRWRPRRRRRRRPARRFRARRRVVRNPCS |
| | NSSSSAAHRGPRSVQAVDPKYNTPEVTWHSWDIRRGLFGKAGIKRMQQES |
| | DALYIPPGPFKRPRRDTNAQDPEEQNESSGFRVQQRLPWVHSSQETQSSQE |
| | ETEAQGSVQDQLLLQLREQRVLRLQLQQLATQVLKVQAGHGIHPLLSSQA |
| | (SEQ ID NO: 214) |
| ORF1/2 | MAWGFWGRRRRWRRWRPRRRRWRPRRRRRRRPARRFRARRRDHSRDLA |
| | ETRTPKTQKSKTKAQVSESSSDSRGSTPAKRRKAPKKKRRRRGRYKTNYSS |
| | SSESSEYSDSSSSNSQPKSSKSKQGTAYTPYYLPKRKQSLYV (SEQ ID NO: |
| | 215) |
| ORF2 | MWQPPAHNVPGIERNWYESCFRSHAAVCGCGDFVGHLSYLATTLGRPPRP |
| | GPPGGPRTPQIRNLPALPAPQGEPGDRAPWRGASGADAAGGDGGDHGAD |
| | GGDPADVGDDALLAAFELVEE (SEQ ID NO: 216) |
| ORF2/2 | MWQPPAHNVPGIERNWYESCFRSHAAVCGCGDFVGHLSYLATTLGRPPRP |
| | GPPGGPRTPQIRNLPALPAPQGEPGDRAPWRGASGADAAGGDGGDHGAD |
| | GGDPADVGDDALLAAFELVEESSGIPAPTPAPPRPIEDLAAYKQLTRNTIPQ |
| | RSRGTRGTSDEDSLAKQVLKECNKNQMLFTFLQDHSRDLAETRTPKTQKS |
| | KTKAQVSESSSDSRGSTPAKRRKAPKKKRRRRGRYKTNYSSSSESSEYSDS |
| | SSSNSQPKSSKSKQGTAYTPYYLPKRKQSLYV (SEQ ID NO: 217) |
| ORF2/3 | MWQPPAHNVPGIERNWYESCFRSHAAVCGCGDFVGHLSYLATTLGRPPRP |
| | GPPGGPRTPQIRNLPALPAPQGEPGDRAPWRGASGADAAGGDGGDHGAD |
| | GGDPADVGDDALLAAFELVEETIQETSQRHERPRPRRAKRKLRFQSPAATP |
| | VGPLQPRDAKLPRRNGGAGVGTRPTTPPAPRAASTPTPAPATRNPSPQSPSR |
| | ARHTPPIIFPSVNKVFMFEPHGPKPIQGYNDWLEEYTACKFWDRPPRKLHT |
| | DLPFYPWAPKPQDQVRVSFKLNFQ (SEQ ID NO: 218) |

### Capsid Proteins (e.g., ORF1 Molecules)

In some embodiments, the anellovector comprises an ORF1 molecule and/or a nucleic acid encoding an ORF1 molecule.

Generally, an ORF1 molecule comprises a polypeptide having the structural features and/or activity of an Anellovirus ORF1 protein (e.g., an Anellovirus ORF1 protein as described herein, e.g., as listed in Table A1-A3), or a functional fragment thereof. In some embodiments, the ORF1 molecule comprises a truncation relative to an Anellovirus ORF1 protein (e.g., an Anellovirus ORF1 protein as described herein, e.g., as listed in Table A1-A3). In some embodiments, the ORF1 molecule is truncated by at least 10, 20, 30, 40, 50, 60, 70, 80, 90, 100, 150, 200, 250, 300, 350, 400, 450, 500, 550, 600, 650, or 700 amino acids of the Anellovirus ORF1 protein. In some embodiments, an ORF1 molecule comprises an amino acid sequence having at least 70%, 75%, 80%, 85%, 90%, 95%, 96%, 97%, 98%, 99%, or 100% sequence identity to an Anellovirus ORF1 protein sequence as shown in Table A1-A3. In some embodiments, an ORF1 molecule comprises an amino acid sequence having at least 75%, 80%, 85%, 90%, 95%, 96%, 97%, 98%, or 99% sequence identity to an Betatorquevirus ORF1 protein, e.g., as described herein. An ORF1 molecule can generally bind to a nucleic acid molecule, such as DNA (e.g., a genetic element, e.g., as described herein). In some embodiments, an ORF1 molecule localizes to the nucleus of a cell. In certain embodiments, an ORF1 molecule localizes to the nucleolus of a cell. In some embodiments, an ORF1 molecule is encoded by an ORF1 nucleic acid. In some embodiments, the ORF1 nucleic acid comprises an antisense strand, which can be directly transcribed to produce mRNA encoding the ORF1 molecule. In some embodiments, the ORF1 nucleic acid comprises a sense strand.

In some embodiments, an ORF1 molecule as described herein comprises an amino acid sequence (e.g., an ORF1 sequence, or an arginine-rich region, jelly-roll domain, HVR, N22, or C-terminal domain sequence) as listed in any of Tables A2, A4, A6, A8, A10, A12, C1-C5, 2, 4, 6, 8, 10, 12, 14, 16, 18, 20-37, or D1-D10 of PCT Publication No. WO2020/123816 (incorporated herein by reference in its entirety), or a sequence having at least 70% 80%, 85%, 90% 95%, 96%, 97%, 98% and 99% nucleotide sequence identity thereto.

Without wishing to be bound by theory, an ORF1 molecule may be capable of binding to other ORF1 molecules, e.g., to form a proteinaceous exterior (e.g., as described herein). Such an ORF1 molecule may be described as having the capacity to form a capsid. In some embodiments, the proteinaceous exterior may encapsidate a nucleic acid molecule (e.g., a genetic element as described herein). In some embodiments, a plurality of ORF1 molecules may form a multimer, e.g., to produce a proteinaceous exterior. In some embodiments, the multimer may be a homomultimer. In other embodiments, the multimer may be a heteromultimer (e.g., comprising a plurality of distinct ORF1 molecules). It is also contemplated that an ORF1 molecule may have replicase activity.

An ORF1 molecule may, in some embodiments, comprise one or more of: a first region comprising an arginine rich region, e.g., a region having at least 60% basic residues (e.g., at least 60%, 65%, 70%, 75%, 80%, 85%, 90%, 95%, or 100% basic residues; e.g., between 60%-90%, 60%-80%, 70%-90%, or 70-80% basic residues), and a second region comprising jelly-roll domain, e.g., at least six beta strands (e.g., 4, 5, 6, 7, 8, 9, 10, 11, or 12 beta strands).

### Arginine-Rich Region

An arginine rich region (e.g., comprised an ORF1 molecule as described herein) has at least 70% (e.g., at least about 70, 80, 90, 95, 96, 97, 98, 99, or 100%) sequence identity to an arginine-rich region sequence described herein or a sequence of at least about 40 amino acids comprising at least 60%, 70%, or 80% basic residues (e.g., arginine, lysine, or a combination thereof).

In some embodiments, an ORF1 molecule as described herein comprises a deletion or truncation of an arginine-rich region. In some embodiments, the entire arginine-rich region is deleted. In some embodiments, a portion of the arginine-rich region (e.g., a N-terminal portion of the structural arginine-rich region) is deleted. In embodiments, the ORF1 molecule does not comprise an Anellovirus ORF1 arginine-rich region, or an amino acid sequence having at least 70%, 75%, 80%, 8%, 90%, 95%, 96%, 97%, 98%, or 99% sequence identity thereto.

In some embodiments, an ORF1 molecule having a deletion or truncation of the arginine-rich region further comprises a deletion or truncation of at least a portion of a C-terminal domain (e.g., as described herein).

### Jelly Roll Domain

A jelly-roll domain or region (e.g., comprised an ORF1 molecule as described herein) comprises (e.g., consists of) a polypeptide (e.g., a domain or region comprised in a larger polypeptide) comprising one or more (e.g., 1, 2, or 3) of the following characteristics:
(i) at least 30% (e.g., at least 30%, 35%, 40%, 45%, 50%, 55%, 60%, 65%, 70%, 75%, 80%, 90%, or more) of the amino acids of the jelly-roll domain are part of one or more β-sheets;
(ii) the secondary structure of the jelly-roll domain comprises at least four (e.g., at least 4, 5, 6, 7, 8, 9, 10, 11, or 12) β-strands; and/or
(iii) the tertiary structure of the jelly-roll domain comprises at least two (e.g., at least 2, 3, or 4) (3-sheets; and/or
(iv) the jelly-roll domain comprises a ratio of β-sheets to α-helices of at least 2:1, 3:1, 4:1, 5:1, 6:1, 7:1, 8:1, 9:1, or 10:1.

In certain embodiments, a jelly-roll domain comprises two β-sheets.

In certain embodiments, one or more (e.g., 1, 2, 3, 4, 5, 6, 7, 8, 9, or 10) of the β-sheets comprises about eight (e.g., 4, 5, 6, 7, 8, 9, 10, 11, or 12) β-strands. In certain embodiments, one or more (e.g., 1, 2, 3, 4, 5, 6, 7, 8, 9, or 10) of the β-sheets comprises eight β-strands. In certain embodiments, one or more (e.g., 1, 2, 3, 4, 5, 6, 7, 8, 9, or 10) of the β-sheets comprises seven β-strands. In certain embodiments, one or more (e.g., 1, 2, 3, 4, 5, 6, 7, 8, 9, or 10) of the β-sheets comprises six β-strands. In certain embodiments, one or more (e.g., 1, 2, 3, 4, 5, 6, 7, 8, 9, or 10) of the β-sheets comprises five β-strands. In certain embodiments, one or more (e.g., 1, 2, 3, 4, 5, 6, 7, 8, 9, or 10) of the β-sheets comprises four β-strands.

In some embodiments, the jelly-roll domain comprises a first β-sheet in antiparallel orientation to a second β-sheet. In certain embodiments, the first β-sheet comprises about four (e.g., 3, 4, 5, or 6) 0-strands. In certain embodiments, the second β-sheet comprises about four (e.g., 3, 4, 5, or 6) β-strands. In embodiments, the first and second β-sheet comprise, in total, about eight (e.g., 6, 7, 8, 9, 10, 11, or 12) β-strands.

In certain embodiments, a jelly-roll domain is a component of a capsid protein (e.g., an ORF1 molecule as described herein). In certain embodiments, a jelly-roll domain has self-assembly activity. In some embodiments, a polypeptide comprising a jelly-roll domain binds to another copy of the polypeptide comprising the jelly-roll domain. In some embodiments, a jelly-roll domain of a first polypeptide binds to a jelly-roll domain of a second copy of the polypeptide.

### Other Subdomains

An ORF1 molecule may also include a third region comprising the structure or activity of an Anellovirus N22 domain (e.g., as described herein, e.g., an N22 domain from an Anellovirus ORF1 protein as described herein).

An ORF1 molecule may also include a fourth region comprising the structure or activity of an Anellovirus C-terminal domain (CTD) (e.g., as described herein, e.g., a CTD from an Anellovirus ORF1 protein as described herein).

In some embodiments, an ORF1 molecule as described herein comprises a deletion or truncation of a C-terminal domain (CTD). In some embodiments, the entire CTD is deleted. In some embodiments, a portion of the CTD (e.g., a C-terminal portion of the CTD) is deleted. In embodiments, the ORF1 molecule does not comprise an Anellovirus ORF1 CTD, or an amino acid sequence having at least 70%, 75%, 80%, 8%, 90%, 95%, 96%, 97%, 98%, or 99% sequence identity thereto. In some embodiments, an ORF1 molecule having a deletion or truncation of the CTD further comprises a deletion or truncation of at least a portion of an arginine-rich region (e.g., as described herein).

In some embodiments, the ORF1 molecule comprises, in N-terminal to C-terminal order, the first, second, third, and fourth regions.

The ORF1 molecule may, in some embodiments, further comprise a hypervariable region (HVR), e.g., an HVR from an Anellovirus ORF1 protein, e.g., as described herein. In some embodiments, the HVR is positioned between the second region and the third region. In some embodiments, the HVR comprises at least about 55 (e.g., at least about 45, 50, 51, 52, 53, 54, 55, 56, 57, 58, 59, 60, or 65) amino acids (e.g., about 45-160, 50-160, 55-160, 60-160, 45-150, 50-150, 55-150, 60-150, 45-140, 50-140, 55-140, or 60-140 amino acids).

In some embodiments, the first region can bind to a nucleic acid molecule (e.g., DNA). In some embodiments, the basic residues are selected from arginine, histidine, or lysine, or a combination thereof. In some embodiments, the first region comprises at least 60%, 65%, 70%, 75%, 80%, 85%, 90%, 95%, or 100% arginine residues (e.g., between 60%-90%, 60%-80%, 70%-90%, or 70-80% arginine residues). In some embodiments, the first region comprises about 30-120 amino acids (e.g., about 40-120, 40-100, 40-90, 40-80, 40-70, 50-100, 50-90, 50-80, 50-70, 60-100, 60-90, or 60-80 amino acids). In some embodiments, the first region comprises the structure or activity of a viral ORF1 arginine-rich region (e.g., an arginine-rich region from an Anellovirus ORF1 protein, e.g., as described herein). In some embodiments, the first region comprises a nuclear localization signal.

In some embodiments, the second region comprises a jelly-roll domain, e.g., the structure or activity of a viral ORF1 jelly-roll domain (e.g., a jelly-roll domain from an Anellovirus ORF1 protein, e.g., as described herein). In some embodiments, the second region is capable of binding to the second region of another ORF1 molecule, e.g., to form a proteinaceous exterior (e.g., capsid) or a portion thereof.

In some embodiments, the fourth region is exposed on the surface of a proteinaceous exterior (e.g., a proteinaceous exterior comprising a multimer of ORF1 molecules, e.g., as described herein).

In some embodiments, the first region, second region, third region, fourth region, and/or HVR each comprise fewer than four (e.g., 0, 1, 2, or 3) beta sheets.

In some embodiments, one or more of the first region, second region, third region, fourth region, and/or HVR may be replaced by a heterologous amino acid sequence (e.g., the corresponding region from a heterologous ORF1 molecule). In some embodiments, the heterologous amino acid sequence has a desired functionality, e.g., as described herein.

In some embodiments, the ORF1 molecule comprises a plurality of conserved motifs (e.g., motifs comprising about 5, 6, 7, 8, 9, 10, 11, 12, 13, 14, 15, 16, 17, 18, 19, 20, 25, 30, 35, 40, 45, 50, 60, 70, 80, 90, 100, or more amino acids). In some embodiments, the conserved motifs may show 60, 70, 80, 85, 90, 95, or 100% sequence identity to an ORF1 protein of one or more wild-type Anellovirus clades (e.g., Betatorquevirus). In some embodiments, the conserved motifs each have a length between 1-1000 (e.g., between 5-10, 5-15, 5-20, 10-15, 10-20, 15-20, 5-50, 5-100, 10-50, 10-100, 10-1000, 50-100, 50-1000, or 100-1000) amino acids. In certain embodiments, the conserved motifs consist of about 2-4% (e.g., about 1-8%, 1-6%, 1-5%, 1-4%, 2-8%, 2-6%, 2-5%, or 2-4%) of the sequence of the ORF1 molecule, and each show 100% sequence identity to the corresponding motifs in an ORF1 protein of the wild-type Anellovirus clade. In certain embodiments, the conserved motifs consist of about 5-10% (e.g., about 1-20%, 1-10%, 5-20%, or 5-10%) of the sequence of the ORF1 molecule, and each show 80% sequence identity to the corresponding motifs in an ORF1 protein of the wild-type Anellovirus clade. In certain embodiments, the conserved motifs consist of about 10-50% (e.g., about 10-20%, 10-30%, 10-40%, 10-50%, 20-40%, 20-50%, or 30-50%) of the sequence of the ORF1 molecule, and each show 60% sequence identity to the corresponding motifs in an ORF1 protein of the wild-type Anellovirus clade.

In some embodiments, an ORF1 molecule comprises at least one difference (e.g., a mutation, chemical modification, or epigenetic alteration) relative to a wild-type ORF1 protein, e.g., as described herein (e.g., as shown in Table A1-A3).

### Conserved ORF1 Motif in N22 Domain

In some embodiments, a polypeptide (e.g., an ORF1 molecule) described herein comprises the amino acid sequence YNPX²DXGX²N (SEQ ID NO: 829), wherein X″ is a contiguous sequence of any n amino acids. For example, X² indicates a contiguous sequence of any two amino acids. In some embodiments, the YNPX²DXGX²N (SEQ ID NO: 829) is comprised within the N22 domain of an ORF1 molecule, e.g., as described herein. In some embodiments, a genetic element described herein comprises a nucleic acid sequence (e.g., a nucleic acid sequence encoding an ORF1 molecule, e.g., as described herein) encoding the amino acid sequence YNPX²DXGX²N (SEQ ID NO: 829), wherein X″ is a contiguous sequence of any n amino acids.

In some embodiments, a polypeptide (e.g., an ORF1 molecule) comprises a conserved secondary structure, e.g., flanking and/or comprising a portion of the YNPX²DXGX²N (SEQ ID NO: 829) motif, e.g., in an N22 domain. In some embodiments, the conserved secondary structure comprises a first beta strand and/or a second beta strand. In some embodiments, the first beta strand is about 5-6 (e.g., 3, 4, 5, 6, 7, or 8) amino acids in length. In some embodiments, the first beta strand comprises the tyrosine (Y) residue at the N-terminal end of the YNPX²DXGX²N (SEQ ID NO: 829) motif. In some embodiments, the YNPX²DXGX²N (SEQ ID NO: 829) motif comprises a random coil (e.g., about 8-9 amino acids of random coil). In some embodiments, the second beta strand is about 7-8 (e.g., 5, 6, 7, 8, 9, or 10) amino acids in length. In some embodiments, the second beta strand comprises the asparagine (N) residue at the C-terminal end of the YNPX²DXGX²N (SEQ ID NO: 829) motif.

### Exemplary ORF1 Sequences

In some embodiments, a polypeptide (e.g., an ORF1 molecule) described herein comprises an amino acid sequence having at least about 70%, 75%, 80%, 85%, 90%, 95%, 96%, 97%, 98%, 99%, or 100% sequence identity to one or more Anellovirus ORF1 subsequences, e.g., as described herein). In some embodiments, an Anelloviridae family vector (e.g., anellovector) described herein comprises an ORF1 molecule comprising an amino acid sequence having at least about 70%, 75%, 80%, 85%, 90%, 95%, 96%, 97%, 98%, 99%, or 100% sequence identity to one or more Anellovirus ORF1 subsequences, e.g., as described herein. In some embodiments, an anellovector described herein comprises a nucleic acid molecule (e.g., a genetic element) encoding an ORF1 molecule comprising an amino acid sequence having at least about 70%, 75%, 80%, 85%, 90%, 95%, 96%, 97%, 98%, 99%, or 100% sequence identity to one or more Anellovirus ORF1 subsequences, e.g., as described herein.

In some embodiments, the one or more Anellovirus ORF1 subsequences comprises one or more of an arginine (Arg)-rich domain, a jelly-roll domain, a hypervariable region (HVR), an N22 domain, or a C-terminal domain (CTD) (e.g., as listed herein), or sequences having at least about 70%, 75%, 80%, 85%, 90%, 95%, 96%, 97%, 98%, 99%, or 100% sequence identity thereto. In some embodiments, the ORF1 molecule comprises a plurality of subsequences from different Anelloviruses. In some embodiments, the ORF1 molecule comprises one or more of an Arg-rich domain, a jelly-roll domain, an N22 domain, and a CTD from one Anelloviridae family virus (e.g., Anellovirus), and an HVR from another. In some embodiments, the ORF1 molecule comprises one or more of a jelly-roll domain, an HVR, an N22 domain, and a CTD from one Anelloviridae family virus (e.g., Anellovirus), and an Arg-rich domain from another. In some embodiments, the ORF1 molecule comprises one or more of an Arg-rich domain, an HVR, an N22 domain, and a CTD from one Anelloviridae family virus (e.g., Anellovirus), and a jelly-roll domain from another. In some embodiments, the ORF1 molecule comprises one or more of an Arg-rich domain, a jelly-roll domain, an HVR, and a CTD from one Anelloviridae family virus (e.g., Anellovirus), and an N22 domain from another. In some embodiments, the ORF1 molecule comprises one or more of an Arg-rich domain, a jelly-roll domain, an HVR, and an N22 domain from one Anelloviridae family virus (e.g., Anellovirus), and a CTD from another.

### Identification of ORF1 Protein Sequences

In some embodiments, an ORF1 protein sequence, or a nucleic acid sequence encoding an ORF1 protein, can be identified from the genome of an Anelloviridae family virus, e.g., an Anellovirus (e.g., a putative Anelloviridae family virus genome identified, for example, by nucleic acid sequencing techniques, e.g., deep sequencing techniques). In some embodiments, an ORF1 protein sequence is identified by one or more (e.g., 1, 2, or all 3) of the following selection criteria:
(i) Length Selection: Protein sequences (e.g., putative ORF1 sequences passing the criteria described in (ii) or (iii) below) may be size-selected for those greater than about 600 amino acid residues to identify putative ORF1 proteins. In some embodiments, an ORF1 protein sequence is at least about 600, 650, 700, 750, 800, 850, 900, 950, or 1000 amino acid residues in length. In some embodiments, an Alphatorquevirus ORF1 protein sequence is at least about 700, 710, 720, 730, 740, 750, 760, 770, 780, 790, 800, 900, or 1000 amino acid residues in length. In some embodiments, a Betatorquevirus ORF1 protein sequence is at least about 650, 660, 670, 680, 690, 700, 750, 800, 900, or 1000 amino acid residues in length. In some embodiments, a Gammatorquevirus ORF1 protein sequence is at least about 650, 660, 670, 680, 690, 700, 750, 800, 900, or 1000 amino acid residues in length. In some embodiments, a nucleic acid sequence encoding an ORF1 protein is at least about 1800, 1900, 2000, 2100, 2200, 2300, 2400, or 2500 nucleotides in length. In some embodiments, a nucleic acid sequence encoding an Alphatorquevirus ORF1 protein sequence is at least about 2100, 2150, 2200, 2250, 2300, 2400, or 2500 nucleotides in length. In some embodiments, a nucleic acid sequence encoding a Betatorquevirus ORF1 protein sequence is at least about 1900, 1950, 2000, 2500, 2100, 2150, 2200, 2250, 2300, 2400, or 2500 or 1000 nucleotides in length. In some embodiments, a nucleic acid sequence encoding a Gammatorquevirus ORF1 protein sequence is at least about 1900, 1950, 2000, 2500, 2100, 2150, 2200, 2250, 2300, 2400, or 2500 or 1000 nucleotides in length.
(ii) Presence of ORF1 motif Protein sequences (e.g., putative ORF1 sequences passing the criteria described in (i) above or (iii) below) may be filtered to identify those that contain the conserved ORF1 motif in the N22 domain described above. In some embodiments, a putative Anellovirus ORF1 sequence comprises the sequence YNPXXDXGXXN (SEQ ID NO: 829). In some embodiments, a putative Anellovirus ORF1 sequence comprises the sequence Y[NCS]PXXDX[GASKR]XX[NTSVAK].
(iii) Presence of arginine-rich region: Protein sequences (e.g., putative ORF1 sequences passing the criteria described in (i) and/or (ii) above) may be filtered for those that include an arginine-rich region (e.g., as described herein). In some embodiments, a putative ORF1 sequence comprises a contiguous sequence of at least about 30, 35, 40, 45, 50, 55, 60, 65, or 70 amino acids that comprises at least 30% (e.g., at least about 20%, 25%, 30%, 35%, 40%, 45%, or 50%) arginine residues. In some embodiments, a putative ORF1 sequence comprises a contiguous sequence of about 35-40, 40-45, 45-50, 50-55, 55-60, 60-65, or 65-70 amino acids that comprises at least 30% (e.g., at least about 20%, 25%, 30%, 35%, 40%, 45%, or 50%) arginine residues. In some embodiments, the arginine-rich region is positioned at least about 30, 40, 50, 60, 70, or 80 amino acids downstream of the start codon of the putative ORF1 protein. In some embodiments, the arginine-rich region is positioned at least about 50 amino acids downstream of the start codon of the putative ORF1 protein.

In some embodiments, an ORF1 protein is identified in an Anellovirus genome sequence as described in Example 36 of PCT Publication No. WO2020/123816 (incorporated herein by reference in its entirety).

### ORF2 Molecules

In some embodiments, the anellovector comprises an ORF2 molecule and/or a nucleic acid encoding an ORF2 molecule. Generally, an ORF2 molecule comprises a polypeptide having the structural features and/or activity of an Anellovirus ORF2 protein (e.g., an Anellovirus ORF2 protein as described herein, e.g., as listed in Table A1-A3)-, or a functional fragment thereof. In some embodiments, an ORF2 molecule comprises an amino acid sequence having at least 70%, 75%, 80%, 85%, 90%, 95%, 96%, 97%, 98%, 99%, or 100% sequence identity to an Anellovirus ORF2 protein sequence as shown in Table A1-A3. In some embodiments, an ORF2 molecule is encoded by an ORF2 nucleic acid. In some embodiments, the ORF2 nucleic acid comprises an antisense strand, which can be directly transcribed to produce mRNA encoding the ORF2 molecule. In some embodiments, the ORF2 nucleic acid comprises a sense strand.

In some embodiments, an ORF2 molecule comprises an amino acid sequence having at least 75%, 80%, 85%, 90%, 95%, 96%, 97%, 98%, or 99% sequence identity to an Alphatorquevirus, Betatorquevirus, or Gammatorquevirus ORF2 protein. In some embodiments, an ORF2 molecule (e.g., an ORF2 molecule having at least 75%, 80%, 85%, 90%, 95%, 96%, 97%, 98%, or 99% sequence identity to an Alphatorquevirus ORF2 protein) has a length of 250 or fewer amino acids (e.g., about 150-200 amino acids). In some embodiments, an ORF2 molecule (e.g., an ORF2 molecule having at least 75%, 80%, 85%, 90%, 95%, 96%, 97%, 98%, or 99% sequence identity to a Betatorquevirus ORF2 protein) has a length of about 50-150 amino acids. In some embodiments, an ORF2 molecule (e.g., an ORF2 molecule having at least 75%, 80%, 85%, 90%, 95%, 96%, 97%, 98%, or 99% sequence identity to a Gammatorquevirus ORF2 protein) has a length of about 100-200 amino acids (e.g., about 100-150 amino acids). In some embodiments, the ORF2 molecule comprises a helix-turn-helix motif (e.g., a helix-turn-helix motif comprising two alpha helices flanking a turn region). In some embodiments, the ORF2 molecule does not comprise the amino acid sequence of the ORF2 protein of TTV isolate TA278 or TTV isolate SANBAN. In some embodiments, an ORF2 molecule has protein phosphatase activity. In some embodiments, an ORF2 molecule comprises at least one difference (e.g., a mutation, chemical modification, or epigenetic alteration) relative to a wild-type ORF2 protein, e.g., as described herein (e.g., as shown in Table A1-A3).

### Conserved ORF2 Motif

In some embodiments, a polypeptide (e.g., an ORF2 molecule) described herein comprises the amino acid sequence [W/F]X⁷HX³CX¹CX⁵H (SEQ ID NO: 949), wherein X″ is a contiguous sequence of any n amino acids. In embodiments, X⁷ indicates a contiguous sequence of any seven amino acids. In some embodiments, X³ indicates a contiguous sequence of any three amino acids. In some embodiments, X¹ indicates any single amino acid. In some embodiments, X⁵ indicates a contiguous sequence of any five amino acids. In some embodiments, the [W/F] can be either tryptophan or phenylalanine. In some embodiments, the [W/F]X⁷HX³CX¹CX⁵H (SEQ ID NO: 949) is comprised within the N22 domain of an ORF2 molecule, e.g., as described herein. In some embodiments, a genetic element described herein comprises a nucleic acid sequence (e.g., a nucleic acid sequence encoding an ORF2 molecule, e.g., as described herein) encoding the amino acid sequence [W/F]X⁷HX³CX¹CX⁵H (SEQ ID NO: 949), wherein X″ is a contiguous sequence of any n amino acids.

### Genetic Elements

In some embodiments, the Anelloviridae family vector (e.g., anellovector) comprises a genetic element. In some embodiments, the genetic element has one or more of the following characteristics: is substantially non-integrating with a host cell's genome, is an episomal nucleic acid, is a single stranded DNA, is circular, is about 1 to 10 kb, exists within the nucleus of the cell, can be bound by endogenous proteins, produces an effector, such as a polypeptide or nucleic acid (e.g., an RNA, iRNA, microRNA) that targets a gene, activity, or function of a host or target cell. In one embodiment, the genetic element is a substantially non-integrating DNA. In some embodiments, the genetic element comprises a packaging signal, e.g., a sequence that binds a capsid protein. In some embodiments, outside of the packaging or capsid-binding sequence, the genetic element has less than 70%, 60%, 50%, 40%, 30%, 20%, 10%, 5% sequence identity to a wild type Anellovirus nucleic acid sequence, e.g., has less than 70%, 60%, 50%, 40%, 30%, 20%, 10%, 5% sequence identity to an Anellovirus nucleic acid sequence, e.g., as described herein. In some embodiments, outside of the packaging or capsid-binding sequence, the genetic element has less than 500, 450, 400, 350, 300, 250, 200, 150, or 100 contiguous nucleotides that are at least 70%, 75%, 80%, 8%, 90%, 95%, 96%, 97%, 98%, 99%, or 100% identical to an Anellovirus nucleic acid sequence. In certain embodiments, the genetic element is a circular, single stranded DNA that comprises a promoter sequence, a sequence encoding a therapeutic effector, and a capsid binding protein. In some embodiments, the genetic element may comprise other sequences that include DNA, RNA, or artificial nucleic acids.

In some embodiments, the genetic element has at least about 70%, 75%, 80%, 8%, 90%, 95%, 96%, 97%, 98%, 99%, or 100% sequence identity to an Anellovirus nucleic acid sequence, e.g., as described herein (e.g., as described in any of Tables N1-N3), or a fragment thereof, or encodes an amino acid sequence having at least about 70%, 75%, 80%, 8%, 90%, 95%, 96%, 97%, 98%, 99%, or 100% sequence identity to an Anellovirus amino acid sequence (e.g., as described in any of Tables A1-A3), or a fragment thereof. In some embodiments, the genetic element comprises a sequence encoding an effector (e.g., an endogenous effector or an exogenous effector, e.g., a payload), e.g., a polypeptide effector (e.g., a protein) or nucleic acid effector (e.g., a non-coding RNA, e.g., a miRNA, siRNA, mRNA, lncRNA, RNA, DNA, an antisense RNA, gRNA).

In some embodiments, the genetic element has a length less than 20 kb (e.g., less than about 19 kb, 18 kb, 17 kb, 16 kb, 15 kb, 14 kb, 13 kb, 12 kb, 11 kb, 10 kb, 9 kb, 8 kb, 7 kb, 6 kb, 5 kb, 4 kb, 3 kb, 2 kb, 1 kb, or less). In some embodiments, the genetic element has, independently or in addition to, a length greater than 1000b (e.g., at least about 1.1 kb, 1.2 kb, 1.3 kb, 1.4 kb, 1.5 kb, 1.6 kb, 1.7 kb, 1.8 kb, 1.9 kb, 2 kb, 2.1 kb, 2.2 kb, 2.3 kb, 2.4 kb, 2.5 kb, 2.6 kb, 2.7 kb, 2.8 kb, 2.9 kb, 3 kb, 3.1 kb, 3.2 kb, 3.3 kb, 3.4 kb, 3.5 kb, 3.6 kb, 3.7 kb, 3.8 kb, 3.9 kb, 4 kb, 4.1 kb, 4.2 kb, 4.3 kb, 4.4 kb, 4.5 kb, 4.6 kb, 4.7 kb, 4.8 kb, 4.9 kb, 5 kb, or greater). In some embodiments, the genetic element has a length of about 2.5-4.6, 2.8-4.0, 3.0-3.8, or 3.2-3.7 kb. In some embodiments, the genetic element has a length of about 1.5-2.0, 1.5-2.5, 1.5-3.0, 1.5-3.5, 1.5-3.8, 1.5-3.9, 1.5-4.0, 1.5-4.5, or 1.5-5.0 kb. In some embodiments, the genetic element has a length of about 2.0-2.5, 2.0-3.0, 2.0-3.5, 2.0-3.8, 2.0-3.9, 2.0-4.0, 2.0-4.5, or 2.0-5.0 kb. In some embodiments, the genetic element has a length of about 2.5-3.0, 2.5-3.5, 2.5-3.8, 2.5-3.9, 2.5-4.0, 2.5-4.5, or 2.5-5.0 kb. In some embodiments, the genetic element has a length of about 3.0-5.0, 3.5-5.0, 4.0-5.0, or 4.5-5.0 kb. In some embodiments, the genetic element has a length of about 1.5-2.0, 2.0-2.5, 2.5-3.0, 3.0-3.5, 3.1-3.6, 3.2-3.7, 3.3-3.8, 3.4-3.9, 3.5-4.0, 4.0-4.5, or 4.5-5.0 kb.

In some embodiments, the genetic element comprises one or more of the features described herein, e.g., a sequence encoding a substantially non-pathogenic protein, a protein binding sequence, one or more sequences encoding a regulatory nucleic acid, one or more regulatory sequences, one or more sequences encoding a replication protein, and other sequences. In some embodiments, the substantially non-pathogenic protein comprises an amino acid sequence or a functional fragment thereof or a sequence having at least about 60%, 65%, 70%, 75%, 80%, 85%, 90% 95%, 96%, 97%, 98%, 99%, or 100% sequence identity to any one of the amino acid sequences described herein, an Anellovirus amino acid sequence, e.g., as listed in any of Tables A1-A3.

In some embodiments, the double-stranded circular DNA and/or the genetic element does not comprise one or more plasmid elements (e.g., an origin of replication or a selectable marker, e.g., a resistance gene). In some embodiments, the double-stranded circular DNA and/or the genetic element does not comprise a plasmid backbone. In some embodiments, the double-stranded circular DNA and/or the genetic element does not comprise one or more bacterial plasmid elements (e.g., a bacterial origin of replication or a selectable marker, e.g., a bacterial resistance gene). In some embodiments, the double-stranded circular DNA and/or the genetic element does not comprise a bacterial plasmid backbone. In some embodiments, the double-stranded circular DNA and/or the genetic element does not comprise one or more mammalian plasmid elements (e.g., a mammalian origin of replication or a selectable marker, e.g., a mammalian resistance gene). In some embodiments, the double-stranded circular DNA and/or the genetic element does not comprise a mammalian plasmid backbone. In some embodiments, the double-stranded circular DNA and/or the genetic element does not comprise one or more insect plasmid elements (e.g., an insect origin of replication or a selectable marker, e.g., an insect resistance gene). In some embodiments, the double-stranded circular DNA and/or the genetic element does not comprise an insect plasmid backbone.

In some embodiments, a genetic element as described herein comprises a sequence (e.g., a TATA box, cap site, transcriptional start site, 5′ UTR, open reading frame (ORF), poly(A) signal, or GC-rich region sequence) as listed in any of Tables A1, A3, A5, A7, A9, A11, B1-B5, 1, 3, 5, 7, 9, 11, 13, 15, or 17 of PCT Publication No. WO2020/123816 (incorporated herein by reference in its entirety), or a sequence having at least 70% 80%, 85%, 90% 95%, 96%, 97%, 98% and 99% nucleotide sequence identity thereto.

In some embodiments, a genetic element comprises a sequence encoding an effector (e.g., an exogenous effector). In some embodiments, the effector-encoding sequence is inserted into an Anellovirus genome sequence (e.g., as described herein). In some embodiments, the effector-encoding sequence replaces a contiguous sequence (e.g., of at least 5, 10, 15, 20, 25, 30, 40, 50, 60, 70, 80, 90, 100, 200, 300, 400, 500, 600, 700, 800, 900, 1000, or more nucleotides) from the Anellovirus genome sequence. In some embodiments, the effector-encoding sequence replaces a TATA box, cap site, transcriptional start site, 5′ UTR, open reading frame (ORF), poly(A) signal, or GC-rich region sequence, or a portion thereof (e.g., a portion consisting of at least 5, 10, 15, 20, 25, 30, 40, 50, 60, 70, 80, 90, 100, 200, 300, 400, 500, 600, 700, 800, 900, 1000, or more nucleotides), e.g., as described herein, or a sequence having at least 70% 80%, 85%, 90% 95%, 96%, 97%, 98% and 99% nucleotide sequence identity thereto.

In some embodiments, the sequence of a first nucleic acid element comprised in a genetic element (e.g., a TATA box, cap site, transcriptional start site, 5′ UTR, open reading frame (ORF), poly(A) signal, or GC-rich region) overlaps with the sequence of a second nucleic acid element (e.g., a TATA box, cap site, transcriptional start site, 5′ UTR, open reading frame (ORF), poly(A) signal, or GC-rich region), e.g., by at least 5, 10, 15, 20, 25, 30, 40, 50, 60, 70, 80, 90, 100, 150, 200, 250, 300, 400, or 500 nucleotides. In some embodiments, the sequence of a first nucleic acid element comprised in a genetic element (e.g., a TATA box, cap site, transcriptional start site, 5′ UTR, open reading frame (ORF), poly(A) signal, or GC-rich region) does not overlap with the sequence of a second nucleic acid element (e.g., a TATA box, cap site, transcriptional start site, 5′ UTR, open reading frame (ORF), poly(A) signal, or GC-rich region).

### Protein Binding Sequence

In some embodiments, the genetic element encodes a protein binding sequence that binds to the substantially non-pathogenic protein. In some embodiments, the protein binding sequence facilitates packaging the genetic element into the proteinaceous exterior. In some embodiments, the protein binding sequence specifically binds an arginine-rich region of the substantially non-pathogenic protein. In some embodiments, the genetic element comprises a protein binding sequence having at least 70%, 80%, 85%, 90%, 95%, 96%, 97%, 98%, 99%, or 100% sequence identity to a 5′ UTR conserved domain or GC-rich domain of an Anellovirus sequence (e.g., to the reverse complement of the sequence annotated in any of Tables N1-N3).

In embodiments, the protein binding sequence has at least about 70%, 75%, 80%, 85%, 90%, 95%, 96%, 97%, 98%, 99%, or 100% sequence identity to the reverse complement of the sequence annotated as 5′ UTR conserved domain nucleotide sequence of any of Tables N1-N3. In embodiments, the protein binding sequence has at least about 70%, 75%, 80%, 85%, 90%, 95%, 96%, 97%, 98%, 99%, or 100% sequence identity to the reverse complement of the sequence annotated as the GC-rich domain nucleotide sequence of any of Tables N1-N3.

### 5′ UTR Conserved Domains

A genetic element may include an Anellovirus 5′ UTR conserved domain. Typically, the negative strand comprising the Anellovirus 5′ UTR conserved domain is packaged into a particle (e.g., an Anelloviridae family vector as described herein. In some embodiments, the Anellovirus 5′ UTR conserved domain is a wild-type Anellovirus 5′ UTR conserved domain. In some embodiments, the Anellovirus 5′ UTR conserved domain is an engineered Anellovirus 5′ UTR conserved domain having a nucleic acid sequence with at least one difference relative to the closest wild-type Anellovirus 5′ UTR conserved domain sequence. In some embodiments, the genetic element comprises a nucleic acid sequence having at least about 70%, 75%, 80%, 85%, 90%, 95%, 96%, 97%, 98%, 99%, or 100% sequence identity to the reverse complement of the sequence annotated as 5′ UTR conserved domain nucleotide sequence of any of Tables N1-N3 or Table 38.

TABLE 38
Exemplary 5′ UTR sequences from Anelloviruses.

| Source | Sequence | SEQ ID NO: |
|---|---|---|
| Consensus | CGGGTGCCGX₁AGGTGAGTTTACACACCGX₂AGT | 105 |
| | CAAGGGGCAATTCGGGCTCX₃GGACTGGCCGGG | |
| | CX₄X₅TGGG | |
| | X₁ = G or T | |
| | X₂ = C or A | |
| | X₃ = G or A | |
| | X₄ = T or C | |
| | X₅ = A, C, or T | |
| Alphatorquevirus | CGGGTGCCGGAGGTGAGTTTACACACCGCAGTC | 112 |
| Consensus 5′ UTR | AAGGGGCAATTCGGGCTCGGGACTGGCCGGGC | |
| | X₁X₂TGGG; wherein X₁ comprises T or C, and | |
| | wherein X₂ comprises A, C, or T. | |

### Identification of 5′ UTR Sequences

In some embodiments, an Anelloviridae family virus (e.g., Anellovirus) 5′ UTR sequence can be identified within the genome of an Anelloviridae family virus (e.g., Anellovirus) (e.g., a putative Anelloviridae family virus genome identified, for example, by nucleic acid sequencing techniques, e.g., deep sequencing techniques). In some embodiments, an Anelloviridae family virus (e.g., Anellovirus) 5′ UTR sequence is identified by one or both of the following steps:
(i) Identification of circularization junction point: In some embodiments, a 5′ UTR will be positioned near a circularization junction point of a full-length, circularized Anelloviridae family virus (e.g., Anellovirus) genome. A circularization junction point can be identified, for example, by identifying overlapping regions of the sequence. In some embodiments, an overlapping region of the sequence can be trimmed from the sequence to produce a full-length Anelloviridae family virus (e.g., Anellovirus) genome sequence that has been circularized. In some embodiments, a genome sequence is circularized in this manner using software. Without wishing to be bound by theory, computationally circularizing a genome may result in the start position for the sequence being oriented in a non-biological. Landmarks within the sequence can be used to re-orient sequences in the proper direction. For example, landmark sequence may include sequences having substantial homology to one or more elements within an Anelloviridae family virus (e.g., Anellovirus) genome as described herein (e.g., one or more of a TATA box, cap site, initiator element, transcriptional start site, 5′ UTR conserved domain, ORF1, ORF1/1, ORF1/2, ORF2, ORF2/2, ORF2/3, ORF2t/3, three open-reading frame region, poly(A) signal, or GC-rich region of an Anelloviridae family virus (e.g., Anellovirus), e.g., as described herein).
(ii) Identification of 5′ UTR sequence: Once a putative Anelloviridae family virus (e.g., Anellovirus) genome sequence has been obtained, the sequence (or portions thereof, e.g., having a length between about 40-50, 50-60, 60-70, 70-80, 80-90, or 90-100 nucleotides) can be compared to one or more Anelloviridae family virus (e.g., Anellovirus) 5′ UTR sequences (e.g., as described herein) to identify sequences having substantial homology thereto. In some embodiments, a putative Anelloviridae family virus (e.g., Anellovirus) 5′ UTR region has at least 50%, 60%, 70%, 75%, 80%, 85%, 90%, 95%, 96%, 97%, 98%, 99%, or 100% sequence identity to an Anelloviridae family virus (e.g., Anellovirus) 5′ UTR sequence as described herein.

### GC-Rich Regions

A genetic element may include an Anellovirus GC-rich region. Typically, the negative strand comprising the Anellovirus GC-rich region is packaged into a particle (e.g., an Anelloviridae family vector as described herein. In some embodiments, the Anellovirus GC-rich region is a wild-type Anellovirus GC-rich region. In some embodiments, the Anellovirus GC-rich region is an engineered Anellovirus GC-rich region having a nucleic acid sequence with at least one difference relative to the closest wild-type Anellovirus GC-rich region sequence. In some embodiments, the Anellovirus GC-rich region comprises a contiguous sequence of at least 20, 25, 30, 31, 32, 33, 34, 35, 36, 37, 38, 39, or 40 consecutive nucleotides having a GC content of at least 70%, 75%, 80%, 85%, 90%, 95%, or 99%. In some embodiments, the genetic element comprises a nucleic acid sequence having at least about 70%, 75%, 80%, 85%, 90%, 95%, 96%, 97%, 98%, 99%, or 100% sequence identity to the reverse complement of the sequence annotated as GC-rich region nucleotide sequence of any of Tables N1-N3 or Table 39.

TABLE 39
Exemplary GC-rich sequences from Anelloviruses.

| | | SEQ |
|---|---|---|
| | | ID |
| Source | Sequence | NO: |
| Consensus | CGGCGGX₁GGX₂GX₃X₄X₅CGCGCTX₆CGCGC | 120 |
| | GCX₇X₈X₉X₁₀CX₁₁X₁₂X₁₃X₁₄GGGGX₁₅X₁₆X₁₇X₁₈ | |
| | X₁₉X₂₀X₂₁GCX₂₂X₂₃X₂₄X₂₅CCCCCCCX₂₆CGCGC | |
| | ATX₂₇X₂₈GCX₂₉CGGGX₃₀CCCCCCCCCX₃₁X₃₂ | |
| | X₃₃GGGGGGCTCCGX₃₄CCCCCCGGCCCCCC | |
| | X₁ = G or C | |
| | X₂ = G, C, or absent | |
| | X₃ = C or absent | |
| | X₄ = G or C | |
| | X₅ = G or C | |
| | X₆ = T, G, or A | |
| | X₇ = G or C | |
| | X₈ = G or absent | |
| | X₉ = C or absent | |
| | X₁₀ = C or absent | |
| | X₁₁ = G, A, or absent | |
| | X₁₂ = G or C | |
| | X₁₃ = C or T | |
| | X₁₄ = G or A | |
| | X₁₅ = G or A | |
| | X₁₆ = A, G, T, or absent | |
| | X₁₇ = G, C, or absent | |
| | X₁₈ = G, C, or absent | |
| | X₁₉ = C, A, or absent | |
| | X₂₀ = C or A | |
| | X₂₁ = T or A | |
| | X₂₂ = G or C | |
| | X₂₃ = G, T, or absent | |
| | X₂₄ = C or absent | |
| | X₂₅ = G, C, or absent | |
| | X₂₆ = G or C | |
| | X₂₇ = G or absent | |
| | X₂₈ = C or absent | |
| | X₂₉ = G or A | |
| | X₃₀ = G or T | |
| | X₃₁ = C, T, or absent | |
| | X₃₂ = G, C, A, or absent | |
| | X₃₃ = G or C | |
| | X₃₄ = C or absent | |

### Effectors

In some embodiments, the genetic element encodes an effector, e.g., an exogenous effector. In some embodiments, the genetic element comprises a therapeutic expression sequence, e.g., a sequence that encodes an effector such as a therapeutic peptide or polypeptide, e.g., an intracellular peptide or intracellular polypeptide, a secreted polypeptide, or a protein replacement therapeutic. In some embodiments, the intracellular polypeptide is a cytosolic polypeptide, a regulatory intracellular peptide, or an anti-apoptotic agent. In some embodiments, the secreted polypeptide is a cytokine, a hormone, a growth factor, an antibody molecule that binds a hormone or a growth factor, a polypeptide that specifically binds to a VEGF, or a clotting-associated factor. In some embodiments, an effector described herein comprises an anti-VEGF antibody molecule. In some embodiments, the protein replacement therapeutic is an enzymatic effector, a non-enzymatic effector, erythropoietin, a micro-dystrophin, or a functional variant of a wild-type protein.

In some embodiments, the genetic element includes a sequence encoding a protein e.g., a therapeutic protein. Some examples of therapeutic proteins may include, but are not limited to, a hormone, a cytokine, an enzyme, an antibody (e.g., one or a plurality of polypeptides encoding at least a heavy chain or a light chain), a transcription factor, a receptor (e.g., a membrane receptor), a ligand, a membrane transporter, a secreted protein, a peptide, a carrier protein, a structural protein, a nuclease, or a component thereof.

Some examples of peptides include, but are not limited to, fluorescent tag or marker, antigen, peptide therapeutic, synthetic or analog peptide from naturally-bioactive peptide, agonist or antagonist peptide, anti-microbial peptide, a targeting or cytotoxic peptide, a degradation or self-destruction peptide, and degradation or self-destruction peptides.

In some embodiments, the effector comprises a regeneration, repair, and fibrosis factor (e.g., a growth factor, an antibody or fragment thereof against such growth factors, or miRNAs that promote regeneration and repair). In some embodiments, the effector comprises a transformation factor (e.g., protein factors or miRNAs that transform fibroblasts into differentiated cells). In some embodiments, the effector comprises a protein that stimulates cellular regeneration. In some embodiments, the effector comprises a secreted STING modulator (e.g., a STING inhibitor or a STING activator).

In some embodiments, the genetic element comprises an effector-encoding sequence. In some embodiments, the effector comprises a regulatory nucleic acid (e.g., miRNA, siRNA, mRNA, lncRNA, dsRNA, shRNA, RNA, DNA, an antisense RNA, or a gRNA).

In some embodiments, the genetic element comprises a sequence that encodes small peptides, peptidomimetics (e.g., peptoids), amino acids, and amino acid analogs.

In some embodiments, the effector mentioned in this section may also be a functional variant, homologue, or fragment thereof.

In some embodiments, the genetic element comprises a sequence encoding an exogenous effector, e.g., as described in PCT publication No. WO/2020/123753 pp. 337-366, PCT publication No. WO/2020/123773 pp. 334-361, and PCT publication No. WO/2020/123795 pp. 333-358, each of which is incorporated by reference herein in its entirety.

### Regulatory Sequences

In some embodiments, the genetic element comprises a regulatory sequence, e.g., a promoter or an enhancer, operably linked to the sequence encoding the effector.

In some embodiments, a promoter includes a DNA sequence that is located adjacent to a DNA sequence that encodes an expression product. A promoter may be linked operatively to the adjacent DNA sequence. A promoter typically increases an amount of product expressed from the DNA sequence as compared to an amount of the expressed product when no promoter exists. A promoter from one organism can be utilized to enhance product expression from the DNA sequence that originates from another organism. For example, a vertebrate promoter may be used for the expression of jellyfish GFP in vertebrates. In addition, one promoter element can increase an amount of products expressed for multiple DNA sequences attached in tandem. Hence, one promoter element can enhance the expression of one or more products. Multiple promoter elements are well-known to persons of ordinary skill in the art.

In some embodiments, a native promoter for a gene or nucleic acid sequence of interest is used. The native promoter may be used when it is desired that expression of the gene or the nucleic acid sequence should mimic the native expression. The native promoter may be used when expression of the gene or other nucleic acid sequence must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In one embodiment, high-level constitutive expression is desired. Examples of such promoters include, without limitation, the retroviral Rous sarcoma virus (RSV) long terminal repeat (LTR) promoter/enhancer, the cytomegalovirus (CMV) immediate early promoter/enhancer (see, e.g., Boshart et al, Cell, 41:521-530 (1985)), the SV40 promoter, the dihydrofolate reductase promoter, the cytoplasmic .beta.-actin promoter and the phosphoglycerol kinase (PGK) promoter.

In another embodiment, inducible promoters may be desired. Inducible promoters are those which are regulated by exogenously supplied compounds, either in cis or in trans. Other types of inducible promoters which may be useful in this context are those which are regulated by a specific physiological state, e.g., temperature, acute phase, or in replicating cells only.

In some embodiments, the genetic element comprises a gene operably linked to a tissue-specific promoter.

The genetic element may include an enhancer, e.g., a DNA sequence that is located adjacent to the DNA sequence that encodes a gene. Enhancer elements are typically located upstream of a promoter element or can be located downstream of or within a coding DNA sequence (e.g., a DNA sequence transcribed or translated into a product or products). Hence, an enhancer element can be located 100 base pairs, 200 base pairs, or 300 or more base pairs upstream or downstream of a DNA sequence that encodes the product. Enhancer elements can increase an amount of recombinant product expressed from a DNA sequence above increased expression afforded by a promoter element. Multiple enhancer elements are readily available to persons of ordinary skill in the art.

### Surface Moieties

An Anelloviridae family vector as described herein may, in some instances, include one or more moieties attached to its surface (e.g., a surface moiety that can act as an effector and/or a targeting agent). In some instances, an Anelloviridae family vector comprises more than one distinct surface moiety (e.g., a first surface moiety having an effector function as described herein and a second surface moiety that targets the Anelloviridae family vector to a cell or tissue of interest). In some instances, the surface moiety is covalently attached to the surface of the Anelloviridae family vector. For example, the surface moiety may be covalently attached to the proteinaceous exterior or a component thereof (e.g., covalently attached to an ORF1 molecule of the proteinaceous exterior). In certain embodiments, the surface moiety is fused to an ORF1 molecule. In some instances, the surface moiety is noncovalently attached to the surface of the Anelloviridae family vector. For example, the surface moiety may be noncovalently bound to the proteinaceous exterior or a component thereof (e.g., noncovalently bound to an ORF1 molecule of the proteinaceous exterior). In certain embodiments, the surface moiety comprises a region that specifically binds to a cognate moiety on or attached to the ORF1 molecule. In an embodiment, the ORF1 molecule comprises a binding moiety (e.g., an antibody molecule) that specifically recognizes an epitope on the region on the surface moiety. In an embodiment, the surface moiety comprises a binding moiety (e.g., an antibody molecule) that specifically recognizes an epitope on the ORF1 molecule.

The surface moiety can, in some instances, comprise a polypeptide. The surface moiety may, in some instances, comprise a nucleic acid molecule (e.g., DNA and/or RNA). The surface moiety may, in some instances, comprise a small molecule. In some instances, a surface moiety comprises an antigen (e.g., an antigen recognized by the immune system of a subject to be delivered the Anelloviridae family vector). In some instances, a surface moiety as described herein comprises a ligand (e.g., a ligand that binds specifically to a receptor on a target cell).

In some instances, the surface moiety comprises an effector function (e.g., as described herein). For example, the surface moiety may modulate a biological activity, e.g., of a target cell or organ. In some instances, the surface moiety induces modulation of the biological activity via binding to a cognate moiety on a target cell. For example, the surface moiety may comprise a ligand that binds to a receptor on the surface of the target cell, e.g., wherein binding of the surface moiety to the receptor initiates a downstream signaling cascade of interest. In some instances, the effector activity comprises increasing or decreasing enzymatic activity, gene expression, cell signaling, and/or cellular or organ function within a target cell or organ. Effector activities may also include binding regulatory proteins to modulate activity of the regulator, such as transcription or translation. Effector activities also may include activator or inhibitor functions.

In some instances, the surface moiety can target the Anelloviridae family vector to a target cell. For example, the surface moiety may specifically bind to a cognate moiety on the surface of the target cell. The cognate moiety on the surface of the target cell may be, for example, a molecule specifically expressed or preferentially expressed by the target cell. The cognate moiety may be, for example, a polypeptide, lipid, sugar, or small molecule. In certain embodiments, the cognate moiety is a transmembrane protein (e.g., comprising an extracellular domain that binds to the surface moiety of the Anelloviridae family vector). In certain embodiments, the cognate moiety is tethered to the surface of the cell (e.g., via a GPI anchor). In some instances, the surface moiety provides a tropism (e.g., to a target tissue or target cell type) for the Anelloviridae family vector.

In an aspect, the disclosure provides an ORF1 molecule comprising: (i) the amino acid sequence of an Anellovirus ORF1 protein, or an amino acid sequence having at least 75%, 80%, 85%, 90%, 95%, 96%, 97%, 98%, or 99% sequence identity thereto; and (ii) a click handle (e.g., an NHS click handle or a maleimide click handle, e.g., as described herein). In certain embodiments, the click handle is covalently attached to the ORF1 molecule. In certain embodiments, the click handle is noncovalently attached to the ORF1 molecule. In certain embodiments, the click handle is used to attach the ORF1 molecule to a surface moiety, e.g., via a click reaction, e.g., as described herein.

A “click handle,” as that term is used herein, refers to a chemical moiety that is capable of reacting with a second click handle in a click reaction. In some embodiments, a click handle comprises an NHS moiety and/or a maleimide moiety. In certain embodiments, a click handle comprises a DBCO moiety. In certain embodiments, a click handle comprises an azide moiety. In some embodiments, a click handle is attached to a polypeptide (e.g., an ORF1 molecule). In other embodiments, a click handle comprises a reactive group capable of forming a covalent bond with a polypeptide (e.g., an ORF1 molecule). A “click reaction”, as that term is used herein, refers to a range of reactions used to covalently link a first and a second moiety, for convenient production of linked products. It typically has one or more of the following characteristics: it is fast, is specific, is high-yield, is efficient, is spontaneous, does not significantly alter biocompatibility of the linked entities, has a high reaction rate, produces a stable product, favors production of a single reaction product, has high atom economy, is chemoselective, is modular, is stereoselective, is insensitive to oxygen, is insensitive to water, is high purity, generates only inoffensive or relatively non-toxic byproducts that can be removed by nonchromatographic methods (e.g., crystallization or distillation), needs no solvent or can be performed in a solvent that is benign or physiologically compatible, e.g., water, stable under physiological conditions. Examples include an alkyne/azide reaction, a diene/dienophile reaction, or a thiol/alkene reaction. Other reactions can be used.

### II. Compositions and Methods for Making Anelloviridae Family Vectors

The present disclosure provides, in some aspects, Anelloviridae family vectors (e.g., anellovectors) and methods thereof for delivering effectors. In some embodiments, the Anelloviridae family vectors (e.g., anellovectors) or components thereof can be made as described below. In some embodiments, the compositions and methods described herein can be used to produce a genetic element or a genetic element construct. In some embodiments, the compositions and methods described herein can be used to produce one or more Anelloviridae family virus capsid proteins (e.g., Anellovirus ORF) molecules (e.g., an ORF1, ORF2, ORF2/2, ORF2/3, ORF1/1, or ORF1/2molecule, or a functional fragment or splice variant thereof). In some embodiments, the compositions and methods described herein can be used to produce a proteinaceous exterior or a component thereof (e.g., an ORF1 molecule), e.g., in a host cell. In some embodiments, the Anelloviridae family vector (e.g., anellovector) or components thereof can be made using a tandem construct, e.g., as described in PCT Publication No. WO 2021252955, which is incorporated herein by reference in its entirety. In some embodiments, the Anelloviridae family vector (e.g., anellovector) or components thereof (e.g., an Anelloviridae family polypeptide, e.g., an ORF1, ORF2, ORF2/2, ORF2/3, ORF1/1, and/or ORF1/2 molecule, or a functional fragment or splice variant thereof) can be made using a bacmid/insect cell system, e.g., as described as described in PCT Publication No. WO 2021/252943, which is incorporated herein by reference in its entirety. Methods of producing an anellovector using a host cell are described, for example, below in the section entitled “Host Cells and Methods of Using Host Cells for Producing an Anellovector”. Method of producing an anellovector in a cell-free system are described, for example, below in the section entitled “In vitro assembly methods”.

Without wishing to be bound by theory, rolling circle amplification may occur via Rep protein binding to a Rep binding site (e.g., comprising a 5′ UTR, e.g., comprising a hairpin loop and/or an origin of replication, e.g., as described herein) positioned 5′ relative to (or within the 5′ region of) the genetic element region. The Rep protein may then proceed through the genetic element region, resulting in the synthesis of the genetic element. The genetic element may then be circularized and then enclosed within a proteinaceous exterior to form an Anelloviridae family vector (e.g., anellovector).

### Genetic Element Constructs, e.g., for Assembly of Anelloviridae Family Vectors

In some methods of making as described herein, the genetic element is made using a genetic element construct, wherein the genetic element construct can act as a template for the production of the genetic element. In some embodiments, a genetic element construct comprises a genetic element region and optionally other sequence such as vector backbone. A genetic element construct may be any nucleic acid construct suitable for delivery of the sequence of the genetic element into a host cell in which the genetic element can be enclosed within a proteinaceous exterior. In some embodiments, the genetic element construct comprises a promoter. In some embodiments, the genetic element construct is a linear nucleic acid molecule. In some embodiments, the genetic element construct is a circular nucleic acid molecule (e.g., a plasmid, viral nucleic acid, bacmid, artificial chromosome, or a minicircle, e.g., as described herein). In some embodiments, a double-stranded circular nucleic acid (e.g., a minicircle) can be excised from a plasmid (e.g., by in vitro circularization). In some embodiments, in vitro circularized DNA constructs can be produced by digesting a genetic element construct (e.g., a plasmid comprising the sequence of a genetic element) to be packaged, such that the genetic element sequence is excised as a linear DNA molecule. The resultant linear DNA can then be ligated, e.g., using a DNA ligase, to form a double-stranded circular DNA (e.g., a minicircle). In embodiments, the double-stranded circular nucleic acid construct (e.g., minicircle) can be introduced into a host cell, in which it can be converted into or used as a template for generating single-stranded circular genetic elements, e.g., as described herein. In some embodiments, the genetic element is generated by a polymerase based on a template sequence in the nucleic acid construct. In some embodiments, the polymerase produces a single-stranded copy of the genetic element sequence, which can optionally be circularized to form a genetic element as described herein.

The genetic element construct may, in some embodiments, be double-stranded. In some embodiments, the genetic element construct comprises RNA. In some embodiments, the genetic element construct comprises one or more modified nucleotides.

### Tandem Constructs

In some embodiments, a genetic element construct comprises a first copy of a genetic element sequence (e.g., the nucleic acid sequence of a genetic element, e.g., as described herein) and at least a portion of a second copy of a genetic element sequence (e.g., the nucleic acid sequence of the same genetic element, or the nucleic acid sequence of a different genetic element), arranged in tandem. Genetic element constructs having such a structure are generally referred to herein as tandem constructs. Such tandem constructs are used for producing an Anelloviridae family vector (e.g., anellovector) genetic element. The first copy of the genetic element sequence and the second copy of the genetic element sequence may, in some instances, be immediately adjacent to each other on the genetic acid construct. In other instances, the first copy of the genetic element sequence and the second copy of the genetic element sequence may be separated, e.g., by a spacer sequence. Without being bound by theory, a tandem construct described herein may, in some embodiments, replicate by rolling circle replication. In some embodiments, a tandem construct is a plasmid. In some embodiments, a tandem construct is circular. In some embodiments, a tandem construct is linear. In some embodiments, a tandem construct is single-stranded. In some embodiments, a tandem construct is double-stranded. In some embodiments, a tandem construct is DNA.

Additional descriptions of tandem constructs that can be used with the invention are described, for example, PCT Publication No. WO 2021252955, incorporated herein by reference in its entirety.

### Cis/Trans Constructs

In some embodiments, a genetic element construct as described herein comprises one or more sequences encoding one or more Anelloviridae family virus ORFs, e.g., proteinaceous exterior components (e.g., polypeptides encoded by an Anellovirus ORF1 nucleic acid, e.g., as described herein). For example, the genetic element construct may comprise a nucleic acid sequence encoding an Anellovirus ORF1 molecule. Such genetic element constructs can be suitable for introducing the genetic element and the Anelloviridae family virus ORF(s) into a host cell in cis. In other embodiments, a genetic element construct as described herein does not comprise sequences encoding one or more Anelloviridae family virus ORFs, e.g., proteinaceous exterior components (e.g., polypeptides encoded by an Anellovirus ORF1 nucleic acid, e.g., as described herein). For example, the genetic element construct may not comprise a nucleic acid sequence encoding an Anellovirus ORF1 molecule. Such genetic element constructs can be suitable for introducing the genetic element into a host cell, with the one or more Anelloviridae family virus ORFs to be provided in trans (e.g., via introduction of a second nucleic acid construct encoding one or more of the Anelloviridae family virus ORFs, or via an Anelloviridae family virus ORF cassette integrated into the genome of the host cell). In some embodiments, an ORF1 molecule is provided in trans, e.g., as described herein. In some embodiments, an ORF2 molecule is provided in trans, e.g., as described herein. In some embodiments, an ORF1 molecule and an ORF2 molecule are both provided in trans, e.g., as described herein.

In some embodiments, the genetic element construct comprises a sequence encoding an Anellovirus ORF1 molecule, or a splice variant or functional fragment thereof (e.g., a jelly-roll region, e.g., as described herein). In embodiments, the portion of the genetic element that does not comprise the sequence of the genetic element comprises the sequence encoding the Anellovirus ORF1 molecule, or splice variant or functional fragment thereof (e.g., in a cassette comprising a promoter and the sequence encoding the Anellovirus ORF1 molecule, or splice variant or functional fragment thereof). In further embodiments, the portion of the construct comprising the sequence of the genetic element comprises a sequence encoding an Anellovirus ORF1 molecule, or a splice variant or functional fragment thereof (e.g., a jelly-roll region, e.g., as described herein). In embodiments, enclosure of such a genetic element in a proteinaceous exterior (e.g., as described herein) produces a replication-component Anelloviridae family vector (e.g., anellovector) (e.g., an Anelloviridae family vector that upon infecting a cell, enables the cell to produce additional copies of the anellovector without introducing further nucleic acid constructs, e.g., encoding one or more Anelloviridae family virus ORFs as described herein, into the cell).

In other embodiments, the genetic element does not comprise a sequence encoding an Anellovirus ORF1 molecule, or a splice variant or functional fragment thereof (e.g., a jelly-roll region, e.g., as described herein). In embodiments, enclosure of such a genetic element in a proteinaceous exterior (e.g., as described herein) produces a replication-incompetent Anelloviridae family vector (e.g., anellovector) (e.g., an Anelloviridae family vector that, upon infecting a cell, does not enable the infected cell to produce additional Anelloviridae family vector, e.g., in the absence of one or more additional constructs, e.g., encoding one or more Anellovirus ORFs as described herein).

### Recombinase-Based Production of Genetic Elements and Anellovectors

A genetic element for an Anelloviridae family vector (e.g., an anellovector) may be produced via site-specific recombination of a genetic element construct to produce a circular nucleic acid molecule comprising the genetic element sequence. In some embodiments, the circular nucleic acid molecule is a double-stranded DNA minicircle. In some embodiments, the circular nucleic acid molecule is in turn converted to a circular single-stranded DNA molecule, which can in turn serve as the genetic element of an Anelloviridae family vector (e.g., an anellovector) as described herein. Generally, the genetic element construct comprises a set of recombinase recognition sequences flanking the sequence of a genetic element of an Anelloviridae family vector. The recombinase recognition sites may be recognized by a site-specific recombinase. The remainder of the genetic element construct may, in some instances, comprise a vector backbone comprising elements for replication of the construct in a cell, such as a mammalian cell.

Contacting the genetic element construct with the site-specific recombinase (e.g., in a host cell) may result in excision of the genetic element sequence from the remainder of the construct and the formation of two circular nucleic acid molecules, one comprising the genetic element sequence and the other comprising the remainder of the vector backbone. In some embodiments, the circular nucleic acid molecule (e.g., a minicircle) comprising the genetic element sequence is converted to cssDNA in the host cell (e.g., a mammalian host cell), and is then encapsulated in a proteinaceous exterior comprising ORF1 molecules (e.g., as described herein) to produce an Anelloviridae family vector.

In some embodiments, a site specific recombinase-based system for producing genetic elements comprises three plasmids: (1) a first plasmid (e.g., a vector plasmid) comprising the sequence of the genetic element, flanked by a pair of recombinase recognition sites; (2) an expression plasmid comprising a cassette encoding a site-specific recombinase (e.g., as described herein) capable of recognizing the recombinase recognition sites; and (3) a plasmid (e.g., a self-replicating rescue (SRR) plasmid) providing one or more Anelloviridae family viral proteins (e.g., Anellovirus ORF1, ORF3, and/or ORF3 molecules), including a capsid protein (e.g., an ORF1 molecule, e.g., as described herein).

In some embodiments, a site specific recombinase-based system for producing genetic elements comprises two plasmids: (1) a first plasmid (e.g., a vector plasmid) comprising the sequence of the genetic element, flanked by a pair of recombinase recognition sites; and (2) a second plasmid (e.g., a self-replicating rescue (SRR) plasmid) providing one or more Anelloviridae family viral proteins (e.g., Anellovirus ORF1, ORF3, and/or ORF3 molecules), including a capsid protein (e.g., an ORF1 molecule, e.g., as described herein).

### Self-Replicating Rescue (SRR) Constructs (e.g., SRR Plasmids)

In some embodiments, a rescue construct can be used to provide one or more Anelloviridae family viral proteins, or functional fragments or variants thereof (e.g., one or more of Anellovirus ORF1, ORF2, and/or ORF3 molecules). In some embodiments, the rescue construct is capable of self-replicating in a host cell (e.g., a self-replicating rescue (SRR) plasmid). In some embodiments, the SRR plasmid can be used in the site-specific recombinase based systems.

In some embodiments, the rescue construct includes an expression cassette comprising the protein coding sequence of an Anelloviridae family virus (e.g., an Anellovirus as described herein), or a sequence having at least 75%, 80%, 85%, 90%, 95%, 96%, 97%, 98%, or 99% identity thereto. In some embodiments, the rescue construct includes a sequence encoding a replication protein (e.g., a large T antigen, a PCV Rep, or a PCV Rep′), or a sequence having at least 75%, 80%, 85%, 90%, 95%, 96%, 97%, 98%, or 99% identity thereto. In some embodiments, the rescue construct includes an exogenous origin of replication (e.g., a viral origin, e.g., an SV40, PCV, or AAV origin). In certain embodiments, the replication protein coding sequence is downstream of an internal ribosome entry site (IRES) positioned downstream of the expression cassette comprising the protein coding sequence of the Anelloviridae family virus. In certain embodiments, the replication protein coding sequence is comprised in a separate cassette from the expression cassette comprising the protein coding sequence of the Anelloviridae family virus. In some embodiments, the rescue construct further comprises one or more additional expression cassettes, e.g., encoding a site-specific recombinase, an Anelloviridae family viral protein (e.g., an Anellovirus ORF1 molecule), a replication protein and/or viral origin (e.g., as described herein), or another transgene of interest.

In some embodiments, the rescue construct comprises a single expression cassette (e.g., as described above). In some embodiments, the rescue construct comprises two expression cassettes. In some embodiments, the rescue construct comprises three expression cassettes. In some embodiments, the rescue construct comprises four expression cassettes. In some embodiments, the rescue construct comprises five or more expression cassettes. The exemplary expression cassettes described herein can be positioned in any order within the rescue construct. In some embodiments, one or more of the expression cassettes is in the opposite orientation relative to one or more of the other expression cassettes. In other embodiments, all of the expression cassettes in a rescue construct are in the same orientation relative to each other.

In some embodiments, the rescue construct does not contain an Anellovirus NCR sequence (e.g. does not contain an Anellovirus 5′ NCR sequence and/or an Anellovirus 3′ NCR sequence). In some embodiments, the rescue construct does not comprise an Anellovirus 5′ UTR conserved domain. In some embodiments, the rescue construct does not comprise an Anellovirus GC-rich region. In some embodiments, the rescue construct does not contain Anellovirus sequences homologous to the vector plasmid (e.g., a contiguous sequence of at least 10, 20, 30, 40, 50, 60, 70, 80, 90, 100, 125, 150, 175, 200, 250, 300, 400, or 500 nucleotides having sequence identity to any Anellovirus sequence of the same length in the vector plasmid).

Exemplary SRR plasmids that can, in some embodiments, be used in a site-specific recombinase system as described herein (e.g., a two-plasmid system or three-plasmid system as described herein) are provided in Table WI.

TABLE W1
Exemplary Ring19 SRR plasmid (pRTx-3525)

| | |
|---|---|
| Name | pRTx-3525 |
| Type | Plasmid |
| Description | phEF1a_Ring19-UTR-FullORF_SVLT_SV40ori. |
| Length | 9391 bp |

| | |
|---|---|
| 1 | TCGCGCGTTT CGGTGATGAC GGTGAAAACC TCTGACACAT GCAGCTCCCG GAGACGGTCA |
| 61 | CAGCTTGTCT GTAAGCGGAT GCCGGGAGCA GACAAGCCCG TCAGGGCGCG TCAGCGGGTG |
| 121 | TTGGCGGGTG TCGGGGCTGG CTTAACTATG CGGCATCAGA GCAGATTGTA CTGAGAGTGC |
| 181 | ACCATATGCG GTGTGAAATA CCGCACAGAT GCGTAAGGAG AAAATACCGC ATCAGGCGCC |
| 241 | ATTCGCCATT CAGGCTGCGC AACTGTTGGG AAGGGCGATC GGTGCGGGCC TCTTCGCTAT |
| 301 | TACGCCAGCT GGCGAAAGGG GGATGTGCTG CAAGGCGATT AAGTTGGGTA ACGCCAGGGT |
| 361 | TTTCCCAGTC ACGACGTTGT AAAACGACGG CCAGAGAATT CGAGCTCGGT ACCTCGCGAA |
| 421 | TACATCTAGA TATGGTTGGC TCCGGTGCCC GTCAGTGGGC AGAGCGCACA TCGCCCACAG |
| 481 | TCCCCGAGAA GTTGTGGGGA GGGGTCGGCA ATTGAACCGG TGCCTAGAGA AGGTGGCGCG |
| 541 | GGGTAAACTG GGAAAGTGAT GTCGTGTACT GGCTCCGCCT TTTTCCCGAG GGTGGGGGAG |
| 601 | AACCGTATAT AAGTGCAGTA GTCGCCGTGA ACGTTCTTTT TCGCAACGGG TTTGCCGCCA |
| 661 | GAACACAGGT AAGTGCCGTG TGTGGTTCCC GCGGGCCTGG CCTCTTTACG GGTTATGGCC |
| 721 | CTTGCGTGCC TTGAATTACT TCCACCTGGC TGCAGTACGT GATTCTTGAT CCCGAGCTTC |
| 781 | GGGTTGGAAG TGGGTGGGAG AGTTCGAGGC CTTGCGCTTA AGGAGCCCCT TCGCCTCGTG |
| 841 | CTTGAGTTGA GGCCTGGCCT GGGCGCTGGG GCCACCGCGT GCGAATCTGG TGGCACCTTC |
| 901 | GCGCCTGTCT CGCTGCTTTC GATAAGTCTC TAGCCATTTA AAATTTTTGA TGACCTGCTG |
| 961 | CGACGCTTTT TTTCTGGCAA GATAGTCTTG TAAATGCGGG CCAAGATCTG CACACTGGTA |
| 1021 | TTTCGGTTTT TGGGGCCGCG GGCGGCGACG GGGCCCGTGC GTCCCAGCGC ACATGTTCGG |
| 1081 | CGAGGCGGGG CCTGCGAGCG CGGCCACCGA GAATCGGACG GGGGTAGTCT CAAGCTGGCC |
| 1141 | GGCCTGCTCT GGTGCCTGGC CTCGCGCCGC CGTGTATCGC CCCGCCCTGG GGGCAAGGC |
| 1201 | TGGCCCGGTC GGCACCAGTT GCGTGAGCGG AAAGATGGCC GCTTCCCGGC CCTGCTGCAG |
| 1261 | GGAGCTCAAA ATGGAGGACG CGGCGCTCGG GAGAGCGGGC GGGTGAGTCA CCCACACAAA |
| 1321 | GGAAAAGGGC CTTTCCGTCC TCAGCCGTCG CTTCATGTGA CTCCACGGAG TACCGGGCGC |
| 1381 | CGTCCAGGCA CCTCGATTAG TTCTCGAGCT TTTGGAGTAC GTCGTCTTTA GGTTGGGGGG |
| 1441 | AGGGGTTTTA TGCGATGGAG TTTCCCCACA CTGAGTGGGT GGAGACTGAA GTTAGGCCAG |
| 1501 | CTTGGCACTT GATGTAATTC TCCTTGGAAT TTGCCCTTTT TGAGTTTGGA TCTTGGTTCA |
| 1561 | TTCTCAAGCC TCAGACAGTG GTTCAAAGTT TTTTTCTTCC ATTTCAGGTG GATGTTTATG |
| 1621 | CCGCCAGACG GAGACGGGAT CACTTCAGTG ACTCCAGGCT GAACTTGGGC GGGAGCCGAA |
| 1681 | GGTGAGTGCA ACCACCGTAG TCTAGGGGCA ATTCGGGCTA GTTCAGTATG GCGGAACGGG |
| 1741 | CAAGAAACTT AAATATTATT ATTTTACAGA TGCAAATACA ACCACCTATT AGAACCTTCA |
| 1801 | AACAAACAAT TTCAGATTGG AAAAACTTAA TTGTCCACGT TCACGACAAC ATTTGCAACT |
| 1861 | GCAATAAACC ATTAGAACAC ACTATTGATA CCTGTATCAC CAATCCAGAT GAATTAAGAT |
| 1921 | TAAACAAATC TACTAAACAA CAACTACAAA AATGCCTTGG TACCCCAGAA GAAGATACCC |
| 1981 | AAGAAGACGT TATCGATGGC TTCGCAGATG GAGAGCTAGA CGCCCTTTTC GCCCAAGATA |
| 2041 | CAGAAGAAGA TACTGGGTAA GAAACTATTC TCGAAAGAGA AAACTATTTA AAATAACAAC |
| 2101 | CAAAGAATGG CAACCAAAAG TTATAAGAAA GACTCATGTA AAGGGCACCT ATCCTTTGTT |
| 2161 | TCTTTGTACA AAGCACAGAA TTAACAATAA TATGATACAA TATTTAGACT CTATAGCTCC |
| 2221 | AGAACACTAT TACGGAGGAG GAGGATTTTC AATAATGCAA TTTTCCTTAC AAGCCTTATA |
| 2281 | TGAAGAATTT ATAAAAGCAA AAAACTGGTG GACTAATACA AACTGCTTTT TACCACTTGT |
| 2341 | AAGATATATG GGTTGCTCAT TCAAATTTTA TAAAACTGAA TTTTATGATT ATATTGTACT |
| 2401 | AATTGAAAGA TGTTATCCAC TTGCTTGTAC TGATGAAATG TACTTATCTA CTCAACCTAG |
| 2461 | TATTATGATG CTTACAAGAA AATGTATTTT TGTACCATGC AAACAAAACA GCAAAGGTAA |
| 2521 | AAAACCTTAC AAAAAAGTTA GAGTAAGACC ACCTTCACAA ATGACTACAG GATGGCATTT |
| 2581 | CTCACAAGAC TTAGCAAACA TGCCACTTGT AGTACTAAAA ACTTCAGTAT GCAGCTTTGA |
| 2641 | CAGATATTAC ACAGACAGTA CAGCTAAATC AACCACAATA GGCTTTAAAA CACTTAACAC |
| 2701 | ACAAACATTT AGATATCATG ACTGGCAGGA ACCACCTACA ACAGGATACA AACCACAAAA |
| 2761 | CCTACTATGG TTTTATGGAG CAGAAAACGG ATCACCAGTA GACCCCAACA ACACAATAGT |
| 2821 | ATCAAACCTA ATATACTTAG GAGGCACAGG ACCTTATGAA AAAGGCACAC CAATAAAAAC |
| 2881 | AAACATAAGC AATTACTTTT CAGAGCCTAA ACTGTGGGGA AATATATTTC ACGATGATTA |
| 2941 | TACATCAGGA ACATCACCCG TGTTTGTTAC AAACAAATCA CCATCAGAAA TTAAAACCGC |
| 3001 | ATGGAACACT ATAAAAGACT TAACTGTTAA AGCTAGCGGT GTATTTACAT TAAGAACAAT |
| 3061 | TCCACTATGG CTACCTTGCA GATACAACCC ATTTGCAGAC AAAGCAACCA ACAACAAAAT |
| 3121 | ATGGCTAGTT TCTATACATT CAGACCACAC AGAATGGAAA CCAATAGACA ATCCATTACT |
| 3181 | ACAACGAACA GACCTTCCTT TATGGTTACT TGTATGGGGT TGGCAAGATT GGCAGAAAAA |
| 3241 | AAACCAACAA ACTTCACAAC CTGATATTAA TTATTTAACA GTAATATCTT CACCATATAT |
| 3301 | ATCATGCTAC CCAAAATTAG ATTACTATGT GTTACTAGAT GAAGGATTTT GGGAGGGTCA |
| 3361 | CTCAACATAC ATAGAGTCAA TTACAGACTC AGACAAAAAA CACTGGTACC CTAAAAATAG |
| 3421 | ATTTCAAATA GAAACACTTA ATCTAATAGC TAACACAGGT CCAGGAACTG TAAAACTAAG |
| 3481 | AGAAAACCAA GCAGCAGAAG GTCACATGGT ATATCGCTTT AATTTTAAGC TTGGAGGATG |
| 3541 | TCCCGCACCG ATGGAAAAAA TATGTGACCC TAGCAAACAA TCCAAATATC CTATTCCCAA |
| 3601 | TAACCAGCAA CAAACAACTT CGTTGCAGAG TCCAGAAAAC CCAATTCAAA CCTATCTCTA |
| 3661 | CGACTTCGAC GAAAGGAGGG GCCTACTTAC AGAAAGAGCT ACAAAAAGAA TCAAACAAGA |
| 3721 | TCACACATCT GAAAAAACTG TTTTGCCATT TACAGGAGCA GCAACAGACC TCCCCATACT |
| 3781 | CCAAACAACA TCACAGGAGG AAAGCTCCTC GGAAGAAGAA GAAGAGCAAC AAGCGGAGAA |
| 3841 | GAAACTACTC CAGCTCCGAA GAAAGCAGCA CCGACTCCGG GAGCGAATCC TCCAGCTATT |
| 3901 | AGACATACAA AATACATAAT AAAACAAAGT ACTGTAAAAA TTGATATGTT TGGAGATACT |
| 3961 | CATGTACCTA ACCGTAGAAT GACCCCAGAA GAATTTGAAC AAGAACTAAT TGTCGCTGGT |
| 4021 | GTTTTTCGCA GACCTCCTTG TTACTATATA AAAGATAGAC CTACTTATCC TTATGTACCA |
| 4081 | AAACCTACTG ATGAAAAATG TATGGTAAAC TTTGACTTAA ACTTTCCTTA ATAAAATGAA |
| 4141 | TGCAATTGTT GTTGTTAACG GGGATCCTCA TCGCGGCCGC TACGTAAATT CCGCCCCCCC |
| 4201 | CCCCCCTCTC CCTCCCCCCC CCCTAACGTT ACTGGCCGAA GCCGCTTGGA ATAAGGCCGG |
| 4261 | TGTGCGTTTG TCTATATGTT ATTTTCCACC ATATTGCCGT CTTTTGGCAA TGTGAGGGCC |
| 4321 | CGGAAACCTG GCCCTGTCTT CTTGACGAGC ATTCCTAGGG GTCTTTCCCC TCTCGCCAAA |
| 4381 | GGAATGCAAG GTCTGTTGAA TGTCGTGAAG GAAGCAGTTC CTCTGGAAGC TTCTTGAAGA |
| 4441 | CAAACAACGT CTGTAGCGAC CCTTTGCAGG CAGCGGAACC CCCCACCTGG CGACAGGTGC |
| 4501 | CTCTGCGGCC AAAAGCCACG TGTATAAGAT ACACCTGCAA AGGCGGCACA ACCCCAGTGC |
| 4561 | CACGTTGTGA GTTGGATAGT TGTGGAAAGA GTCAAATGGC TCTCCTCAAG CGTATTCAAC |
| 4621 | AAGGGGCTGA AGGATGCCCA GAAGGTACCC CATTGTATGG GATCTGATCT GGGGCCTCGG |
| 4681 | TGCACATGCT TTACATGTGT TTAGTCGAGG TTAAAAAAAC GTCTAGGCCC CCCGAACCAC |
| 4741 | GGGGACGTGG TTTTCCTTTG AAAAACACGA TGATAATATG GCCACAACCA TGGATAAAGT |
| 4801 | TTTAAACAGA GAGGAATCTT TGCAGCTAAT GGACCTTCTA GGTCTTGAAA GGAGTGCCTG |
| 4861 | GGGGAATATT CCTCTGATGA GAAAGGCATA TTTAAAAAAA TGCAAGGAGT TTCATCCTGA |
| 4921 | TAAAGGAGGA GATGAAGAAA AAATGAAGAA AATGAATACT CTGTACAAGA AAATGGAAGA |
| 4981 | TGGAGTAAAA TATGCTCATC AACCTGACTT TGGAGGCTTC TGGGATGCAA CTGAGATTCC |
| 5041 | AACCTATGGA ACTGATGAAT GGGAGCAGTG GTGGAATGCC TTTAATGAGG AAAACCTGTT |
| 5101 | TTGCTCAGAA GAAATGCCAT CTAGTGATGA TGAGGCTACT GCTGACTCTC AACATTCTAC |
| 5161 | TCCTCCAAAA AAGAAGAGAA AGGTAGAAGA CCCCAAGGAC TTTCCTTCAG AATTGCTAAG |
| 5221 | TTTTTTGAGT CATGCTGTGT TTAGTAATAG AACTCTTGCT TGCTTTGCTA TTTACACCAC |
| 5281 | AAAGGAAAAA GCTGCACTGC TATACAAGAA AATTATGGAA AAATATTCTG TAACCTTTAT |
| 5341 | AAGTAGGCAT AACAGTTATA ATCATAACAT ACTGTTTTTT CTTACTCCAC ACAGGCATAG |
| 5401 | AGTGTCTGCT ATTAATAACT ATGCTCAAAA ATTGTGTACC TTTAGCTTTT TAATTTGTAA |
| 5461 | AGGGGTTAAT AAGGAATATT TGATGTATAG TGCCTTGACT AGAGATCCAT TTTCTGTTAT |
| 5521 | TGAGGAAAGT TTGCCAGGTG GGTTAAAGGA GCATGATTTT AATCCAGAAG AAGCAGAGGA |
| 5581 | AACTAAACAA GTGTCCTGGA AGCTTGTAAC AGAGTATGCA ATGGAAACAA AATGTGATGA |
| 5641 | TGTGTTGTTA TTGCTTGGGA TGTACTTGGA ATTTCAGTAC AGTTTTGAAA TGTGTTTAAA |
| 5701 | ATGTATTAAA AAAGAACAGC CCAGCCACTA TAAGTACCAT GAAAAGCATT ATGCAAATGC |
| 5761 | TGCTATATTT GCTGACAGCA AAAACCAAAA AACCATATGC CAACAGGCTG TTGATACTGT |
| 5821 | TTTAGCTAAA AAGCGGGTTG ATAGCCTACA ATTAACTAGA GAACAAATGT TAACAAACAG |
| 5881 | ATTTAATGAT CTTTTGGATA GGATGGATAT AATGTTTGGT TCTACAGGCT CTGCTGACAT |
| 5941 | AGAAGAATGG ATGGCTGGAG TTGCTTGGCT ACACTGTTTG TTGCCCAAAA TGGATTCAGT |
| 6001 | GGTGTATGAC TTTTTAAAAT GCATGGTGTA CAACATTCCT AAAAAAAGAT ACTGGCTGTT |
| 6061 | TAAAGGACCA ATTGATAGTG GTAAAACTAC ATTAGCAGCT GCTTTGCTTG AATTATGTGG |
| 6121 | GGGGAAAGCT TTAAATGTTA ATTTGCCCTT GGACAGGCTG AACTTTGAGC TAGGAGTAGC |
| 6181 | TATTGACCAG TTTTTAGTAG TTTTTGAGGA TGTAAAGGGC ACTGGAGGGG AGTCCAGAGA |
| 6241 | TTTGCCTTCA GGTCAGGGAA TTAATAACCT GGACAATTTA AGGGATTATT TGGATGGCAG |
| 6301 | TGTTAAGGTA AACTTAGAAA AGAAACACCT AAATAAAAGA ACTCAAATAT TTCCCCCTGG |
| 6361 | AATAGTCACC ATGAATGAGT ACAGTGTGCC TAAAACACTG CAGGCCAGAT TTGTAAAACA |
| 6421 | AATAGATTTT AGGCCCAAAG ATTATTTAAA GCATTGCCTG GAACGCAGTG AGTTTTTGTT |
| 6481 | AGAAAAGAGA ATAATTCAAA GTGGCATTGC TTTGCTTCTT ATGTTAATTT GGTACAGACC |
| 6541 | TGTGGCTGAG TTTGCTCAAA GTATTCAGAG CAGAATTGTG GAGTGGAAAG AGAGATTGGA |
| 6601 | CAAAGAGTTT AGTTTGTCAG TGTATCAAAA AATGAAGTTT AATGTGGCTA TGGGAATTGG |
| 6661 | AGTTTTAGAT TGGCTAAGAA ACAGTGATGA TGATGATGAA GACAGCCAGG AAAATGCTGA |
| 6721 | TAAAAATGAA GATGGTGGGG AGAAGAACAT GGAAGACTCA GGGCATGAAA CAGGCATTGA |
| 6781 | TTCACAGTCC CAAGGCTCAT TTCAGGCCCC TCAGTCCTCA CAGTCTGTTC ATGATCATAA |
| 6841 | TCAGCCATAC CACATTTGTA GAGGTTTTAC TTGCTTTAAA AAACCTCCCA CACCTCCCCC |
| 6901 | TGAACCTGAA ACATAATAAG CTTGCGGCCG CTTCGAGCAG ACATGATAAG ATACATTGAT |
| 6961 | GAGTTTGGAC AAACCACAAC TAGAATGCAG TGAAAAAAAT GCTTTATTTG TGAAATTTGT |
| 7021 | GATGCTATTG CTTTATTTGT AACCATTATA AGCTGCAATA AACAAGTTTC ATGTCTGGCT |
| 7081 | CTAGCTATCC CGCCCCTAAC TCCGCCCATC CCGCCCCTAA CTCCGCCCAG TTCCGCCCAT |
| 7141 | TCTCCGCCCC ATGGCTGACT AATTTTTTTT ATTTATGCAG AGGCCGAGGC CGCCTCGGCC |
| 7201 | TCTGAGCTAT TCCAGAAGTA GTGAGGAGGC TTTTTTGGAG GCCATCGGAT CCCGGGCCCG |
| 7261 | TCGACTGCAG AGGCCTGCAT GCAAGCTTGG TGTAATCATG GTCATAGCTG TTTCCTGTGT |
| 7321 | GAAATTGTTA TCCGCTCACA ATTCCACACA ACATACGAGC CGGAAGCATA AAGTGTAAAG |
| 7381 | CCTGGGGTGC CTAATGAGTG AGCTAACTCA CATTAATTGC GTTGCGCTCA CTGCCCGCTT |
| 7441 | TCCAGTCGGG AAACCTGTCG TGCCAGCTGC ATTAATGAAT CGGCCAACGC GCGGGGAGAG |
| 7501 | GCGGTTTGCG TATTGGGCGC TCTTCCGCTT CCTCGCTCAC TGACTCGCTG CGCTCGGTCG |
| 7561 | TTCGGCTGCG GCGAGCGGTA TCAGCTCACT CAAAGGCGGT AATACGGTTA TCCACAGAAT |
| 7621 | CAGGGGATAA CGCAGGAAAG AACATGTGAG CAAAAGGCCA GCAAAAGGCC AGGAACCGTA |
| 7681 | AAAAGGCCGC GTTGCTGGCG TTTTTCCATA GGCTCCGCCC CCCTGACGAG CATCACAAAA |
| 7741 | ATCGACGCTC AAGTCAGAGG TGGCGAAACC CGACAGGACT ATAAAGATAC CAGGCGTTTC |
| 7801 | CCCCTGGAAG CTCCCTCGTG CGCTCTCCTG TTCCGACCCT GCCGCTTACC GGATACCTGT |
| 7861 | CCGCCTTTCT CCCTTCGGGA AGCGTGGCGC TTTCTCATAG CTCACGCTGT AGGTATCTCA |
| 7921 | GTTCGGTGTA GGTCGTTCGC TCCAAGCTGG GCTGTGTGCA CGAACCCCCC GTTCAGCCCG |
| 7981 | ACCGCTGCGC CTTATCCGGT AACTATCGTC TTGAGTCCAA CCCGGTAAGA CACGACTTAT |
| 8041 | CGCCACTGGC AGCAGCCACT GGTAACAGGA TTAGCAGAGC GAGGTATGTA GGCGGTGCTA |
| 8101 | CAGAGTTCTT GAAGTGGTGG CCTAACTACG GCTACACTAG AAGAACAGTA TTTGGTATCT |
| 8161 | GCGCTCTGCT GAAGCCAGTT ACCTTCGGAA AAAGAGTTGG TAGCTCTTGA TCCGGCAAAC |
| 8221 | AAACCACCGC TGGTAGCGGT GGTTTTTTTG TTTGCAAGCA GCAGATTACG CGCAGAAAAA |
| 8281 | AAGGATCTCA AGAAGATCCT TTGATCTTTT CTACGGGGTC TGACGCTCAG TGGAACGAAA |
| 8341 | ACTCACGTTA AGGGATTTTG GTCATGAGAT TATCAAAAAG GATCTTCACC TAGATCCTTT |
| 8401 | TAAATTAAAA ATGAAGTTTT AAATCAAGCC CAATCTGAAT AATGTTACAA CCAATTAACC |
| 8461 | AATTCTGATT AGAAAAACTC ATCGAGCATC AAATGAAACT GCAATTTATT CATATCAGGA |
| 8521 | TTATCAATAC CATATTTTTG AAAAAGCCGT TTCTGTAATG AAGGAGAAAA CTCACCGAGG |
| 8581 | CAGTTCCATA GGATGGCAAG ATCCTGGTAT CGGTCTGCGA TTCCGACTCG TCCAACATCA |
| 8641 | ATACAACCTA TTAATTTCCC CTCGTCAAAA ATAAGGTTAT CAAGTGAGAA ATCACCATGA |
| 8701 | GTGACGACTG AATCCGGTGA GAATGGCAAA AGTTTATGCA TTTCTTTCCA GACTTGTTCA |
| 8761 | ACAGGCCAGC CATTACGCTC GTCATCAAAA TCACTCGCAT CAACCAAACC GTTATTCATT |
| 8821 | CGTGATTGCG CCTGAGCGAG ACGAAATACG CGATCGCTGT TAAAAGGACA ATTACAAACA |
| 8881 | GGAATCGAAT GCAACCGGCG CAGGAACACT GCCAGCGCAT CAACAATATT TTCACCTGAA |
| 8941 | TCAGGATATT CTTCTAATAC CTGGAATGCT GTTTTTCCGG GGATCGCAGT GGTGAGTAAC |
| 9001 | CATGCATCAT CAGGAGTACG GATAAAATGC TTGATGGTCG GAAGAGGCAT AAATTCCGTC |
| 9061 | AGCCAGTTTA GTCTGACCAT CTCATCTGTA ACATCATTGG CAACGCTACC TTTGCCATGT |
| 9121 | TTCAGAAACA ACTCTGGCGC ATCGGGCTTC CCATACAAGC GATAGATTGT CGCACCTGAT |
| 9181 | TGCCCGACAT TATCGCGAGC CCATTTATAC CCATATAAAT CAGCATCCAT GTTGGAATTT |
| 9241 | AATCGCGGCC TCGACGTTTC CCGTTGAATA TGGCTCATAA CACCCCTTGT ATTACTGTTT |
| 9301 | ATGTAAGCAG ACAGTTTTAT TGTTCATGAT GATATATTTT TATCTTGTGC AATGTAACAT |
| 9361 | CAGAGATTTT GAGACACGGG CCAGAGCTGC A (SEQ ID NO: 500) |

Annotations:

| Region/Element | Base range |
|---|---|
| pHEf1A promoter | 436-1609 |
| EF-1-alpha core promoter | 457-668 |
| EF-1-alpha intron A | 669-1607 |
| Initiator element | 1614-1618 |
| 5′ UTR conserved domain | 1670-1740 |
| Intron 1 | 1682-1769 |
| ORF2/3 coding sequence | 1770-2056, 3756-4131 |
| ORF2/2 coding sequence | 1770-2056, 3629-3902 |
| ORF2 coding sequence | 1770-2060 |
| ORF1 coding sequence | 1952-3919 |
| ORF1/1 coding sequence | 1952-2056, 3629-3919 |
| ORF1/2 coding sequence | 1952-2056, 3756-3902 |
| IRES-SV Large T Antigen-SV40 ori-pUC57-Kan | 4131-9391, 1-435 |
| concatenated sequence 1 | |
| EMCV internal ribosome entry site (IRES) | 4203-4789 |
| SV40 Large T Antigen coding sequence | 4790-6916 |
| SV40 polyA sequence (complement) | 6947-7068 |
| SV40 origin of replication | 7108-7243 |
| LacI repressor protein binding site (complement) | 7325-7341 |
| Lac operon promoter (complement) | 7349-7379 |
| CAP binding site (complement) | 7394-7415 |
| pUC origin of replication (complement) | 7644-8317 |
| ColE1/pMB1/pBR322/pUC origin of replication | 7703-8291 |
| Aminoglycoside phosphotransferase (Kan/G418 | 8469-9278 |
| resistance protein) coding sequence (complement) | |

### Exemplary SV40 Large T Antigen Amino Acid Sequence:

(SEQ ID NO: 504)
MDKVLNREESLQLMDLLGLERSAWGNIPLMRKAYLKKCKEFHPDKGGDEEKMKKMNTLYKK
MEDGVKYAHQPDFGGFWDATEIPTYGTDEWEQWWNAFNEENLFCSEEMPSSDDEATADSQHS
TPPKKKRKVEDPKDFPSELLSFLSHAVESNRTLACFAIYTTKEKAALLYKKIMEKYSVTFISRHNS
YNHNILFFLTPHRHRVSAINNYAQKLCTFSFLICKGVNKEYLMYSALTRDPFSVIEESLPGGLKEH
DFNPEEAEETKQVSWKLVTEYAMETKCDDVLLLLGMYLEFQYSFEMCLKCIKKEQPSHYKYHE
KHYANAAIFADSKNQKTICQQAVDTVLAKKRVDSLQLTREQMLTNRFNDLLDRMDIMFGSTGS
ADIEEWMAGVAWLHCLLPKMDSVVYDFLKCMVYNIPKKRYWLFKGPIDSGKTTLAAALLELC
GGKALNVNLPLDRLNFELGVAIDQFLVVFEDVKGTGGESRDLPSGQGINNLDNLRDYLDGSVKV
NLEKKHLNKRTQIFPPGIVTMNEYSVPKTLQARFVKQIDFRPKDYLKHCLERSEFLLEKRIIQSGIA
LLLMLIWYRPVAEFAQSIQSRIVEWKERLDKEFSLSVYQKMKFNVAMGIGVLDWLRNSDDDDE
DSQENADKNEDGGEKNMEDSGHETGIDSQSQGSFQAPQSSQSVHDHNQPYHICRGFTCFKKPPT
PPPEPET

### Exemplary Site-Specific Recombinases and Recombinase Recognition Sites

The recombinase-based systems described herein utilize site-specific recombinases to induce recombination of vector plasmids at recombinase recognition sites, thereby producing double-stranded DNA molecules (e.g., minicircles) comprising the sequence between the recombinase recognition sites (e.g., comprising the sequence of a genetic element for an Anelloviridae family vector as described herein).

In some embodiments, the site-specific recombinase comprises a Cre recombinase (e.g., as described herein, e.g., in Table V2), or an amino acid sequence having at least 75%, 80%, 85%, 90%, 95%, 96%, 97%, 98%, or 99% identity thereto. In some embodiments, the site-specific recombinase comprises the amino acid sequence of Cre as listed in Table V2, or an amino acid sequence having at least 75%, 80%, 85%, 90%, 95%, 96%, 97%, 98%, or 99% identity thereto. In some embodiments, the site-specific recombinase comprises the amino acid sequence of SV40-NLS-iCre as listed in Table V2, or an amino acid sequence having at least 75%, 80%, 85%, 90%, 95%, 96%, 97%, 98%, or 99% identity thereto.

In certain embodiments, at least one (e.g., one or both) of the recombinase recognition sites comprises a lox66 site as listed in Table V3, or a nucleic acid sequence having at least 75%, 80%, 85%, 90%, 95%, 96%, 97%, 98%, or 99% identity thereto. In certain embodiments, at least one (e.g., one or both) of the recombinase recognition sites comprises a lox71 site as listed in Table V3, or a nucleic acid sequence having at least 75%, 80%, 85%, 90%, 95%, 96%, 97%, 98%, or 99% identity thereto. In certain embodiments, a circular double-stranded nucleic acid molecule (e.g., minicircle) produced after a Cre recombination event (e.g., as described herein) comprises a loxP site as listed in Table V3, or a nucleic acid sequence having at least 75%, 80%, 85%, 90%, 95%, 96%, 97%, 98%, or 99% identity thereto. In certain embodiments, a circular double-stranded nucleic acid molecule (e.g., minicircle) produced after a Cre recombination event (e.g., as described herein) comprises a lox72 site as listed in Table V3, or a nucleic acid sequence having at least 75%, 80%, 85%, 90%, 95%, 96%, 97%, 98%, or 99% identity thereto.

In some embodiments, the site-specific recombinase comprises a Bxb1 recombinase (e.g., as described herein, e.g., in Table V2), or an amino acid sequence having at least 75%, 80%, 85%, 90%, 95%, 96%, 97%, 98%, or 99% identity thereto. In some embodiments, the site-specific recombinase comprises the amino acid sequence of Bxb1 as listed in Table V2, or an amino acid sequence having at least 75%, 80%, 85%, 90%, 95%, 96%, 97%, 98%, or 99% identity thereto. In some embodiments, the site-specific recombinase comprises the amino acid sequence of SV40-NLS-HA_Bxb1 as listed in Table V2, or an amino acid sequence having at least 75%, 80%, 85%, 90%, 95%, 96%, 97%, 98%, or 99% identity thereto.

In certain embodiments, at least one of the recombinase recognition sites comprises an attB site as listed in Table V4, or a nucleic acid sequence having at least 75%, 80%, 85%, 90%, 95%, 96%, 97%, 98%, or 99% identity thereto. In certain embodiments, at least one of the recombinase recognition sites comprises an attP site as listed in Table V4, or a nucleic acid sequence having at least 75%, 80%, 85%, 90%, 95%, 96%, 97%, 98%, or 99% identity thereto. In certain embodiments, one recombinase recognition site comprises an attB site as listed in Table V4, or a nucleic acid sequence having at least 75%, 80%, 85%, 90%, 95%, 96%, 97%, 98%, or 99% identity thereto, and the other recombinase recognition site comprises an attP site as listed in Table V4, or a nucleic acid sequence having at least 75%, 80%, 85%, 90%, 95%, 96%, 97%, 98%, or 99% identity thereto. In certain embodiments, a circular double-stranded nucleic acid molecule (e.g., minicircle) produced after a Bxb1 recombination event (e.g., as described herein) comprises an attL site as listed in Table V4, or a nucleic acid sequence having at least 75%, 80%, 85%, 90%, 95%, 96%, 97%, 98%, or 99% identity thereto. In certain embodiments, a circular double-stranded nucleic acid molecule (e.g., minicircle) produced after a Bxb1 recombination event (e.g., as described herein) comprises an attR site as listed in Table V4, or a nucleic acid sequence having at least 75%, 80%, 85%, 90%, 95%, 96%, 97%, 98%, or 99% identity thereto.

TABLE V2
Exemplary site-specific recombinase polypeptides

| Site-specific | Amino Acid Sequence |
|---|---|
| recombinase | (Underline = SV40 NLS sequence; bolded italics = HA tag sequence) |
| SV40- | MVPKKKRKVSNLLTVHQNLPALPVDATSDEVRKNLMDMFRDRQAFSEHTWKMLLSVCRS |
| NLS_iCre | WAAWCKLNNRKWFPAEPEDVRDYLLYLQARGLAVKTIQQHLGQLNMLHRRSGLPRPSDS |
| | NAVSLVMRRIRKENVDAGERAKQALAFERTDFDQVRSLMENSDRCQDIRNLAFLGIAYN |
| | TLLRIAEIARIRVKDISRTDGGRMLIHIGRTKTLVSTAGVEKALSLGVTKLVERWISVS |
| | GVADDPNNYLFCRVRKNGVAAPSATSQLSTRALEGIFEATHRLIYGAKDDSGQRYLAWS |
| | GHSARVGAARDMARAGVSIPEIMQAGGWINVNIVMNYIRNLDSETGAMVRLLEDGD* |
| | (SEQ ID NO: 1101) |
| Cre (same as | MSNLLTVHQNLPALPVDATSDEVRKNLMDMFRDRQAFSEHTWKMLLSVCRSWAAWCKLN |
| iCre) | NRKWFPAEPEDVRDYLLYLQARGLAVKTIQQHLGQLNMLHRRSGLPRPSDSNAVSLVMR |
| | RIRKENVDAGERAKQALAFERTDFDQVRSLMENSDRCQDIRNLAFLGIAYNTLLRIAEI |
| | ARIRVKDISRTDGGRMLIHIGRTKTLVSTAGVEKALSLGVTKLVERWISVSGVADDPNN |
| | YLFCRVRKNGVAAPSATSQLSTRALEGIFEATHRLIYGAKDDSGQRYLAWSGHSARVGA |
| | ARDMARAGVSIPEIMQAGGWTNVNIVMNYIRNLDSETGAMVRLLEDGD (SEQ ID |
| | NO: 1102) |
| SV40-NLS- | MPKKKRKVYPYDVPDYAGSRALVVIRLSRVTDATTSPERQLESCQQLCAQRGWDVVGVA |
| HA_Bxb1 | EDLDVSGAVDPFDRKRRPNLARWLAFEEQPFDVIVAYRVDRLTRSIRHLQQLVHWAEDH |
| | KKLVVSATEAHFDTTTPFAAVVIALMGTVAQMELEAIKERNRSAAHFNIRAGKYRGSLP |
| | PWGYLPTRVDGEWRLVPDPVQRERILEVYHRVVDNHEPLHLVAHDLNRRGVLSPKDYFA |
| | QLQGREPQGREWSATALKRSMISEAMLGYATLNGKTVRDDDGAPLVRAEPILTREQLEA |
| | LRAELVKTSRAKPAVSTPSLLLRVLFCAVCGEPAYKFAGGGRKHPRYRCRSMGFPKHCG |
| | NGTVAMAEWDAFCEEQVLDLLGDAERLEKVWVAGSDSAVELAEVNAELVDLTSLIGSPA |
| | YRAGSPQREALDARIAALAARQEELEGLEARPSGWEWRETGQRFGDWWREQDTAAKNTW |
| | LRSMNVRLTFDVRGGLTRTIDFGDLQEYEQHLRLGSVVERLHTGMS* (SEQ ID NO: |
| | 1103) |
| Bxb1 | MRALVVIRLSRVTDATTSPERQLESCQQLCAQRGWDVVGVAEDLDVSGAVDPFDRKRRP |
| | NLARWLAFEEQPFDVIVAYRVDRLTRSIRHLQQLVHWAEDHKKLVVSATEAHFDTTTPF |
| | AAVVIALMGTVAQMELEAIKERNRSAAHFNIRAGKYRGSLPPWGYLPTRVDGEWRLVPD |
| | PVQRERILEVYHRVVDNHEPLHLVAHDLNRRGVLSPKDYFAQLQGREPQGREWSATALK |
| | RSMISEAMLGYATLNGKTVRDDDGAPLVRAEPILTREQLEALRAELVKTSRAKPAVSTP |
| | SLLLRVLFCAVCGEPAYKFAGGGRKHPRYRCRSMGFPKHCGNGTVAMAEWDAFCEEQVL |
| | DLLGDAERLEKVWVAGSDSAVELAEVNAELVDLTSLIGSPAYRAGSPQREALDARIAAL |
| | AARQEELEGLEARPSGWEWRETGQRFGDWWREQDTAAKNTWLRSMNVRLTFDVRGGLTR |
| | TIDFGDLQEYEQHLRLGSVVERLHTGMS (SEQ ID NO: 1104) |

TABLE V3
Exemplary recombinase recognition sequences and recombinase hybrid sites
for Cre recombinases

| Recombinase | |
|---|---|
| Recognition Site Name | Nucleic acid sequence |
| loxP | ATAACTTCGTATAGCATACATTATACGAAGTTAT (SEQ |
| | ID NO: 1105) |
| lox66 | Ataacttcgtatagcatacattatacgaacggta (SEQ |
| | ID NO: 1106) |
| lox71 | Taccgttcgtatagcatacattatacgaagttat (SEQ |
| | ID NO: 1107) |
| lox72 (hybrid) | TACCGTTCGTATAGCATACATTATACGAACGGTA (SEQ |
| | ID NO: 1108) |

TABLE V4
Exemplary recombinase recognition sequences for Bxb1 recombinases

| Recombinase | |
|---|---|
| Recognition | |
| Site Name | Nucleic acid sequence |
| attB | TCGGCCGGCTTGTCGACGACGGCGGTCTCCGTCGTCAGGATCATCCGGGC (SEQ ID |
| | NO: 1109) |
| attP | GTCGTGGTTTGTCTGGTCAACCACCGCGGTCTCAGTGGTGTACGGTACAAACCCCGAC |
| | (SEQ ID NO: 1110) |
| attL | TCGGCCGGCTTGTCGACGACGGCGGTCTCAGTGGTGTACGGTACAAACCCCGAC (SEQ |
| | ID NO: 1111) |
| attR | GTCGTGGTTTGTCTGGTCAACCACCGCGGTCTCCGTCGTCAGGATCATCCGGGC (SEQ |
| | ID NO: 1112) |

As used herein, the term “recombinase hybrid site” refers to a DNA site having a sequence that, when in double stranded form, is capable of being produced by a site-specific recombinase that recombines two recombinase recognition sites. No particular process of making is implied: a recombinase hybrid site can be produced by a site specific recombinase or another method, such as DNA replication of an existing sequence. In some embodiments, a single stranded DNA that comprises a recombinase hybrid site was produced by a method wherein a site-specific recombinase generated a double stranded DNA comprising a recombinase hybrid site, followed by conversion of the double stranded DNA to a single stranded DNA. In some embodiments (e.g., with Cre recombinase) the recombinase hybrid site has the same sequence as one of the corresponding recombinase recognition sites. In some embodiments (e.g., with Bxb1 recombinase), the recombinase hybrid site has a different site from either of the two corresponding recombinase recognition sites. In some embodiments, the recombinase hybrid site is a loxP site, an attL site, or an attR site.

As used herein, the term “recombinase recognition site” refers to a DNA site having a sequence that is capable of being recognized by a site-specific recombinase and recombined with a second recombinase recognition site, thereby producing a recombinase hybrid site. In some embodiments, the two recombinase recognition sites recognized by the recombinase have the same sequence, and in other embodiments, they have different sequences. In some embodiments, the recombinase recognition site is a loxP site, an attB site, or an attP site.

### Host Cells and Methods of Using Host Cells for Producing an Anellovector

The Anelloviridae family vector (e.g., anellovector) described herein can be produced, for example, in a host cell. Generally, a host cell is provided that comprises an Anelloviridae family vector (e.g., anellovector) genetic element and the components of an Anelloviridae family vector (e.g., anellovector) proteinaceous exterior (e.g., a polypeptide encoded by an Anellovirus ORF1 nucleic acid, or an Anellovirus ORF1 molecule). For example, in some embodiments, the host cell comprises a nucleic acid sequence encoding an Anellovirus ORF1 molecule, e.g., a splice variant or a functional fragment of an Anellovirus ORF1 polypeptide (e.g., a wild-type Anellovirus ORF1 protein or a polypeptide encoded by a wild-type Anellovirus ORF1 nucleic acid, e.g., as described herein). In embodiments, the nucleic acid sequence encoding the Anellovirus ORF1 molecule is comprised in a nucleic acid construct (e.g., a plasmid, viral vector, virus, minicircle, bacmid, or artificial chromosome) comprised in the host cell. In embodiments, the nucleic acid sequence encoding the Anellovirus ORF1 molecule is integrated into the genome of the host cell.

Producing an Anelloviridae family vector (e.g. anellovector) using the compositions or methods described herein may also involve expression of an Anellovirus ORF2 molecule (e.g., as described herein), or a splice variant or functional fragment thereof. In some embodiments, the anellovector does not comprise an ORF2 molecule, or a splice variant or functional fragment thereof, and/or a nucleic acid encoding an ORF2 molecule, or a splice variant or functional fragment thereof. In some embodiments, producing the anellovector comprises expression of an ORF2 molecule, or a splice variant or functional fragment thereof, but the ORF2 molecule is expressed from a nucleic acid other than Anelloviridae family vector.

The host cell is then incubated under conditions suitable for enclosure of the genetic element within the proteinaceous exterior (e.g., culture conditions as described herein). In some embodiments, the host cell is further incubated under conditions suitable for release of the Anelloviridae family vector (e.g., anellovector) from the host cell, e.g., into the surrounding supernatant. In some embodiments, the host cell is lysed for harvest of Anelloviridae family vector (e.g., anellovector) from the cell lysate. In some embodiments, an Anelloviridae family vector (e.g., anellovector) may be introduced to a host cell line grown to a high cell density. In some embodiments, a host cell is an Expi-293 cell.

In an aspect, the present disclosure provides a host cell (e.g., as described herein). In some embodiments, the host or host cell is a plant, insect, bacteria, fungus, vertebrate, mammal (e.g., human), or other organism or cell.

Introduction of Genetic Elements into Host Cells

The genetic element, or a nucleic acid construct comprising the sequence of a genetic element, may be introduced into a host cell. In some embodiments, the genetic element itself is introduced into the host cell. In some embodiments, a genetic element construct comprising the sequence of the genetic element (e.g., as described herein) is introduced into the host cell. A genetic element or genetic element construct can be introduced into a host cell, for example, using methods known in the art. For example, a genetic element or genetic element construct can be introduced into a host cell by transfection (e.g., stable transfection or transient transfection). In embodiments, the genetic element or genetic element construct is introduced into the host cell by lipofectamine transfection. In embodiments, the genetic element or genetic element construct is introduced into the host cell by calcium phosphate transfection. In some embodiments, the genetic element or genetic element construct is introduced into the host cell by electroporation. In some embodiments, the genetic element or genetic element construct is introduced into the host cell using a gene gun. In some embodiments, the genetic element or genetic element construct is introduced into the host cell by nucleofection. In some embodiments, the genetic element or genetic element construct is introduced into the host cell by PEI transfection. In some embodiments, the genetic element is introduced into the host cell by contacting the host cell with an Anelloviridae family vector (e.g., anellovector) comprising the genetic element. In some embodiments, cells are suspended in 2S Chica buffers.

In embodiments, the genetic element construct is capable of replication once introduced into the host cell. In embodiments, the genetic element can be produced from the genetic element construct once introduced into the host cell. In some embodiments, the genetic element is produced in the host cell by a polymerase, e.g., using the genetic element construct as a template.

In some embodiments, the genetic elements or vectors comprising the genetic elements are introduced (e.g., transfected) into cell lines that express a viral polymerase protein in order to achieve expression of the Anelloviridae family vector (e.g., anellovector). To this end, cell lines that express an Anelloviridae family vector (e.g., anellovector) polymerase protein may be utilized as appropriate host cells. Host cells may be similarly engineered to provide other viral functions or additional functions.

To prepare the Anelloviridae family vector (e.g., anellovector) disclosed herein, a genetic element construct may be used to transfect cells that provide Anelloviridae family vector (e.g., anellovector) proteins and functions required for replication and production. Alternatively, cells may be transfected with a second construct (e.g., a virus) providing Anelloviridae family vector (e.g., anellovector) proteins and functions before, during, or after transfection by the genetic element or vector comprising the genetic element disclosed herein. In some embodiments, the second construct may be useful to complement production of an incomplete viral particle. The second construct (e.g., virus) may have a conditional growth defect, such as host range restriction or temperature sensitivity, e.g., which allows the subsequent selection of transfectant viruses. In some embodiments, the second construct may provide one or more replication proteins utilized by the host cells to achieve expression of the Anelloviridae family vector (e.g., anellovector). In some embodiments, the host cells may be transfected with vectors encoding viral proteins such as the one or more replication proteins. In some embodiments, the second construct comprises an antiviral sensitivity.

The genetic element or vector comprising the genetic element disclosed herein can, in some instances, be replicated and produced into Anelloviridae family vectors (e.g., anellovectors) using techniques known in the art. For example, various viral culture methods are described, e.g., in U.S. Pat. Nos. 4,650,764; 5,166,057; 5,854,037; European Patent Publication EP 0702085A1; U.S. patent application Ser. No. 09/152,845; International Patent Publications PCT WO97/12032; WO96/34625; European Patent Publication EP-A780475; WO 99/02657; WO 98/53078; WO 98/02530; WO 99/15672; WO 98/13501; WO 97/06270; and EPO 780 47SA1, each of which is incorporated by reference herein in its entirety.

### Exemplary Cell Types

Exemplary host cells suitable for production of Anelloviridae family vector (e.g., anellovector) include, without limitation, mammalian cells, e.g., human cells and insect cells. In some embodiments, the host cell is a human cell or cell line. In some embodiments, the cell is an immune cell or cell line, e.g., a T cell or cell line, a cancer cell line, a hepatic cell or cell line, a neuron, a glial cell, a skin cell, an epithelial cell, a mesenchymal cell, a blood cell, an endothelial cell, an eye cell (e.g., a photoreceptor cell, a retinal cell, a cell of the posterior eye cup (PEC), retinal ganglion cell, a cell of the optic nerve, a cell of the optic nerve head, or a retinal pigmented epithelium (RPE) cell), a gastrointestinal cell, a progenitor cell, a precursor cell, a stem cell, a lung cell, a cardiac cell, or a muscle cell. In some embodiments, the host cell is an animal cell (e.g., a mouse cell, rat cell, rabbit cell, or hamster cell, or insect cell).

In some embodiments, the host cell is a human cell. In embodiments, the host cell is a HEK293T cell, HEK293F cell, A549 cell, Jurkat cell, Raji cell, Chang cell, HeLa cell Phoenix cell, MRC-5 cell, NCI-H292 cell, or Wi38 cell. In some embodiments, the host cell is a non-human primate cell (e.g., a Vero cell, CV-1 cell, or LLCMK2 cell). In some embodiments, the host cell is a murine cell (e.g., a McCoy cell). In some embodiments, the host cell is a hamster cell (e.g., a CHO cell or BHK 21 cell). In some embodiments, the host cell is a MARC-145, MDBK, RK-13, or EEL cell. In some embodiments, the host cell is an epithelial cell (e.g., a cell line of epithelial lineage).

In some embodiments, the host cell is a lymphoid cell. In some embodiments, the host cell is a T cell or an immortalized T cell. In embodiments, the host cell is a Jurkat cell. In embodiments, the host cell is a MOLT cell (e.g., a MOLT-4 or a MOLT-3 cell). In embodiments, the host cell is a MOLT-4 cell. In embodiments, the host cell is a MOLT-3 cell. In some embodiments, the host cell is an acute lymphoblastic leukemia (ALL) cell, e.g., a MOLT cell, e.g., a MOLT-4 or MOLT-3 cell. In some embodiments, the host cell is a B cell or an immortalized B cell. In some embodiments, the host cell comprises a genetic element construct (e.g., as described herein).

In some embodiments, the host cell is a MOLT cell (e.g., a MOLT-4 or a MOLT-3 cell).

In some embodiments, the host cell is an acute lymphoblastic leukemia (ALL) cell, e.g., a MOLT cell, e.g., a MOLT-4 or MOLT-3 cell.

In some embodiments, the host cell is a 293 cell (e.g., a HEK293 cell, a HEK293T cell, or an Expi-293 cell). In some embodiments, the host cell is an Expi-293F cell.

In an aspect, the present disclosure provides a method of manufacturing an Anelloviridae family vector (e.g., anellovector) comprising a genetic element enclosed in a proteinaceous exterior, the method comprising providing a Expi-293 cell comprising an Anelloviridae family vector (e.g., anellovector) genetic element, and incubating the Expi-293 cell under conditions that allow the Anelloviridae family vector (e.g., anellovector) genetic element to become enclosed in a proteinaceous exterior in the Expi-293 cell. In some embodiments, the Expi-293 cell further comprises one or more Anellovirus proteins (e.g., an Anellovirus ORF1 molecule) that form part or all of the proteinaceous exterior. In some embodiments, the Anelloviridae family vector (e.g., anellovector) genetic element is produced in the Expi-293 cell, e.g., from a genetic element construct (e.g., as described herein). In some embodiments, the method further comprises introducing the Anelloviridae family vector (e.g., anellovector) genetic element construct into the Expi-293 cell.

In an aspect, the present disclosure provides a method of manufacturing an Anelloviridae family vector (e.g., anellovector) comprising a genetic element enclosed in a proteinaceous exterior, the method comprising providing a MOLT-4 cell comprising an Anelloviridae family vector (e.g., anellovector) genetic element, and incubating the MOLT-4 cell under conditions that allow the Anelloviridae family vector (e.g., anellovector) genetic element to become enclosed in a proteinaceous exterior in the MOLT-4 cell. In some embodiments, the MOLT-4 cell further comprises one or more Anellovirus proteins (e.g., an Anellovirus ORF1 molecule) that form part or all of the proteinaceous exterior. In some embodiments, the Anelloviridae family vector (e.g., anellovector) genetic element is produced in the MOLT-4 cell, e.g., from a genetic element construct (e.g., as described herein). In some embodiments, the method further comprises introducing the Anelloviridae family vector (e.g., anellovector) genetic element construct into the MOLT-4 cell.

In an aspect, the present disclosure provides a method of manufacturing an Anelloviridae family vector (e.g., anellovector) comprising a genetic element enclosed in a proteinaceous exterior, the method comprising providing a MOLT-3 cell comprising an Anelloviridae family vector (e.g., anellovector) genetic element, and incubating the MOLT-3 cell under conditions that allow the Anelloviridae family vector (e.g., anellovector) genetic element to become enclosed in a proteinaceous exterior in the MOLT-3 cell. In some embodiments, the MOLT-3 cell further comprises one or more Anellovirus proteins (e.g., an Anellovirus ORF1 molecule) that form part or all of the proteinaceous exterior. In some embodiments, the Anelloviridae family vector (e.g., anellovector) genetic element is produced in the MOLT-3 cell, e.g., from a genetic element construct (e.g., as described herein). In some embodiments, the method further comprises introducing the Anelloviridae family vector (e.g., anellovector) genetic element construct into the MOLT-3 cell.

In some embodiments, the Anelloviridae family vector (e.g., anellovector) is cultivated in continuous animal cell line (e.g., immortalized cell lines that can be serially propagated).

### Culture Conditions

Host cells comprising a genetic element and components of a proteinaceous exterior can be incubated under conditions suitable for enclosure of the genetic element within the proteinaceous exterior, thereby producing an Anelloviridae family vector (e.g., anellovector). In some embodiments, the host cells are incubated in liquid media (e.g., Grace's Supplemented (TNM-FH), IPL-41, TC-100, Schneider's Drosophila, SF-900 II SFM, or and EXPRESS-FIVE™ SFM). In some embodiments, the host cells are incubated in adherent culture. In some embodiments, the host cells are incubated in suspension culture. In some embodiments, the host cells are incubated in a tube, bottle, microcarrier, or flask. In some embodiments, the host cells are incubated in a dish or well (e.g., a well on a plate). In some embodiments, the host cells are incubated under conditions suitable for proliferation of the host cells. In some embodiments, the host cells are incubated under conditions suitable for the host cells to release Anelloviridae family vectors (e.g., anellovectors) produced therein into the surrounding supernatant.

The production of Anelloviridae family vector (e.g., anellovector)-containing cell cultures according to the present invention can be carried out in different scales (e.g., in flasks, roller bottles or bioreactors). The media used for the cultivation of the cells to be infected generally comprise the standard nutrients required for cell viability, but may also comprise additional nutrients dependent on the cell type. Optionally, the medium can be protein-free and/or serum-free. Depending on the cell type the cells can be cultured in suspension or on a substrate. In some embodiments, different media is used for growth of the host cells and for production of Anelloviridae family vectors (e.g., anellovectors).

### Harvest

Anelloviridae family vectors (e.g., anellovectors) produced by host cells can be harvested, e.g., according to methods known in the art. For example, Anelloviridae family vectors (e.g., anellovectors) released into the surrounding supernatant by host cells in culture can be harvested from the supernatant. In some embodiments, the supernatant is separated from the host cells to obtain the Anelloviridae family vectors (e.g., anellovectors). In some embodiments, the host cells are lysed before or during harvest. In some embodiments, the Anelloviridae family vectors (e.g., anellovectors) are harvested from the host cell lysates. In some embodiments, the Anelloviridae family vectors (e.g., anellovectors) are harvested from both the host cell lysates and the supernatant. In some embodiments, the purification and isolation of Anelloviridae family vectors (e.g., anellovectors) is performed according to known methods in virus production, for example, as described in Rinaldi, et al., DNA Vaccines: Methods and Protocols (Methods in Molecular Biology), 3rd ed. 2014, Humana Press (incorporated herein by reference in its entirety). In some embodiments, the Anelloviridae family vector (e.g., anellovector) may be harvested and/or purified by separation of solutes based on biophysical properties, e.g., ion exchange chromatography or tangential flow filtration, prior to formulation with a pharmaceutical excipient.

### In Vitro Assembly Methods

An Anelloviridae family vector (e.g., anellovector) may be produced, e.g., by in vitro assembly, e.g., in a cell-free suspension or in a supernatant. In some embodiments, the genetic element is contacted to an ORF1 molecule in vitro, e.g., under conditions that allow for assembly.

In some embodiments, baculovirus constructs are used to produce Anelloviridae family virus (e.g., Anellovirus) proteins. These proteins may then be used, e.g., for in vitro assembly to encapsidate a genetic element, e.g., a genetic element comprising RNA. In some embodiments, a polynucleotide encoding one or more Anelloviridae family virus (e.g., Anellovirus) protein is fused to a promoter for expression in a host cell, e.g., an insect or animal cell. In some embodiments, the polynucleotide is cloned into a baculovirus expression system. In some embodiments, a host cell, e.g., an insect cell is infected with the baculovirus expression system and incubated for a period of time. In some embodiments, an infected cell is incubated for about 1, 2, 3, 4, 5, 10, 15, or 20 days. In some embodiments, an infected cell is lysed to recover the Anelloviridae family virus (e.g., Anellovirus) protein.

In some embodiments, an isolated Anelloviridae family virus (e.g., Anellovirus) protein is purified. In some embodiments, an Anellovirus protein is purified using purification techniques including but not limited to chelating purification, heparin purification, gradient sedimentation purification, and/or SEC purification. In some embodiments, a purified Anelloviridae family virus (e.g., Anellovirus) protein is mixed with a genetic element to encapsidate the genetic element, e.g., a genetic element comprising RNA. In some embodiments, a genetic element is encapsidated using an ORF1 protein, ORF2 protein, or modified version thereof. In some embodiments two nucleic acids are encapsidated. For instance, the first nucleic acid may be an mRNA e.g., chemically modified mRNA, and the second nucleic acid may be DNA.

In some embodiments, DNA encoding Anellovirus (AV) ORF1 (e.g., wildtype ORF1 protein, ORF1 proteins harboring mutations, e.g., to improve assembly efficiency, yield or stability, chimeric ORF1 protein, or fragments thereof) are expressed in insect cell lines (e.g., Sf9 and/or HighFive), animal cell lines (e.g., chicken cell lines (MDCC)), bacterial cells (e.g., E. coli) and/or mammalian cell lines (e.g., 293expi and/or MOLT4). In some embodiments, DNA encoding AV ORF1 may be untagged. In some embodiments, DNA encoding AV ORF1 may contain tags fused N-terminally and/or C-terminally. In some embodiments, DNA encoding AV ORF1 may harbor mutations, insertions or deletions within the ORF1 protein to introduce a tag, e.g., to aid in purification and/or identity determination, e.g., through immunostaining assays (including but not limited to ELISA or Western Blot). In some embodiments, DNA encoding AV ORF1 may be expressed alone or in combination with any number of helper proteins. In some embodiments, DNA encoding AV ORF1 is expressed in combination with AV ORF2 and/or ORF3 proteins.

In some embodiments, ORF1 proteins harboring mutations to improve assembly efficiency may include, but are not limited to, ORF1 proteins that harbor mutations introduced into the N-terminal Arginine Arm (ARG arm) to alter the pI of the ARG arm permitting pH sensitive nucleic acid binding to trigger particle assembly. In some embodiments, ORF1 proteins harboring mutations that improve stability may include mutations to an interprotomer contacting beta strands F and G of the canonical jellyroll beta-barrel to alter hydrophobic state of the protomer surface and improve thermodynamic favorability of capsid formation.

In some embodiments, the present disclosure describes a method of making an anellovector, the method comprising: (a) providing a mixture comprising: (i) a genetic element, and (ii) an ORF1 molecule and (b) incubating the mixture under conditions suitable for enclosing the genetic element within a proteinaceous exterior comprising the ORF1 molecule, thereby making an anellovector; optionally wherein the mixture is not comprised in a cell. In some embodiments, the method further comprises, prior to the providing of (a), expressing the ORF1 molecule, e.g., in a host cell (e.g., an insect cell or a mammalian cell). In some embodiments, the expressing comprises incubating a host cell (e.g., an insect cell or a mammalian cell) comprising a nucleic acid molecule (e.g., a baculovirus expression vector) encoding the ORF1 molecule under conditions suitable for producing the ORF1 molecule. In some embodiments, the method further comprises, prior to the providing of (a), purifying the ORF1 molecule expressed by the host cell. In some embodiments, the method is performed in a cell-free system. In some embodiments, the present disclosure describes a method of manufacturing an anellovector composition, comprising: (a) providing a plurality of anellovectors or compositions according to any of the preceding embodiments; (b) optionally evaluating the plurality for one or more of: a contaminant described herein, an optical density measurement (e.g., OD 260), particle number (e.g., by HPLC), infectivity (e.g., particle:infectious unit ratio, e.g., as determined by fluorescence and/or ELISA); and (c) formulating the plurality of anellovectors, e.g., as a pharmaceutical composition suitable for administration to a subject, e.g., if one or more of the parameters of (b) meet a specified threshold.

### Enrichment and Purification

Harvested Anelloviridae family vectors can be purified and/or enriched, e.g., to produce an anellovector preparation. In some embodiments, the harvested anellovectors are isolated from other constituents or contaminants present in the harvest solution, e.g., using methods known in the art for purifying viral particles (e.g., purification by sedimentation, chromatography, and/or ultrafiltration). In some embodiments, the purification steps comprise removing one or more of serum, host cell DNA, host cell proteins, particles lacking the genetic element, and/or phenol red from the preparation. In some embodiments, the harvested Anelloviridae family vectors are enriched relative to other constituents or contaminants present in the harvest solution, e.g., using methods known in the art for enriching viral particles.

In some embodiments, the resultant preparation or a pharmaceutical composition comprising the preparation will be stable over an acceptable period of time and temperature, and/or be compatible with the desired route of administration and/or any devices this route of administration will require, e.g., needles or syringes.

### III. Pharmaceutical Compositions

The Anelloviridae family vector, anellovector, or other vector described herein may also be included in pharmaceutical compositions with a pharmaceutical excipient, e.g., as described herein. In some embodiments, the pharmaceutical composition comprises at least 10⁵, 10⁶, 10⁷, 10⁸, 10⁹, 10¹⁰, 10¹¹, 10¹², 10¹³, 10¹⁴, or 10¹⁵ Anelloviridae family vectors. In some embodiments, the pharmaceutical composition comprises about 10⁵-10¹⁵, 10⁵-10¹⁰, or 10¹⁰-10¹⁵ Anelloviridae family vectors. In some embodiments, the pharmaceutical composition comprises about 10⁸ (e.g., about 10⁵, 10⁶, 10⁷, 10⁸, 10⁹, or 10¹⁰) genomic equivalents/mL of the Anelloviridae family vector. In some embodiments, the pharmaceutical composition comprises 10⁵-10¹⁰, 10⁶-10¹⁰, 10⁷-10¹⁰, 10⁸-10¹⁰, 10⁹-10¹⁰, 10⁵-10⁶, 10⁵-10⁷, 10⁵-10⁸, 10⁵-10⁹, 10⁵-10¹¹, 10⁵-10¹², 10⁵-10¹¹, 10⁵-10¹⁴, 10⁵-10¹⁵, or 10¹⁰-10¹⁵ genomic equivalents/mL of the Anelloviridae family vector. In some embodiments, the pharmaceutical composition comprises sufficient Anelloviridae family vectors to deliver at least 1, 2, 5, or 10, 100, 500, 1000, 2000, 5000, 8,000, 1×10⁴, 1×10⁵, 1×10⁶, 1×10⁷ or greater copies of a genetic element comprised in the Anelloviridae family vectors per cell to a population of the eukaryotic cells. In some embodiments, the pharmaceutical composition comprises sufficient Anelloviridae family vectors to deliver at least about 1×10⁴, 1×10⁵, 1×10⁶, 1× or 10⁷, or about 1×10⁴-1×10⁵, 1×10⁴-1×10⁶, 1×10⁴-1×10⁷, 1×10⁵-1×10⁶, 1×10⁵-1×10⁷, or 1×10⁶-1×10⁷ copies of a genetic element comprised in the Anelloviridae family vectors per cell to a population of the eukaryotic cells.

In some embodiments, the pharmaceutical composition has one or more of the following characteristics: the pharmaceutical composition meets a pharmaceutical or good manufacturing practices (GMP) standard; the pharmaceutical composition was made according to good manufacturing practices (GMP); the pharmaceutical composition has a pathogen level below a predetermined reference value, e.g., is substantially free of pathogens; the pharmaceutical composition has a contaminant level below a predetermined reference value, e.g., is substantially free of contaminants; or the pharmaceutical composition has low immunogenicity or is substantially non-immunogenic, e.g., as described herein.

In some embodiments, the pharmaceutical composition comprises below a threshold amount of one or more contaminants. Exemplary contaminants that are desirably excluded or minimized in the pharmaceutical composition include, without limitation, host cell nucleic acids (e.g., host cell DNA and/or host cell RNA), animal-derived components (e.g., serum albumin or trypsin), replication-competent viruses, non-infectious particles, free viral capsid protein, adventitious agents, and aggregates. In embodiments, the contaminant is host cell DNA. In embodiments, the composition comprises less than about 10 ng of host cell DNA per dose. In embodiments, the level of host cell DNA in the composition is reduced by filtration and/or enzymatic degradation of host cell DNA. In embodiments, the pharmaceutical composition consists of less than 10% (e.g., less than about 10%, 5%, 4%, 3%, 2%, 1%, 0.5%, or 0.1%) contaminant by weight.

In one aspect, the invention described herein includes a pharmaceutical composition comprising:
a) an Anelloviridae family vector (e.g., anellovector) comprising a genetic element comprising (i) a sequence encoding a non-pathogenic exterior protein, (ii) an exterior protein binding sequence that binds the genetic element to the non-pathogenic exterior protein, and (iii) a sequence encoding a regulatory nucleic acid; and a proteinaceous exterior that is associated with, e.g., envelops or encloses, the genetic element; and
b) a pharmaceutical excipient.

### IV. Methods of Use

The Anelloviridae family vectors, e.g., anellovectors, and compositions comprising Anelloviridae family vectors, e.g., anellovectors, described herein may be used in methods of treating a disease, disorder, or condition, e.g., in a subject (e.g., a mammalian subject, e.g., a human subject) in need thereof. Administration of a pharmaceutical composition described herein may be, for example, by way of parenteral administration. In some embodiments, an Anelloviridae family vector, e.g., anellovector, or pharmaceutical composition as described herein is administered subretinally. In some embodiments, an Anelloviridae family vector, e.g., anellovector, or pharmaceutical composition as described herein is administered intravitreally. In some embodiments, an Anelloviridae family vector, e.g., anellovector, or pharmaceutical composition as described herein is administered suprachoroidally. The anellovectors may be administered alone or formulated as a pharmaceutical composition.

The Anelloviridae family vector (e.g., anellovector) may be administered in the form of a unit-dose composition, such as a unit dose parenteral composition. Such compositions are generally prepared by admixture and can be suitably adapted for parenteral administration. Such compositions may be, for example, in the form of injectable and infusable solutions or suspensions or suppositories or aerosols.

In some embodiments, administration of an Anelloviridae family vector (e.g., anellovector) or composition comprising same, e.g., as described herein, may result in delivery of a genetic element comprised by the Anelloviridae family vector (e.g., anellovector) to a target cell, e.g., in a subject.

An Anelloviridae family vector (e.g., anellovector) or composition thereof described herein, e.g., comprising an effector (e.g., an endogenous or exogenous effector), may be used to deliver the effector to a cell, tissue, or subject. In some embodiments, the effector is a therapeutic effector. In some embodiments, the Anelloviridae family vector (e.g., anellovector) or composition thereof is used to deliver the effector to the eye of a subject, e.g., a mammalian subject, e.g., a human subject. In some embodiments, the Anelloviridae family vector (e.g., anellovector) or composition thereof is used to deliver the effector to a cell of the eye of a subject, e.g., a mammalian subject, e.g., a human subject. In certain embodiments, the cell of the eye is a photoreceptor cell, a retinal cell, a cell of the posterior eye cup (PEC), retinal ganglion cell, a cell of the optic nerve, a cell of the optic nerve head, or a retinal pigmented epithelium (RPE) cell. In some embodiments, the Anelloviridae family vector (e.g., anellovector) or composition thereof is used to deliver the effector to bone marrow, blood, heart, GI or skin. Delivery of an effector by administration of an Anelloviridae family vector (e.g., anellovector) composition described herein may modulate (e.g., increase or decrease) expression levels of a noncoding RNA or polypeptide in the cell, tissue, or subject. Modulation of expression level in this fashion may result in alteration of a functional activity in the cell to which the effector is delivered. In some embodiments, the modulated functional activity may be enzymatic, structural, or regulatory in nature.

In some embodiments, the Anelloviridae family vector (e.g., anellovector), or copies thereof, are detectable in a cell 24 hours (e.g., 1 day, 2 days, 3 days, 4 days, 5 days, 6 days, 1 week, 2 weeks, 3 weeks, 4 weeks, 30 days, or 1 month) after delivery into a cell. In embodiments, an Anelloviridae family vector (e.g., anellovector) or composition thereof mediates an effect on a target cell, and the effect lasts for at least 1, 2, 3, 4, 5, 6, or 7 days, 2, 3, or 4 weeks, or 1, 2, 3, 6, or 12 months. In some embodiments (e.g., wherein the Anelloviridae family vector (e.g., anellovector) or composition thereof comprises a genetic element encoding an exogenous protein), the effect lasts for less than 1, 2, 3, 4, 5, 6, or 7 days, 2, 3, or 4 weeks, or 1, 2, 3, 6, or 12 months.

### V. Redosing

The Anelloviridae family vector (e.g., anellovector) described herein can, in some instances, be used as a delivery vehicle that can be administered in multiple doses (e.g., doses administered separately). While not wishing to be bound by theory, in some embodiments, an Anelloviridae family vector (e.g., anellovector) (e.g., as described herein) induces a relatively low immune response (as measured, for example, as 50% GMT values), e.g., allowing for repeated dosing of a subject with one or more Anelloviridae family vectors (e.g., anellovectors) (e.g., multiple doses of the same Anelloviridae family vector (e.g., anellovector) or different Anelloviridae family vectors (e.g., anellovectors)). In an aspect, the invention provides a method of delivering an effector, comprising administering to a subject a first plurality of Anelloviridae family vectors (e.g., anellovectors) and then a second plurality of Anelloviridae family vectors (e.g., anellovectors). In some embodiments, the second plurality of Anelloviridae family vectors (e.g., anellovectors) comprise the same proteinaceous exterior as the Anelloviridae family vectors (e.g., anellovectors) of the first plurality. In another aspect, the invention provides a method of selecting a subject (e.g., a human subject) to receive an effector, wherein the subject previously received, or was identified as having received, a first plurality of Anelloviridae family vectors (e.g., anellovectors) comprising a genetic element encoding an effector, in which the method involves selecting the subject to receive a second plurality of Anelloviridae family vectors (e.g., anellovectors) comprising a genetic element encoding an effector (e.g., the same effector as that encoded by the genetic element of the first plurality of Anelloviridae family vectors (e.g., anellovectors), or a different effector as that encoded by the genetic element of the first plurality of Anelloviridae family vectors (e.g., anellovectors)). In another aspect, the invention provides a method of identifying a subject (e.g., a human subject) as suitable to receive a second plurality of Anelloviridae family vectors (e.g., anellovectors), the method comprising identifying the subject has having previously received a first plurality of Anelloviridae family vectors (e.g., anellovectors) comprising a genetic element encoding an effector, wherein the subject being identified as having received the first plurality of Anelloviridae family vectors (e.g., anellovectors) is indicative that the subject is suitable to receive the second plurality of Anelloviridae family vectors (e.g., anellovectors).

All references and publications cited herein are hereby incorporated by reference.

The following examples are provided to further illustrate some embodiments of the present invention, but are not intended to limit the scope of the invention; it will be understood by their exemplary nature that other procedures, methodologies, or techniques known to those skilled in the art may alternatively be used.

### EXAMPLES

### Table of Contents

Example 1: Anellovector production for in vivo transduction of the CNS
Example 2: In vivo transduction of the brain via intracerebroventricular (ICV) administration with an Anellovector (Study #1)
Example 3: In vivo delivery of an Anellovector to the spinal cord via intrathecal (IT) administration (Study #2)
Example 4: In vivo delivery of an Anellovector to the spinal cord and redosing via IT administration
Example 5: In vivo delivery of an Anellovector to brain and redosing via ICV administration

### Example 1: Anellovector Production for In Vivo Transduction of the CNS

This example describes the production of the Anellovector and AAV control vector used for Study #1 (Example 2) and Study #2 (Example 3) below for in vivo transduction of the CNS.

### AAV9-fCMV-eGFP Control

AAV9-fCMV-eGFP is an adeno-associated virus based on the plasmid pRTx-2770 (SEQ ID NO: 501) with a payload comprising from the 5′ to 3′ direction an AAV2 ITR, Ring2 5′ NCR, CMV promoter, eGFP, SV40pA, Ring2 3′ NCR, and AAV2 ITR (Table Z6). The payload was packaged into AAV9 by Packgene Biotech Inc (Houston, Texas) and generated at a titer of 1×10¹³ viral genomes (vgs)/ml. Prior to injection, AAV9-fCMV-eGFP was diluted in sterile 1×PBS to the desired titer.

TABLE Z6
pRTx-2770

| | |
|---|---|
| Name | pRTx-2770 |
| Type | Plasmid |
| Length | 5128 bp |

| | |
|---|---|
| 1 | GTCAGTGAGC GAGGAAGCGG AAGAGCGCCC AATACGCAAA CCGCCTCTCC CCGCGCGTTG |
| 61 | GCCGATTCAT TAATGCAGCT GGCACGACAG GTTTCCCGAC TGGAAAGCGG GCAGTGAGCG |
| 121 | CAACGCAATT AATGTGAGTT AGCTCACTCA TTAGGCACCC CAGGCTTTAC ACTTTATGCT |
| 181 | TCCGGCTCGT ATGTTGTGTG GAATTGTGAG CGGATAACAA TTTCACACAG GAAACAGCTA |
| 241 | TGACCATGAT TACGCCAAGC TTGCATGCCC TGCAGGCAGC TGCGCGCTCG CTCGCTCACT |
| 301 | GAGGCCGCCC GGGCAAAGCC CGGGCGTCGG GCGACCTTTG GTCGCCCGGC CTCAGTGAGC |
| 361 | GAGCGAGCGC GCAGAGAGGG AGTGGCCAAC TCCATCACTA GGGGTTCCTG CGAAAGATAT |
| 421 | CTAATAAATA TTCAACAGGA AAACCACCTA ATTTAAATTG CCGACCACAA ACCGTCACTT |
| 481 | AGTTCCCCTT TTTGCAACAA CTTCTGCTTT TTTCCAACTG CCGGAAAACC ACATAATTTG |
| 541 | CATGGCTAAC CACAAACTGA TATGCTAATT AACTTCCACA AAACAACTTC CCCTTTTAAA |
| 601 | ACCACACCTA CAAATTAATT ATTAAACACA GTCACATCCT GGGAGGTACT ACCACACTAT |
| 661 | AATACCAAGT GCTAATCCGA ATGGCTGAGT TTATGCCGCT AGACGGAGAA CGCATCAGTT |
| 721 | ACTGACTGCG GACTGAACTT GGGCGGGTGC CGAAGGTGAG TGAAACCACC GAAGTCAAGG |
| 781 | GGCAATTCGG GCTAGTTCAG TCTAGCGGAA CGGGCAAGAA ACTTAAAATT ATTTTATTTT |
| 841 | TCAGATGGAT GGCTGATCGA GTGTAGCCAG ATCTGCGATC GACATTGATT ATTGACTAGT |
| 901 | TATTAATAGT AATCAATTAC GGGGTCATTA GTTCATAGCC CATATATGGA GTTCCGCGTT |
| 961 | ACATAACTTA CGGTAAATGG CCCGCCTGGC TGACCGCCCA ACGACCCCCG CCCATTGACG |
| 1021 | TCAATAATGA CGTATGTTCC CATAGTAACG CCAATAGGGA CTTTCCATTG ACGTCAATGG |
| 1081 | GTGGAGTATT TACGGTAAAC TGCCCACTTG GCAGTACATC AAGTGTATCA TATGCCAAGT |
| 1141 | ACGCCCCCTA TTGACGTCAA TGACGGTAAA TGGCCCGCCT GGCATTATGC CCAGTACATG |
| 1201 | ACCTTATGGG ACTTTCCTAC TTGGCAGTAC ATCTACGTAT TAGTCATCGC TATTACCATG |
| 1261 | GTGATGCGGT TTTGGCAGTA CATCAATGGG CGTGGATAGC GGTTTGACTC ACGGGGATTT |
| 1321 | CCAAGTCTCC ACCCCATTGA CGTCAATGGG AGTTTGTTTT GGCACCAAAA TCAACGGGAC |
| 1381 | TTTCCAAAAT GTCGTAACAA CTCCGCCCCA TTGACGCAAA TGGGCGGTAG GCGTGTACGG |
| 1441 | TGGGAGGTCT ATATAAGCAG AGCTCTCTGG CTAACTGGAT CTACAAAAAA GCAGATCCAC |
| 1501 | CGGTCGCCAC CATGGTGAGC AAGGGCGAGG AGCTGTTCAC CGGGGTGGTG CCCATCCTGG |
| 1561 | TCGAGCTGGA CGGCGACGTA AACGGCCACA AGTTCAGCGT GTCCGGCGAG GGCGAGGGCG |
| 1621 | ATGCCACCTA CGGCAAGCTG ACCCTGAAGT TCATCTGCAC CACCGGCAAG CTGCCCGTGC |
| 1681 | CCTGGCCCAC CCTCGTGACC ACCCTGACCT ACGGCGTGCA GTGCTTCAGC CGCTACCCCG |
| 1741 | ACCACATGAA GCAGCACGAC TTCTTCAAGT CCGCCATGCC CGAAGGCTAC GTCCAGGAGC |
| 1801 | GCACCATCTT CTTCAAGGAC GACGGCAACT ACAAGACCCG CGCCGAGGTG AAGTTCGAGG |
| 1861 | GCGACACCCT GGTGAACCGC ATCGAGCTGA AGGGCATCGA CTTCAAGGAG GACGGCAACA |
| 1921 | TCCTGGGGCA CAAGCTGGAG TACAACTACA ACAGCCACAA CGTCTATATC ATGGCCGACA |
| 1981 | AGCAGAAGAA CGGCATCAAG GTGAACTTCA AGATCCGCCA CAACATCGAG GACGGCAGCG |
| 2041 | TGCAGCTCGC CGACCACTAC CAGCAGAACA CCCCCATCGG CGACGGCCCC GTGCTGCTGC |
| 2101 | CCGACAACCA CTACCTGAGC ACCCAGTCCG CCCTGAGCAA AGACCCCAAC GAGAAGCGCG |
| 2161 | ATCACATGGT CCTGCTGGAG TTCGTGACCG CCGCCGGGAT CACTCTCGGC ATGGACGAGC |
| 2221 | TGTACAAGTA ATAAGCTTGC GGCCGCTTCG AGCAGACATG ATAAGATACA TTGATGAGTT |
| 2281 | TGGACAAACC ACAACTAGAA TGCAGTGAAA AAAATGCTTT ATTTGTGAAA TTTGTGATGC |
| 2341 | TATTGCTTTA TTTGTAACCA TTATAAGCTG CAATAAACAA GTTAACAACA ACAATTGCAT |
| 2401 | GAATGAATAA AGGCCAGCAT TAATTCACTT AAGGAGTCTG TTTATTTAAG TTAAACCTTA |
| 2461 | ATAAACGGTC ACCGCCTCCC TAATACGCAG GCGCAGAAAG GGGGCTCCGC CCCCTTTAAC |
| 2521 | CCCCAGGGGG CTCCGCCCCC TGAAACCCCC AAGGGGGCTA CGCCCCCTTA CACCCCCGAT |
| 2581 | ATCCCCGCAG GAACCCCTAG TGATGGAGTT GGCCACTCCC TCTCTGCGCG CTCGCTCGCT |
| 2641 | CACTGAGGCC GGGCGACCAA AGGTCGCCCG ACGCCCGGGC TTTGCCCGGG CGGCCTCAGT |
| 2701 | GAGCGAGCGA GCGCGCAGCT GCCTGCAGGG GCGCCTGGGT ACCGAGCTCG AATTCACTGG |
| 2761 | CCGTCGTTTT ACAACGTCGT GACTGGGAAA ACCCTGGCGT TACCCAACTT AATCGCCTTG |
| 2821 | CAGCACATCC CCCTTTCGCC AGCTGGCGTA ATAGCGAAGA GGCCCGCACC GATCGCCCTT |
| 2881 | CCCAACAGTT GCGCAGCCTG AATGGCGAAT GGCGCCTGAT GCGGTATTTT CTCCTTACGC |
| 2941 | ATCTGTGCGG TATTTCACAC CGCATATGGT GCACTCTCAG TACAATCTGC TCTGATGCCG |
| 3001 | CATAGTTAAG CCAGCCCCGA CACCCGCCAA CACCCGCTGA CGCGCCCTGA CGGGCTTGTC |
| 3061 | TGCTCCCGGC ATCCGCTTAC AGACAAGCTG TGACCGTCTC CGGGAGCTGC ATGTGTCAGA |
| 3121 | GGTTTTCACC GTCATCACCG AAACGCGCGA GACGAAAGGG CCTCGTGATA CGCCTATTTT |
| 3181 | TATAGGTTAA TGTCATGATA ATAATGGTTT CTTAGACGTC AGGTGGCACT TTTCGGGGAA |
| 3241 | ATGTGCGCGG AACCCCTATT TGTTTATTTT TCTAAATACA TTCAAATATG TATCCGCTCA |
| 3301 | TGAGACAATA ACCCTGATAA ATGCTTCAAT AATATTGAAA AAGGAAGAGT ATGAGTATTC |
| 3361 | AACATTTCCG TGTCGCCCTT ATTCCCTTTT TTGCGGCATT TTGCCTTCCT GTTTTTGCTC |
| 3421 | ACCCAGAAAC GCTGGTGAAA GTAAAAGATG CTGAAGATCA GTTGGGTGCA CGAGTGGGTT |
| 3481 | ACATCGAACT GGATCTCAAC AGCGGTAAGA TCCTTGAGAG TTTTCGCCCC GAAGAACGTT |
| 3541 | TTCCAATGAT GAGCACTTTT AAAGTTCTGC TATGTGGCGC GGTATTATCC CGTATTGACG |
| 3601 | CCGGGCAAGA GCAACTCGGT CGCCGCATAC ACTATTCTCA GAATGACTTG GTTGAGTACT |
| 3661 | CACCAGTCAC AGAAAAGCAT CTTACGGATG GCATGACAGT AAGAGAATTA TGCAGTGCTG |
| 3721 | CCATAACCAT GAGTGATAAC ACTGCGGCCA ACTTACTTCT GACAACGATC GGAGGACCGA |
| 3781 | AGGAGCTAAC CGCTTTTTTG CACAACATGG GGGATCATGT AACTCGCCTT GATCGTTGGG |
| 3841 | AACCGGAGCT GAATGAAGCC ATACCAAACG ACGAGCGTGA CACCACGATG CCTGTAGCAA |
| 3901 | TGGCAACAAC GTTGCGCAAA CTATTAACTG GCGAACTACT TACTCTAGCT TCCCGGCAAC |
| 3961 | AATTAATAGA CTGGATGGAG GCGGATAAAG TTGCAGGACC ACTTCTGCGC TCGGCCCTTC |
| 4021 | CGGCTGGCTG GTTTATTGCT GATAAATCTG GAGCCGGTGA GCGTGGGTCT CGCGGTATCA |
| 4081 | TTGCAGCACT GGGGCCAGAT GGTAAGCCCT CCCGTATCGT AGTTATCTAC ACGACGGGGA |
| 4141 | GTCAGGCAAC TATGGATGAA CGAAATAGAC AGATCGCTGA GATAGGTGCC TCACTGATTA |
| 4201 | AGCATTGGTA ACTGTCAGAC CAAGTTTACT CATATATACT TTAGATTGAT TTAAAACTTC |
| 4261 | ATTTTTAATT TAAAAGGATC TAGGTGAAGA TCCTTTTTGA TAATCTCATG ACCAAAATCC |
| 4321 | CTTAACGTGA GTTTTCGTTC CACTGAGCGT CAGACCCCGT AGAAAAGATC AAAGGATCTT |
| 4381 | CTTGAGATCC TTTTTTTCTG CGCGTAATCT GCTGCTTGCA AACAAAAAAA CCACCGCTAC |
| 4441 | CAGCGGTGGT TTGTTTGCCG GATCAAGAGC TACCAACTCT TTTTCCGAAG GTAACTGGCT |
| 4501 | TCAGCAGAGC GCAGATACCA AATACTGTTC TTCTAGTGTA GCCGTAGTTA GGCCACCACT |
| 4561 | TCAAGAACTC TGTAGCACCG CCTACATACC TCGCTCTGCT AATCCTGTTA CCAGTGGCTG |
| 4621 | CTGCCAGTGG CGATAAGTCG TGTCTTACCG GGTTGGACTC AAGACGATAG TTACCGGATA |
| 4681 | AGGCGCAGCG GTCGGGCTGA ACGGGGGGTT CGTGCACACA GCCCAGCTTG GAGCGAACGA |
| 4741 | CCTACACCGA ACTGAGATAC CTACAGCGTG AGCTATGAGA AAGCGCCACG CTTCCCGAAG |
| 4801 | GGAGAAAGGC GGACAGGTAT CCGGTAAGCG GCAGGGTCGG AACAGGAGAG CGCACGAGGG |
| 4861 | AGCTTCCAGG GGGAAACGCC TGGTATCTTT ATAGTCCTGT CGGGTTTCGC CACCTCTGAC |
| 4921 | TTGAGCGTCG ATTTTTGTGA TGCTCGTCAG GGGGGCGGAG CCTATGGAAA AACGCCAGCA |
| 4981 | ACGCGGCCTT TTTACGGTTC CTGGCCTTTT GCTGGCCTTT TGCTCACATG TTCTTTCCTG |
| 5041 | CGTTATCCCC TGATTCTGTG GATAACCGTA TTACCGCCTT TGAGTGAGCT GATACCGCTC |
| 5101 | GCCGCAGCCG AACGACCGAG CGCAGCGA (SEQ ID NO: 501) |

Annotations:

| Region/Element | Base range |
|---|---|
| E. coli CAP protein binding site | 130..151 |
| Lac operon promoter | 166..196 |
| LacI repressor protein binding site | 204..220 |
| AAV2 inverted terminal repeat | 269..409 |
| Ring2 5′NCR | 421..844 |
| CMV enhancer | 881..1260 |
| Full CMV promoter | 896..1465 |
| CMV promoter | 1261..1464 |
| Inert 5′ UTR | 1477..1507 |
| Kozak sequence | 1506..1515 |
| eGFP coding sequence | 1512..2231 |
| SV40 poly(A) sequence | 2262..2383 |
| Ring2 3′NCR | 2411..2575 |
| AAV2 inverted terminal repeat | 2589..2729 |
| AmpR promoter | 3246..3350 |
| Beta lactamase ampicillin resistance | 3351..4211 |
| coding sequence | |
| High-copy-number | 4382..4970 |
| ColE1/pMB1/pBR322/pUC origin of | |
| replication | |

Exemplary eGFP Amino Acid Sequence (e.g., Encoded by the Nucleotides 1512-2231 of Table Z6):

(SEQ ID NO: 1113)
MVSKGEELFTGVVPILVELDGDVNGHKFSVSGEGEGDATYGKLTLKFIC
TTGKLPVPWPTLVTTLTYGVQCFSRYPDHMKQHDFFKSAMPEGYVQERT
IFFKDDGNYKTRAEVKFEGDTLVNRIELKGIDFKEDGNILGHKLEYNYN
SHNVYIMADKQKNGIKVNFKIRHNIEDGSVQLADHYQQNTPIGDGPVLL
PDNHYLSTQSALSKDPNEKRDHMVLLEFVTAAGITLGMDELYK

### Exemplary Beta Lactamase Amino Acid Sequence (e.g., Encoded by of Nucleotides 3351-4211 of Table Z6):

(SEQ ID NO: 1114)
MSIQHFRVALIPFFAAFCLPVFAHPETLVKVKDAEDQLGARVGYIELDL
NSGKILESFRPEERFPMMSTFKVLLCGAVLSRIDAGQEQLGRRIHYSQN
DLVEYSPVTEKHLTDGMTVRELCSAAITMSDNTAANLLLTTIGGPKELT
AFLHNMGDHVTRLDRWEPELNEAIPNDERDTTMPVAMATTLRKLLTGEL
LTLASRQQLIDWMEADKVAGPLLRSALPAGWFIADKSGAGERGSRGIIA
ALGPDGKPSRIVVIYTTGSQATMDERNRQIAEIGASLIKHW

### Ring19-fCMV-eGFP Anellovector

### Anellovirus Vector Production: Parental Cell Culture

MOLT-4 cells were obtained from the National Cancer Institute. Cells were scaled-up and maintained in suspension culture in complete growth medium (Gibco's RPMI 1640 with 10% fetal bovine serum (FBS), supplemented with 1 mM sodium pyruvate, Pluronic F-68 [0.1%], and 2 mM L-glutamine) at 37° C. with 5% CO₂. Cells were seeded into shake flasks (2-L, flat-bottomed, Erlenmeyer flask), each with a working volume of 800 mL, at a density of 0.1E+06 viable cells/mL and cultured in an orbital shaker (New Brunswick Innova 2100, 19-mm circular orbit) at 37° C. and 90 rpm with >85% relative humidity (RH) for 4 days.

### Anellovirus Vector Production: Transfection of MOLT-4 Cells

Ring19-fCMV-eGFP Anellovector was produced using a distinct combination of three plasmids listed in Table E0. MOLT-4 cells were transfected with the three plasmids via electroporation.

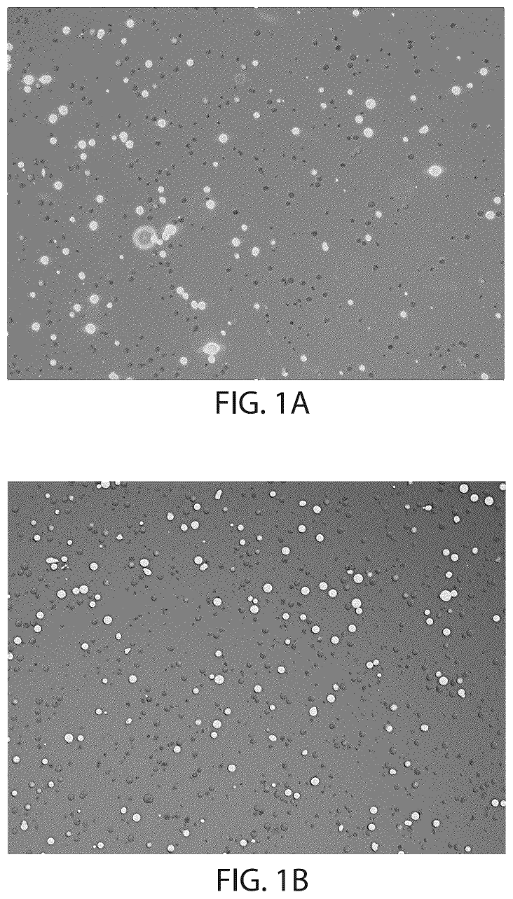

For each of the viral vector production culture preps, 5E+08 MOLT-4 cells were pelleted and resuspended in Opti-MEM I Reduced Serum Medium. 800 μg of the plasmids were added to each of the resuspended cells and electroporated using a MaxCyte STx electroporator and CL1.1 electroporator assemblies (MaxCyte catalog #SCL1). Each batch of electroporated cells were then transferred to separate flasks containing pre-warmed complete growth medium. Transfected cells were allowed to incubate at 37° C. with 5% CO₂ and harvested via centrifugation 72 hours post-electroporation. 10× images were captured with an EVOS (M7000, Invitrogen by ThermoFisher Scientific) (FIG. 1A and FIG. 1B).

TABLE E0
Plasmids transfected to produce
Ring19-fCMV-eGFP Anellovector

| Anellovector | Plasmids |
|---|---|
| Ring19-fCMV-eGFP | pRTx-2847 (R19-eGFP vector) |
| (“R19-eGFP”) | (SEQ ID NO: 502) |
| | pRTx-2848 (iCre plasmid) |
| | (SEQ ID NO: 503) |
| | pRTx-3525 (R19 SRR) |
| | (SEQ ID NO: 500) |

### Genetic Element Construct Plasmid

The Ring19 genetic element construct, pRTX-2847 (SEQ ID NO: 502), was designed with lox66 and lox 71 sites flanking a RING19 non-coding region (NCR) and a CMV::egfp::WPRE::bGH-pA payload cassette. The sequence for the construct is provided below.

TABLE Z1
Exemplary floxed Ring19 vector plasmid for three-plasmid systems (pRTx-2847)

| | |
|---|---|
| Name | pRTx-2847 |
| Type | Plasmid |
| Description | pLox-Ring19AORF::fCMV_EGFP_WPRE_bGH-pA-Rand100. |
| Length | 5452 bp |

| | |
|---|---|
| 1 | TCGCGCGTTT CGGTGATGAC GGTGAAAACC TCTGACACAT GCAGCTCCCG GAGACGGTCA |
| 61 | CAGCTTGTCT GTAAGCGGAT GCCGGGAGCA GACAAGCCCG TCAGGGCGCG TCAGCGGGTG |
| 121 | TTGGCGGGTG TCGGGGCTGG CTTAACTATG CGGCATCAGA GCAGATTGTA CTGAGAGTGC |
| 181 | ACCATATGCG GTGTGAAATA CCGCACAGAT GCGTAAGGAG AAAATACCGC ATCAGGCGCC |
| 241 | ATTCGCCATT CAGGCTGCGC AACTGTTGGG AAGGGCGATC GGTGCGGGCC TCTTCGCTAT |
| 301 | TACGCCAGCT GGCGAAAGGG GGATGTGCTG CAAGGCGATT AAGTTGGGTA ACGCCAGGGT |
| 361 | TTTCCCAGTC ACGACGTTGT AAAACGACGG CCAGAGAATT CGAGCTCGGT ACCTCGCGAA |
| 421 | TACATCTAGA TTACCGTTCG TATAGCATAC ATTATACGAA GTTATTAAAC TACGCCTGCA |
| 481 | AACTTTCACT CTCGGTGTCC ATTTATATAA GATAAAACTT AAATAAACAT CCACCACTCT |
| 541 | CCCAAATACG CAGGCGCACA AGGGGGCTCC GCCCCCTTAA ACCCCCAAGG GGGCTCCGCC |
| 601 | CCCTTAAACC CCCAAGGGGG CTCCGCCCCC TTACACCCCC TAATAAATAT TCAACAGGAA |
| 661 | AACCACCTAA TTAGAATTGC CGACCACAAA CCGTCACTTA CTTCTCCTTT TTGCACTTAC |
| 721 | TTCCTCTTTT ACTTATTATT ATTCATTACA TTAATTAATA ATCACTGTAA TTCCGGGGAG |
| 781 | GAGCTAACAA TCTATATAAC TAACTACACT TCCGAATGGC TGAGTTTATG CCGCCAGACG |
| 841 | GAGACGGGAT CACTTCAGTG ACTCCAGGCT GAACTTGGGC GGGAGCCGAA GGTGAGTGCA |
| 901 | ACCACCGTAG TCTAGGGGCA ATTCGGGCTA GTTCAGTATG GCGGAACGGG CAAGAAACTT |
| 961 | AAATATTATT ATTTTACAGA TGGGCGTTGA CATTGATTAT TGACTAGTTA TTAATAGTAA |
| 1021 | TCAATTACGG GGTCATTAGT TCATAGCCCA TATATGGAGT TCCGCGTTAC ATAACTTACG |
| 1081 | GTAAATGGCC CGCCTGGCTG ACCGCCCAAC GACCCCCGCC CATTGACGTC AATAATGACG |
| 1141 | TATGTTCCCA TAGTAACGCC AATAGGGACT TTCCATTGAC GTCAATGGGT GGAGTATTTA |
| 1201 | CGGTAAACTG CCCACTTGGC AGTACATCAA GTGTATCATA TGCCAAGTAC GCCCCCTATT |
| 1261 | GACGTCAATG ACGGTAAATG GCCCGCCTGG CATTATGCCC AGTACATGAC CTTATGGGAC |
| 1321 | TTTCCTACTT GGCAGTACAT CTACGTATTA GTCATCGCTA TTACCATGGT GATGCGGTTT |
| 1381 | TGGCAGTACA TCAATGGGCG TGGATAGCGG TTTGACTCAC GGGGATTTCC AAGTCTCCAC |
| 1441 | CCCATTGACG TCAATGGGAG TTTGTTTTGG CACCAAAATC AACGGGACTT TCCAAAATGT |
| 1501 | CGTAACAACT CCGCCCCATT GACGCAAATG GGCGGTAGGC GTGTACGGTG GGAGGTCTAT |
| 1561 | ATAAGCAGAG CTCTCTGGCT AACTGGATCT ACAAAAAAGC AGATCCACCG GTCGCCACCA |
| 1621 | TGGTGAGCAA GGGCGAGGAG CTGTTCACCG GGGTGGTGCC CATCCTGGTC GAGCTGGACG |
| 1681 | GCGACGTAAA CGGCCACAAG TTCAGCGTGT CCGGCGAGGG CGAGGGCGAT GCCACCTACG |
| 1741 | GCAAGCTGAC CCTGAAGTTC ATCTGCACCA CCGGCAAGCT GCCCGTGCCC TGGCCCACCC |
| 1801 | TCGTGACCAC CCTGACCTAC GGCGTGCAGT GCTTCAGCCG CTACCCCGAC CACATGAAGC |
| 1861 | AGCACGACTT CTTCAAGTCC GCCATGCCCG AAGGCTACGT CCAGGAGCGC ACCATCTTCT |
| 1921 | TCAAGGACGA CGGCAACTAC AAGACCCGCG CCGAGGTGAA GTTCGAGGGC GACACCCTGG |
| 1981 | TGAACCGCAT CGAGCTGAAG GGCATCGACT TCAAGGAGGA CGGCAACATC CTGGGGCACA |
| 2041 | AGCTGGAGTA CAACTACAAC AGCCACAACG TCTATATCAT GGCCGACAAG CAGAAGAACG |
| 2101 | GCATCAAGGT GAACTTCAAG ATCCGCCACA ACATCGAGGA CGGCAGCGTG CAGCTCGCCG |
| 2161 | ACCACTACCA GCAGAACACC CCCATCGGCG ACGGCCCCGT GCTGCTGCCC GACAACCACT |
| 2221 | ACCTGAGCAC CCAGTCCGCC CTGAGCAAAG ACCCCAACGA GAAGCGCGAT CACATGGTCC |
| 2281 | TGCTGGAGTT CGTGACCGCC GCCGGGATCA CTCTCGGCAT GGACGAGCTG TACAAGTAAT |
| 2341 | AAAATCAACC TCTGGATTAC AAAATTTGTG AAAGATTGAC TGGTATTCTT AACTATGTTG |
| 2401 | CTCCTTTTAC GCTATGTGGA TACGCTGCTT TAATGCCTTT GTATCATGCT ATTGCTTCCC |
| 2461 | GTATGGCTTT CATTTTCTCC TCCTTGTATA AATCCTGGTT GCTGTCTCTT TATGAGGAGT |
| 2521 | TGTGGCCCGT TGTCAGGCAA CGTGGCGTGG TGTGCACTGT GTTTGCTGAC GCAACCCCCA |
| 2581 | CTGGTTGGGG CATTGCCACC ACCTGTCAGC TCCTTTCCGG GACTTTCGCT TTCCCCCTCC |
| 2641 | CTATTGCCAC GGCGGAACTC ATCGCCGCCT GCCTTGCCCG CTGCTGGACA GGGGCTCGGC |
| 2701 | TGTTGGGCAC TGACAATTCC GTGGTGTTGT CGGGGAAGCT GACGTCCTTT CCATGGCTGC |
| 2761 | TCGCCTGTGT TGCCACCTGG ATTCTGCGCG GGACGTCCTT CTGCTACGTC CCTTCGGCCC |
| 2821 | TCAATCCAGC GGACCTTCCT TCCCGCGGCC TGCTGCCGGC TCTGCGGCCT CTTCCGCGTC |
| 2881 | TTCGCCTTCG CCCTCAGACG AGTCGGATCT CCCTTTGGGC CGCCTCCCCG CCTGGTTCCG |
| 2941 | ACTGTGCCTT CTAGTTGCCA GCCATCTGTT GTTTGCCCCT CCCCCGTGCC TTCCTTGACC |
| 3001 | CTGGAAGGTG CCACTCCCAC TGTCCTTTCC TAATAAAATG AGGAAATTGC ATCGCATTGT |
| 3061 | CTGAGTAGGT GTCATTCTAT TCTGGGGGGT GGGGTGGGGC AGGACAGCAA GGGGGAGGAT |
| 3121 | TGGGTAGACA ATAGCAGGCA TGCTGGGGAT GCGGTGGGCT CTATGGTCGA GTTAGTTTGC |
| 3181 | TCCAGTAAAG TTGTTTATAA TAACTACTAA ATCCGCATGT TACGGAATTT CTTATTAATT |
| 3241 | TTTTTTTCGT AAGGAACAAC GGATCTTGAA ATAACTTCGT ATAGCATACA TTATACGAAC |
| 3301 | GGTAATCGGA TCCCGGGCCC GTCGACTGCA GAGGCCTGCA TGCAAGCTTG GTGTAATCAT |
| 3361 | GGTCATAGCT GTTTCCTGTG TGAAATTGTT ATCCGCTCAC AATTCCACAC AACATACGAG |
| 3421 | CCGGAAGCAT AAAGTGTAAA GCCTGGGGTG CCTAATGAGT GAGCTAACTC ACATTAATTG |
| 3481 | CGTTGCGCTC ACTGCCCGCT TTCCAGTCGG GAAACCTGTC GTGCCAGCTG CATTAATGAA |
| 3541 | TCGGCCAACG CGCGGGGAGA GGCGGTTTGC GTATTGGGCG CTCTTCCGCT TCCTCGCTCA |
| 3601 | CTGACTCGCT GCGCTCGGTC GTTCGGCTGC GGCGAGCGGT ATCAGCTCAC TCAAAGGCGG |
| 3661 | TAATACGGTT ATCCACAGAA TCAGGGGATA ACGCAGGAAA GAACATGTGA GCAAAAGGCC |
| 3721 | AGCAAAAGGC CAGGAACCGT AAAAAGGCCG CGTTGCTGGC GTTTTTCCAT AGGCTCCGCC |
| 3781 | CCCCTGACGA GCATCACAAA AATCGACGCT CAAGTCAGAG GTGGCGAAAC CCGACAGGAC |
| 3841 | TATAAAGATA CCAGGCGTTT CCCCCTGGAA GCTCCCTCGT GCGCTCTCCT GTTCCGACCC |
| 3901 | TGCCGCTTAC CGGATACCTG TCCGCCTTTC TCCCTTCGGG AAGCGTGGCG CTTTCTCATA |
| 3961 | GCTCACGCTG TAGGTATCTC AGTTCGGTGT AGGTCGTTCG CTCCAAGCTG GGCTGTGTGC |
| 4021 | ACGAACCCCC CGTTCAGCCC GACCGCTGCG CCTTATCCGG TAACTATCGT CTTGAGTCCA |
| 4081 | ACCCGGTAAG ACACGACTTA TCGCCACTGG CAGCAGCCAC TGGTAACAGG ATTAGCAGAG |
| 4141 | CGAGGTATGT AGGCGGTGCT ACAGAGTTCT TGAAGTGGTG GCCTAACTAC GGCTACACTA |
| 4201 | GAAGAACAGT ATTTGGTATC TGCGCTCTGC TGAAGCCAGT TACCTTCGGA AAAAGAGTTG |
| 4261 | GTAGCTCTTG ATCCGGCAAA CAAACCACCG CTGGTAGCGG TGGTTTTTTT GTTTGCAAGC |
| 4321 | AGCAGATTAC GCGCAGAAAA AAAGGATCTC AAGAAGATCC TTTGATCTTT TCTACGGGGT |
| 4381 | CTGACGCTCA GTGGAACGAA AACTCACGTT AAGGGATTTT GGTCATGAGA TTATCAAAAA |
| 4441 | GGATCTTCAC CTAGATCCTT TTAAATTAAA AATGAAGTTT TAAATCAAGC CCAATCTGAA |
| 4501 | TAATGTTACA ACCAATTAAC CAATTCTGAT TAGAAAAACT CATCGAGCAT CAAATGAAAC |
| 4561 | TGCAATTTAT TCATATCAGG ATTATCAATA CCATATTTTT GAAAAAGCCG TTTCTGTAAT |
| 4621 | GAAGGAGAAA ACTCACCGAG GCAGTTCCAT AGGATGGCAA GATCCTGGTA TCGGTCTGCG |
| 4681 | ATTCCGACTC GTCCAACATC AATACAACCT ATTAATTTCC CCTCGTCAAA AATAAGGTTA |
| 4741 | TCAAGTGAGA AATCACCATG AGTGACGACT GAATCCGGTG AGAATGGCAA AAGTTTATGC |
| 4801 | ATTTCTTTCC AGACTTGTTC AACAGGCCAG CCATTACGCT CGTCATCAAA ATCACTCGCA |
| 4861 | TCAACCAAAC CGTTATTCAT TCGTGATTGC GCCTGAGCGA GACGAAATAC GCGATCGCTG |
| 4921 | TTAAAAGGAC AATTACAAAC AGGAATCGAA TGCAACCGGC GCAGGAACAC TGCCAGCGCA |
| 4981 | TCAACAATAT TTTCACCTGA ATCAGGATAT TCTTCTAATA CCTGGAATGC TGTTTTTCCG |
| 5041 | GGGATCGCAG TGGTGAGTAA CCATGCATCA TCAGGAGTAC GGATAAAATG CTTGATGGTC |
| 5101 | GGAAGAGGCA TAAATTCCGT CAGCCAGTTT AGTCTGACCA TCTCATCTGT AACATCATTG |
| 5161 | GCAACGCTAC CTTTGCCATG TTTCAGAAAC AACTCTGGCG CATCGGGCTT CCCATACAAG |
| 5221 | CGATAGATTG TCGCACCTGA TTGCCCGACA TTATCGCGAG CCCATTTATA CCCATATAAA |
| 5281 | TCAGCATCCA TGTTGGAATT TAATCGCGGC CTCGACGTTT CCCGTTGAAT ATGGCTCATA |
| 5341 | ACACCCCTTG TATTACTGTT TATGTAAGCA GACAGTTTTA TTGTTCATGA TGATATATTT |
| 5401 | TTATCTTGTG CAATGTAACA TCAGAGATTT TGAGACACGG GCCAGAGCTG CA (SEQ ID NO: 502) |

Annotations:

| Region/Element | Base range |
|---|---|
| Lox71 (loxP site) | 432-465 |
| GC-rich region | 529-640 |
| Initiator element | 813-828 |
| 5′ UTR conserved domain | 880-950 |
| Intron 1 | 892-979 |
| Full CMV promoter | 1004-1573 |
| Inert 5′ UTR | 1585-1619 |
| eGFP coding sequence | 1620-2339 |
| WPRE | 2343-2934 |
| bGHpA terminator sequence | 2939-3166 |
| 100 bp random stuffer sequence | 3167-3266 |
| Lox66 (loxP site) | 3271-3304 |
| LacI repressor binding site (complement) | 3384-3406 |
| Lac operon operator (lacO) | 3386-3402 |
| Lac operon promoter (complement) | 3410-3440 |
| C-tag | 3418-3429 |
| E coli catabolite activator protein binding site | 3455-3476 |
| (complement) | |
| Origin of replication (pUC origin) (complement) | 3705-4378 |
| Kanamycin resistance (KanR) CDS (complement) | 4530-5339 |

### Cre Expression Plasmid

pRTX-2848 (SEQ ID NO: 503) expresses iCre from the CMV promoter. The sequence of this construct is provided below.

TABLE Z5
Exemplary Cre recombinase expression plasmid (pRTx-2848)

| | |
|---|---|
| Name | pRTx-2848 |
| Type | Plasmid |
| Description | CMV_iCre_pcDNA3.1(+). |
| Length | 6473 bp |

| | |
|---|---|
| 1 | GACGGATCGG GAGATCTCCC GATCCCCTAT GGTGCACTCT CAGTACAATC TGCTCTGATG |
| 61 | CCGCATAGTT AAGCCAGTAT CTGCTCCCTG CTTGTGTGTT GGAGGTCGCT GAGTAGTGCG |
| 121 | CGAGCAAAAT TTAAGCTACA ACAAGGCAAG GCTTGACCGA CAATTGCATG AAGAATCTGC |
| 181 | TTAGGGTTAG GCGTTTTGCG CTGCTTCGCG ATGTACGGGC CAGATATACG CGTTGACATT |
| 241 | GATTATTGAC TAGTTATTAA TAGTAATCAA TTACGGGGTC ATTAGTTCAT AGCCCATATA |
| 301 | TGGAGTTCCG CGTTACATAA CTTACGGTAA ATGGCCCGCC TGGCTGACCG CCCAACGACC |
| 361 | CCCGCCCATT GACGTCAATA ATGACGTATG TTCCCATAGT AACGCCAATA GGGACTTTCC |
| 421 | ATTGACGTCA ATGGGTGGAG TATTTACGGT AAACTGCCCA CTTGGCAGTA CATCAAGTGT |
| 481 | ATCATATGCC AAGTACGCCC CCTATTGACG TCAATGACGG TAAATGGCCC GCCTGGCATT |
| 541 | ATGCCCAGTA CATGACCTTA TGGGACTTTC CTACTTGGCA GTACATCTAC GTATTAGTCA |
| 601 | TCGCTATTAC CATGGTGATG CGGTTTTGGC AGTACATCAA TGGGCGTGGA TAGCGGTTTG |
| 661 | ACTCACGGGG ATTTCCAAGT CTCCACCCCA TTGACGTCAA TGGGAGTTTG TTTTGGCACC |
| 721 | AAAATCAACG GGACTTTCCA AAATGTCGTA ACAACTCCGC CCCATTGACG CAAATGGGCG |
| 781 | GTAGGCGTGT ACGGTGGGAG GTCTATATAA GCAGAGCTCT CTGGCTAACT AGAGAACCCA |
| 841 | CTGCTTACTG GCTTATCGAA ATTAATACGA CTCACTATAG GGAGACCCAA GCTGGCTAGC |
| 901 | GTTTAAACTT AAGCTTGGTA CCGAGCTCGG ATCCACTAGT CCAGTGTGGT GGAATTCGCC |
| 961 | ACCATGGTGC CCAAGAAGAA GAGGAAAGTC TCCAACCTGC TGACTGTGCA CCAAAACCTG |
| 1021 | CCTGCCCTCC CTGTGGATGC CACCTCTGAT GAAGTCAGGA AGAACCTGAT GGACATGTTC |
| 1081 | AGGGACAGGC AGGCCTTCTC TGAACACACC TGGAAGATGC TCCTGTCTGT GTGCAGATCC |
| 1141 | TGGGCTGCCT GGTGCAAGCT GAACAACAGG AAATGGTTCC CTGCTGAACC TGAGGATGTG |
| 1201 | AGGGACTACC TCCTGTACCT GCAAGCCAGA GGCCTGGCTG TGAAAACCAT CCAACAGCAC |
| 1261 | CTGGGCCAGC TCAACATGCT GCACAGGAGA TCTGGCCTGC CTCGCCCTTC TGACTCCAAT |
| 1321 | GCTGTGTCCC TGGTGATGAG GAGAATCAGA AAGGAGAATG TGGATGCTGG GGAGAGAGCC |
| 1381 | AAGCAGGCCC TGGCCTTTGA ACGCACTGAC TTTGACCAAG TCAGATCCCT GATGGAGAAC |
| 1441 | TCTGACAGAT GCCAGGACAT CAGGAACCTG GCCTTCCTGG GCATTGCCTA CAACACCCTG |
| 1501 | CTGCGCATTG CCGAAATTGC CAGAATCAGA GTGAAGGACA TCTCCCGCAC CGATGGTGGG |
| 1561 | AGAATGCTGA TCCACATTGG CAGGACCAAG ACCCTGGTGT CCACAGCTGG TGTGGAGAAG |
| 1621 | GCCCTGTCCC TGGGGGTTAC CAAGCTGGTG GAGAGATGGA TCTCTGTGTC TGGTGTGGCT |
| 1681 | GATGACCCCA ACAACTACCT GTTCTGCCGG GTCAGAAAGA ATGGTGTGGC TGCCCCTTCT |
| 1741 | GCCACCTCCC AACTGTCCAC CCGCGCCCTG GAAGGGATCT TTGAGGCCAC CCACCGCCTG |
| 1801 | ATCTATGGTG CCAAGGATGA CTCTGGGCAG AGATACCTGG CCTGGTCTGG CCACTCTGCC |
| 1861 | AGAGTGGGTG CTGCCAGGGA CATGGCCAGG GCTGGTGTGT CCATCCCTGA AATCATGCAG |
| 1921 | GCTGGTGGCT GGACCAATGT GAACATAGTG ATGAACTACA TCAGAAACCT GGACTCTGAG |
| 1981 | ACTGGGGCCA TGGTGAGGCT GCTCGAGGAT GGGGACTGAG CGGCCGCTCG AGTCTAGAGG |
| 2041 | GCCCGTTTAA ACCCGCTGAT CAGCCTCGAC TGTGCCTTCT AGTTGCCAGC CATCTGTTGT |
| 2101 | TTGCCCCTCC CCCGTGCCTT CCTTGACCCT GGAAGGTGCC ACTCCCACTG TCCTTTCCTA |
| 2161 | ATAAAATGAG GAAATTGCAT CGCATTGTCT GAGTAGGTGT CATTCTATTC TGGGGGGTGG |
| 2221 | GGTGGGGCAG GACAGCAAGG GGGAGGATTG GGAAGACAAT AGCAGGCATG CTGGGGATGC |
| 2281 | GGTGGGCTCT ATGGCTTCTG AGGCGGAAAG AACCAGCTGG GGCTCTAGGG GGTATCCCCA |
| 2341 | CGCGCCCTGT AGCGGCGCAT TAAGCGCGGC GGGTGTGGTG GTTACGCGCA GCGTGACCGC |
| 2401 | TACACTTGCC AGCGCCCTAG CGCCCGCTCC TTTCGCTTTC TTCCCTTCCT TTCTCGCCAC |
| 2461 | GTTCGCCGGC TTTCCCCGTC AAGCTCTAAA TCGGGGGCTC CCTTTAGGGT TCCGATTTAG |
| 2521 | TGCTTTACGG CACCTCGACC CCAAAAAACT TGATTAGGGT GATGGTTCAC GTAGTGGGCC |
| 2581 | ATCGCCCTGA TAGACGGTTT TTCGCCCTTT GACGTTGGAG TCCACGTTCT TTAATAGTGG |
| 2641 | ACTCTTGTTC CAAACTGGAA CAACACTCAA CCCTATCTCG GTCTATTCTT TTGATTTATA |
| 2701 | AGGGATTTTG CCGATTTCGG CCTATTGGTT AAAAAATGAG CTGATTTAAC AAAAATTTAA |
| 2761 | CGCGAATTAA TTCTGTGGAA TGTGTGTCAG TTAGGGTGTG GAAAGTCCCC AGGCTCCCCA |
| 2821 | GCAGGCAGAA GTATGCAAAG CATGCATCTC AATTAGTCAG CAACCAGGTG TGGAAAGTCC |
| 2881 | CCAGGCTCCC CAGCAGGCAG AAGTATGCAA AGCATGCATC TCAATTAGTC AGCAACCATA |
| 2941 | GTCCCGCCCC TAACTCCGCC CATCCCGCCC CTAACTCCGC CCAGTTCCGC CCATTCTCCG |
| 3001 | CCCCATGGCT GACTAATTTT TTTTATTTAT GCAGAGGCCG AGGCCGCCTC TGCCTCTGAG |
| 3061 | CTATTCCAGA AGTAGTGAGG AGGCTTTTTT GGAGGCCTAG GCTTTTGCAA AAAGCTCCCG |
| 3121 | GGAGCTTGTA TATCCATTTT CGGATCTGAT CAAGAGACAG GATGAGGATC GTTTCGCATG |
| 3181 | ATTGAACAAG ATGGATTGCA CGCAGGTTCT CCGGCCGCTT GGGTGGAGAG GCTATTCGGC |
| 3241 | TATGACTGGG CACAACAGAC AATCGGCTGC TCTGATGCCG CCGTGTTCCG GCTGTCAGCG |
| 3301 | CAGGGGCGCC CGGTTCTTTT TGTCAAGACC GACCTGTCCG GTGCCCTGAA TGAACTGCAG |
| 3361 | GACGAGGCAG CGCGGCTATC GTGGCTGGCC ACGACGGGCG TTCCTTGCGC AGCTGTGCTC |
| 3421 | GACGTTGTCA CTGAAGCGGG AAGGGACTGG CTGCTATTGG GCGAAGTGCC GGGGCAGGAT |
| 3481 | CTCCTGTCAT CTCACCTTGC TCCTGCCGAG AAAGTATCCA TCATGGCTGA TGCAATGCGG |
| 3541 | CGGCTGCATA CGCTTGATCC GGCTACCTGC CCATTCGACC ACCAAGCGAA ACATCGCATC |
| 3601 | GAGCGAGCAC GTACTCGGAT GGAAGCCGGT CTTGTCGATC AGGATGATCT GGACGAAGAG |
| 3661 | CATCAGGGGC TCGCGCCAGC CGAACTGTTC GCCAGGCTCA AGGCGCGCAT GCCCGACGGC |
| 3721 | GAGGATCTCG TCGTGACCCA TGGCGATGCC TGCTTGCCGA ATATCATGGT GGAAAATGGC |
| 3781 | CGCTTTTCTG GATTCATCGA CTGTGGCCGG CTGGGTGTGG CGGACCGCTA TCAGGACATA |
| 3841 | GCGTTGGCTA CCCGTGATAT TGCTGAAGAG CTTGGCGGCG AATGGGCTGA CCGCTTCCTC |
| 3901 | GTGCTTTACG GTATCGCCGC TCCCGATTCG CAGCGCATCG CCTTCTATCG CCTTCTTGAC |
| 3961 | GAGTTCTTCT GAGCGGGACT CTGGGGTTCG AAATGACCGA CCAAGCGACG CCCAACCTGC |
| 4021 | CATCACGAGA TTTCGATTCC ACCGCCGCCT TCTATGAAAG GTTGGGCTTC GGAATCGTTT |
| 4081 | TCCGGGACGC CGGCTGGATG ATCCTCCAGC GCGGGGATCT CATGCTGGAG TTCTTCGCCC |
| 4141 | ACCCCAACTT GTTTATTGCA GCTTATAATG GTTACAAATA AAGCAATAGC ATCACAAATT |
| 4201 | TCACAAATAA AGCATTTTTT TCACTGCATT CTAGTTGTGG TTTGTCCAAA CTCATCAATG |
| 4261 | TATCTTATCA TGTCTGTATA CCGTCGACCT CTAGCTAGAG CTTGGCGTAA TCATGGTCAT |
| 4321 | AGCTGTTTCC TGTGTGAAAT TGTTATCCGC TCACAATTCC ACACAACATA CGAGCCGGAA |
| 4381 | GCATAAAGTG TAAAGCCTGG GGTGCCTAAT GAGTGAGCTA ACTCACATTA ATTGCGTTGC |
| 4441 | GCTCACTGCC CGCTTTCCAG TCGGGAAACC TGTCGTGCCA GCTGCATTAA TGAATCGGCC |
| 4501 | AACGCGCGGG GAGAGGCGGT TTGCGTATTG GGCGCTCTTC CGCTTCCTCG CTCACTGACT |
| 4561 | CGCTGCGCTC GGTCGTTCGG CTGCGGCGAG CGGTATCAGC TCACTCAAAG GCGGTAATAC |
| 4621 | GGTTATCCAC AGAATCAGGG GATAACGCAG GAAAGAACAT GTGAGCAAAA GGCCAGCAAA |
| 4681 | AGGCCAGGAA CCGTAAAAAG GCCGCGTTGC TGGCGTTTTT CCATAGGCTC CGCCCCCCTG |
| 4741 | ACGAGCATCA CAAAAATCGA CGCTCAAGTC AGAGGTGGCG AAACCCGACA GGACTATAAA |
| 4801 | GATACCAGGC GTTTCCCCCT GGAAGCTCCC TCGTGCGCTC TCCTGTTCCG ACCCTGCCGC |
| 4861 | TTACCGGATA CCTGTCCGCC TTTCTCCCTT CGGGAAGCGT GGCGCTTTCT CATAGCTCAC |
| 4921 | GCTGTAGGTA TCTCAGTTCG GTGTAGGTCG TTCGCTCCAA GCTGGGCTGT GTGCACGAAC |
| 4981 | CCCCCGTTCA GCCCGACCGC TGCGCCTTAT CCGGTAACTA TCGTCTTGAG TCCAACCCGG |
| 5041 | TAAGACACGA CTTATCGCCA CTGGCAGCAG CCACTGGTAA CAGGATTAGC AGAGCGAGGT |
| 5101 | ATGTAGGCGG TGCTACAGAG TTCTTGAAGT GGTGGCCTAA CTACGGCTAC ACTAGAAGAA |
| 5161 | CAGTATTTGG TATCTGCGCT CTGCTGAAGC CAGTTACCTT CGGAAAAAGA GTTGGTAGCT |
| 5221 | CTTGATCCGG CAAACAAACC ACCGCTGGTA GCGGTGGTTT TTTTGTTTGC AAGCAGCAGA |
| 5281 | TTACGCGCAG AAAAAAAGGA TCTCAAGAAG ATCCTTTGAT CTTTTCTACG GGGTCTGACG |
| 5341 | CTCAGTGGAA CGAAAACTCA CGTTAAGGGA TTTTGGTCAT GAGATTATCA AAAAGGATCT |
| 5401 | TCACCTAGAT CCTTTTAAAT TAAAAATGAA GTTTTAAATC AATCTAAAGT ATATATGAGT |
| 5461 | AAACTTGGTC TGACAGTTAC CAATGCTTAA TCAGTGAGGC ACCTATCTCA GCGATCTGTC |
| 5521 | TATTTCGTTC ATCCATAGTT GCCTGACTCC CCGTCGTGTA GATAACTACG ATACGGGAGG |
| 5581 | GCTTACCATC TGGCCCCAGT GCTGCAATGA TACCGCGAGA CCCACGCTCA CCGGCTCCAG |
| 5641 | ATTTATCAGC AATAAACCAG CCAGCCGGAA GGGCCGAGCG CAGAAGTGGT CCTGCAACTT |
| 5701 | TATCCGCCTC CATCCAGTCT ATTAATTGTT GCCGGGAAGC TAGAGTAAGT AGTTCGCCAG |
| 5761 | TTAATAGTTT GCGCAACGTT GTTGCCATTG CTACAGGCAT CGTGGTGTCA CGCTCGTCGT |
| 5821 | TTGGTATGGC TTCATTCAGC TCCGGTTCCC AACGATCAAG GCGAGTTACA TGATCCCCCA |
| 5881 | TGTTGTGCAA AAAAGCGGTT AGCTCCTTCG GTCCTCCGAT CGTTGTCAGA AGTAAGTTGG |
| 5941 | CCGCAGTGTT ATCACTCATG GTTATGGCAG CACTGCATAA TTCTCTTACT GTCATGCCAT |
| 6001 | CCGTAAGATG CTTTTCTGTG ACTGGTGAGT ACTCAACCAA GTCATTCTGA GAATAGTGTA |
| 6061 | TGCGGCGACC GAGTTGCTCT TGCCCGGCGT CAATACGGGA TAATACCGCG CCACATAGCA |
| 6121 | GAACTTTAAA AGTGCTCATC ATTGGAAAAC GTTCTTCGGG GCGAAAACTC TCAAGGATCT |
| 6181 | TACCGCTGTT GAGATCCAGT TCGATGTAAC CCACTCGTGC ACCCAACTGA TCTTCAGCAT |
| 6241 | CTTTTACTTT CACCAGCGTT TCTGGGTGAG CAAAAACAGG AAGGCAAAAT GCCGCAAAAA |
| 6301 | AGGGAATAAG GGCGACACGG AAATGTTGAA TACTCATACT CTTCCTTTTT CAATATTATT |
| 6361 | GAAGCATTTA TCAGGGTTAT TGTCTCATGA GCGGATACAT ATTTGAATGT ATTTAGAAAA |
| 6421 | ATAAACAAAT AGGGGTTCCG CGCACATTTC CCCGAAAAGT GCCACCTGAC GTC (SEQ ID NO: 503) |

Annotations:

| Region/ Element | Base range |
|---|---|
| CMV enhancer | 235-614 |
| CMV promoter | 615-818 |
| T7 RNA polymerase promoter | 863-880 |
| iCre coding sequence | 964-2019 |
| bGHpA terminator sequence | 2070-2293 |
| Origin of replication | 2962-3097 |
| SV40 polyA sequence | 4146-4267 |
| Lac operon operator (lacO) | 4340-4356 |
| Lac operon promoter (complement) | 4364-4394 |
| C-tag | 4372-4383 |
| Catabolite activator protein binding site | 4409-4430 |
| Origin of replication (complement) | 4718-5306 |
| Ampicillin resistance gene promoter | 6338-6442 |
| (complement) | |

### Anellovirus Vector Production: Cell Lysis

Cell pellets were resuspended in lysis buffer containing 50 mM Tris pH 8.0, 0.5% Triton-X100, 100 mM NaCl, 100× Halt protease inhibitor cocktail (Thermo Fisher Scientific catalog #78439), and 200 U of mSAN nuclease (ArcticZyme catolog #NC1920045). The cell lysates were clarified by centrifugation at 12,500×g for 30 minutes at 4° C.

### Anellovirus Vector Production: Isopycnic Centrifugation

To prepare iodixanol linear gradients, 13 mL of 60% OptiPrep (Sigma-Aldrich catalog #D1556) was overlayed with 13 mL of 20% OptiPrep in 26.3-mL polycarbonate tubes, which were then spun at a 46-degree angle and a speed of 20 rpm for 16 minutes using Gradient Master (BioComp). Following the generation of the iodixanol linear gradient, 2 mL of iodixanol were removed from the top of each gradient, and 2 mL of clarified lysate was added on top of the gradient. The sample-containing tubes were spun at 347,000×g and 20° C. for 3 hours using Type 70 Ti rotor (Beckman Coulter). 1-mL fractions were collected from the top of the tubes and transferred to a 96-well 2.2 ml capacity plate. Each fraction was then subjected to DNase-protected qPCR assay as described below.

### Anellovirus Vector Production: Concentration of Material

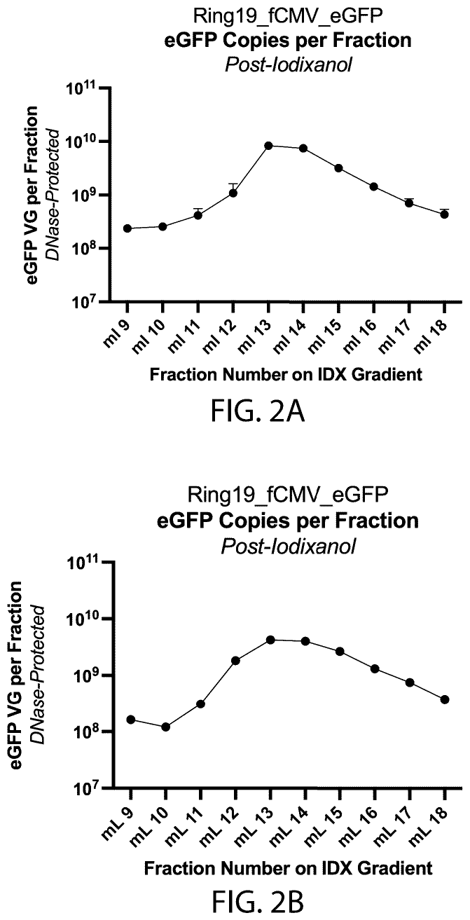

Fractions of interest were determined based on the viral titer (FIG. 2A and FIG. 2B). The pooled iodixanol fractions were then diluted 1:50 in formulation buffer (1×DPBS, 0.001% Pluronic F-68) and concentrated using Vivaspin 20 100K MWCO centrifugal filter units (Fisher Scientific catalog #1455810).

### Anellovirus Vector Production: DNase-Protection Assay on Final Material

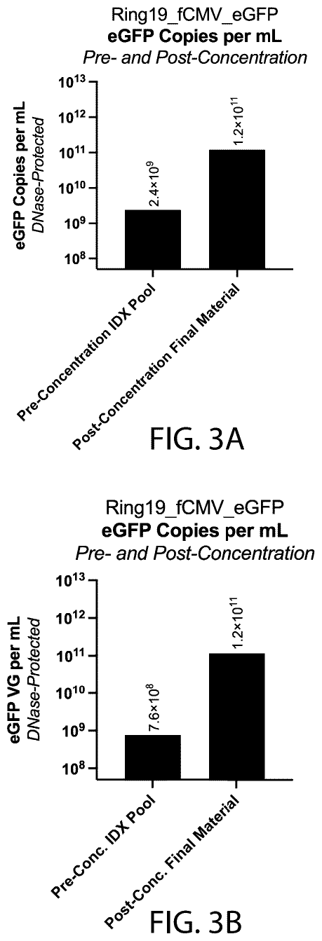

5 μl of the sample to be titered was incubated with 20 U of DNAse I endonuclease (Thermo Fisher Scientific catalog #18047019) in a 20-μl reaction. The reaction was incubated at 37° C. for 30 minutes. Following DNase-treatment, each sample was subjected to Proteinase K (Fisher Scientific catalog #FERE00491) and proteinase K buffer (1% SDS, 0.1M EDTA, 0.1M Tris pH 8.0, 0.1% Pluronic F-68). The reaction was incubated at 37° C. for 30 minutes, followed by proteinase K inactivation at 95° C. for 15 minutes. 4 μl of the 1:10 diluted DNase reaction was subjected to qPCR analysis in a 20-μl reaction using TaqMan Fast Universal PCR Master Mix (Thermo Fisher Scientific catalog #44-449-63) according to the manufacturer's protocol (FIG. 3A and FIG. 3B). Primer and probe sequences are listed in Table E1.

TABLE E1
Primer and probes designed to quantify eGFP

| Target | Label | Sequence (5′→3′) |
|---|---|---|
| eGFP | Forward Primer | GAACCGCATCGAGCTGAA |
| | | (SEQ ID NO: 1115) |
| | Reverse Primer | TGCTTGTCGGCCATGATATAG |
| | | (SEQ ID NO: 1116) |
| | Probe (FAM) | ATCGACTTCAAGGAGGACGGCAAC |
| | | (SEQ ID NO: 1117) |

### Anellovirus Vector Production: Endotoxin Test on Final Material

2.241 of the sample was diluted 1:50 in formulation buffer (1×DPBS, 0.001% Pluronic F-68) and the sample was subjected to the LAL detection test (Charles River) according to the manufacturer's protocol.

### Anellovirus Vector Production: SDS-PAGE and Coomassie Stain on Final Material

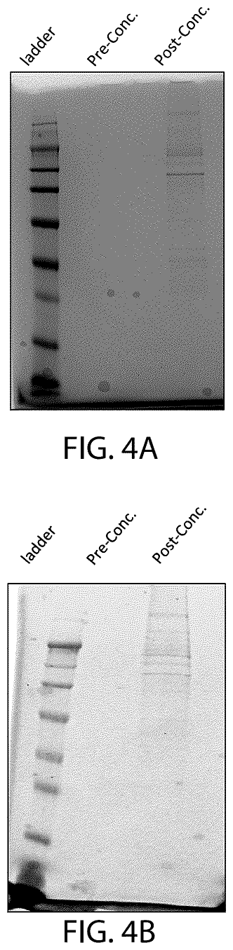

2 μl of the sample was diluted 1:10 in formulation buffer (1×DPBS, 0.001% Pluronic F-68) and the sample was mixed 1:1 with loading dye and Bolt sample reducing agent (Thermo Fisher Scientific catalog #B0009), followed by boiling at 95° C. for 10 minutes. Proteins were separated on Bolt 4-12% Bis-Tris gel in 1×Bolt MOPS SDS running buffer (Thermo Fisher Scientific catalog #B0001). Separated proteins were stained using InstantBlue Coomassie Protein Stain (ABCAM catalog #ab119211) according to the manufacturers protocol. Following the stain, the gel was washed three times with diH2O. The stained gel was visualized using Chemidoc Imaging System (BioRad) (FIGS. 4A and 4B).

### Anellovirus Vector Production: Bicinchonic Acid Assay (BCA)

5 μl of the sample was diluted 1:10 in formulation buffer (1×DPBS, 0.001% Pluronic F-68) and the sample was subjected to the Pierce BCA Protein Assay Kit (ThermoScientific catalogue #23227) according to the manufacturer's protocol.

### Example 2: In Vivo Transduction of the Brain Via Intracerebroventricular (ICV) Administration with an Anellovector (Study #1)

This example demonstrates that an Anellovector transduces the brain in vivo via ICV administration.

### Materials and Methods

The production of the AAV9-fCMV-eGFP control and Ring19-fCMV-eGFP Anellovector used in this Study #1 are described in Example 1.

### Care and Use of Animals

All mouse studies were approved and governed by the Ring Therapeutics Institutional Animal Care and Use Committee. Female FVB/NJ 7-8 weeks of age were obtained from Jackson Laboratories for use in these studies.

### Intracerebroventricular Injections

Mice were anesthetized with 2%-3% isoflurane and placed with a heat pad on the stereotaxic frame (Neurotar GmbH). Eye ointment was applied to both eyes. After applying aseptic betadine and 70% alcohol, an incision was made to expose the skull. A craniotomy was made by a micro-driller. A 5-10 μL Hamilton syringe was slowly moved to the target coordinates (lateral ventricle, AP+0.25 mm, ML, ±0.7 mm, DV, −2 mm. 0.5-3 μL of test article was injected with the speed approximately 0.02 μL/min. Test articles were injected bilaterally or unilaterally into the specific brain regions using established mouse brain coordinates. The incision was sutured with absorbable sutures (Suture CT-13.0 coated Vicryl 27”) or by applying surgical glue on skin incision or sutures. Each animal received ICV injection once and SC injection of buprenorphine once after the ICV injection for analgesic purpose during the week of the procedure.

### Tissue Harvest for DNA and RNA Extraction

Brains were dissected out and then cut in half in sagittal orientation. Half of the brain was used for DNA processing and the other half of the brain from the same animal was used for RNA processing. Brains were frozen and stored in 2 ml reinforced bead homogenizer tubes for both DNA and RNA extraction. Spinal cord samples were also collected and stored in All-prep reagent for All-prep DNA and RNA extraction (Qiagen).

### DNA Isolation

Frozen tissue samples were lysed with an automated tissue homogenizer (Geno/Grinder SPEX Sample Prep) in Buffer ATL (Qiagen, USA) and proteinase K (Qiagen, USA) at 1250 rpm for (2) 30-second rounds. Homogenized tissues were digested on heat block at 56 C for about 4 hours. Genomic DNA was precipitated with Buffer AL (Qiagen, USA) and ethanol, then isolated with Qiagen DNeasy 96 Blood & Tissue Kit. Isolated DNA was quantified using a NanoDrop 8000 Spectrophotometer (Thermofisher, USA).

qPCR

Genomic DNA was assayed by qPCR on the QuantStudio 5—Real-Time PCR System (Thermo Fisher, USA) using TaqMan Gene Expression Master Mix (Thermofisher, USA). The sequence detection primers and FAM custom probes that were used in this study were synthesized by Integrated DNA Technologies, USA. eGFP primer/probe sequences are in Table EL.

All reactions including the DNA samples and different dilutions of a known quantity of the linearized eGFP and Ring19 plasmid standards were run in triplicate on the same plate. The standard curve method was used to calculate the amount of viral/vector DNA, which was normalized with the total amount of genomic DNA for each sample (quantified using nanodrop as described above).

### RNA Isolation

Frozen tissue samples were lysed with an automated tissue homogenizer (Geno/Grinder SPEX Sample Prep, USA) in QIAzol lysis reagent (Qiagen, USA) at 1250 rpm for (2) 30-second rounds. RNA was isolated in aqueous phase by addition of phenol chloroform (Thermofisher, USA) and centrifuged at 6000 rpm for 15 minutes at 4 C. The upper aqueous phase was transferred into a fresh S-block (Qiagen, USA). RNA was then precipitated with the addition of 1 volume of 70% Ethanol and isolated with the Qiagen RNeasy 96 kit. RNA concentration was quantified via the Qubit RNA High Sensitivity Assay Kit (Thermofisher, USA)

### All-Prep DNA and RNA Extraction

To extract DNA and RNA from the spinal cord samples, 10 uL BME per 1 mL Buffer RLT Plus were added to the sample. Tissue was homogenized with 350 uL RLT Plus/BME. Spex tubes were spun down and lysates were transferred into clean Eppendorf tubes. Lysate were spun at 6000 rpm for 4 minutes at RT. 150 μL of supernatant was transferred into an All-Prep DNA Mini Column (Qiagen) and the remaining supernatant lysate was transferred into a freshly labelled Eppendorf for long-term storage at −80 C. DNA columns were spun at max speed for 30 seconds. DNA columns were then placed into new collection tubes and stored at 4 C for later DNA purification. 14 uL Proteinase K was added to the flowthrough from the last step. 58 uL 100% ethanol was added to flow through and samples were mixed well and incubated at RT for 10 min. 115 μL of 100% ethanol was added and mixed well. All contents were transferred into RNeasy Mini Spin columns. After centrifugation, 500 uL Buffer RPE was added to the RNeasy Mini Spin Column. 80 μL of DNase I was transferred and mixed into the RNeasy column with incubation at RT for 15 min. FRN, buffer RPE, and 100% ethanol were added in order to wash the columns. 30 uL RNase-free water was added directly to the column and incubated for 2-5 minutes at RT. Samples were spun for 1 minute at max speed to elute the RNA.

### One-Step RT-ddPCR

RNA was diluted in nuclease-free water and combined with the reagents from the One-Step RT-ddPCR Advanced Kit for Probes (Bio-Rad, USA; Catalog #1864022) and eGFP primer/probe set with final primer concentrations of 900 nM and probe concentrations of 250 nM to measure transgene expression. After the RT-ddPCR reaction setup, each reaction was converted to droplets using the Automated Droplet Generator (Bio-Rad, USA) according to the manufacturer's instructions. After droplet generation, the droplets were subjected to endpoint PCR thermocycling with the following cycling conditions: 1 cycle of 48 C for 1 hour for reverse transcription followed by 1 cycle of 95 C for 10 mins; 40 cycles of 95 C for 30 sec, 60 C for 1 min; and 1 cycle of 98 C for 10 min and finally a 4 C hold. The cycled plate was then transferred to the QX200 Droplet Reader (Bio-Rad, USA) and analyzed using QX Manager Software (Bio-Rad, USA).

### Tissue Harvest for Immunohistochemistry

Brains were collected in 4% PFA and fixed for 24 hours. Brain samples were changed from PFA to 30% sucrose and stayed in sucrose for at least 2 nights until the tissue sunk to the bottom. Brains were embedded in OCT compound for sectioning. Brains were sectioned at 30 m in a Leica cryostat and mounted immediately on poly-L-Lysine coated slides. Sections were stored at −80° C. prior to processing.

### Immunohistochemistry

Slides were air-dried for 20 minutes and then washed with 1×PBS for 5 minutes three times. Slides were incubated at room temperature with 10% goat serum in TBST for 2 hours followed by the primary antibody at 1:500 (GFP recombinant rabbit monoclonal antibody, ThermoFisher G10362, TBST with 10% goat serum) for 48 hours at 4 C. Slides were washed with PBS for 5 minutes three times and incubated in 1:2000 secondary antibody (goat anti-rabbit IgG (H+L) cross-absorbed secondary antibody Alexa-fluor 488 (ThermoFisher A11008) TBST with 10% goat serum) for 2 hours at room temperature, avoiding light. Slides were washed with PBS for 5 minutes three times. DAPI mounting media (ThermoFisher P36966) was added to the slides and covered with a coverslip. 2× images were captured with an EVOS (M7000, Invitrogen by ThermoFisher Scientific), and 20× images were captured with a confocal microscope (Zeiss LSM900).

### Results

### Infectivity of Anellovector R19-fCMV-eGFP (“R19-eGFP”) in the Brain

The prepared virus preparations were administered to mice by intracerebroventricular (ICV) injection as shown in Table E2. Mice were injected with PBS, R19-eGFP, or dose-matched AAV9-eGFP. 21 days after injection, the brain and spinal cord were collected and processed.

TABLE E2
Study design #1 for ICV administration.

| | Treatment | Dose/eye | Route | N | Terminal |
|---|---|---|---|---|---|
| Group | Day 0 | (vg) | (volume) | (mice) | Day |
| 1 | PBS | 0 | ICV (2 × 2 ul) | 7 | 21 |
| 2 | R19-fCMV-eGFP (“R19-eGFP”) | 3.6e+8 | ICV (2 × 2 ul) | 7 | 21 |
| 3 | AAV9-fCMV-eGFP (“AAV9-eGFP”) | 3.6e+8 | ICV (2 × 2 ul) | 7 | 21 |

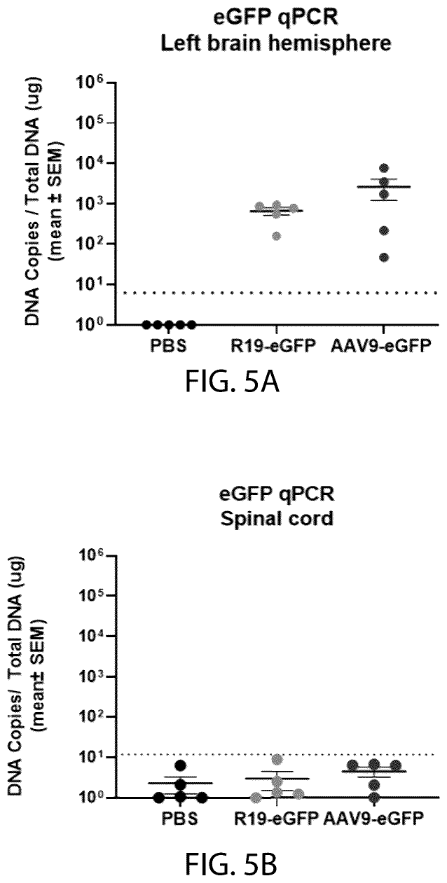

DNA was collected from the left brain hemisphere. eGFP genomes were detected by qPCR in the brain transduced with R19-eGFP Anellovector and AAV9-eGFP, whereas no eGFP genomes were detected in the PBS control group (FIG. 5A). In contrast, the level of eGFP genomes were below the limit of detection by qPCR in the spinal cord in the PBS, R19-eGFP Anellovector, and AAV9-eGFP groups at the doses administered (FIG. 5B).

Administration of Ring19-eGFP Anellovector by ICV Injection Induces eGFP mRNA Expression in the Brain

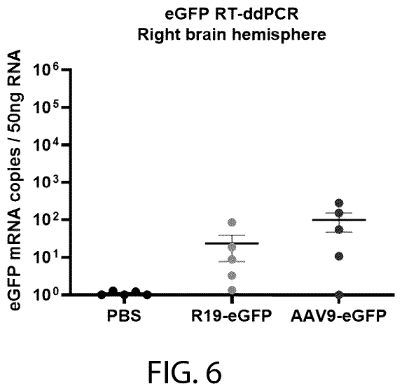

To determine whether administration of R19-eGFP Anellovector can induce eGFP mRNA expression, RNA was collected from the right brain hemisphere at 21 days after infection (n=5 brain hemispheres per group). eGFP mRNA was then quantified by RT-ddPCR. eGFP mRNA was detected in the brain 21 days after infection with R19-eGFP and AAV9-eGFP, while no eGFP mRNA was detected in the PBS control group (FIG. 6).

Administration of R19-eGFP Anellovector by ICV Injection Induces eGFP Protein Expression in the Brain

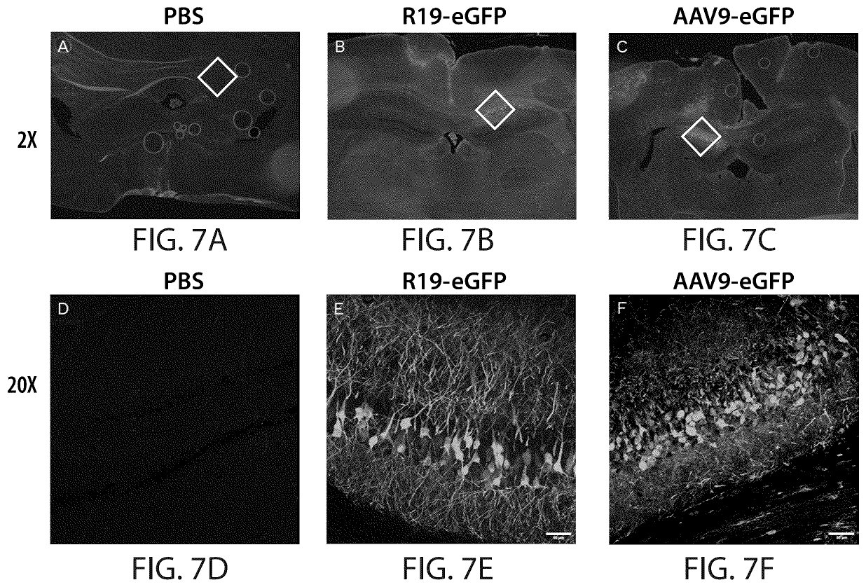

To determine whether administration of R19-eGFP Anellovector can produce eGFP protein in vivo in the brain, brain tissue was collected at day 21 after infection for fixation and immunohistochemistry (n=2 brains per group). Representative brain sections of ICV-injected mice were stained for eGFP. FIGS. 7A, 7B, and 7C show images of brain sections stained for eGFP from the PBS control, R19-eGFP, and AAV9-eGFP groups, respectively, at 2× magnification. The area in the red squares was magnified to 20× (FIGS. 7D, 7E, and 7F). While the PBS control showed no eGFP staining (FIG. 7D), eGFP-positive neurons were detected in the hippocampus of both R19-eGFP (FIG. 7E) and AAV9-eGFP (FIG. 7F)-treated brains. These data show that administration of a R19-eGFP Anellovector resulted in expression of eGFP protein in the brain.

### Example 3: In Vivo Delivery of an Anellovector to the Spinal Cord Via Intrathecal (IT) Administration (Study #2)

This example describes the in vivo delivery of a Ring19-eGFP Anellovector to the spinal cord.

### Materials and Methods

The production of the AAV9-fCMV-eGFP control and Ring19-fCMV-eGFP Anellovector used in this Study #2 are described in Example 1.

All other methods are according to Example 2 above, except that intrathecal injections were performed as described below.

### Intrathecal Injections

The mice were anesthetized with 3% isoflurane, until they showed no signs of righting reflex. In addition, tail and/or paw pinch reflex were checked to further ensure the state of anesthesia. The posterior end of the animal, near the base of the tail, was shaved in an area around 2 cm² to facilitate better visualization during needle insertion. The mouse was placed in a nose cone for continued isoflurane administration during the procedure. During the procedure, the isoflurane was reduced to 1.5% and the eyes of the mouse were covered with eye lubricant to prevent eye damage. A 10-50 μl Hamilton with a 26 gauge needle was used to locate the L5-L6 region of the spinal cord. The needle was inserted between the groove of the L5 and L6 vertebrae. A tail flick indicated successful entry of the needle into the intradural space. Once a tail flick was observed, immediately, but carefully, the needle position was secured with one hand and the desired volume of the substance was injected with the other hand slowly. No more than 5 μl was injected per mouse. Once the injection was performed, the mouse was moved back to the cage to recover from anesthesia. Each animal received once weekly IT injections for 2 weeks followed by one time subcutaneous injection of an analgesic drug during the procedure week as needed.

The virus preparations were administered into mice by intrathecal (IT) injection as shown in Table E3. Mice were injected with PBS, R19-eGFP, or dose-matched AAV9-eGFP. 21 days after injection, the spinal cords and brain were collected and processed.

TABLE E3
Study design #2 for IT administration.

| | Treatment | Dose/eye | Route | N | Terminal |
|---|---|---|---|---|---|
| Group | Day 0 | (vg) | (volume) | (mice) | Day |
| 1 | PBS | 0 | IT (50 uL) | 7 | 21 |
| 2 | R19-fCMV-eGFP | 9.6e+9 | IT (50 uL) | 7 | 21 |
| 3 | AAV9-fCMV-eGFP | 9.6e+9 | IT (50 uL) | 7 | 21 |

### Results

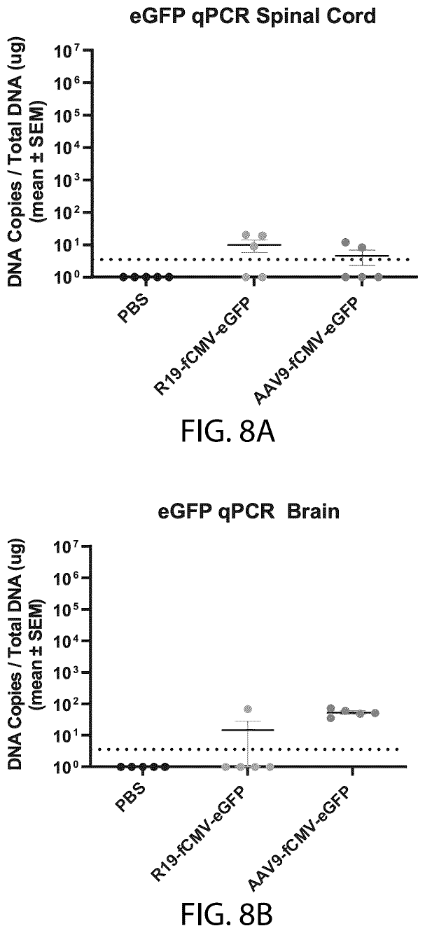

DNA was collected from the spinal cord and the brain as described in Example 2 and eGFP genomes were detected by qPCR in the spinal cord (FIG. 8A) and in the brain (FIG. 8B). eGFP genomes were transduced with R19-eGFP Anellovector at similar levels as the dose-matched AAV9-eGFP, while no eGFP genomes were detected in the PBS control.

### Example 4: In Vivo Delivery of an Anellovector to the Spinal Cord and Redosing Via IT Administration

This example describes the in vivo redosing of a Ring19-eGFP Anellovector to the spinal cord.

### Materials and Methods

The AAV9-fCMV-eGFP control was produced as described above.

The R19-fCMV-eGFP anellovector was produced by transfecting MOLT-4 cells with the three plasmids shown in Table E0 (pRTx-3525; pRTx-2847; pRTx-2848) via electroporation. The transfected cells were harvested 72 hours post-electroporation and centrifuged. The supernatant was discarded, and the pellet was frozen at −80°. The cells were thawed and resuspended in buffer. The lysate was initially clarified by centrifugation, then sterile filtered. The clarified harvest pool was buffer exchanged using tangential flow filtration (TFF). After buffer exchange, the pool was sterile filtered.

The buffer-exchanged clarified harvest pool was divided into 2 equal volume pools to perform two chromatography cycles on a CIMultus DEAE Monolith (Sartorius AG). The monolith was equilibrated and the first pool was loaded onto the monolith. After elution of the vector, the column was stripped and the chromatography process was then repeated with the second half of the harvest pool, and each resulting elution was pooled for further processing.

Triton phase separation was performed on the above elution pool to remove endotoxin from the pool using a Triton buffer. The pool was centrifuged and the top aqueous phase was collected from each tube via pipetting and pooled.

The post-triton phase separation pool was further purified via heparin affinity chromatography. Vector was eluted and the resulting 50 mL eluate pool was collected for further processing.

The heparin eluate pool was loaded onto a 100 kDa TFF cartridge (Formulatrix) and buffer exchange was performed. A final 2.5× concentration was then performed to a 4 mL final volume. The buffer exchanged TFF pool was sterile filtered and aliquoted for storage at −80° C.

### Experimental Design

PBS control, AAV9-fCMV-eGFP (“AAV9-eGFP” or “AAV-eGFP”), and Ring19-fCMV-eGFP (“Ring19-eGFP”) were administered into FVB/NJ mice by intrathecal (IT) injection in accordance with Table E4. DNA, RNA, and immunohistochemistry for eGFP were analyzed as described in Example 2.

TABLE E4
Study Design

| | Treatment | Treatment | Dose Vector | Dose/animal | | N | Terminal |
|---|---|---|---|---|---|---|---|
| Group | Day 0 | Day 21 | (vg/ml) | (vg) | Route | (animals) | Day |
| 1 | PBS | Takedown | 0 | 0 | IT | 5 | 21 |
| | | | | | (50 ul) | | |
| 2 | PBS | NA | 0 | 0 | IT | 5 | 42 |
| | | | | | (50 ul) | | |
| 3 | PBS | PBS | 0/0 | 0/0 | IT | 5 | 42 |
| | | | | | (50 ul) | | |
| 4 | RING19-fCMV- | Takedown | 1.55E+10 | 7.75E+8 | IT | 5 | 21 |
| | eGFP | | | | (50 ul) | | |
| 5 | RING19-fCMV- | NA | 1.55E+10 | 7.75E+8 | IT | 5 | 42 |
| | eGFP | | | | (50 ul) | | |
| 6 | RING19-fCMV- | RING19-fCMV- | 2.4E+10/2.4E+10 | 7.75E+8/7.75E+8 | IT | 5 | 42 |
| | eGFP | eGFP | | | (50 ul) | | |
| 7 | AAV9-fCMV- | Takedown | 2.4E+10 | 1.2E+9 | IT | 5 | 21 |
| | eGFP | | | | (50 ul) | | |
| 8 | AAV9-fCMV- | NA | 2.4E+10 | 1.2E+9 | IT | 5 | 42 |
| | eGFP | | | | (50 ul) | | |
| 9 | AAV9-fCMV- | AAV9-fCMV- | 2.4E+10/2.4E+10 | 1.2E+9/1.2E+9 | IT | 5 | 42 |
| | eGFP | eGFP | | | (50 ul) | | |

### Results

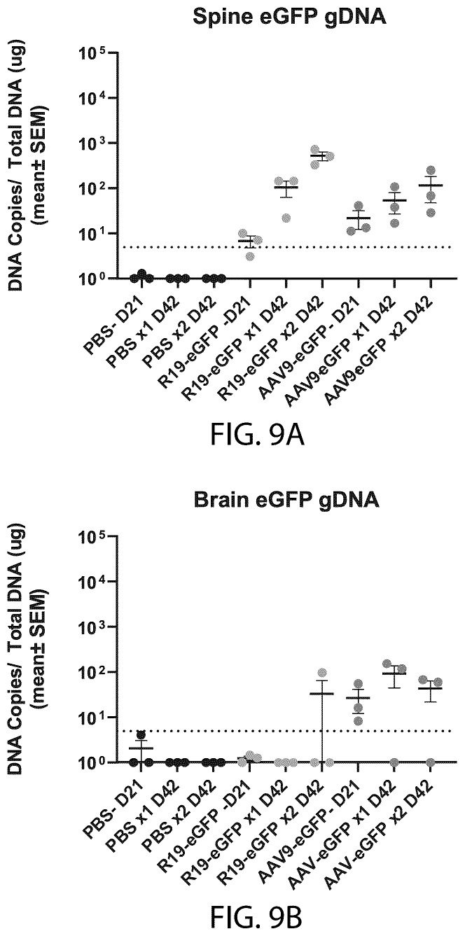

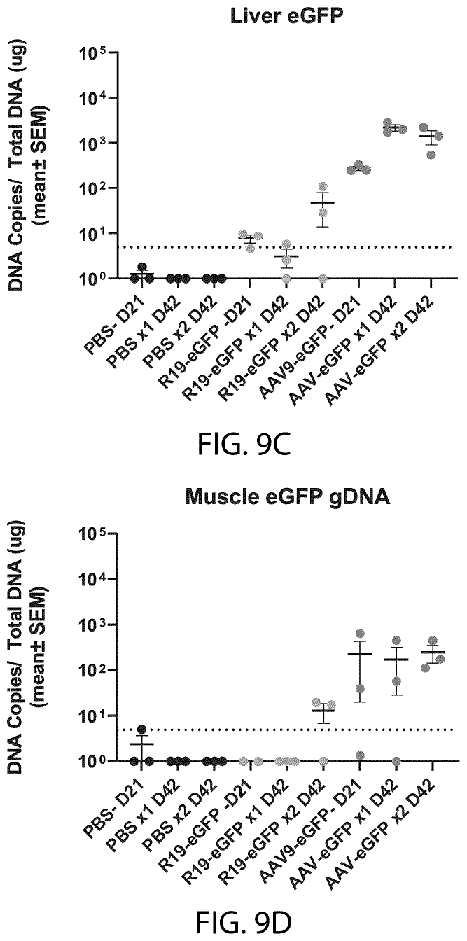

DNA was collected from the spinal cord, brain, liver, and muscle on either day 21 or day 42 after treatment as outlined in Table E4 and eGFP genomes were detected by qPCR. As shown in FIG. 9A, no eGFP genomes were detected in the spinal cord of any of the animals that received PBS controls, while eGFP genomes were detected at day 21 (“R19-eGFP-D21”) and day 42 (“R19-eGFP×1 D42”) in the spinal cords in animals administered with a single dose of Ring19-eGFP anellovector, with an increase in genome levels detected on day 42. In the animals that received a second dose of Ring19-eGFP 21 days after the initial treatment with Ring19-eGFP (“R19-eGFP×2 D42”), the eGFP genome levels were greater in the spinal cord than in the animals that received only a single dose. While the animals received a slightly lower dose of Ring19-eGFP anellovector compared to AAV9-eGFP, the eGFP genome levels were higher in the spinal cords of animals on day 42 that received a single dose of R19-eGFP anellovector (“R19-eGFP×1 D42”) and animals that received two doses of Ring19-eGFP (“R19-eGFP×2 D42”) compared to animals that received a single dose of AAV9-eGP (“AAV9-eGFP×1 D42”) and animals that received two doses of AAV9-eGFP (“AAV9 eGFP×2 D42”).

As shown in FIG. 9B, no eGFP genomes were detected in the brain in any of the animals that received PBS controls or in the brain of the animals at day 21 (“R19-eGFP-D21”) and day 42 (“R19-eGFP×1 D42”) administered with a single dose of Ring19-eGFP anellovector. However, some eGFP genomes were detected in the brain of one animal that received a second dose of Ring19-eGFP 21 days after the initial treatment with Ring19-eGFP (“R19-eGFP×2 D42”), similar to eGFP genome levels in animals that received a second dose of AAV9-eGFP (“AAV-eGFP×2 D42”).

As shown in FIG. 9C, no eGFP genomes were detected in the liver of any of the animals that received PBS controls. eGFP genomes were detected in the liver at day 21 (“R19-eGFP-D21”) and day 42 (“R19-eGFP×1 D42”) in the liver of animals administered with a single dose of Ring19-eGFP anellovector. The level of eGFP genomes detected in the liver in the animals that received a second dose of Ring19-eGFP 21 days after the initial treatment with Ring19-eGFP (“R19-eGFP×2 D42”) was higher than in the animals that received a single dose of Ring19-eGFP anellovector.

As shown in FIG. 9D, no eGFP genomes were detected in the muscle of any of the animals that received PBS controls or in the muscle of the animals at day 21 (“R19-eGFP-D21”) and day 42 (“R19-eGFP×1 D42”) administered with a single dose of Ring9-eGFP anellovector. However, some eGFP genomes were detected in the muscle of animals that received a second dose of Ring19-eGFP 21 days after the initial treatment with Ring19-eGFP (“R19-eGFP×2 D42”). While eGFP genomes were detected in the muscle of animals that received a single dose (“AAV9-eGFP-D21” and “AAV-eGFP×1 D42”) of AAv9-eGFP, the eGFP genome levels did not increase with administration of a second dose of AAV9-eGFP (“AAV-eGFP×2 D42”).

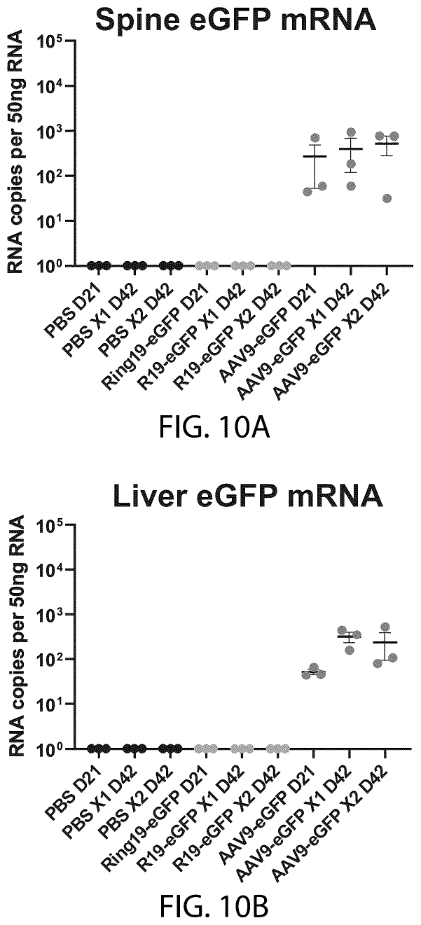

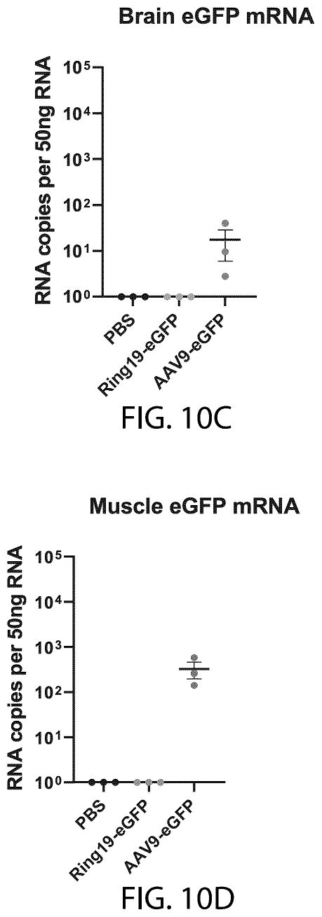

RNA was collected from the spinal cord and liver on either day 21 or day 42 after treatment for eGFP mRNA detection by RT-ddPCR. As shown in FIG. 10A, the level of eGFP mRNA was below the limit of detection in both the PBS controls and in the spinal cord of mice treated with Ring19-eGFP at the doses administered, while eGFP mRNA was detected in the spinal cord of mice treated with AAV9-eGFP. Similarly, as shown in FIG. 10B, the level of eGFP mRNA was below the limit of detection in both the PBS controls and in the liver of mice treated with Ring19-eGFP, while eGFP mRNA was detected in the liver of mice treated with AAV9-eGFP.

RNA was collected from the brain and muscle from mice in Groups 4 and 7 on day 21 after treatment for eGFP mRNA detection by RT-ddPCR. As shown in FIG. 10C, eGFP mRNA was below the limit of detection in both the PBS controls and in the brain of mice treated with Ring19-eGFP at the doses administered, while eGFP mRNA was detected in the brain of mice treated with AAV9-eGFP. Similarly, as shown in FIG. 10D, eGFP mRNA was below the limit of detection in both PBS controls and in the muscle of mice treated with Ring19-eGFP at the doses administered, while eGFP mRNA was detected in the muscle of mice treated with AAV9-eGFP.

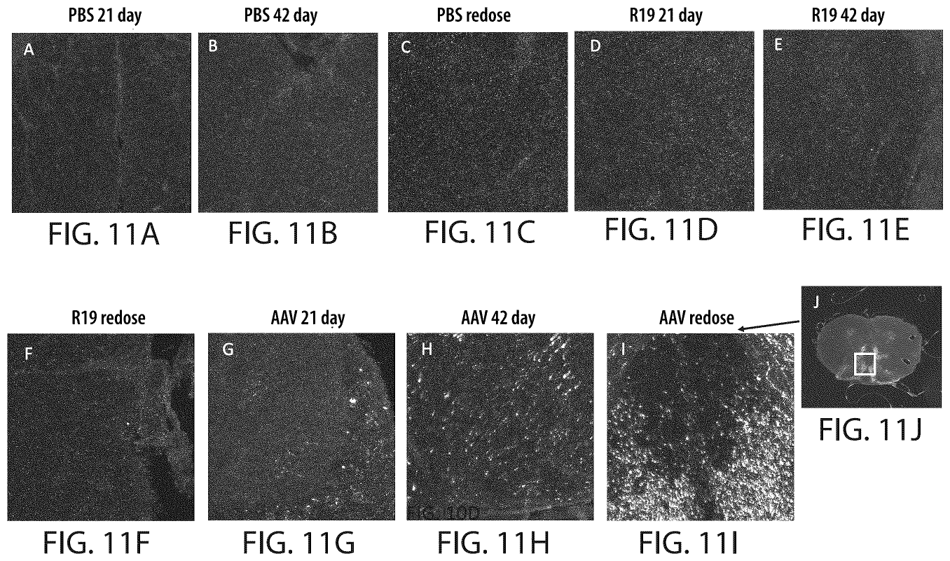

The spinal cord was also collected at day 21 or day 42 after treatment for fixation and immunohistochemistry. Representative spinal cord sections of treated mice were stained for eGFP. FIGS. 11A, 11B, and 11C show images of spinal cord sections stained for eGFP from the PBS control (Groups 1, 2, and 3, respectively) at 20× magnification. FIGS. 11D, 11E, and 11F show images of spinal cord stained for eGFP from mice treated with Ring19-eGRP in Groups 4, 5, and 6, respectively, at 20× magnification. FIGS. 11G, 11H, and 11I show images of spinal cord stained for eGFP from mice treated with AAV9-eGFP in Groups 7, 8, and 9, respectively, at 20× magnification. The area in the red square in FIG. 11J (2× magnification) shows the area that was magnified to 20× in FIG. 11I. No eGFP staining was detected in any of the PBS controls (FIGS. 11A-C). eGFP staining was not detected in the Ring19-eGFP-treated mice after a single dose at the doses administered (FIGS. 11D and 11E). eGFP protein was detected in the spinal cord of AAV9-eGFP-treated mice (FIGS. 11G-11I).

### Example 5: In Vivo Delivery of an Anellovector to Brain and Redosing Via ICV Administration

This example describes the in vivo redosing of a Ring19-eGFP Anellovector to the brain.

### Materials and Methods

AAV9-fCMV-eGFP is an adeno-associated virus based on the plasmid pRTx-2770 as described above and is packaged into AAV9. Ring19-fCMV-eGFP is produced by transfecting MOLT-4 cells with the three plasmids (pRTx-3525; pRTx-2847; pRTx-2848) shown in Table E0.

PBS control, AAV9-fCMV-eGFP (“AAV9-eGFP”), and Ring19-fCMV-eGFP (“Ring19-eGFP”) are administered into FVB/NJ mice by intracerebroventricular (ICV) injection in accordance with Table E5. DNA, RNA, and immunohistochemistry for eGFP in the brain are analyzed as similarly described in Example 2.

TABLE E5
Study Design

| Group | Treatment Day 0 | Treatment Day 21 | Dose/animal (vg) | Route | N (animals) | Terminal Day |
|---|---|---|---|---|---|---|
| 1 | PBS | Takedown | 0 | ICV | ~6 | 21 |
| 2 | PBS | NA | 0 | ICV | ~5 | 42 |
| 3 | PBS | PBS | 0/0 | ICV | ~5 | 42 |
| 4 | RING19-fCMV-eGFP | Takedown | E7-E10 | ICV | ~6 | 21 |
| 5 | RING19-fCMV-eGFP | NA | E7-E10 | ICV | ~5 | 42 |
| 6 | RING19-fCMV-eGFP | RING19-fCMV-eGFP | E7-E10/E7-E10 | ICV | ~5 | 42 |
| 7 | AAV9-fCMV-eGFP | Takedown | E7-E10 | ICV | ~6 | 21 |
| 8 | AAV9-fCMV-eGFP | NA | E7-E10 | ICV | ~5 | 42 |
| 9 | AAV9-fCMV-eGFP | AAV9-fCMV-eGFP | E7-E10/E7-E10 | ICV | ~5 | 42 |

In accordance with the schedule shown in Table E5, on day 21 or 42 after initial administration, the animals are taken down and DNA and RNA from the brains are collected and processed. qPCR is used to detect eGFP genomes in the DNA samples and RT-ddPCR is used to detect eGFP RNA in the RNA samples. Immunohistochemistry may also be performed to detect eGFP protein.

1. A method of delivering an exogenous effector to the central nervous system (CNS) of a subject, the method comprising administering to the CNS of the subject an Anelloviridae family vector.

2. (canceled)

3. The method of claim 1, which comprises delivery of the Anelloviridae family vector to the brain of the subject.

4. The method of claim 1, which comprises production of the exogenous effector and/or DNA capable of encoding the exogenous effector in the brain of the subject.

5.-7. (canceled)

8. A method of treating a CNS disease or disorder in a subject in need thereof, the method comprising administering to the subject an Anelloviridae family vector.

9.-11. (canceled)

12. The method of claim 1, which results in delivery of anellovector DNA in one or more cell types selected from: neurons, glial cells, microglia, oligodendroglia, and astrocytes.

13.-14. (canceled)

15. The method of claim 1, which results in delivery of anellovector DNA in one or more of: cerebellum, frontotemporal lobe, parietal lobe, brain stem, and spinal cord.

16.-17. (canceled)

18. The method of claim 1, which results in greater delivery of the exogenous effector and/or the DNA encoding the exogenous effector to the brain than to the spinal cord.

19. The method of claim 1, wherein the Anelloviridae family vector is administered according to a route of administration chosen from: intrathecal (IT), intracerebroventricular (ICV), intra cisterna magna (ICM), or intraparenchymal (IPa).

20. (canceled)

21. The method of claim 1, wherein the Anelloviridae family vector is an anellovector comprising a proteinaceous exterior comprising an ORF1 molecule and a genetic element enclosed by the proteinaceous exterior, wherein the genetic element comprises a promoter element operably linked to a nucleic acid sequence encoding the exogenous effector.

22. The method of claim 21, wherein the ORF1 molecule:
(i) has an ORF1 sequence as listed in any of Tables A1-A3, or a polypeptide comprising an amino acid sequence having at least about 70% sequence identity thereto; and/or
(ii) comprises a polypeptide encoded by an Anellovirus ORF1 nucleic acid sequence as listed in Table N1-N3, or a polypeptide encoded by a nucleic acid sequence having at least about 70% sequence identity to the Anellovirus ORF1 nucleic acid sequence.

23. The method of claim 22, wherein the ORF1 molecule comprises at least one difference relative to the wild-type ORF1 protein of Table A1-A3.

24.-27. (canceled)

28. The method of claim 1, wherein the genetic element comprises;
(A) (i) an Anellovirus 5′ UTR conserved domain having a sequence of the reverse complement of nucleotides 323-393 of SEQ ID NO: 54, or a nucleic acid sequence having at least 70% sequence identity thereto, or a functional portion thereof; and/or
(ii) an Anellovirus GC-rich region having a sequence of the reverse complement of nucleotides 2868-2929 of SEQ ID NO: 54, or a nucleic acid sequence having at least 70% sequence identity thereto, or a functional portion thereof; or
(B) (i) an Anellovirus 5′ UTR conserved domain having a sequence of the reverse complement of nucleotides 1-71 of SEQ ID NO: 1, or a nucleic acid sequence having at least 70% sequence identity thereto, or a functional portion thereof; and/or
(ii) an Anellovirus GC-rich region having a sequence of the reverse complement of nucleotides 2515-2615 of SEQ ID NO: 1, or a nucleic acid sequence having at least 70% sequence identity thereto, or a functional portion thereof.

29.-32. (canceled)

33. The method of claim 1, wherein the genetic element is:
(i) DNA;
(ii) circular, single stranded DNA; or
(iii) mRNA.

34.-35. (canceled)

36. The method of claim 1, wherein the exogenous effector comprises: an intracellular peptide or intracellular polypeptide, a secreted polypeptide, or a protein replacement therapeutic.

37. The method of claim 1, wherein the Anelloviridae family vector is a Betatorquevirus.

38. The method of claim 1, which further comprises administering an additional dose of an Anelloviridae family vector to the CNS of the subject.

39. The method of claim 38, wherein the Anelloviridae family vector and the Anelloviridae family vector of the additional dose are the same.

40. (canceled)

41. The method of claim 1, which results in greater delivery of the DNA encoding the exogenous effector to:
(i) the CNS than to muscle;
(ii) the CNS than to liver;
(iii) the spinal cord than to muscle; and/or
(iv) the spinal cord than to liver.

42.-44. (canceled)

45. The method of claim 41, wherein the greater delivery is 2 times, 5 times, or 10 times higher.

46. A delivery system suitable for delivery to the CNS, wherein the delivery system comprises an Anelloviridae family vector.
